(19) United States
(12) Patent Application Publication
(10) Pub. No.: US 2008/0261300 A1
(43) Pub. Date: Oct. 23, 2008

(54) Synthetic genes

(75) Inventors: Daniel V. Santi, San Francisco, CA (US); Ralph C. Reid, San Rafael, CA (US); Sarah J. Kodumal, Oakland, CA (US); Sebastian Jayaraj, Berkeley, CA (US)

Correspondence Address: TOWNSEND AND TOWNSEND AND CREW, LLP, TWO EMBARCADERO CENTER, EIGHTH FLOOR, SAN FRANCISCO, CA, 94111-3834, US

(73) Assignee: Kosan Biosciences, Inc., Hayward, CA (US)

(21) Appl. No.: 11/894,753

(22) Filed: Aug. 20, 2007

Related U.S. Application Data
(63) Continuation of application No. 10/672,396, filed on Sep. 26, 2003.
(60) Provisional application No. 60/414,085, filed on Sep. 26, 2002.

(51) Int. Cl. C12N 15/63; C12M 1/40

(52) U.S. Cl. 435/320.1; 435/283.1

(57) ABSTRACT

The invention provides strategies, methods, vectors, reagents, and systems for production of synthetic genes, production of libraries of such genes, and manipulation and characterization of the genes and corresponding encoded polypeptides. In one aspect, the synthetic genes can encode polyketide synthase polypeptides and facilitate production of therapeutically or commercially important polyketide compounds.

### CROSS-REFERENCE TO RELATED APPLICATIONS

This application claims benefit under 35 U.S.C. § 119(e) of provisional application No. 60/414,085, filed 26 Sep. 2002, the contents of which are incorporated herein by reference.

### STATEMENT CONCERNING GOVERNMENT SUPPORT

Subject matter disclosed in this application was made, in part, with government support under National Institute of Standards and Technology ATP Grant No. 70NANB2H3014. As such, the United States government may have certain rights in this invention.

### FIELD OF THE INVENTION

The invention provides strategies, methods, vectors, reagents, and systems for production of synthetic genes, production of libraries of such genes, and manipulation and characterization of the genes and corresponding encoded polypeptides. In one aspect, the synthetic genes can encode polyketide synthase polypeptides and facilitate production of therapeutically or commercially important polyketide compounds. The invention finds application in the fields of human and veterinary medicine, pharmacology, agriculture, and molecular biology.

### BACKGROUND

Polyketides represent a large family of compounds produced by fungi, mycelial bacteria, and other organisms. Numerous polyketides have therapeutically relevant and/or commercially valuable activities. Examples of useful polyketides include erythromycin, FK-506, FK-520, megalomycin, narbomycin, oleandomycin, picromycin, rapamycin, spinocyn, and tylosin.

Polyketides are synthesized in nature from 2-carbon units through a series of condensations and subsequent modifications by polyketide synthases (PKSs). Polyketide synthases are multifunctional enzyme complexes composed of multiple large polypeptides. Each of the polypeptide components of the complex is encoded by a separate open reading frame, with the open reading frames corresponding to a particular PKS typically being clustered together on the chromosome. The structure of PKSs and the mechanisms of polyketide synthesis are reviewed in Cane et al., 1998, “Harnessing the biosynthetic code: combinations, permutations, and mutations” Science 282:63-8.

PKS polypeptides comprise numerous enzymatic and carrier domains, including acyltransferase (AT), acyl carrier protein (ACP), and beta-ketoacylsynthase (KS) activities, involved in loading and condensation steps; ketoreductase (KR), dehydratase (DH), and enoylreductase (ER) activities, involved in modification at β-carbon positions of the growing chain, and thioesterase (TE) activities involved in release of the polyketide from the PKS. Various combinations of these domains are organized in units called “modules.” For example, the 6-deoxyerythronolide B synthase (“DEBS”), which is involved in the production of erythromycin, comprises 6 modules on three separate polypeptides (2 modules per polypeptide). The number, sequence, and domain content of the modules of a PKS determine the structure of the polyketide product of the PKS.

Given the importance of polyketides, the difficulty in producing polyketide compounds by traditional chemical methods, and the typically low production of polyketides in wild-type cells, there has been considerable interest in finding improved or alternate means for producing polyketide compounds. This interest has resulted in the cloning, analysis and manipulation by recombinant DNA technology of genes that encode PKS enzymes. The resulting technology allows one to manipulate a known PKS gene cluster to produce the polyketide synthesized by that PKS at higher levels than occur in nature, or in hosts that otherwise do not produce the polyketide. The technology also allows one to produce molecules that are structurally related to, but distinct from, the polyketides produced from known PKS gene clusters by inactivating a domain in the PKS and/or by adding a domain not normally found in the PKS though manipulation of the PKS gene.

While the detailed understanding of the mechanisms by which PKS enzymes function and the development of methods for manipulating PKS genes have facilitated the creation of novel polyketides, there are presently limits to the creation of novel polyketides by genetic engineering. One such limit is the availability of PKS genes. Many polyketides are known but only a relatively small portion of the corresponding PKS genes have been cloned and are available for manipulation. Moreover, in many instances the organism producing an interesting polyketide is obtainable only with great difficulty and expense, and techniques for its growth in the laboratory and, production of the polyketide it produces are unknown or difficult or time-consuming to practice. Also, even if the PKS genes for a desired polyketide have been cloned, those genes may not serve to drive the level of production desired in a particular host cell.

If there was a method to produce a desired polyketide without having to access the genes that encode the PKS (that produces the polyketide, then many of these difficulties could be ameliorated or avoided altogether. The present invention meets this and other needs.

### BRIEF SUMMARY OF THE INVENTION

In one aspect, the invention provides a synthetic gene encoding a polypeptide segment that corresponds to a reference polypeptide segment encoded by a naturally occurring gene. The polypeptide segment-encoding sequence of the synthetic gene is different from the polypeptide segment-encoding sequence of the naturally occurring gene. In one aspect, the polypeptide segment-encoding sequence of the synthetic gene is less than about 90% identical to the polypeptide segment-encoding sequence of the naturally occurring gene, or in some embodiments, less than about 85% or less than about 80% identical. In one aspect, the polypeptide segment-encoding sequence of the synthetic gene comprises at least one (and in other embodiments, more than one, e.g., at least two, at least three, or at least four) unique restriction sites that are not present or are not unique in the polypeptide segment-encoding sequence of the naturally occurring gene. In an aspect, the polypeptide segment-encoding sequence of the synthetic gene is free from at least one restriction site that is present in the polypeptide segment-encoding sequence of the naturally occurring gene. In an embodiment of the invention, the polypeptide segment encoded by the synthetic gene corresponds to at least 50 contiguous amino acid residues encoded by the naturally occurring gene.

In an embodiment, the polypeptide segment is from a polyketide synthase (PKS) and may be or include a PKS domain (e.g., AT, ACP, KS, KR, DH, ER, and/or TE) or one or more PKS modules. In some embodiments, the synthetic PKS gene has, at most, one copy per module-encoding sequence of a restriction enzyme recognition site selected from the group consisting of Spe I, Mfe I, Afi II, Bsi WI, Sac II, Ngo MIV, Nhe I, Kpn I, Msc I, Bgl II, Bss HII, Sac II, Age I, Pst I, Kas I, Mlu I, Xba I, Sph I, Bsp E, and Ngo MIV recognition sites. In an embodiment, the polypeptide segment-encoding sequence of the synthetic gene is free from at least one Type IIS enzyme restriction site (e.g., Bci VI, Bmr I, Bpm I, Bpu EI, Bse RI, Bsg I, Bsr Di, Bts I, Eci I, Ear I, Sap I, Bsm BI, Bsp MI, Bsa I, Bbs I, Bfu AI, Fok I and Alw I) present in the polypeptide segment-encoding sequence of the naturally occurring gene.

In a related embodiment, the invention provides a synthetic gene encoding a polypeptide segment that corresponds to a reference polypeptide segment encoded by a naturally occurring PKS gene, where the polypeptide segment-encoding sequence of the synthetic gene is different from the polypeptide segment encoding sequence of the naturally occurring PKS gene and comprises at least two of (a) a Spe I site near the sequence encoding the amino-terminus of the module; (b) a Mfe I site near the sequence encoding the amino-terminus of a KS domain; (c) a Kpn I site near the sequence encoding the carboxy-terminus of a KS domain; (d) a Msc I site near the sequence encoding the amino-terminus of an AT domain; (e) a Pst I site near the sequence encoding the carboxy-terminus of an AT domain; (f) a Bsr BI site near the sequence encoding the amino-terminus of an ER domain; (g) an Age I site near the sequence encoding the amino-terminus of a KR domain; and (h) an Xba I site near the sequence encoding the amino-terminus of an ACP domain.

In related aspects, the invention provides a vector (e.g., cloning or expression vector) comprising a synthetic gene of the invention. In an embodiment, the vector comprises an open reading frame encoding a first PKS module and one or more of (a) a PKS extension module; (b) a PKS loading module; (c) a releasing (e.g., thioesterase) domain; and (d) an interpolypeptide linker.

Cells that comprise or express a gene or vector of the invention are provided, as well as a cell comprising a polypeptide encoded by the vector or, a functional polyketide synthase, wherein the PKS comprises a polypeptide encoded by the vector. In one aspect, a PKS polypeptide having a non-natural amino sequence is provided, such as a polypeptide characterized by a KS domain comprising the dipeptide Leu-Gln at the carboxy-terminal edge of the domain; and/or an ACP domain comprising the dipeptide Ser-Ser at the carboxy-terminal edge of the domain. A method is provided for making a polyketide comprising culturing a cell comprising a synthetic DNA of the invention under conditions in which a polyketide is produced, wherein the polyketide would not be produced by the cell in the absence of the vector.

In one aspect, the invention provides a method for high throughput synthesis of a plurality of different DNA units comprising different polypeptide encoding sequences comprising: for each DNA unit, performing polymerase chain reaction (PCR) amplification of a plurality of overlapping oligonucleotides to generate a DNA unit encoding a polypeptide segment and adding UDG-containing linkers to the 5′ and 3′ ends of the DNA unit by PCR amplification, thereby generating a linkered DNA unit, wherein the same UDG-containing linkers are added to said different DNA units. In embodiments, the plurality comprises more than 50 different DNA units, more than 100 different DNA units, or more than 500 different DNA units (synthons). In a related aspect, the invention provides a method for producing a vector comprising a polypeptide encoding sequence comprising cloning the linkered DNA unit into a vector using a ligation-independent-cloning method.

The invention provides gene libraries. In one embodiment, a gene library is provided that contains a plurality of different PKS module-encoding genes, where the module-encoding genes in the library have at least one (or more than one, such as at least 3, at least 4, at least 5 or at least 6) restriction site(s) in common, the restriction site is found no more than one time in each module, and the modules encoded in the library correspond to modules from five or more different polyketide synthase proteins. Vectors for gene libraries include cloning and expression vectors. In some embodiments, a library includes open reading frames that contain an extension module and at least one of a second PKS extension module, a PKS loading module, a thioesterase domain, and an interpolypeptide linker.

In a related aspect, the invention provides a method for synthesis of an expression library of PKS module-encoding genes by making a plurality of different PKS module-encoding genes as described above and cloning each gene into an expression vector. The library may include, for example, at least about 50 or at least about 100 different module-encoding genes.

The invention provides a variety of cloning vectors useful for stitching (e.g., a vector comprising, in the order shown, SM4-SIS-SM2-R₁ or L-SIS-SM2-R₁ where SIS is a synthon insertion site, SM2 is a sequence encoding a first selectable marker, SM4 is a sequence encoding a second selectable marker different from the first, R₁ is a recognition site for a restriction enzyme, and L is a recognition site for a different restriction enzyme. The invention further provides vectors comprising synthon sequences, e.g. comprising, in the order shown, SM4-2S₁-Sy₁-2S₂-SM2-R₁ or L-2S₁-Sy₂-2S₂-SM2-R₁ where 2S₁ is a recognition site for first Type IIS restriction enzyme, 2S₂ is a recognition site for a different Type IIS restriction enzyme, and Sy is synthon coding region. Also provided are compositions of a vector and a Type IIS or other restriction enzyme that recognizes a site on the vector, compositions comprising cognate pairs of vectors, kits, and the like.

In one embodiment, the invention provides a vector comprising a first selectable marker, a restriction site (R₁) recognized by a first restriction enzyme, and a synthon coding region that is flanked by a restriction site recognized by a first Type IIS restriction enzyme and a restriction site recognized by a second Type IIS restriction enzyme, wherein digestion of the vector with the first restriction enzyme and the first Type IIS restriction enzyme produces a fragment comprising the first selectable marker and the synthon coding region, and digestion of the vector with the first restriction enzyme and the second Type IIS restriction enzyme produces a fragment comprising the synthon coding region and not comprising the first selectable marker. In an embodiment, the vector comprising a second selectable marker wherein digestion of the vector with the first restriction enzyme and the first Type IIS restriction enzyme produces a fragment comprising the first selectable marker and the synthon coding region, and not comprising the second selectable marker, digestion of the vector with the first restriction enzyme and the second Type IIS restriction enzyme produces a fragment comprising the second selectable marker and the synthon coding region, and not comprising the first selectable marker. The invention provides methods of stitching adjacent DNA units (synthons) to synthesize a larger unit. For example, the invention provides a method for making a synthetic gene encoding a PKS module by producing a plurality (i.e., at least 3) of DNA units by assembly PCR, wherein each DNA unit encodes a portion of the PKS module and combining the plurality of DNA units in a predetermined sequence to produce PKS module-encoding gene. In an embodiment, the method includes combining the module-encoding gene in-frame with a nucleotide sequence encoding a PKS extension module, a PKS loading module, a thioesterase domain, or an PKS interpolypeptide linker, to produce a PKS open reading frame.

In a related embodiment, the invention provides a method for joining a series of DNA units using a vector pair by a) providing a first set of DNA units, each in a first-type selectable vector comprising a first selectable marker and providing a second set of DNA units, each in a second-type selectable vector comprising a second selectable marker different from the first, wherein the first-type and second-type selectable vectors can be selected based on the different selectable markers, b) recombinantly joining a DNA unit from the first set with an adjacent DNA unit from the second set to generate a first-type selectable vector comprising a third DNA unit, and obtaining a desired clone by selecting for the first selectable marker c) recombinantly joining the third DNA unit with an adjacent DNA unit from the second set to generate a first-type selectable vector comprising a fourth DNA unit, and obtaining a desired clone by selecting for the first selectable marker, or recombinantly joining the third DNA unit with an adjacent DNA unit from the second set to generate a second-type selectable vector comprising a fourth DNA unit, and obtaining a desired clone by selecting for the second selectable marker. In an embodiment, the step (c) comprises recombinantly joining the third DNA unit with an adjacent DNA unit from the second set to generate a first-type selectable vector comprising a fourth DNA unit, and obtaining a desired clone by selecting for the first selectable marker, the method further comprising recombinantly combining the fourth DNA unit with an adjacent DNA unit from the second set to generate a first-type selectable vector comprising a fifth DNA unit, and obtaining a desired clone by selecting for the first selection marker, or recombinantly combining the third DNA unit with an adjacent DNA unit from the second set to generate a second-type selectable vector comprising a fifth DNA unit, and obtaining a desired clone by selecting for the second selection marker. In an embodiment, step (c) comprises recombinantly joining the third DNA unit with an adjacent DNA unit from the second series to generate a second-type selectable vector comprising a fourth DNA unit, and obtaining a desired clone by selecting for the second selectable marker, the method further comprising recombinantly joining the fourth DNA unit with an adjacent DNA unit from the first set to generate a first-type selectable vector comprising a fifth DNA unit, and obtaining a desired clone by selecting for the first selection marker, or recombinantly joining the third DNA unit with an adjacent DNA unit from the second set to generate a first-type selectable vector comprising a fifth DNA unit and obtaining a desired clone by selecting for the first selection marker.

In a related aspect, the invention provides a method for joining a series of DNA units to generate a DNA construct by (a) providing a first plurality of vectors, each comprising a DNA unit and a first selectable marker; (b) providing a second plurality of vectors, each comprising a DNA unit and a second selectable marker; (c) digesting a vector from (a) to produce a first fragment containing a DNA unit and at least one additional fragment not containing the DNA unit; (d) digesting a DNA from (b) to produce a second fragment containing a DNA unit and at least one additional fragment not containing the DNA unit, where only one of the first and second fragments contains an origin of replication; ligating the fragments to generate a product vector comprising a DNA unit from (c) ligated to a DNA unit from (d); selecting the product vector by selecting for either the first or second selectable marker; (e) digesting the product vector to produce a third fragment containing a DNA unit and at least one additional fragment not containing the DNA unit; (d) digesting a DNA from (a) or (b) to produce a fourth fragment containing a DNA unit and at least one additional fragment not containing the DNA unit, where only one of the third and fourth fragments contains an origin of replication; (f) ligating the third and fourth fragments to generate a product vector comprising a DNA unit from (e) ligated to a DNA unit from (d) and selecting the product vector by selecting for either the first or second selectable marker.

In another aspect, an open reading frame vector is provided, which has an internal type {4-[7-*]-[*-8]-3}, left-edge type {4-[7-1]-[*-8]-3} or right-edge type {4-[7-*]-[6-8]-3} architecture where 7 and 8 are recognition sites for Type IIS restriction enzymes which cut to produce compatible overhangs “*”; 1 and 6 are Type II restriction enzyme sites that are optionally present; and 3 and 4 are recognition sites for restriction enzymes with 8-base pair recognition sites. In various embodiments, 1 is Nde I and/or 6 is Eco RI and/or 4 is Not I and/or 3 is Pac I.

In another aspect, a method for identifying restriction enzyme recognition sites useful for design of synthetic genes is provided. The method includes the steps of obtaining amino acid sequences for a plurality of functionally related polypeptide segments; reverse-translating the amino acid sequences to produce multiple polypeptide segment-encoding nucleic acid sequences for each polypeptide segment; and identifying restriction enzyme recognition sites that are found in at least one polypeptide segment-encoding nucleic acid sequence of at least about 50% of the polypeptide segments. In certain embodiments, the functionally related polypeptide segments are polyketide synthase modules or domains, such as regions of high homology in PKS modules or domains.

In a method for designing a synthetic gene in accordance with the present invention a reference amino acid sequence is provided and reverse translated to a randomized nucleotide sequence which encodes the amino acid sequence using a random selection of codons which, optionally, have been optimized for a codon preference of a host organism. One or more parameters for positions of restriction sites on a sequence of the synthetic gene are provided and occurrences of one or more selected restriction sites from the randomized nucleotide sequence are removed. One or more selected restriction sites are inserted at selected positions in the randomized nucleotide sequence to generate a sequence of the synthetic gene.

In one aspect of the invention, a set of overlapping oligonucleotide sequences which together comprise a sequence of the synthetic gene are generated.

In another aspect of the invention, one or more parameters for positions of restriction sites on a sequence of the synthetic gene comprise one or more preselected restriction sites at selected positions.

In another aspect of the invention, the selected position of the preselected restrictions site corresponds to a positions selected from the group consisting of a synthon edge, a domain edge and a module edge.

In another aspect of the invention, providing one or more parameters for positions of restriction sites on a sequence of the synthetic gene is followed by predicting all possible restriction sites that can be inserted in the randomized nucleotide sequence and optionally, identifying one or more unique restriction sites.

In another aspect of the invention, the sequence of the synthetic gene is divided into a series of synthons of selected length and then a set of overlapping oligonucleotide sequences is generated which together comprise a sequence of each synthon.

In another aspect of the invention, the set of overlapping oligonucleotide sequences comprise (a) oligonucleotide sequences which together comprise a synthon coding region corresponding to the synthetic gene, and (b) oligonucleotide sequences which comprise one or more synthon flanking sequences.

In another aspect of the invention, one or more quality tests are performed on the set of overlapping oligonucleotide sequences, wherein the tests are selected from the group consisting of: translational errors, invalid restriction sites, incorrect positions of restriction sites, and aberrant priming.

In another aspect of the invention, each oligonucleotide sequence is of a selected length and comprises an overlap of a predetermined length with adjacent oligonucleotides of the set of oligonucleotides which together comprise the sequence of the synthetic gene.

In another aspect of the invention, each oligonucleotide is about 40 nucleotides in length and comprises overlaps of between about 17 and 23 nucleotides with adjacent oligonucleotides.

In another aspect of the invention, a set of overlapping oligonucleotide sequences are selected wherein each oligonucleotide anneals with its adjacent oligonucleotide within a selected temperature range.

In another aspect of the invention, generating a set of overlapping oligonucleotide sequences includes providing an alignment cutoff value for sequence specificity, aligning each oligonucleotide sequence with the sequence of the synthetic gene and determining its alignment value, and identifying and rejecting oligonucleotides comprising alignment values lower than the alignment cutoff value.

In another aspect of the invention, a region of error in a rejected oligonucleotide is identified and optionally, one or more nucleotides in the region of error are substituted such that the alignment value of the rejected oligonucleotide is raised above the alignment cutoff value.

In another aspect of the invention, an order list of oligonucleotides which comprise a synthetic gene or a synthon is generated.

In another aspect of the invention, removing of restriction sites includes identifying positions of preselected restriction sites in the randomized nucleotide sequence, identifying an ability of one or more codons comprising the nucleotide sequence of the restriction site for accepting a substitution in the nucleotide sequence of the restriction site wherein such substitution will (a) remove the restriction site and (b) create a codon encoding an amino acid identical to the codon whose sequence has been changed, and changing the sequence of the restriction site at the identified codon.

In another aspect of the invention, inserting of restriction sites includes identifying selected positions for insertion of a selected restriction site in the randomized nucleotide sequence, performing a substitution in the nucleotide sequence at the selected position such that the selected restriction site sequence is created at the selected position, translating the substituted sequence to an amino acid sequence, and accepting a substitution wherein the translated amino acid sequence is identical to the reference amino acid sequence at the selected position and rejecting a substitution wherein the translated amino acid sequence is different from the reference amino acid sequence at the selected position.

In another aspect of the invention, a translated amino acid sequence identical to the reference amino acid sequence comprises substitution of an amino acid with a similar amino acid at the selected position.

In another aspect of the invention, the synthetic gene encodes a PKS module.

In another aspect of the invention, the reference amino acid sequence is of a naturally occurring polypeptide segment.

In another aspect of the invention, one or more steps of the method may performed by a programmed computer.

In another aspect of the invention, a computer readable storage medium contains computer executable code for carrying out the method of the present invention.

In a method for analyzing a nucleotide sequence of a synthon in accordance with the present invention, a sequence of a synthetic gene is provided, wherein the synthetic gene is divided into a plurality of synthons. Sequences of a plurality of synthon samples are also provided wherein each synthon of the plurality of synthons is cloned in a vector. And, a sequence of the vector without an insert is provided. Vector sequences from the sequence of the cloned synthon are eliminated and a contig map of sequences of the plurality of synthons is constructed. The contig map of sequences is aligned with the sequence of the synthetic gene; and a measure of alignment for each of the plurality of synthons is identified.

In another aspect of the invention, errors in one or more synthon sequences are identified; and one or more informations are reported, the informations selected from the group consisting of: a ranking of synthon samples by degree of alignment, an error in the sequence of a synthon sample, and identity of a synthon that can be repaired.

In another aspect of the invention, a statistical report on a plurality of alignment errors is prepared.

A system for high through-put synthesis of synthetic genes in accordance with the present invention includes a source microwell plate containing oligonucleotides for assembly PCR, a first source for amplification mixture including polymerase and buffers useable for assembly PCR, a second source for LIC extension primer mixture, and a PCR microwell plate for amplification of oligonucleotides. A liquid handling device retrieves a plurality of predetermined sets of oligonucleotides from the source microwell plate(s), combines the predetermined sets and the amplification mixture in wells of the PCR microwell plate, LIC extension primer mixture, and combines the LIC extension primer mixture and amplicons in a well of the PCR microwell plate. The system also includes a heat source for PCR amplification configured to accept the at least one PCR microwell plate.

### BRIEF DESCRIPTION OF THE FIGURES

FIG. 1 shows a UDG-cloning cassette (“cloning linker”) and a scheme of vector preparation for ligation-independent cloning (LIC) using the nicking endonuclease N. BbvC IA. FIG. 1A. UDG-cloning cassette. Sac I and nicking enzyme sites used in vector preparation are labeled. FIG. 1B. Scheme of vector preparation for LIC using nicking endonuclease N. BbvC IA.

FIG. 2 illustrates the Method S joining method using Bbs I and Bsa I as the Type IIS restriction enzymes.

FIG. 3A shows the Method S joining method using Vector Pair I. FIG. 3B shows the Method S joining using Vector Pair II. 2S₁₋₄ are recognition sites for Type IIS restriction enzymes, and A, B, B and C, respectively, are the cleavage sites for the enzymes.

FIG. 4 shows a vector pair useful for stitching. FIG. 4A: Vector pKos293-172-2. FIG. 4B: Vector pKos293-172-A76. Both vectors contain a UDG-cloning cassette with N.Bbv C IA recognition sites, a “right restriction site” common to both vectors (Xho I site), a “left restriction site” different for each vector (e.g., Eco RV or Stu I site), a first selection marker common to both vectors (carbenicillin resistance marker) and second selection markers that are different in each vector (chloramphenicol resistance marker or kanamycin resistance marker).

FIG. 5 shows the Method R joining using Vector Pair II.

FIG. 6A shows a composite restriction map with a complete complement of six PKS domains as in ery module 4. Approximate sizes are KS=1.2, KS/AT linker=0.3, AT=1.0, AT/DH linker=0.03, DH=0.6, DH/ER linker=0.8, ER=0.8, ER/KR linker=0.02, KR=0.8, KR/ACP linker=0.2, ACP=0.2. 1 Unit=1 kb; FIG. 6B shows exemplary restriction sites for synthon edges with reference to DEBS2.

FIG. 7 shows a non-pairwise selection strategy for stitching of synthons 1-9 to make module 1-2-3-4-5-6-7-8-9. Parentheticals show the selection marker (K=kanamycin resistant, Cm=chloramphenicol resistant) and the left restriction sites, L and L′, (S=Stu I restriction site, E=Eco RV restriction site) for the vector in which the synthon or desired multisynthon is cloned. The synthons are joined at the following cohesive ends: 1-2 NgoM IV; 2-3 Nhe I; 3-4 Kpn I; 4-5 Bgl II; 5-6 Age I/Ngo MIV; 6-7 Pst I; 7-8 Age I; 8-9 Bgl II.

FIG. 8 is a flowchart showing the GeMS process.

FIG. 9 is a flowchart showing a GeMS algorithm.

FIG. 10A is a flowchart showing generation of codon preference table for a synthetic gene; and FIG. 10B is a flowchart showing an algorithm for generating a randomized and codon optimized gene sequence.

FIG. 11 is a flowchart showing a restriction site removal algorithm.

FIG. 12 is a flowchart showing a restriction site insertion algorithm.

FIG. 13 is a flowchart showing an algorithm for oligonucleotide design.

FIG. 14 is a flowchart showing an algorithm for rapid analysis of synthon DNA sequences.

FIG. 15 shows a PAGE analysis of DEBS. Soluble protein extracts from synthetic (sMod2) and natural sequence (nMod2) Mod2 strains were sampled 42 h after induction and analyzed by 3-8% SDS-PAGE. Positions of MW standards are indicated at the right. The gel was stained with Sypro Red (Molecular Probes).

FIG. 16 shows restriction sites and synthons used in construction of a synthetic DEBS gene. 16A DEBS1 ORF; 16B, DEBS2 ORF, 16C DEBS3 ORF.

FIG. 17 shows the stitching and selection strategy for construction of synthetic DEBS genes. A=synthon cloning vector 293-172-A76; B=synthon cloning vector 293-172-2. (A) Mod006 (DEBS mod1); (B) Mod007 (DEBS mod3); (C) Mod008 (DEBS mod4); (D) Mod009 (DEBS mod5); (E) Mod010 (DEBS mod6).

FIG. 18 shows restriction sites and synthons used in construction of a synthetic Epothilone PKS gene.

FIG. 19 shows an automated system for high throughput gene synthesis and analysis.

### DETAILED DESCRIPTION

The outline below is provided to assist the reader. The organization of the disclosure below is for convenience, and disclosure of an aspect of the invention in a particular section, does not imply that the aspect is not related to disclosure in other, differently labeled, sections.

1. Definitions

2. Introduction

3. Design of Synthetic Genes

4. Synthesis of Genes
4.1 Synthesis of Synthons
4.2 Synthesis of Module Genes (Stitching)
   4.2.1 Cloning Synthons In Assembly Vectors
   4.2.2 Validation of Synthons
   4.2.3 Method S: Joining Strategies, Assembly Vectors, & Selection Schemes
      4.2.3.1 Joining Strategies
      4.2.3.2 Assembly Vectors
      4.2.3.3 Selection Schemes
   4.2.4 Method R: Joining Strategies, Assembly Vectors, & Selection Schemes
      4.2.4.1 Joining Strategies
      4.2.4.2 Assembly Vectors
      4.2.4.3 Selection Schemes

5. Gene Design and Gems (Gene Morphing System) Algorithm
5.1 Gems—Overview
5.2 Gems Algorithms
5.3 Software Implementation

6. Multimodule Constructs And Libraries
6.1 Introduction
6.2 Exemplary Uses Of ORF Vector Libraries
6.3 Module And Linker Combinations
6.4 Exemplary Orf Vector Constructs
   6.4.1 Orf Vectors Comprising Amino- And- Carboxy Terminal Accessory Units or Other Polypeptide Sequences
   6.4.2 Orf Vector Synthesis
   6.4.3 Exemplary Orf Vector Construction Methods

7. Multimodule Design Based On Naturally Occurring Combinations

8. Domain Substitution

9. Exemplary Products
9.1 Synthetic PKS Module Genes
9.2 Vectors
9.3 Libraries
9.4 Databases

10. High Throughput Synthon Synthesis And Analysis

10.1 Automation of Synthesis

10.2 Rapid Analysis of Chromatograms (Racoon)

11. Examples
1. Gene Assembly and Amplification Protocols
2. Ligation Independent Cloning
3. Characterization and Correction of Cloned Synthons
4. Identification of Useful Restriction Sites in PKS Modules
5. Synthesis of Debs Module 2
6. Expression of Synthetic Debs Module 2 In E. Coli
7. Synthetic DEBS Gene Expression In E. Coli
8. Method for Quantitative Determination of Relative Amounts of Two Proteins
9. Synthesis of Epothilone Synthase Genes

### 1. DEFINITIONS

As used herein, a “protein” or “polypeptide” is a polymer of amino acids of any length, but usually comprising at least about 50 residues.

As used herein, the term “polypeptide segment” can be used to refer a polypeptide sequence of interest. A polypeptide segment can correspond to a naturally occurring polypeptide (e.g., the product of the DEBS ORF 1 gene), to a fragment or region of a naturally occurring polypeptide (e.g., a DEBS module 1, the KS domain of DEBS module 1, linkers, functionally defined regions, and arbitrarily defined regions not corresponding to any particular function or structure), or a synthetic polypeptide not necessarily corresponding to a naturally occurring polypeptide or region. A “polypeptide segment-encoding sequence” can be the portion of a nucleotide sequence (either in isolated form or contained within a longer nucleotide sequence) that encodes a polypeptide segment (for example, a nucleotide sequence encoding a DEBS1 KS domain); the polypeptide segment can be contained in a larger polypeptide or an entire polypeptide. In general, the term “polypeptide segment-encoding sequence” is intended to encompass any polypeptide-encoding nucleotide sequence that can be made using the methods of the present invention.

As used herein, the terms “synthon” and “DNA unit” refer to a double-stranded polynucleotide that is combined with other double-stranded polynucleotides to produce a larger macromolecule (e.g., a PKS module-encoding polynucleotide). Synthons are not limited to polynucleotides synthesized by any particular method (e.g., assembly PCR), and can encompass synthetic, recombinant, cloned, and naturally occurring DNAs of all types. In some cases, three different regions of a synthon can be distinguished (a coding region and two flanking regions). The portion of the synthon that is incorporated into the final DNA product of synthon stitching (e.g., a module gene) can be referred to as the “synthon coding region.” The regions of the synthon that flank the synthon coding region, and which do not become part of the product DNA can be referred to as the “synthon flanking regions.” As is described below, the synthon flanking regions are physically separated from the synthon coding region during stitching by cleavage using restriction enzymes.

As used herein, “multisynthon” refers to a polynucleotide formed by the combination (e.g., ligation) of two or more synthons (usually four or more synthons). A “multisynthon” can also be referred to as a “synthon” (see definition above).

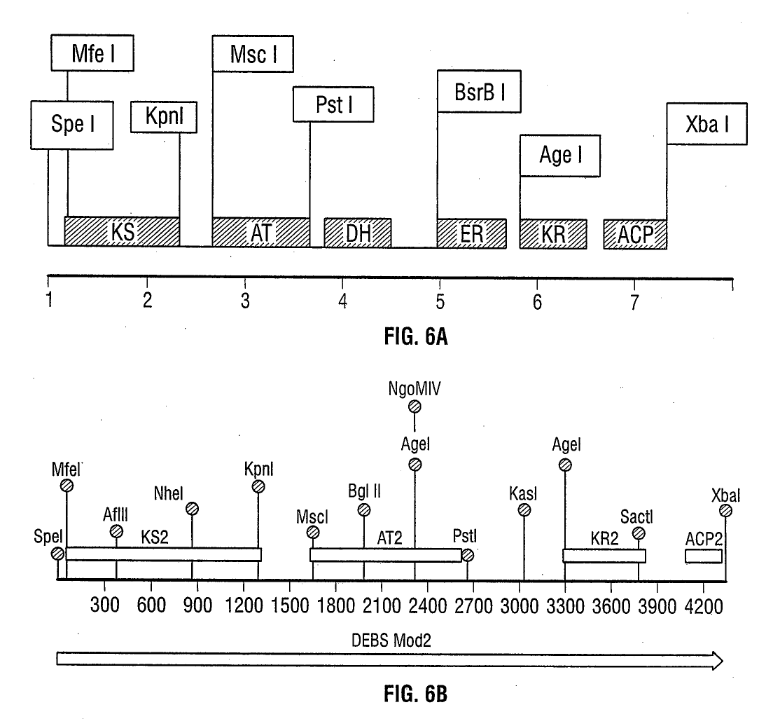

As used herein, a “module” is functional unit of a polypeptide. As used herein, “PKS module” refers to a naturally occurring, artificial or hybrid PKS extension module. PKS extension modules comprise KS and ACP domains (usually one KS and one ACP per module), often comprise an AT domain (usually one AT domain and sometimes two AT domains) where the AT activity is not supplied in trans or from an adjacent module, and sometimes comprising one or more of KR, DH, ER, MT (methyltransferase), A (adenylation), or other domains. In describing a naturally occurring PKS extension module other than at the amino terminus of a polypeptide, the term “module” can refer to the set of domains and interdomain linking regions extending approximately from the C terminus of one ACP domain to the C terminus of the next ACP domain (i.e., including a sequence linking the modules, corresponding to the Spe I-Mfe I region of the module shown in FIG. 6) linker or, alternatively can refer to the set not including the linker sequence (e.g., corresponding roughly to the Mfe I-Xba I region of the module shown in FIG. 6).

As used herein, the term “module” is more general than “PKS module” in two senses. First, “module” can be any type of functional unit including units that are not from a PKS. Second, when from a PKS, a “module” can encompass functional units of a PKS polypeptide, such as linkers, domains (including thioesterase or other releasing domains) not usually referred to in the PKS art as “PKS modules.”

As used herein, “multimodule” refers to a single polypeptide comprising two or more modules.

As used herein, the term “PKS accessory unit” (or “accessory unit”) refers to regions or domains of PKS polypeptides (or which function in polyketide synthesis) other than extension modules or domains of extension modules. Examples of PKS accessory units include loading modules, interpolypeptide linkers, and releasing domains. PKS accessory units are known in the art. The sequences for PKS loading domains are publicly available (see Table 12). Generally, the loading module is responsible for binding the first building block used to synthesize the polyketide and transferring it to the first extension module. Exemplary loading modules consists of an acyltransferase (AT) domain and an acyl carrier protein (ACP) domain (e.g., of DEBS); an KS^{Q} domain, an AT domain, and an ACP domain (e.g., of tylosin synthase or oleandolide synthase); a CoA ligase activity domain (avermectin synthase, rapamycin or FK-520 PKS) or a NRPS-like module (e.g., epothilone synthase). Linkers, both naturally occurring and artificial are also known. Naturally occurring PKS polypeptides are generally viewed as containing two types of linkers: “interpolypeptide linkers” and “intrapolypeptide linkers.” See, e.g., Broadhurst et al., 2003, “The structure of docking domains in modular polyketide synthases” Chem. Biol. 10:723-31; Wu et al. 2002, “Quantitative analysis of the relative contributions of donor acyl carrier proteins, acceptor ketosynthases, and linker regions to intermodular transfer of intermediates in hybrid polyketide synthases” Biochemistry 41:5056-66; Wu et al., 2001, “Assessing the balance between protein-protein interactions and enzyme-substrate interactions in the channeling of intermediates between polyketide synthase modules,” J Am Chem. Soc. 123:6465-74; Gokhale et al., 2000, “Role of linkers in communication between protein modules” Curr Opin Chem Biol. 4:22-7. For convenience, certain intrapolypeptide sequences linking extension modules (e.g., corresponding to the Spe I-Mfe I region of the module shown in FIG. 6) are referred to as the “ACP-KS Linker Region” or AKL. The thioesterase domain (TE) can be any found in most naturally occurring PKS molecules, e.g. in DEBS, tylosin synthase, epothilone synthase, pikromycin synthase, and soraphen synthase. Other chain-releasing activities are also accessory units, e.g. amino acid-incorporating activities such as those encoded by the rapP gene from the rapamycin cluster and its homologs from FK506, FK520, and the like; the amide-forming activities such as those found in the rifamycin and geldanamycin PKS; and hydrolases or linear ester-forming enzymes.

As used herein, a “gene” is a DNA sequence that encodes a polypeptide or polypeptide segment. A gene may also comprise additional sequences, such as for transcription regulatory elements, introns, 3′-untranslated regions, and the like.

As used herein, a “synthetic gene” is a gene comprising a polypeptide segment-encoding sequence not found in nature, where the polypeptide segment-encoding sequence encodes a polypeptide or fragment or domain at least about 30, usually at least about 40, and often at least about 50 amino acid residues in length.

As used herein, “module gene” or “module-encoding gene” refers to a gene encoding a module; a “PKS module gene” refers to a gene encoding PKS module.

As used herein, “multimodule gene” refers to a gene encoding a multimodule.

A “naturally occurring” PKS, PKS module, PKS domain, and the like is a PKS, module, or domain having the amino acid sequence of a PKS found in nature.

A “naturally occurring” PKS gene or PKS module gene or PKS domain gene is a gene having the nucleotide sequence of a PKS gene found in nature. Sequences of exemplary naturally occurring PKS genes are known (see, e.g., Table 12).

A “gene library” means a collection of individually accessible polynucleotides of interest. The polynucleotides can be maintained in vectors (e.g., plasmid or phage), cells (e.g., bacterial cells), as purified DNA, or in other forms. Library members (variously referred to as clones, constructs, polynucleotides, etc.) can be stored in a variety of ways for retrieval and use, including for example, in multiwell culture or microtiter plates, in vials, in a suitable cellular environment (e.g., E. coli cells), as purified DNA compositions on suitable storage media (e.g., the Storage IsoCode® ID™ DNA library card; Schleicher & Schuell BioScience), or a variety of other art-known library forms. Typically a library has at least about 10 members, more often at least about 100, preferably at least about 500, and even more preferably at least about 1000 members. By “individually accessible” is meant that the location of the selected library member is known such that the member can be retrieved from the library.

As used herein, the terms “corresponds” or “corresponding” describe a relationship between polypeptides. A polypeptide (e.g., a PKS module or domain) encoded by a synthetic gene corresponds to a naturally occurring polypeptide when it has substantially the same amino acid sequence. For example, a KS domain encoded by a synthetic gene would correspond to the KS domain of module 1 of DEBS if the KS domain encoded by a synthetic gene has substantially the same amino acid sequence as the KS domain of module 1 of DEBS.

As used herein, when describing recombinant manipulations of polynucleotides “joined to,” “combined with,” and grammatical equivalents of each, refer to ligation (i.e., the formation of covalent 5′ to 3′ nucleic acid linkage) of two DNA molecules (or two ends of the same DNA molecule).

As used herein, “adjacent,” when referring to adjacent DNA units such as adjacent synthons, refers to sequences that are contiguous (or overlapping) in a naturally occurring or synthetic gene. In the case of “adjacent synthons,” the sequences of the synthon coding regions are contiguous or overlapping in the synthetic gene encoded in the synthons.

As used herein, “edge,” in the context of a polynucleotide or a polypeptide segment, refers to the region at the terminus of a polynucleotide or a polypeptide (i.e., physical edge) or near a boundary delimiting a region of the polypeptide (e.g., domain) or polynucleotide (e.g., domain-encoding sequence).

The term “junction edge” is used to describe the region of a synthon that is joined to an adjacent synthon (e.g., by formation of compatible ligatable ends in each synthon). Thus, reference to “a ligatable end at a junction end” of a synthon means the end that is (or will become) ligated to the compatible ligatable end of the adjacent synthon. It will be appreciated that in a construct with five or more synthons, most synthons will have two junction edges. The junction edge(s) being referred to will be apparent from context. A sequence motif or restriction enzyme site is “near” the nucleotide sequence encoding an amino- or carboxy-terminus of a PKS domain in a module when the motif or site is closer to the specified terminus (boundary) than to the terminus (boundary) of any other domain in the module. A sequence motif or restriction enzyme site is “near” the nucleotide sequence encoding an amino- or carboxy-terminus of a PKS module when the motif or site is closer to the specified terminus (boundary) than to the terminus of any domain in the module. The boundaries of PKS domains can be determined by methods known in the art by aligning the sequence of a subject domain with the sequences of other PKS domains of a similar type (e.g., KS, ER, etc.) and identifying boundaries between regions of relatively high and relatively low sequence identity. See Donadio and Katz, 1992, “Organization of the enzymatic domains in the multifunctional polyketide synthase involved in erythromycin formation in Saccharopolyspora erythraea” Gene 111:51-60. Programs such as BLAST, CLUSTALW and those available at http://www.nii.res.in/pksdb.html can be used for alignment. In some embodiments, a motif or restriction enzyme site that is near a boundary is not more than about 20 amino acid residues from the boundary.

As used herein, “overhang” when referring to a double-stranded polynucleotide, has its usual meaning and refers to a unpaired single-strand extension at the terminus of a double-stranded polynucleotide.

A “sequence-specific nicking endonuclease” or “sequence-specific nicking enzyme” is an enzyme that recognizes a double-stranded DNA sequence, and cleaves only one strand of DNA. Exemplary nicking endonucleases are described in U.S. Patent Application 20030100094 A1 “Method for engineering strand-specific, sequence-specific, DNA-nicking enzymes.” Exemplary nicking enzymes include N.Bbv C IA, N.BstNB I and N.Alw I (New England Biolabs).

As used herein, “restriction endonuclease” or “restriction enzyme” has its usual meaning in the art. Restriction endonucleases can be referred to by describing their properties and/or using a standard nomenclature (see Roberts et al., 2002, “A nomenclature for restriction enzymes, DNA methyltransferases, homing endonucleases and their genes,” Nucleic Acids Res. 31:1805-12). Generally, “Type II” restriction endonucleases recognize specific DNA sequences and cleave at constant positions at or close to that sequence to produce 5′-phosphates and 3′-hydroxyls. “Type II” restriction endonucleases that recognize palindromic sequences are sometimes referred to herein as “conventional restriction endonucleases.” “Type IIA” restriction endonucleases are a subset of type II in which the recognition site is asymmetric. Generally, “Type IIS” restriction endonucleases is a subset of type IIA in which at least one cleavage site is outside the recognition site. As used herein, reference to “Type IIS” restriction enzymes, unless otherwise noted, refers to those Type IIS enzymes for which both DNA strands are cut outside the recognition site and on the same side of the restriction site. In one embodiment of the invention, Type IIS enzymes are selected that produce an overhang of 2 to 4 bases. Exemplary restriction endonucleases include Aat II, Acl I, Afe I, Afl II, Age I, Ahd I, Alw 26I, Alw NI, Apa I, Apa LI, Asc I, Ase I, Avr II, Bam HI, Bbs I, Bbv CI, Bci VI, Bcl I, Bfu AI, Bgl I, Bgl II, Blp I, Bpl I, Bpm I, Bpu 10I, Bsa I, Bsa BI, Bsa MI, Bse RI, Bsg I, Bsi WI, Bsm BI, Bsm I, Bsp EI, Bsp HI, Bsr BI, Bsr DI, Bsr GI, Bss HII, Bss SI, Bst API, Bst BI, Bst EII, Bst XI, Bsu 36I, Cla I, Dra I, Dra III, Dtd I, Eag I, Ear I, Eco NI, Eco RI, Eco RV, Fse I, Fsp I, Hin dIII, Hpa I, Kas I, Kpn I, Mfe I, Mlu I, Msc I, Nco I, Nde I, Ngo MIV, Nhe I, Not I, Nru I, Nsi I, Pac I, Pci I, Pfl MI, Pme I, Pml I, Psh AI, Psi I, Pst I, Pvu I, Pvu II, Rsr II, Sac I, Sac II, Sal I, San DI, Sap I, Sbf I, Sca I, Sex AI, Sfi I, Sgf I, Sgr AI, Sma I, Smi I, Sml I, Sna BI, Spe I, Sph I, Srf I, Ssp I, Stu I, Sty I, Swa I, Tat I, Tsp 509I, Tth 111I, Xba I, Xcm I, Xho I, Xmn I, those listed in Table 2, and others. e.g., http://rebase.neb.com).

As used herein, the terms “ligatable ends” refers to ends of two DNA fragments.o ends of the same molecule) that can be ligated. “Ligatable ends” include blunt ends and “cohesive ends” (having single-stranded overhangs). Two cohesive ends are “compatible” when they can be anneal and be ligated (e.g., when each overhang is of the 3′-hydroxyl end; each is of the same length, e.g., 4 nucleotide units, and the sequences of the two overhangs are reverse complements of each other).

As used herein, unless otherwise indicated or apparent from context, a “restriction site” refers to a recognition site that is at least 5, and usually at least 6 basepairs in length.

As used herein, a “unique restriction site” refers to a restriction site that exists only once in a specified polynucleotide (e.g., vector) or specified region of a polynucleotide (e.g., module-encoding portion, specified vector region, etc.).

As used herein, a “useful restriction site” refers to a restriction site that is either unique or, if not unique, exists in a pattern and number in a specified polynucleotide or specified region of a polynucleotide such that digestion at all the of the sites in a specified polynucleotide (e.g., vector) or specified region of a polynucleotide (e.g., module gene) would achieve essentially the same result as if the site was unique.

As used herein, “vector” refers to polynucleotide elements that are used to introduce recombinant nucleic acid into cells for either expression or replication and which have an origin of replication and appropriate transcriptional and/or translational control sequences, such as enhancers and promoters, and other elements for vector maintenance. In one embodiment vectors are self-replicating circular extrachromosomal DNAs. Selection and use of such vehicles is routine in the art. An “expression vector” includes vectors capable of expressing a DNA inserted into the vector (e.g., a DNA sequence operatively linked with regulatory sequences, such as promoter regions). Thus, an expression vector refers to a recombinant DNA or RNA construct, such as a plasmid, a phage, recombinant virus or other vector that, upon introduction into an appropriate host cell, results in expression of the cloned DNA.

As used herein, a specified amino acid is “similar” to a reference amino acid in a protein when substitution of the specified amino acid for the reference amino does not substantially modify the function (e.g., biological activity) of the protein. Amino acids that are similar are often conservative substitutions for each other. The following six groups contain amino acids that are conservative substitutions for one another: [alanine; serine; threonine]; [aspartic acid, glutamic acid], [asparagine, glutamine], [arginine, lysine], [isoleucine, leucine, methionine, valine], and [phenylalanine, tyrosine, and tryptophan]. Also see Creighton, 1984, PROTEINS, W.H. Freeman and Company.

A nonribosomal peptide synthase, or “NRPS” is an enzyme that produces a peptide product by joining individual amino acids through a ribosome-independent process. Examples of NRPS include gramicidin synthetase, cyclosporin synthetase, surfactin synthetase, and others. For reviews, see Weber and Marahiel, 2001, “Exploring the domain structure of modular nonribosomal peptide synthetases” Structure (Camb). 9:R3-9; Mootz et al., 2002, “Ways of assembling complex natural products on modular nonribosomal peptide synthetases” Chembiochem. 3:490-504.

### Conventions

Use of the terms “for example,” “such as, “exemplary,” “examples include,” “exempli gratia (e.g.),” “typically,” and the like are intended to illustrate aspects of the invention but are not intended to limit the invention to the particular examples described. Thus, each instance of such phrases can be read as if the phase “but not for limitation,” (e.g., “for example, but not for limitation, . . . ”) is present.

The terms “module” and “domain” generally refers to polypeptides or regions of polypeptides, while the terms “module gene” and “domain gene,” or grammatical equivalents, refer to a DNA encoding the protein. Inadvertent exceptions to this convention will be apparent from context. For example, it will be clear that “restriction sites at module edges” refers to restriction sites in the region of the module gene encoding the edge of the module polypeptide sequence.

### 2. INTRODUCTION

The present invention relates to strategies, methods, vectors, reagents, and systems for synthesis of genes, production of libraries of such genes, and manipulation and characterization of the genes and corresponding encoded polypeptides. In particular, the invention provides new methods and tools for synthesis of genes encoding large polypeptides. Examples of genes that may be synthesized include those encoding domains, modules or polypeptides of a polyketide synthase (PKS), genes encoding domains, modules or polypeptides of a non-ribosomal peptide synthase (NRPS), hybrids containing elements of both PKSs and NRPSs, viral genomes, and others. Genes encoding polyketide synthase modules are of particular interest and, for convenience, throughout this disclosure reference will often be made to design and synthesis of genes encoding PKS modules, domains and polypeptides. However, unless stated or otherwise apparent from context, aspects of the invention are not limited to any single class of genes or polypeptides. It will be understood by the reader that the methods of the present invention are useful for the design and synthesis of a large variety of polynucleotides.

The methods of the invention for producing synthetic genes encoding polypeptides of interest can include the following steps:

a). Designing a gene that encodes a polypeptide segment of interest;

b) Designing component polypeptide for synthesis of the gene;

c) Synthesizing the oligopeptide-segment encoding gene by:
i) making synthons encoding portions of the module gene; and,
ii) “stitching” synthons together to produce multisynthons (i.e., larger DNA units) that encode the polypeptide segment of interest. It will be appreciated by the reader that the polypeptide of interest can be expressed, recombinantly manipulated, and the like.

The methods and tools disclosed herein have particular application for the synthesis of polyketide synthase genes, and provide a variety of new benefits for synthesis of polyketides. As is discussed above, the order, number and domain content of modules in a polyketide synthase determine the structure of its polyketide product. Using the methods disclosed herein, genes encoding polypeptides comprising essentially any combination of PKS modules (themselves comprising a variety of combinations of domains) can be synthesized, cloned, and evaluated, and used for production of functional polyketide synthases. Such polyketide synthases can be used for production of naturally occurring polyketides without cloning and sequencing the corresponding gene cluster (useful in cases where PKS genes are inaccessible, as from unculturable or rare organisms); production of novel polyketides not produced (or not known to be produced by any naturally occurring PKS); more efficient production of analogs of known polyketides; production of gene libraries, and other uses.

In a related aspect, the invention relates to a universal design of genes encoding PKS modules (or other polypeptides) in which useful restriction sites flank functionally defined coding regions (e.g., sequence encoding modules, domains, linker regions, or combinations of these). The design allows numerous different modules to be cloned into a common set of vectors for or manipulation (e.g., by substitution of domains) and/or expression of diverse multi-modular proteins.

In a related aspect, the invention provides large libraries of PKS modules.

In a related aspect, the invention provides vectors and methods useful for gene synthesis.

In a related aspect, the invention provides algorithms useful for design of synthetic genes.

In a related aspect, the invention provides automated systems useful for gene synthesis.

The invention provides a method for making a synthetic gene encoding a PKS module by producing a plurality of DNA units by assembly PCR or other method (where each DNA unit encodes a portion of the PKS module) and combining the DNA units in a predetermined sequence to produce a PKS module-encoding gene. In one embodiment, the method includes combining the module-encoding gene in-frame with a nucleotide sequence encoding a PKS extension module, a PKS loading module, a thioesterase domain, or an PKS interpolypeptide linker, thereby producing a PKS open reading frame.

The methods of the invention for synthesis of genes encoding PKS modules can include the following steps:
a) Designing a PKS module (e.g., for production of a specific polyketide, or for inclusion in a library of modules);
b) Designing a synthetic gene encoding the desired PKS module;
c) Designing component oligonucleotides for synthesis of the gene;
d) Synthesizing the module gene by:
   i) making synthons encoding portions of the module gene; and,
   ii) “stitching” synthons together;
e) modifying module genes;
   making open reading frames comprising module gene(s) and/or accessory unit gene(s);
   producing libraries of module-encoding genes;
f) expressing a module gene from (d) or (e) in a host cell, optionally in combination with other polypeptides.
   Each of these steps is described in detail in the following sections.

### 3. DESIGN OF SYNTHETIC GENES

The nucleotide sequence of a synthetic gene of the invention will vary depending on the nature and intended uses of the gene. In general, the design of the genes will reflect the amino acid sequence of the polypeptide or fragment (e.g., PKS module or domain) to be encoded by the gene, and all or some of:
a) the codon preference of intended expression host(s).
b) the presence (introduction) of useful restriction sites in specified locations of the synthetic gene.
c) the absence (removal) of undesired restriction sites in the gene or in specified regions of the gene.
d) compatibility with synthetic methods disclosed herein, especially high-throughput methods.

A variety of criteria are available to the practitioner for selecting the gene(s) to be synthesized by the methods of the invention. The chief consideration is usually the protein encoded by the gene. For example, a gene can be synthesized that encodes a protein at least a portion of which has a sequence the same or substantially the same as a naturally occurring domain, module, linker, or other polypeptide unit, or combinations of the foregoing.

Having selected the polypeptide of interest, numerous nucleic acid sequences that encode the protein can be determined by reverse-translating the amino acid sequence. Methods for reverse translation are well known. As described below, according to the invention, reverse translation can be carried out in a fashion that “randomizes” the codon usage and optionally reflects a selected codon preference or bias. Since the synthetic genes of the invention may be expressed in a variety of hosts consideration of the codon preferences of the intended expression host may be have benefits for the efficiency of expression.

In considering codon preferences, preference tables may be obtained from publicly available sources or may be generated by the practitioner. Codon preference tables can be generated based on all reported or predicted sequences for an organism, or, alternatively, for a subset of sequences (e.g., housekeeping genes). Codon preference tables for a wide variety of species are publicly available. Tables for many organisms are available at through links from a site maintained at the Kazusa DNA Research Institute (http://www.kazusa.orjp/codon/). An exemplary codon preference for E. coli is shown in Table 1. Codon tables for Saccharomyces cerevisiae can be found in http://www.yeastgenome.org/codon_usage.shtml. In the event that no codon table is available for a particular host, the table(s) available for the most closely related organism(s) can be used.

TABLE 1
E. COLI CODON PREFERENCES*

| | | | |
|---|---|---|---|
| UUU 22.4 (35982) | UCU 8.5 (13687) | UAU 16.3 (26266) | UGU 5.2 (8340) |
| UUC 16.6 (26678) | UCC 8.6 (13849) | UAC 12.3 (19728) | UGC 6.4 (10347) |
| UUA 13.9 (22376) | UCA 7.2 (11511) | UAA 2.0 (3246) | UGA 0.9 (1468) |
| UUG 13.7 (22070) | UCG 8.9 (14379) | UAG 0.2 (378) | UGG 15.3 (24615) |
| CUU 11.0 (17754) | CCU 7.1 (11340) | CAU 12.9 (20728) | CGU 21.0 (33694) |
| CUC 11.0 (17723) | CCC 5.5 (8915) | CAC 9.7 (15595) | CGC 22.0 (35306) |
| CUA 3.9 (6212) | CCA 8.5 (13707) | CAA 15.4 (24835) | CGA 3.6 (5716) |
| CUG 52.7 (84673) | CCG 23.2 (37328) | CAG 28.8 (46319) | CGG 5.4 (8684) |
| AUU 30.4 (48818) | ACU 9.0 (14397) | AAU 17.7 (28465) | AGU 8.8 (14092) |
| AUC 25.0 (40176) | ACC 23.4 (37624) | AAC 21.7 (34912) | AGC 16.1 (25843) |
| AUA 4.3 (6962) | ACA 7.1 (11366) | AAA 33.6 (54097) | AGA 2.1 (3337) |
| AUG 27.7 (44614) | ACG 14.4 (23124) | AAG 10.2 (16401) | AGG 1.2 (1987) |
| GUU 18.4 (29569) | GCU 15.4 (24719) | GAU 32.2 (51852) | GGU 24.9 (40019) |
| GUC 15.2 (24477) | GCC 25.5 (40993) | GAC 19.0 (30627) | GGC 29.4 (47309) |
| GUA 10.9 (17508) | GCA 20.3 (32666) | GAA 39.5 (63517) | GGA 7.9 (12776) |
| GUG 26.2 (42212) | GCG 33.6 (53988) | GAG 17.7 (28522) | GGG 11.0 (17704) |

*fields: [triplet] [frequenCy: per thousand] [(number)]

In addition to accounting for the codon preferences of a specified host (expression) organism, the nucleotide acid sequence of the synthetic gene may be designed to avoid clusters of adjacent rare codons, or regions of sequence duplication.

Suitable expression hosts will depend on the protein encoded. For PKS proteins, suitable hosts include cells that natively produce modular polyketides or have been engineered so as to be capable of producing modular polyketides. Hosts include, but are not limited to, actinomycetes such as Streptomyces coelicolor, Streptomyces venezuelae, Streptomyces fradiae, Streptomyces ambofaciens, and Saccharopolyspora erythraea, eubacteria such as Escherichia coli, myxobacteria such as Myxococcus xanthus, and yeasts such as Saccharomyces cerevisiae. See, for example, Kealey et al., 1998, “Production of a polyketide natural product in nonpolyketide-producing prokaryotic and eukaryotic hosts” Proc Natl Acad Sci USA 95:505-9; Dayem et al, 2002, “Metabolic engineering of a methylmalonyl-CoA mutase-epimerase pathway for complex polyketide biosynthesis in Escherichia coli” Biochemistry 41:5193-201.

Codon optimization may be employed throughout the gene, or, alternatively, only in certain regions (e.g., the first few codons of the encoded polypeptide). In a different embodiment, codon optimization for a particular host is not considered in design of the gene, but codon randomization is used.

In an alternative embodiment, the DNA sequence of a naturally occurring gene encoding the protein is used to design the synthetic gene. In this embodiment the naturally occurring DNA sequence is modified as described below (e.g., to remove and introduce restriction sites) to provide the sequence of the synthetic gene.

The design of synthetic genes of the invention also involves the inclusion of desired restriction sites at certain locations in the gene, and exclusion of undesired restriction sites in the gene or in specified regions of the gene, as well as compatibility with synthetic methods used to make the gene(s). Often, an “undesired” restriction site (e.g., Eco RI site) is removed from one location to ensure that the same site is unique (for example) in another location of the gene, synthon, etc. These considerations will be more easily described and understood following a description of methods and tools employed in the synthesis and use of the synthetic genes of the invention. These methods and tools are described, in part, in Section 4, below, and further aspects of gene design are discussed in Section 5.

### 4. SYNTHESIS OF GENES

This section describes methods for production of synthetic genes. As noted above, in one aspect of the invention production of synthetic genes comprises combining (“stitching”) two or more double-stranded, polynucleotides (referred to here as “synthons”) to produce larger DNA units (i.e., multisynthons). The larger DNA unit can be virtually any length clonable in recombinant vectors but usually has a length bounded by a lower limit of about 500, 1000, 2000, 3000, 5000, 8000, or 10000 base pairs and an independently selected upper limit of about 5000, 10000, 20000 or 50000 base pairs (where the upper limit is greater than the lower limit). For purposes of illustration, the following discussion generally refers to production of synthetic genes in which the larger DNA units encode PKS modules. However, it is contemplated that the methods and materials described herein may be used for synthesis of any number of polypeptide-segment encoding nucleotide sequences, including sequences encoding NRPS modules and synthetic variants, polypeptide segments of other modular proteins, polypeptide segments from other protein families, or any functional or structural DNA unit of interest.

According to the invention, typically, synthetic PKS module genes are produced by combining synthons ranging in length from about 300 to about 700 bp, more often from about 400 to about 600 bp, and usually about 500 bp. In the case of PKS modules, naturally occurring PKS module genes (and corresponding synthetic genes) are in the neighborhood of about 5000 bp in length. More generally, modules produce by synthon Allowing for some overlap between sequences of adjacent synthons, ten to twelve 500-bp synthons are typically combined to produce a 5000 bp module gene encoding a naturally occurring module or variant thereof. In various aspects of the invention, the number of synthons that are “stitched” together can be at least 2, at least 3, at least 4, at least 5, at least 6, at least 7, at least 8, at least 9, or at least 10, or can be a range delimited by a first integer selected from 2, 3, 4, 5, 6, 7, 8, 9, or 10 and a second selected from 5, 10, 20, 30 or 50 (where the second integer is greater than the first integer).

The next section describes synthon production. The following section, §4.2, describes the synthesis of module genes by stitching synthons, as well as vectors useful for stitching.

4.1 Synthesis of Synthons

Synthons can be produced in a variety of ways. Just as module genes are produced by combining several synthons, synthons are generally produced by combining several shorter polynucleotides (i.e. oligonucleotides). Generally synthons are produced using assembly PCR methods. Useful assembly PCR strategies are known and involve PCR amplification of a set of overlapping single-stranded polynucleotides to produce a longer double-stranded polynucleotide (see e.g., Stemmer et al., 1995, “Single-step assembly of a gene and entire plasmid from large numbers of oligodeoxyribonucleotides” Gene 164:49-53; Withers-Martinez et al., 1999, “PCR-based gene synthesis as an efficient approach for expression of the A+T-rich malaria genome” Protein Eng. 12:1113-20; and Hoover and Lubkowski, 2002, “DNAWorks: An automated method for designing oligonucleotides for PCR-based gene synthesis” Nucleic Acids Res. 30:43). Alternatively, synthons can be prepared by other methods, such as ligase-based methods (e.g., Chalmer and Curnow, 2001, “Scaling Up the Ligase Chain Reaction-Based Approach to Gene Synthesis” Biotechniques 30:249-252).

It will become apparent to the reader that the sequences of the oligonucleotide components of a synthon determines the sequence of the synthon, and ultimately the synthetic gene generated using the synthon. Thus, the sequences of the oligonucleotide components (1) encode the desired amino acid sequence, (2) usually reflect the codon preferences for the expression host, (3) contain restriction sites used during synthesis or desired in the synthetic gene, (4) are designed to exclude from the synthetic gene restriction sites that are not desired, (5) have annealing, priming and other characteristics consistent with the synthetic method (e.g. assembly PCR), and (6) reflect other design considerations described herein.

Synthons about 500 bp in length are conveniently prepared by assembly amplification of about twenty-five 40-base oligonucleotides (“40-mers”). In some embodiments of the invention, uracil-containing oligonucleotides are added to the ends of synthons (i.e., synthon flanking regions) to facilitate ligation independent cloning. (See Example 1). The oligonucleotides themselves are designed according to the principles described herein, can be prepared using by conventional methods (e.g., phosphoramidite synthesis) and/or can be obtained from a number of commercial sources (e.g., Sigma-Genosys, Operon). Although purified oligonucleotides can be used for synthon assembly, for high-throughput methods the oligonucleotide preparation usually is desalted but not gel purified (See Example 1). Assembly and amplification conditions are selected to minimize introduction of mutations (sequence errors).

4.2 Synthesis of Module Genes (Stitching)

The process of combining synthons to produce module genes is referred to as “stitching.” Usually at least three synthons are combined, more often at least five synthons, and most often at least eight synthons are combined. The stitching methods of the invention are suitable for high-throughput systems, avoid the need for purification of synthon fragments, and have other advantages. As previously noted, although stitching is described in the context of synthesis of PKS gene modules (ca. 5000 bp) it can be used for synthesis of any large gene. For example, stitching can be used to combine two or more PKS module genes to prepare multimodule genes or to combine any of a variety of other combinations of polynucleotides (e.g., a promoter sequence and a RNA encoding sequence).

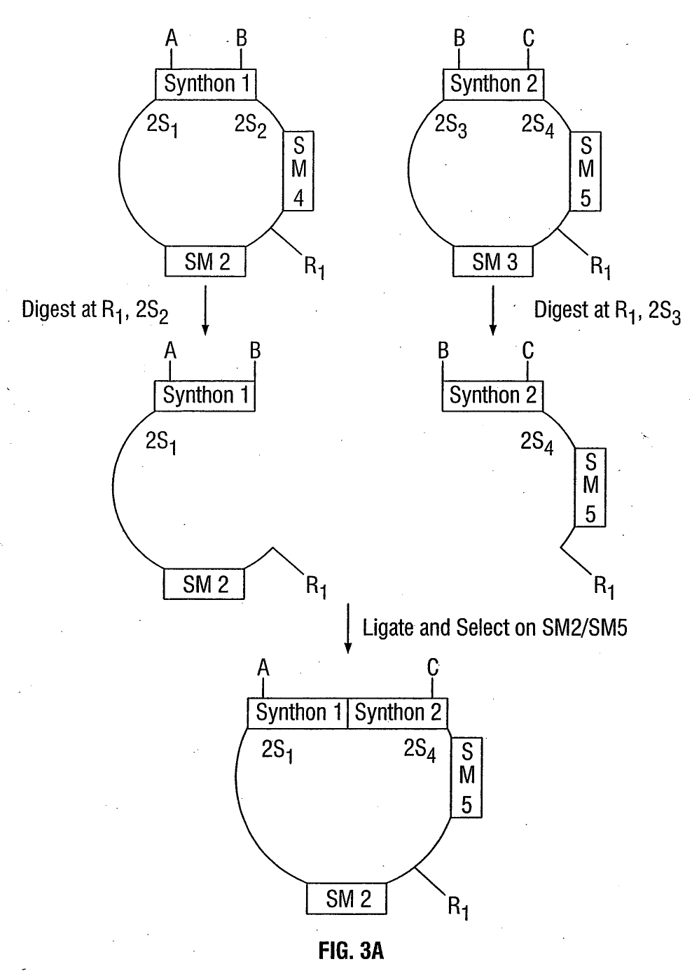

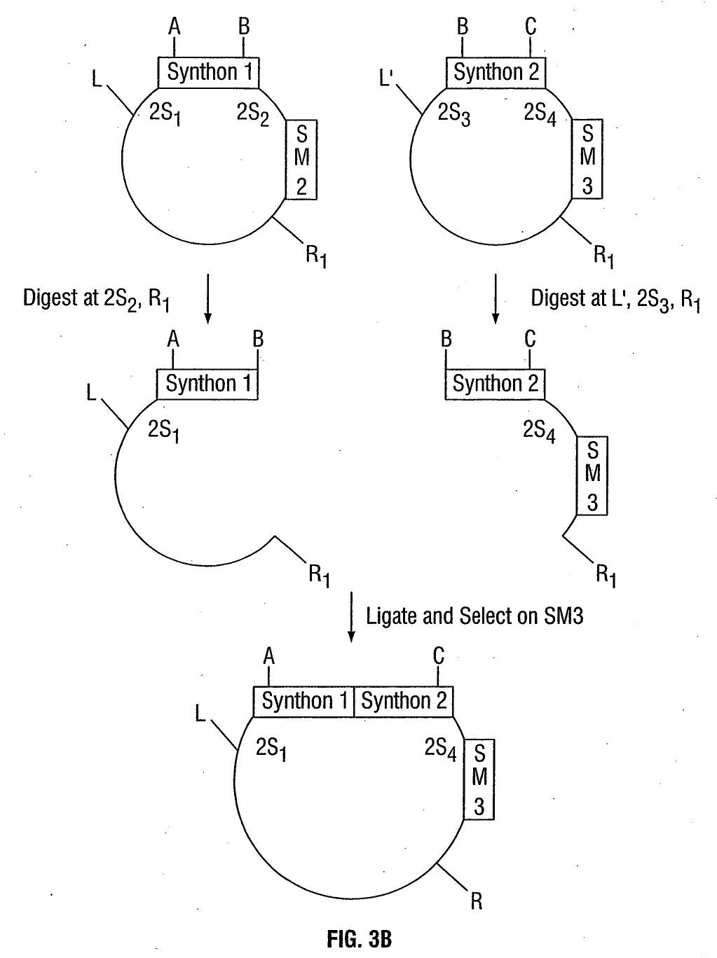

Stitching involves joining adjacent DNA units (e.g., synthons) by a process in which a first DNA unit (e.g., a first synthon or multisynthon) in a first vector is combined with an adjacent DNA unit (e.g., an adjacent synthon or multisynthon) in a second vector that is differently selectable from the first vector. Each of the two vectors contains an origin of replication (as used herein, reference to a “vector” indicates the presence of an origin of replication). The two vectors containing the adjacent DNA units (hereinafter, “synthons”) are sometimes referred to as a “cognate pair” or as the “donor” and “acceptor” vectors. In the stitching process, each of the two vectors is digested with restriction enzymes to generate fragments with compatible (usually cohesive) ligatable ends in the synthon sequences (allowing the synthons to be joined by ligation) and to generate compatible (usually cohesive) ligatable ends outside the synthon sequences such that the two synthon-containing vector fragments can be ligated to generate a new, selectable, vector containing the joined synthon sequences (multisynthon). As described in detail below, the invention provides methods for rapid cloning of large genes without the need for fragment purification steps during synthesis. Stitching methods are described below and illustrated in FIGS. 3, 5 and 7.

In one aspect of the invention, a method is provided for joining several DNA units in sequence, the method by
a) carrying out a first round of stitching comprising ligating an acceptor vector fragment comprising a first synthon SA₀, a ligatable end LA₀ at the junction end of synthon SA₀ and an adjacent synthon SD₀, and another ligatable end la₀, and a donor vector fragment comprising a second synthon SD₀, a ligatable end LD₀ at the junction end of synthon SD₀ and synthon SA₀, wherein LD₀ and LA₀ are compatible, another ligatable end ld₀, wherein ld₀ and la₀ are compatible, and a selectable marker, wherein LA₀ and LD₀ are ligated and la₀ and ld₀ are ligated, thereby joining the first and second synthons, and thereby generating a first vector comprising synthon coding sequence S₁;
b) selecting for the first vector by selecting for the selectable marker in (a); and,
c) carrying out a number n additional rounds of stitching, wherein n is an integer from 1 to 20, wherein Sₙ is the synthon coding sequence generated by joining synthons in the previous round of stitching, and wherein each round n of stitching comprises: 1) designating the first or a subsequent vector as either an acceptor vector Aₙ or a donor vector Dₙ; 2) digesting acceptor vector Aₙ with restriction enzymes to produce an acceptor vector fragment comprising a synthon coding sequence Sₙ, a ligatable end LAₙ at the junction end of synthon Sₙ and an adjacent synthon SDₙ₊₁₀₀, and another ligatable end laₙ; and, ligating the acceptor vector fragment to a donor vector fragment comprising synthon SDₙ₊₁₀₀, a ligatable end LDₙ₊₁₀₀ at the junction end of synthon SDₙ₊₁₀₀ and synthon Sₙ, wherein LAₙ and LDₙ₊₁₀₀ are compatible. another ligatable end ldₙ₊₁₀₀ wherein laₙ and ldₙ₊₁₀₀ are compatible, and a selectable marker, wherein LAₙ and LDₙ₊₁₀₀ are ligated and laₙ and ldₙ₊₁₀₀ are ligated, thereby generating a subsequent vector, or digesting donor vector Dₙ with restriction enzymes to produce a donor vector fragment comprising a synthon coding sequence Sₙ, a ligatable end LDₙ at the junction end of synthon Sₙ and an adjacent synthon SAₙ₊₁₀₀, another ligatable end ldₙ, and a selectable marker; and ligating the donor vector fragment to an acceptor vector fragment comprising synthon SAₙ₊₁₀₀, a ligatable end LAₙ₊₁₀₀ at the junction end of synthon SAₙ₊₁₀₀ and synthon Sₙ, and another ligatable end laₙ₊₁₀₀ wherein LAₙ₊₁₀₀ and LDₙ are compatible and are ligated and laₙ₊₁₀₀ and ldₙ are compatible and are ligated, thereby generating a subsequent vector

d) selecting the subsequent vector by selecting for the selectable marker of the donor vector fragment of step (c)

e) repeating steps (c) and (d) n−1 times thereby producing a multisynthon.

In various embodiments, the selectable marker of step (d) is not the same as the selectable marker of the preceding stitching step and/or is not the same as the selectable marker of the subsequent stitching step; la₀, ld₀, laₙ, ldₙ are the same and/or La₀, Ld₀, Laₙ, and Ldₙ are created by a Type IIS restriction enzyme; the synthons SA₀, SD₀, SAn₊₁₀₀, and SDn₊₁₀₀ are synthetic DNAs; any one or more of synthons SA₀, SD₀, SAn₊₁₀₀, or SDn₊₁₀₀ is a multisynthon; and/or the multisynthon product of step (e) encodes a polypeptide comprising a PKS domain.

Two related approaches for stitching have been used by the inventors, each involving (1) cloning synthons into assembly vectors, (2) joining adjacent synthons, and (3) selecting desired constructs. The first stitching approach, referred to as “Method S,” is facilitated by use of recognition sites for Type IIS restriction enzymes (as defined above). The second stitching approach, referred to as “Method R,” is facilitated by recognition sites for conventional (Type II) restriction enzymes.

The two stitching approaches described here differ in the joining step, but use similar methods for cloning into assembly vectors and selection. Each of these steps is discussed below.

4.2.1 Cloning Synthons in Assembly Vectors

The term “assembly vector” is used to refer to vectors used for the stitching step of gene synthesis. In one aspect of the invention, an assembly vector has a site, the “synthon insertion site” or “SIS,” into which synthons can be cloned (inserted). The structure of the SIS will depend on the cloning method used. An assembly vector comprising a synthon sequence can be called an “occupied” assembly vector. An assembly vector into which no synthon sequence has been cloned can be called an “empty” assembly vector.

Although any method of cloning the synthon can be used to introduce the synthon into the SIS of the vector, for automated high-throughput cloning, ligation-independent cloning (LIC) methods are preferred. Several methods for LIC are known, including single-strand extension based methods and topoisomerase-based methods (see, e.g., Chen et al., 2002, “Universal Restriction Site-Free Cloning Method Using Chimeric Primers” BioTech 32:516-20; Rashtchian et al., 1992, “Uracil DNA glycosylase-mediated cloning of polymerase chain reaction-amplified DNA: application to genomic and cDNA cloning” Anal Biochem 206:91-97; and TOPO-cloning by Invitrogen Corp.). One LIC method involves creating single-strand complementary overhangs sufficiently long for annealing to each other (often 12 to 20 bases) on (a) the synthon and (b) the vector. When the synthon and vector are annealed and transformed into a host (e.g., E. coli) a closed, circular plasmid is generated with high efficiency.

In one embodiment, 3′-overhangs, or “LIC extensions” are introduced to the synthon using PCR primers that are later partially destroyed. This can be accomplished by incorporating uracil (U) residues (instead of thymidine) into a PCR primer, linking the primer onto the 3′ ends of the product of assembly PCR described above, and digesting with Uracil-DNA Glycosidase (UDG). UDG cleaves the uracil residues from the sugar backbone, leaving the bases of the other strand free to interact with the complementary strand on the vector (see, e.g., Rashtchian et al., 1992). An alternative method involves incorporating a primer containing a ribonucleotide that is cleaved with mild base or RNAse.

Because the sequences at synthon edges can be controlled by the practitioner, a single pair of UDG primers can be used for LIC of a large number of different synthons allowing automated and high-throughput LIC cloning of synthons.

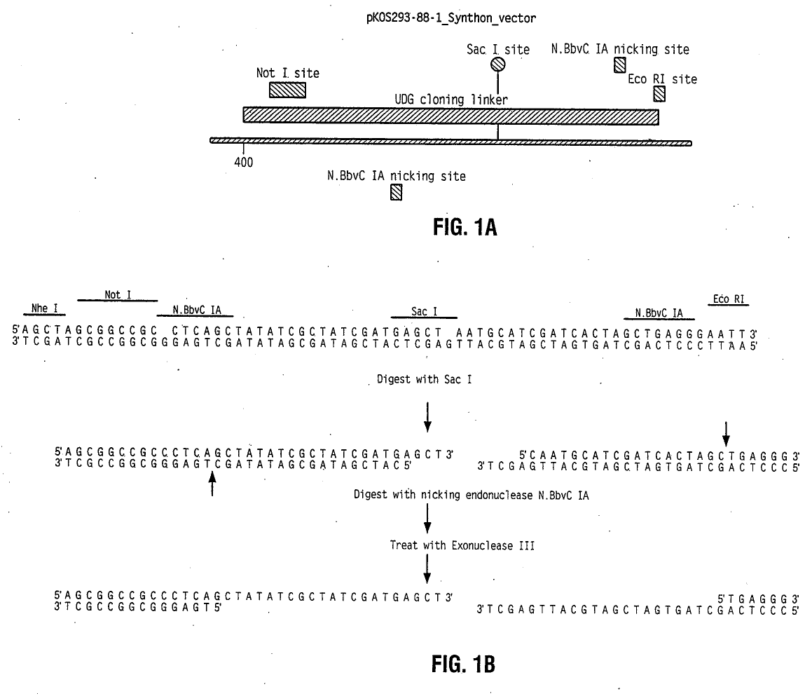

There are also several options for generating the 3′-overhang on the vector. As above, it can be produced using primers containing U instead of T to replicate the entire plasmid, followed by treatment with UDG. Alternatively, a double-stranded fragment containing U's on one strand can be ligated to the vector followed by treatment with UDG. A particularly useful method for producing an LIC extension by digesting an appropriately designed SIS with a restriction enzyme that cleaves double-stranded DNA and with sequence-specific nicking endonuclease(s). FIG. 1 illustrates this technique using, as an example, the UDG-LIC synthon insertion site from the vector pKOS293-88-1. Also see Example 2. The nicked, linearized, DNA is treated with exonuclease III to remove the small oligonucleotides (exonuclease III cleaves 3′→5′, providing there are no 3′-overhangs). In an alternative method, the 3′-overhang on the vector is generated by the action of endonuclease VIII (see Example 2). The “central” restriction site is positioned such that cleavage with the restriction endonuclease and nicking endonuclease(s), followed by digestion with the exo- or endo-nuclease results in 3′ overhangs suitable for annealing to a fragment with complementary 3′ overhangs. Usually the central restriction site is a single, unique, site in the vector. However, the reader will immediately recognize that pairs or combinations of restriction sites can be used to accomplish the same result.

In an alternative embodiment, the SIS can have other recognition sites for one or more restriction enzymes that cleave both strands (e.g., a conventional “polylinker”) and synthons can be inserted by ligase-mediated cloning.

4.2.2 Validation of Synthons

High-throughput synthesis of libraries of large genes requires an enormous number of synthetic steps (beginning, for example, with synthesis of oligonucleotides). To maximize the frequency of a successful outcome (i.e., a gene having the desired sequence) the present invention provides optional validation steps throughout the synthetic process. To identify clones containing a synthon having the expected sequence (e.g. following oligonucleotide synthesis, assembly PCR, and LIC), assembly vector DNA is usually isolated from several (typically five or more) clones and sequenced. See Example 3. Synthon samples can be sequenced until a clone with the desired sequence is found. Alternatively, clones with a small number of errors (e.g., only 1 or 2 point mutations) can be corrected using site-directed mutagenesis (SDM). One method for SDM is PCR-based site-directed mutagenesis using the 40-mer oligonucleotides used in the original gene synthesis.

4.2.3 Method S: Joining Strategies, Assembly Vectors, & Selection Schemes

As noted above, two different stitching methods, “Method S” and “Method R,” have been used by the inventors. This section describes Method S.

4.2.3.1 Joining Strategies

Method S entails the use of Type IIS restriction enzyme recognition sites (as defined above) usually outside the coding sequences of the synthons (i.e., in the synthon flanking region). In Method S, recognition sites for Type IIS restriction enzymes can be incorporated into the synthon flanking regions (e.g., during assembly PCR). The sites are positioned so that addition of the corresponding restriction enzyme results in cleavage in the synthon coding region and creation of ligatable ends. For illustration and not limitation, this is diagrammed below (R1, R2, R3, and R4=recognition sites for Type IIS restriction enzymes and digestion with R2 and R3 produce compatible cohesive ends [(same length and orientation) overhangs], vvvvvvv=assembly vector region, ssssssss=synthon coding region, s=sequence that is the same in the two synthons, ooo=synthon flanking regions).

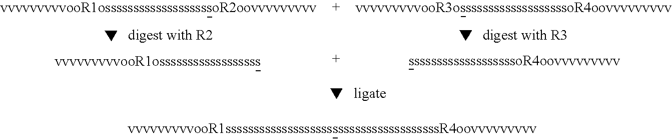

In one embodiment of this method, R1 and R3 are the same and R2 and R4 are the same. This approach simplifies the design of the vectors used and the stitching process. In an alternative embodiment, the Type IIS recognition sites can be present in the synthon coding region, rather than the flanking regions, provided the sites can be introduced consistent with the codon requirements of the coding region.

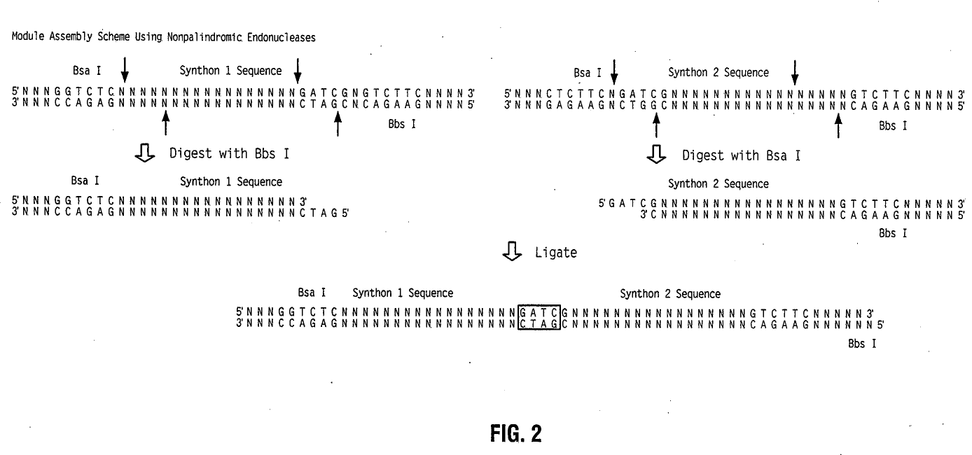

The sequence that is the same in the two synthons (“s”) usually comprises at least 3 base pairs, and often comprises at least 4 base pairs. In an embodiment, the sequence is 5′-GATC-3′. Table 2 shows exemplary Type IIS restriction enzymes and recognition sites. FIG. 2 illustrates the Method S joining method using Bbs I and Bsa I as enzymes.

TABLE 2

EXEMPLARY TYPE IIS RESTRICTION
ENZYMES AND RECOGNITION SITES

| | | | |
|---|---|---|---|
| RestriCtion | | | |
| Enzymes | ReCognition Site | Cut Site | Overhang |

| | | | |
|---|---|---|---|
| BCIV I | GTATCC | N6, N5 | −1 |
| Bmr I | ACTGGG | N5, N4 | −1 |
| Bpm I | CTGGAG | N16, N14 | −2 |
| BpuEI | CTTGAG | N16, N14 | −2 |
| BseR I | GAGGAG | N10, N8 | −2 |
| Bsg I | GTATCC | N16, N14 | −2 |
| BsrDi | GCAATG | N2, N0 | −2 |
| Bts I | GCAGTG | N2, N0 | −2 |
| ECi I | GGCGGA | N11, N9 | −2 |
| Ear I | CTCTTC | N1, N4 | 3 |
| Sap I | GCTCTTC | N1, N4 | 3 |
| BsmB I | CGTCTC | N1, N5 | 4 |
| BspM I | ACCTGC | N4, N8 | 4 |
| Bsa I | GGTCTC | N1, N5 | 4 |
| Bbs I | GAAGAC | N2/N6 | 4 |
| BfuA I | ACCTGC | N4, N8 | 4 |
| Fok I | GGATG | N9/N13 | |
| Alw I | GGATC | N4/N5 | |

4.2.3.2 Assembly Vectors

FIG. 3 illustrates how the joining method described above can be combined with a selection strategy to efficiently link a series of adjacent synthons. In this embodiment, pairs of adjacent synthons (or adjacent multisynthons) are cloned into the SIS sites of cognate pairs of vectors, where the two members of the pair are differently selectable. These selection strategies are discussed in greater detail in the next section (4.3.2.3). In this section, exemplary cognate vector pairs that can be used in stitching are described, as well as certain intermediates (occupied assembly vectors) created during the stitching process.

Vector Pair I

In one embodiment, the stitching vectors have i) a synthon insertion site (SIS); ii) a “right” restriction site (R₁) common to both vectors or, alternatively, that is different in each vector but which produce compatible ends; iii) a first selection marker (SM2 or SM3) that is different in each vector; iv) a second selection marker (SM4 or SM5) that is different in each vector; and, v) optionally a third selection marker (SM1) common to both vectors. The convention used here is that SM2 and SM4 lie on the first vector of the pair, and SM3 and SM5 lie on the second vector of the pair, and none of SM2-5 are the same.

The spatial arrangement of these elements can be

(SM2 or SM3)-SIS—(SM4 or SM5)-R₁ [I]

In Vector I, the right restriction site is usually a unique site in the vector. In cases in which there is more than one site, the additional sites are positioned so that the additional copies do not interfere with the strategy described below and illustrated in FIG. 3A. [For example, in an acceptor vector, the R₁ site can be unique or, if not unique, absent from the portion of the vector containing the SIS (or synthon), the SM2/SM3, and delimited by the SIS (or the junction edge of the synthon) and the R₁ site (i.e., the R₁ that is cleaved to result in the ligatable end). In a donor vector, the R₁ site can be unique or, if not unique, absent from the portion of the vector containing the SIS (or synthon) and the SM4/SM5 site, and delimited by the SIS (or the junction edge of the synthon) and the R₁ site (e.g., the R₁ that is cleaved to result in the ligatable end)].

The R₁ site can be a recognition sites for any Type II restriction enzyme that forms a ligatable end (e.g., usually cohesive ends). Usually the recognition sequence is at least 5-bp, and often is at least 6-bp. In one embodiment, the right restriction site is about 1 kb downstream of the SIS. In one embodiment of the invention, the R₁ sites of the donor and acceptor vectors are not the same, but simply produce compatible cohesive ends when each is cleaved by a restriction enzyme.

In one embodiment of the invention, the SIS is a site suitable for LIC having a sequence with a pair of nicking sites recognized by a site-specific nicking endonuclease (usually the same endonuclease recognizes both nicking sites) and, positioned between the nicking sites, a restriction site recognized by a restriction endonuclease (to linearize the nicked SIS, consistent with the LIC strategy described above). In one embodiment, the nicking endonuclease is N.BbvC IA, which recognizes the sequence (^{▴}=nicking site):

5′ . . . GC^{▾}TGAGG . . . 3′
3′ . . . CGACTCC . . . 5′

Accordingly, in one embodiment, a Vector Pair I vector has the following structure, where N₁ and N₂ are recognition sites for nicking enzymes (usually the same enzyme), R₂ is an SIS restriction site as discussed above, and R₁ and SM1-5 are as described above, e.g.,

(SM2 or SM3)-N₁—R₂—N₂—(SM4 or SM5)-R₁ [II]

In one embodiment of the invention, a Vector Pair I vector is “occupied” by a synthon, and has the following structure, where 2S₁ and 2S₂ are recognition sites for Type IIS restriction enzymes, Sy is synthon coding region, and R₁ and SM1-5 are as described above, e.g.,

(SM2 or SM3)-2S₁-Sy-2S₂—(SM4 or SM5)-R₁ [III]

This is an intermediate construct useful for stitching.

Vector Pair II

Vector pair II requires only one unique selectable marker on each vector in the pair (i.e., an SM found on one vector and not the other) although additional selectable markers may optionally be included. In one embodiment, the stitching vectors have

i) a synthon insertion site (SIS);

ii) a “right” restriction site (R₁) as described above for Vector I, usually common to both vectors;

iii) a “left restriction site” on each vector that may be the same or different (L or L′);

iv) a first selection marker (SM2 or SM3) that is different in each vector

vi) optionally a second selection marker (SM4 or SM5) that is different in each vector; and,

vi) optionally a third selection marker (SM1), common to both vectors.

The spatial arrangement of these elements can be

(SM4 or SM5)-(L or L′)-SIS—(SM2 or SM3)-R₁ [IV]

In this embodiment, the right restriction site (R₁) and left restriction site (L or L′) are usually unique sites in the vector. In cases in which they are not unique, the additional sites are positioned so they do not interfere with the strategy described below and illustrated in FIG. 3B. Recognition sites for any Type II restriction enzyme may be used, although typically the recognition sequence is at least 5-bp, often at least 6-bp. In one embodiment, the right restriction site is about 1 kb downstream of the SIS.

The vectors also contain the conventional elements required for vector function in the host cell or useful for vector maintenance (for example, they may contain one or more of an origin of replication, transcriptional and/or translational control sequences, such as enhancers and promoters, and other elements).

In one embodiment of the invention, the SIS is a site suitable for LIC having a sequence with a pair of nicking sites recognized by a site-specific nicking endonuclease as described above in the description of Vector Pair I. Accordingly, in one embodiment, a Vector Pair II vector has the following structure, where N₁ and N₂, R₁, R₂, L, L′, and SM2 and 3 and SM1-5 are as described above, e.g.,

(L or L′)-N₁—R₂—N₂—(SM2 or SM3)-R₁ [V]

In one embodiment of the invention, a Vector Pair II vector comprises a synthon cloned at the SIS site and has the following structure, where 2S, and 2S₂, Sy, R₁, L, L′, SM2 and 3 are described above, e.g.,

(L or L′)-2S₁-Sy-2S₂—(SM2 or SM3)-R₁ [VI]

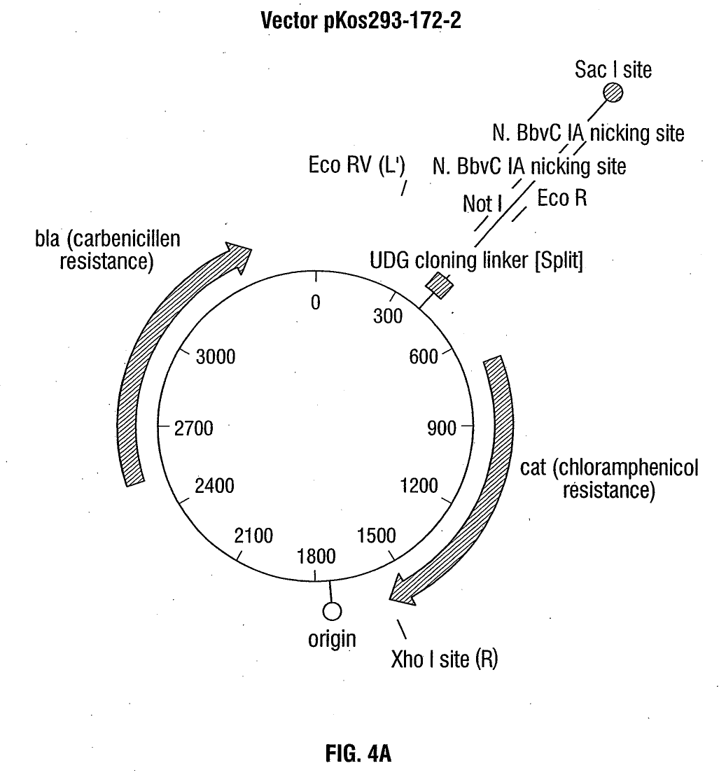

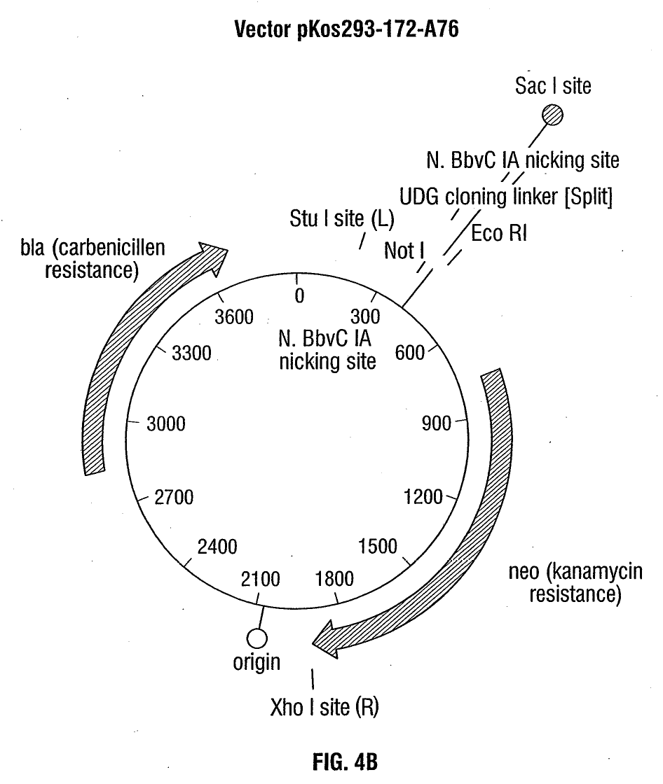

FIG. 4 is a diagram of exemplary stitching vectors pKos293-172-2 and pKos293-172-A76.

4.2.3.3 Selection Schemes

Two-Selection Marker Scheme

As noted, FIG. 3 illustrates how the joining method shown above can be combined with a selection strategy to efficiently link a series of adjacent synthons (or other DNA units). Using Vector Pair I (FIG. 3A), the vectors of the pair into which adjacent synthons have been cloned are digested with R₁ (e.g., Xho I) and with either 2S₁ or 2S₂ (the site closest to the junction edges), and the products ligated. Thus, the vector containing the first synthon (acceptor vector) is restricted at the 3′-synthon edge and R₁ downstream of the 3′ synthon edge). The vector containing the second, 3′ adjacent synthon (donor vector) is restricted at the 5′-synthon edge and R₁. The resulting products are ligated to reconstruct the vector containing 2 synthons, and selection is by antibiotic resistance markers SM2 and SM5. By selecting for positive clones with a unique selection marker from both the donor and the acceptor plasmid, only the correct clones will have the two markers.

By running parallel reactions, four 2-synthon vectors are prepared simultaneously to prepare four 2-synthon vectors. Next, using the same approach, four 2-synthon fragments are stitched to make two 4-synthon fragments, and then the two 4 synthon fragments are stitched together to make an 8-synthon product. For illustration, consider a vector pair each having two unique SMs (SM2, SM4 and SM3, SM5). To make a hypothetical 8-synthon module of sequence S1-S2-S3-S4-S5-S6-S7-S8 where S1-8 are synthons, synthons 1, 4, 6, and 7 can be cloned into the vector with the SM2+SM4 markers, and 2, 3, 5, and 8 can be cloned into the vector with the SM3+SM5 markers as summarized in Table 3.

TABLE 3
SELECTION STRATEGY

| | Synthon | | | | | | | |
|---|---|---|---|---|---|---|---|---|
| | 1 | 2 | 3 | 4 | 5 | 6 | 7 | 8 |
| 1-syn¹ | SM2 | SM3 | SM3 | SM2 | SM3 | SM2 | SM2 | SM3 |
| | SM4 | SM5 | SM5 | SM4 | SM5 | SM4 | SM4 | SM5 |

| | | | | |
|---|---|---|---|---|
| 2-syn² | SM2 + SM5 | SM3 + SM4 | SM 3 + SM4 | SM2 + SM5 |

| | | |
|---|---|---|
| 4-syn² | SM2 + SM4 | SM3 + SM5 |

| | |
|---|---|
| 8-syn² | SM2 + SM5 |

¹Shows unique marker of vector into which synthon is cloned.
²Shows marker selected for after of synthons are combined.

The same procedure is applied to the two vectors containing synthon 3 (SM3, SM5) and synthon 4 (SM2, SM4). This would produce a 2-synthon vector containing SM3 and SM4 and selectable for these markers. Next, the 2-synthon insert containing synthons 3 and 4 are cloned into the first 2-synthon containing synthons 1 and 2 to give a 4-synthon product (1-2-3-4) in a SM2+SM4 vector. This could be repeated with the synthons 5, 6, 7, and 8 to give a 4-synthon insert (5-6-7-8) in a SM3+SM5 vector. The two would then be combined as before to give an 8-synthon module in an SM3 vector.

It can be seen that by designing modules to contain 2ⁿ synthons, and parallel-processing the synthon stitching reactions, a complete module can be assembled in n operations.

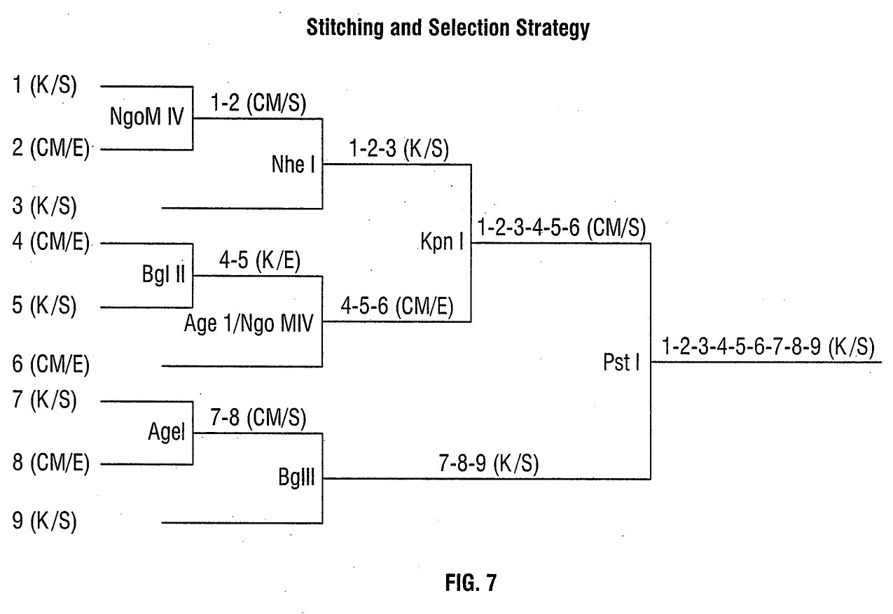

Although pairwise combining minimizes ligation steps, and is thus particularly efficient, other combination strategies, such as that illustrated in FIG. 7 for Method R, can be used.

A wide variety of selection markers and selection methods are known in molecular biology and can be used for selection. Typically, the marker is a gene for drug resistance such as carb (carbenicillin resistance), tet (tetracycline resistance), kan (kanamycin resistance), strep (streptomycin resistance) or cm (chloramphenicol resistance). Other suitable selection markers include counterselectable markers (csm) such as sacB (sucrose sensitivity), araB (ribulose sensitivity), and tetAR (codes for tetracycline resistance/fusaric acid hypersensitivity). Many other selectable markers are known in the art and could be employed.

One-Marker Scheme

An alternative selection strategy uses Vector Pair II. According to this strategy, at each round, the two vectors are mixed in equal amounts, and simultaneously digested to completion with restriction enzymes R₁, L (or L′), and the Type IIS enzyme corresponding to the restriction site at the two synthon edges to be joined, followed by ligation. In FIG. 3B, the vector containing synthon 1+SM2 is cut at right edge of the synthon and at R, and the vector containing synthon 2+SM3 is cut at the left edge of the synthon and at R₁ and at L′. Cleavage at L′ is intended to prevent re-ligation of this fragment. The mixture of fragments are ligated, transformed, and cells grown on antibiotics to select for SM1 and SM3. Under these selection conditions, the predominant clones are the desired 2-synthon product.

Table 3 shows a selection scheme for stitching a hypothetical 8-synthon module of sequence 1-2-3-4-5-6-7-8 using Vector Pair II. Synthons 1, 4, 6, and 7 can be cloned into the vector with the SM2 marker, and 2, 3, 5, and 8 can be cloned into the vector with the SM3 marker as summarized in Table 4.

TABLE 4
SELECTION STRATEGY

| | Synthon | | | | | | | |
|---|---|---|---|---|---|---|---|---|
| | 1 | 2 | 3 | 4 | 5 | 6 | 7 | 8 |
| 1-syn | SM2 | SM3 | SM3 | SM2 | SM3 | SM2 | SM2 | SM3 |

| | | | | |
|---|---|---|---|---|
| 2-syn | SM3 | SM2 | SM2 | SM3 |

| | | |
|---|---|---|
| 4-syn | SM2 | SM3 |

| | |
|---|---|
| 8-syn | SM3 |

4.2.4 Method R: Assembly Vectors, Joining Strategies, & Selection Schemes

4.2.4.1 Joining Strategies

Method R entails the use of recognition sites for Type II restriction enzymes at the edges of the coding sequences of the synthons. Compatible (e.g. identical) restriction sites at the edges of adjacent synthons are cleaved and ligated together. For illustration and not limitation, this is diagrammed below (R1, R2 and R3=recognition sites for different Type II restriction enzymes, vvvvvvv=assembly vector region, ssssssss=synthon coding region, ooo=synthon flanking regions).

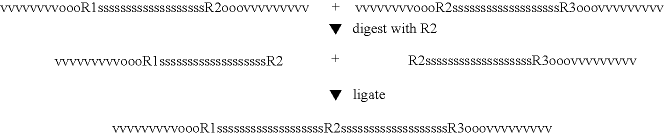

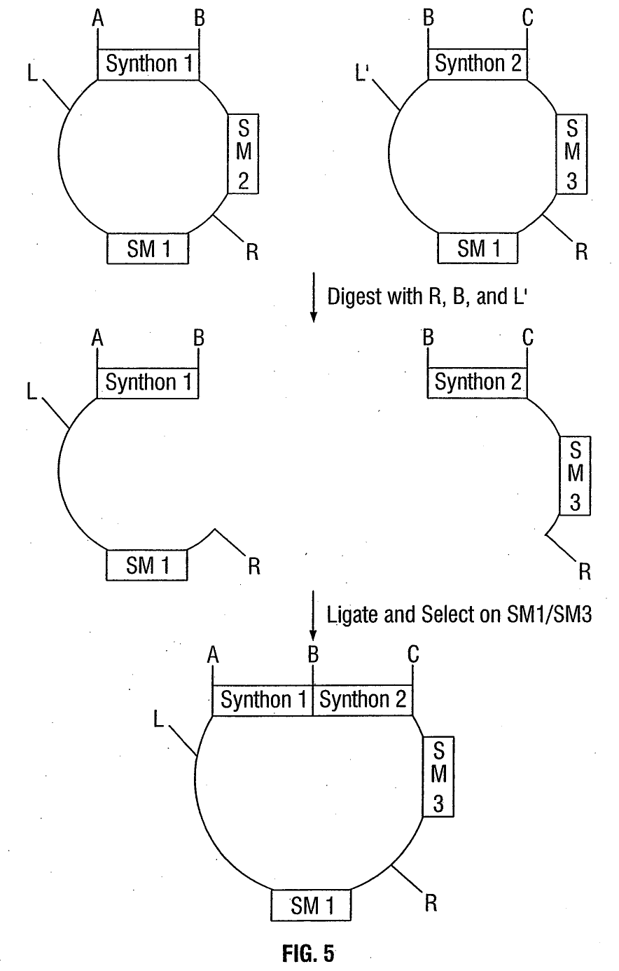

Both the association of specific synthons (depending on their position in the module) with SM2 or SM3 and the selection of restriction sites in the synthons is important. As noted above, synthons are designed with useful restriction sites at both the left and right edges of the synthons, and the sites are selected so that adjacent synthon edges share a common (or compatible) restriction site. For example, to prepare a module with a sequence 1-2-3-4-5-6-7-8 by stitching of synthons comprising the sequences 1, 2, 3, 4, 5, 6, 7, and 8, the adjacent synthon edges can share common sites B, C, D, E, F, G and H as follows: A-1-B, B-2-C, C-3-D, D-4-E, E-5-F, F-6-G, G-7-H, H-8-X. See FIG. 5.

The basis for this method is the design of synthons (and component oligonucleotides) that contain unique restriction sites at the edges of the synthon. This requires both the presence (insertion) of useful restriction sites (at the synthon edges) and absence (removal) of these sites in the interior of the synthon. Example 4 describes a strategy for identifying useful restriction sites that can be engineered at synthon and module without resulting in a disruptive change in the module amino acid sequence, and provides and exemplary results from an analysis of 140 PKS modules (see FIG. 6 and Tables 8-12). Section 5, below, describes computer implementable algorithms for the design of oligonucleotides that can be used to produce synthons with the desired patterns of restriction sites.

4.2.4.2 Assembly Vectors

Method R can be carried out using the same vector pairs as are useful for Method S. Using Method R, a Vector Pair I vector comprises a synthon cloned at the SIS site can have the following structure (where R₃ and R₄ are restriction sites at the edges of the synthon, and the other abbreviations are as described previously):

—(SM4 or SM5)-R₃-Sy-R₄—(SM2 or SM3)-R₁ [VII]

This is an intermediate construct useful for stitching.

4.2.4.3 Selection Schemes

The selection schemes described for Method S can be used for Method R. It will be appreciated that the restrictions sites at the ends of synthons must be designed so they are compatible with the digestion at vector restriction sites L and L′.

### 5. GENE DESIGN AND GEMS (GENE MORPHING SYSTEM) ALGORITHM

Design of the synthetic genes of the invention, as well as the design of oligonucleotides that can be used for gene synthesis, requires concomitant consideration of a large number of factors. For example, the synthetic module genes of the invention will encode a polypeptide with a desired amino acid sequence and/or activity, and typically
use the codon preference of a specified expression host,
are free from restriction sites that are inconsistent with the stitching method (e.g., the Type IIS sites used in stitching Method S) and/or are comprised of synthons free from restriction sites that are inconsistent with the stitching method (e.g., the Type II sites used in stitching Method R) and/or are free from restriction sites that are inconsistent with the construction of open reading frames and gene libraries (as described below),
contain useful (e.g., unique) restriction sites or sequence motifs at specific locations (e.g., region encoding domain edges, synthon edges, module boundaries, and within synthons). Without limitation, restriction sites within synthons are used for correction of errors in gene synthesis or other modifications of large genes; restriction sites and/or sequence motifs at synthon edges are used for LIC cloning (e.g., addition of UDG-linkers), stitching; restriction sites at domain edges are used for domain “swaps;” restriction sites at module edges are useful for cloning module genes into vectors and synthesis of multimodule genes. By incorporating these sites into a number of different PKS module-encoding genes, the “modules” can readily be cloned into a common set of vectors, domains (or combinations of domains) can be readily moved between modules, and other gene modifications can be made.

Challenges encountered during synthetic design of large genes include efficient codon optimization for the host organism, restriction site insertion and elimination without affecting protein sequence and design of high quality oligonucleotide components for synthesis.

A computer implementable algorithm for design of synthetic genes (and component synthons and oligonucleotides) is described in this section. A Gene Morphing System (“GeMS”) is aimed at simplifying the gene design process.

5.1 GeMS—Overview

The GeMS process was initially developed for designing PKS genes is described below. The process includes components for the design of any gene. For convenience, the GeMS process will be described with reference to a gene encoding a specified polypeptide segment. The polypeptide segment can be a complete protein, a structurally or functionally defined fragment (e.g., module or domain), a segment encoded by the synthon coding region of a particular synthon, or any other useful segment of a polypeptide of interest.

A GeMS process generically applicable to the design of any gene has several of the following features: (i) restriction site prediction algorithms; (ii) host organism based codon optimization; (iii) automated assignment of restriction sites; (iv) ability to accept DNA or protein sequence as input; (v) oligonucleotide design and testing algorithm; (vi) input generation for robotic systems; and (vii) generation of spreadsheets of oligonucleotides.

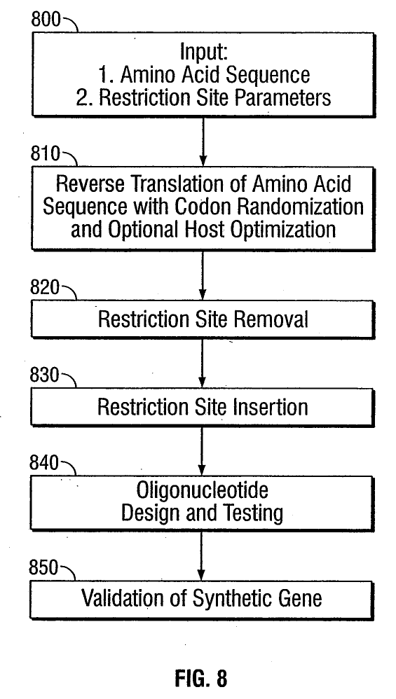

GeMS executes several steps to build a synthetic gene and generate oligonucleotides for in vitro assembly. Each of these steps are closely connected in the overall program execution pipeline. This allows the gene design to be executed in a high-throughput process as shown in FIG. 8.

Briefly, a GeMS process initiates with an input 800 of (i) an amino acid sequence of a reference polypeptide and (ii) parameters for positioning and identity of restriction sites or desired sequence motifs. In one embodiment a DNA sequence of the reference polypeptide is input and translated to the corresponding amino acid sequence. While the amino acid/DNA sequence are input from publicly available databases (e.g, GenBank), in one embodiment the sequence is verified (by independent sequencing) for accuracy prior to input in the GeMS process. In the example of FIG. 8, a GeMS process according to the present invention comprises a first series of steps 810 wherein the amino acid sequence is used as a reference to generate a corresponding nucleotide sequence which encodes the reference polypeptide (“reverse translated”). Further processes in the first series of steps include codon randomization wherein additional nucleotide sequences are generated which encode a same (or similar) amino acid sequence as the reference polypeptide using a random selection of degenerate codons for each amino acid at a position in the sequence. The process may optionally include optimization of codon usage based on a known bias of a host expression organism for codon usage. The codon-randomized DNA sequence generated by the software is further processed for introduction of restriction sites at specific location, and removal of undesired occurrences of sites in subsequent steps.

A series of steps 820 and 830 comprise restriction site removal and insertion in response to a selection of restriction sites and identification of their positions in the sequence. In one embodiment, the process uses the GeMS restriction site prediction algorithms to predict all possible restriction sites in the sequence. Based on a combination of pre-determined parameters, user input and internal decisions, the algorithm suggests optimally positioned (or spaced) restriction sites that can be introduced into the nucleic acid sequence. These sites may be unique (within the entire gene, or a portion of the gene) or useful based on position and spacing (e.g., sites useful for synthon stitching using Method R, which need not necessarily be unique). In another embodiment, an user inputs positions of preferred restriction sites in the sequence.

In a series of steps 820 the GeMS software removes occurrences of restriction sites from unwanted locations. This process preserves the unique positions of certain restriction sites in the sequence. Following removal, a third series of steps 830 inserts selected restriction sites at specific locations in the sequence. The nucleotide sequence is then divided into a series of overlapping oligonucleotides which are synthesized for assembly in vitro into a series of synthons which are then stitched together to comprise the final synthetic gene. The design of the oligonucleotides in step 840 and synthons are guided by a number of criteria that are discussed in greater detail below. Following design the oligonucleotide sequences are tested in step 840 for their ability to meet the criteria. In the event of a failure of an oligo or synthon to pass the stringent quality tests of GeMS, the entire gene sequence is re-optimized to produce a unique new sequence which is subjected to the various design stages.

Successful designs are validated in step 850 by verifying sequence integrity relative to the amino acid sequence of the reference polypeptide, restriction site errors and silent mutations. The software also produces a spreadsheet of the oligonucleotides that are in a format that can be used for commercial orders and as input to automated systems.

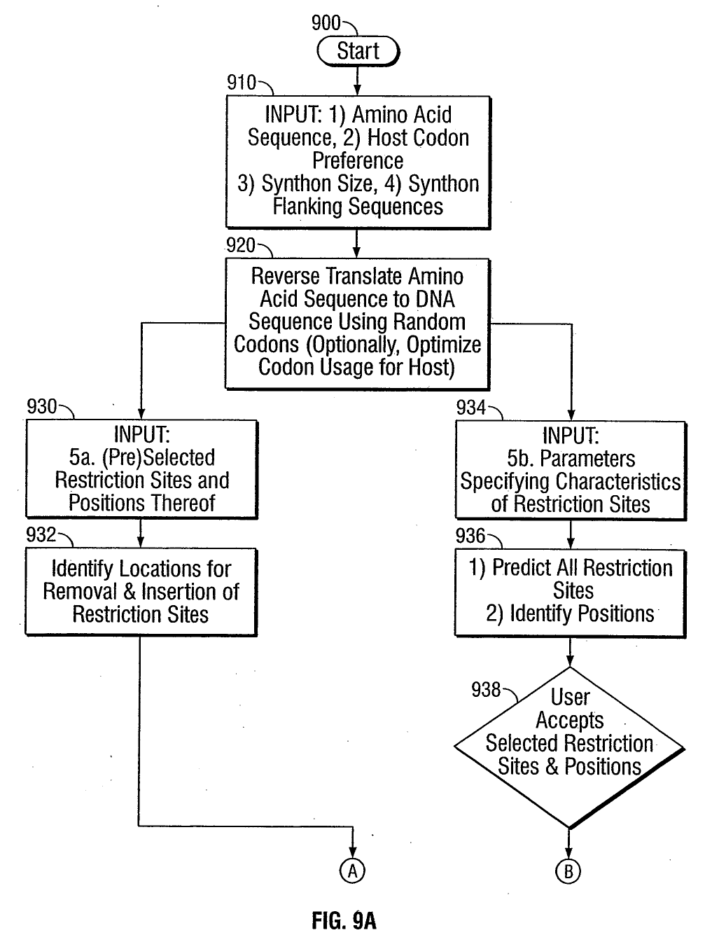

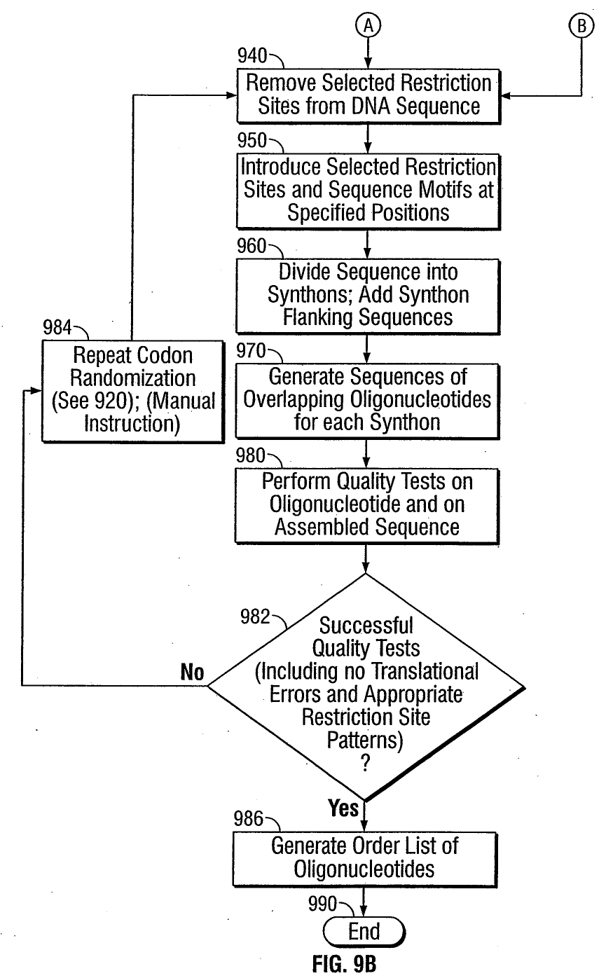

The overall scheme for synthon design by GeMS software is shown in the flow diagram of FIG. 9. The inputs 910 for the GeMS software include a file (e.g., GenBank derived information) containing the amino acid sequence of a reference polypeptide segment (or a DNA sequence encoding a polypeptide segment, usually the sequence of a naturally occurring gene). When a DNA sequence is input into GeMS, a translation of the open reading frame (ORF) to the corresponding amino acid sequence is performed. The input optionally comprises the identity of an appropriate host organism for expression of the synthetic gene and its preference for codon usage. The input may optionally include one or more lists of annotated restriction sites or other sequence motifs desired to be incorporated in the nucleotide sequence of the gene (e.g., at module/domain/synthon edges), and annotated restriction sites to be removed or excluded from the gene (e.g., recognition sites for Type IIS enzymes used in stitching). The user may input acceptable ranges of synthon sizes (typically about 300 to about 700 basepairs), number of synthons (e.g., 2n, where n=2-5), and synthon flanking sequences (e.g., sequences useful for ligation independent cloning, for example, annealing of “universal” UDG primers).

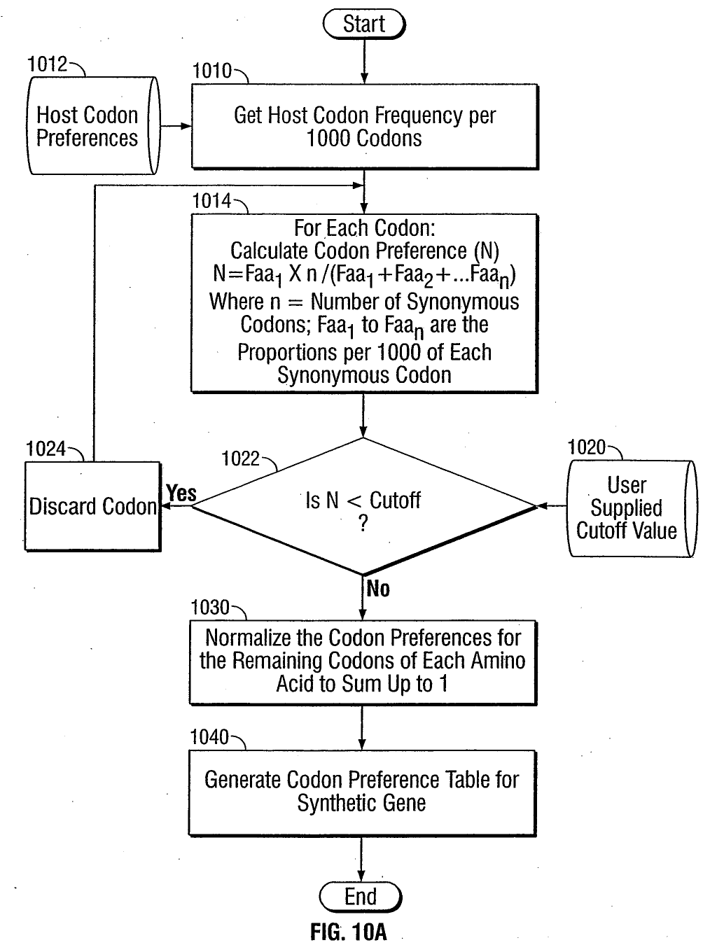

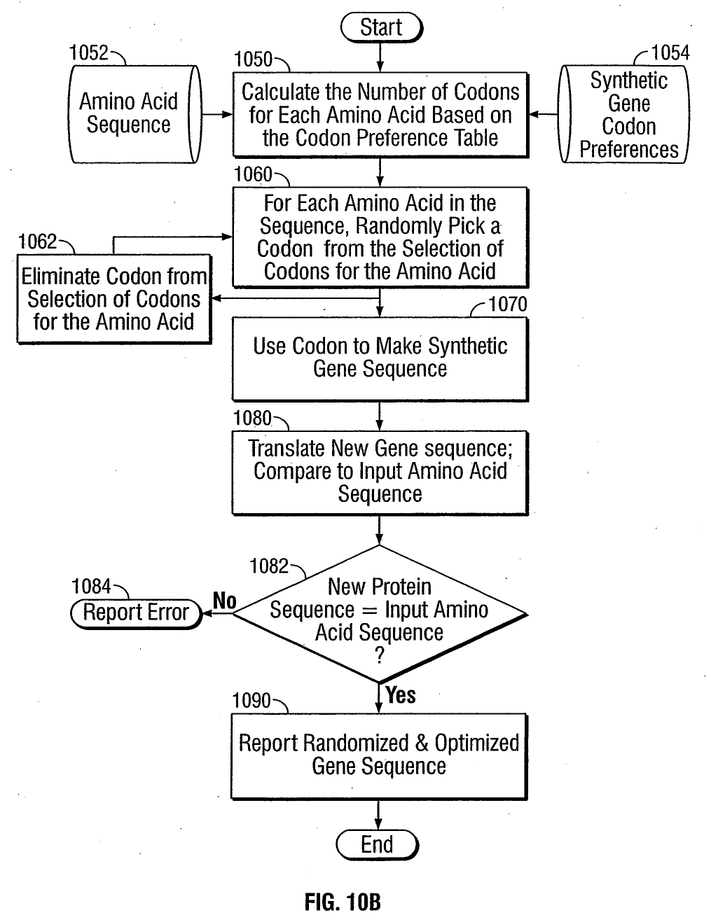

In step 920, the amino acid sequence of the reference polypeptide segment is converted (reverse-translated) to a DNA sequence using randomly selected codons, such that the second DNA sequence codes for essentially the same protein (i.e., coding for the same or a similar amino acids at corresponding positions). In one embodiment, the random choice of codons reflects a codon preference of the selected host organism. In one embodiment, the codon optimization and randomization are omitted and the DNA sequence derived from the database is directly processed in the subsequent steps. The codon randomization and optimization processes are described in greater detail in FIGS. 10A and 10B and the accompanying text.

In one embodiment, preselected restriction sites and their positions are input in step 930. In step 932, the GeMS program then identifies positions for insertions of the specified sites and identifies positions from which unwanted occurrences of specific restriction sites are to be removed. In another embodiment following step, one or more parameters for positions of restriction sites and specified characteristics of the sites are input in step 934. GeMS identifies all possible restriction sites within the sequence in step 936. The program also suggests a unique set of restriction sites according to the predetermined parameters (such as spacing, recognition site, type, etc.) in step 936. In one embodiment, the regions suggested are selected for their presence within or adjacent to synthon fragment boundaries. Common unique restriction sites or related defined sequences for modules, domain ends, synthon junctions and their positions (based on the above design principles) are identified by the program in step 936. The user accepts or rejects the suggested restrictions sites and positions in step 938. In one embodiment, the user may manually input proposed restriction sites.

In step 940 uniqueness of restriction sites at specific positions (e.g., the edges) is preserved by eliminating all unwanted occurrences of these sites in the sequence. Selected codons at specified positions are replaced with alternate codons specifying the same (or similar) amino acid to remove undesirable restriction sites.

This step is followed by insertion of selected codons at the specified positions to create restriction sites in step 950. In one embodiment, the user retains the option to include additional sites and/or to eliminate specific sites from the DNA sequence.

The DNA sequence generated following removal and insertion of restriction sites is then divided in step 960 into fragments of synthon coding regions having predetermined size and number. Synthon flanking sequences are added for determination of each synthon sequence addition of sequence motifs for addition of LIC primers, restriction sites or other motifs.

In one embodiment, specific intra-synthon sites are introduced into the DNA sequence in step 950 which are unique within the synthon. These may be used for repairs within a synthon, or for future mutagenesis. Each synthon sequence is generated as overlapping oligonucleotides of a specified length with a specified amount of overlap with its two adjacent oligonucleotides in step 970. Several factors enter into the determination of the length of the oligonucleotides and the length of the overlap (e.g., efficiency of synthesis, annealing conditions, aberrant priming, etc.). The length of the oligonucleotides may be about 10, 15, 20, 30, 40, 50, 60, 70, 80, 90 or 100 nucleotides. The length of the overlap may be about 5, 10, 15, 20, 25, 30, 35, 40 or 50 nucleotides. the lengths of the overlap may not be precise and a variation by 1, 2, 3, 4 or 5 between several oligonucleotides comprising adjacent synthons is acceptable. In one embodiment, each synthon is designed as oligonucleotides of overlapping 40-mers with about a 20 base overlap among adjacent oligonucleotides. The overlap may vary between 17 and 23 nucleotides throughout the set of oligonucleotides. An option to design these oligonucleotides based on an uniform annealing temperature is also available.

As discussed in detail below, each set of oligonucleotides used for synthesis of a synthon (synthon coding region and synthon flanking sequence) can be subjected to one or more quality tests in step 980. The oligonucleotides are tested under one or more criteria of primer specificity including absence of secondary structure predicted to interfere with amplification, and fidelity with respect to the reference sequence. As discussed below, validation is also carried out for the assembled gene.

Any failures trigger a user-selected choice of two strategies in step 982: 1) repeat the random codon generation protocol 984 and continue the process from codon removal 940 and insertion 950; and/or 2) manually adjust the sequence to conform better to the predetermined parameters in the problematic region in step 984. The process may be repeated (starting with the codon optimization and randomization step 920) for a particular synthon that does not pass the test or may be run de novo for the entire polypeptide segment sequence. The candidate oligonucleotide sequences generated by this process are in turn tested again. When an entire set of oligonucleotides for 10 to 12 synthon sequences has been successfully generated, the entire candidate module sequence can be checked in any way desired (repeats, etc.), with the possibility of triggering redesign of individual synthons. Optionally, duplicated regions are removed although the random choice procedure makes occurrence of substantial repeats unlikely. Optionally, the software also edits the sequence to remove clustered positioning of rare codons. Since each redesign uses a random set of codons, synthon fragments pass these tests in relatively few iterations.

Once all fragments have passed the tests, GeMS reassembles the fragments in predetermined order and validates the restriction sites and DNA sequence by comparison with the original input sequence. This integrity check ensures that the target sequence is in accord with the intended design and no unwanted sites appear in the finished DNA sequence. Implementation of the method of FIG. 9 allows the oligonucleotides for each fragment to be saved in separate files representing each synthon or as a complete set representing the synthetic gene. The software can also produce spreadsheets of the oligonucleotides in step 986 that are in a format that can be used for commercial orders, and as input to the robots of an automated system. Spreadsheets input to an automated system can include (a) oligonucleotide location (e.g., identity such as barcode number of a 96-well plate and position of a well on the plate); (b) name or designation of oligonucleotide; (c) name or designation of module(s) synthesized using oligonucleotide; (d) identity of synthon(s) synthesized using oligonucleotide (identifying those oligonucleotides to be pooled for PCR assembly); (e) the number of synthons within the module; (f) the number of oligonucleotides within the synthon; (g) the length of the oligonucleotide; (h) the sequence of oligonucleotide. The entire gene design process involving user interaction can be achieved in a few minutes. GeMS achieves end to end integration using a high-throughput pipeline structure. In one embodiment, GeMS is implemented through a web browser program and has a graphical interface.

At least one set of rules to guide the design process are input and stored in the memory of the system. The design software operates by means of a series of discrete and independently operable routines each processing a discrete step in the design system and comprised of one or more sub-routines.

These functions are described in greater detail below. Successful designs are rechecked for sequence integrity, restriction site errors and silent mutations.

5.2 GeMS Algorithms

A method in accordance with the present invention comprises algorithms capable of performing one or more of the following subroutines:

1. Codon Randomization and Optimization—GeMS uses codon randomization and optimization sub-routines a schematic example of which are shown in FIGS. 10A and 10B. In one embodiment the optimization-randomization program can be bypassed with a manual selection of codons or acceptance of the natural nucleotide sequence.

A codon optimization process shown in the schematic of FIG. 10A starts with an input 1010 of host codon frequencies (Faa=frequency per 1000 codons) of different amino acids from a codon preference database 1012 of a selected host organism. Then the codon preference (N) for each codon is calculated in step 1014. In one known codon optimization routine (CODOP) the codon preference N is calculated as follows: N=Faa₁×n/(Faa₁+Faa₂+Faa₃ . . . +Faaₙ), where n is the number of synonymous codons (codons for the same amino acid) and Faa₁ to Faaₙ are the proportions per 1000 codons of each synonymous codon. (see Withers-Martinez et al., 1992, Protein Eng 12:1113-20.) A cut-off value for codon optimization is selected by an user in step 1020. In one embodiment, the value is 0.6. The cut-off value can vary based on the GC-richness of the host expression system or can be different for each amino acid based on metabolic and biochemical characteristics. The rationale is to choose a cut-off value that eliminates most rare codons. In one embodiment, this is done by visual inspection of the modified codon tables and selecting a cut-off value that eliminates most rare codons without affecting the preferred codons. Each codon is tested for a codon preference value above the cut-off value in step 1022. All codons with N below the user-defined cut-off value are rejected in step 1024. For each amino acid, codons with N values above the cut-off value are pooled and the N values normalized in step 1030 such that the sum of the N values is one (1). A codon preference table for the synthetic gene is generated in step 1040.

Use of the optimized codons in generating a randomized and optimized synthetic gene sequence is shown in the schematic of FIG. 10B. For an input amino acid sequence 1052, the number of codons for each amino acid is calculated in step 1050 based on the synthetic gene codon preference table 1054. For each amino acid in the sequence 1052, a codon is randomly picked in step 1060 from the selection of optimized codons for the amino acid. The randomly selected codon is used to generate a new synthetic gene sequence in step 1070. Each time a codon is used in the synthetic gene sequence it is eliminated in step 1062 from the selection of optimized codons for the amino acid in the synthetic gene codon preference table 1054. The synthetic gene sequence is validated by comparison of its translated amino acid sequence with the input amino acid sequence in step 1080. If the sequences are identical 1082, the randomized and optimized synthetic gene sequence is reported in step 1090. If the sequences are not identical, the errors in the synthetic gene sequence are reported in step 1084. In one embodiment, the user has the option to accept a substitution of a similar amino acid. In another embodiment, the errors are analyzed for implementation in correcting subsequent randomization routines.

2. Restriction site prediction—In one embodiment, a restriction enzyme prediction routine is performed at this stage. The restriction site prediction routine predicts all restriction sites in a nucleotide sequence for all possible valid codon combinations for the corresponding amino acid sequence. The program automatically identifies unique restriction sites along a DNA sequence at user-specified positions or intervals. This routine is used in the initial design of the modules and/or synthons and optionally in checking errors in the predicted sequences.

Following execution of these routines the user indicates acceptance of the output according to one embodiment. If the list of restriction sites generated are accepted by the user, the process is transferred to the GeMS codon-optimization routine. If the result is not acceptable to the user, the sub-routine is repeated while allowing the user to modify the parameters manually. The process is repeated until a signal indicating acceptance is received from the user. After the user accepts the restriction sites, the sequence is transferred to the next routine in the GeMS module to perform the subsequent procedures.

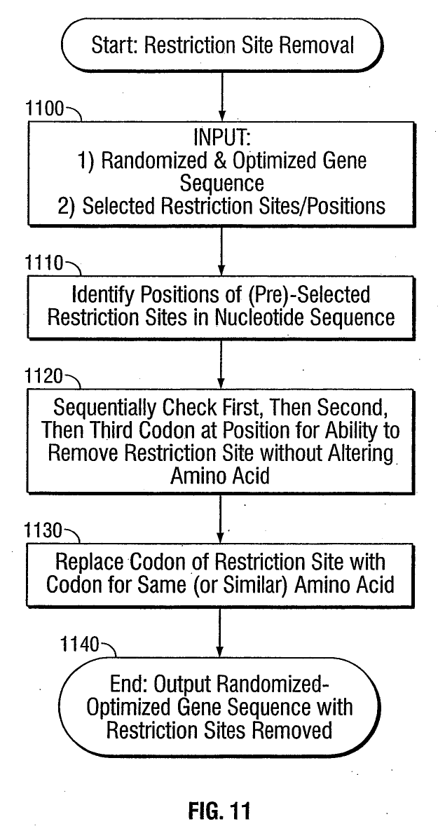

3. Removal of Restriction Sites—Restriction sites that are selected in steps 932 or 938 of the GeMS program (see FIG. 9) are cleared from the codon optimized gene sequence as shown schematically in FIG. 11.

A sub-routine of the present process removes selected restriction sites that are specified and input 1100 with the randomized-optimized gene sequence. The sub-routine identifies the pre-selected restriction sites in the codon-optimized gene sequence and identifies their positions in step 1110. At each given position the open-reading frames comprising the recognition site are examined for the ability to alter the sequence and remove the restriction site without altering the amino acid encoded by the affected codon at the restriction site in step 1120. If the reading frame is open, the first codon of the recognition site is replaced with a codon encoding the same or a similar amino acid in a manner that removes the restriction site sequence. If however, the first codon is unsuitable for replacement, the sub-routine shifts to the next available codon and continues until the restriction site is removed. Since a restriction site may encompass up to 6 nucleotides, removal of a site may involve analysis of up to three amino acid codons. Removal of restriction sites is performed in a manner which retains the identity of the encoded amino acid in step 1130. The sub-routine generates a randomized-optimized gene sequence from which selected restriction sites have been removed without altering the amino acid sequence 1140.

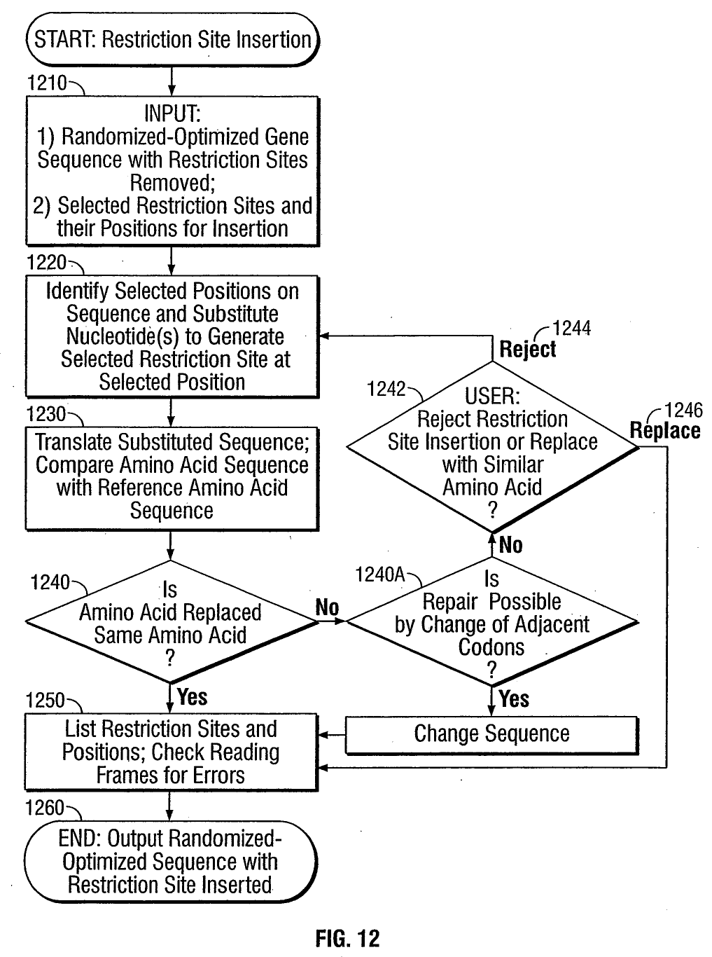

4. Insertion of Restriction Sites—The next sub-routine performed by the process introduces restriction sites. This step substitutes nucleotide bases at selected positions to generate the recognition sites of selected restriction enzymes without altering the amino acid sequence as shown in the schematic of FIG. 12. In this sub-routine a randomized-optimized gene sequence from which selected restriction sites have been removed is input along with selected restriction sites and their positions for insertion into the sequence in step 1210. The selected insertion positions are identified in the sequence and nucleotide(s) are substituted to generate in step 1220 the selected restriction site at the selected position. In one embodiment, only the sequence of an overhang created by a restriction site is inserted instead of a restriction site. When a such sequence is present in the synthon, it can be cleaved remotely by a Type IIS restriction enzyme and the overhang thus generated is available for ligation with a DNA fragment which has been cleaved with a Type II restriction enzyme to generate the complementary overhang. The substituted sequence is translated and the resulting amino acid sequence is compared in step 1230 with the sequence of the reference amino acid (see 1052 in FIG. 10B). The substituted sequence is translated and the resulting amino acid sequence is compared in step 1230 with the sequence of the reference amino acid (see 1052 in FIG. 10B), comparing the sequences for identity of the amino acid sequences. If in step 1240, the amino acid specificity of a codon overlapping the substituted sequence is found to be changed, the codon table may be reexamined in step 1240A for codons compatible with both the amino acid sequence and the substituted sequence, and compatible with the desired pattern of restriction sites and sequence motifs or other patterns. If any compatible codons are found, one is chosen from the list of such codons according to user preference (for example, by use of relative probabilities in a codon table), and inserted as replacement for the undesired codon; the program returns to step 1240. If the amino acid sequence is altered, and not repairable by the procedure described in step 1240A, the program proceeds to step 1242. The user in step 1242 has the option of rejecting the output in step 1244 and repeating the process of nucleotide substitutions at the selected position. In one embodiment the user replaces in step 1246 an amino acid with a similar amino acid and manually accepts the output. The sequence generated following introduction of the restriction sites is then checked for translational errors in step 1250. A randomized-optimized synthetic gene sequence with selected restriction sites removed and other selected restriction sites inserted is provided in step 1260. As noted above, sequence motifs other than restriction sites can be “inserted” or “removed” (i.e., the oligonucleotides, synthons and genes can be designed to include or omit the sequence motifs from particular locations). For example, regions of sequence identity are useful for construction of multisynthons (see, e.g., Exemplary Construction Method 2 in Section 6.4.3, below) and can be included at specified locations of synthetic genes).

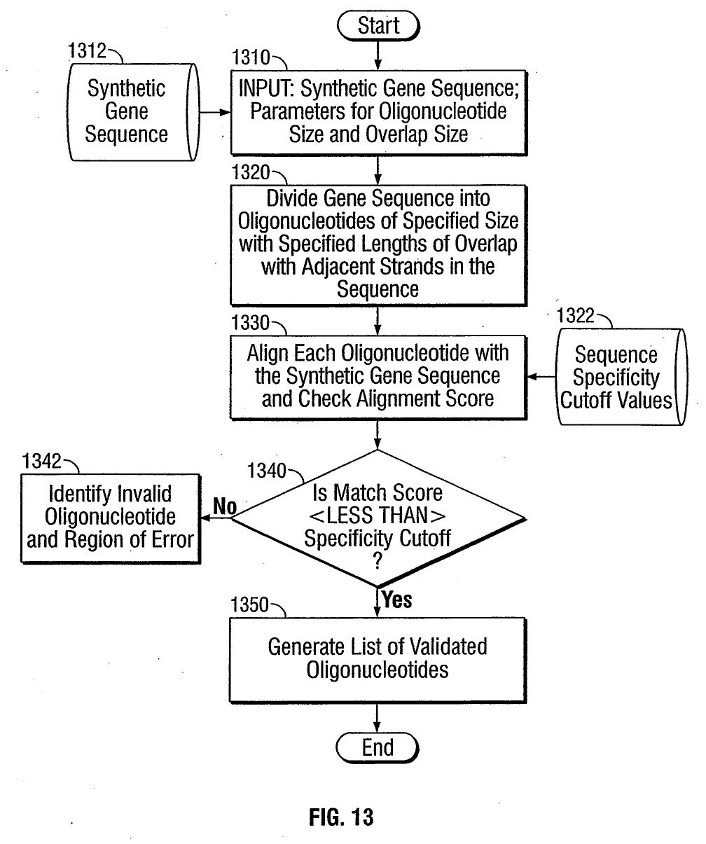

5. Generation of Oligonucleotides to Comprise Synthetic Genes or Synthons—The input to GeMS has each of the restriction sites tagged as either a domain edge or synthon edge along with their positions. Based on these criteria, this step 1320 (see FIG. 13) of the program pipeline divides the entire gene sequence into a number of synthons in one embodiment. In another embodiment, a preferred synthon size is input. Overlapping oligonucleotide sequences are generated in step 1320 to comprise the synthon coding region as well as the synthon flanking sequences.

The generation of oligonucleotides for a synthetic gene is shown in the schematic of FIG. 13. A synthetic gene sequence 1312 is input along with parameters in step 1310 specifying lengths of oligonucleotides and the extent of overlap between adjacent oligonucleotides. The synthetic gene sequence is divided in step 1320 into a plurality of oligonucleotide sequences of specified length with overlaps allowing a selected number of bases to pair with adjacent strands. Each oligonucleotide is aligned with the synthetic gene sequence 1312 and the extent of alignment is determined in step 1330. The extent of alignment (match score) is compared in step 1332 to a predetermined sequence specificity cutoff value for acceptable degree of alignment. A decision is made based on the match of the sequences in step 1340. If the match score is less than the specificity cutoff value the invalid oligonucleotide is identified and the errors are identified in step 1342. The output may be discarded or adjusted manually. In one embodiment, the lengths of the oligonucleotides are increased or decreased to adjust the overall extent of alignment of the oligonucleotide. If the match score exceeds the specificity cutoff, a list of validated oligonucleotides are generated.

In one embodiment, the synthetic gene is a synthon. Oligonucleotides comprising a synthon include oligonucleotides specific for the synthon coding region as well as the synthon flanking sequences. Each synthon is comprised of oligonucleotides designed as a set of oligonucleotides each having overlaps of complementary sequences with its two adjacent oligonucleotides on either side. The selection of the length of oligonucleotides take into account several factors including, the efficiency and accuracy of synthesis of oligonucleotides of specific lengths, the efficiency of priming during assembly PCR, annealing temperatures and translational efficiency. In a preferred embodiment, a 40-mer size of each oligonucleotide is selected with an overlap of about 20 nucleotides with adjacent oligonucleotides. Each oligonucleotide is designed as two approximately equal halves (in this instance, two 20-mer sections), wherein each half must meet the criteria for interactions (e.g., annealing, priming) with the two adjacent oligonucleotides that overlap with either half. the selection of a 40-mer sequence further reflects the accuracy of chemical synthesis of oligonucleotides of that length.

While the present invention relates to assembly of the overlapping oligonucleotides by a PCR reaction, it is contemplated that the oligonucleotides may be assembled enzymatically by a combination of DNA ligase and DNA polymerase enzymes. In such an embodiment, longer oligonucleotides may be used with shorter overlaps. It is contemplated that the overlaps may leave gaps of 5, 10, 15, 20 or more nucleotides between the regions of an oligonucleotide that are complementary to its two adjacent oligonucleotides. Such gaps can be repaired by a DNA polymerase enzyme and the synthon comprised by the oligonucleotides can then be assembled by a DNA ligase mediated reaction.

6. Oligonucleotide Design Criteria: The design of suitable oligonucleotide sets are based on a number of criteria. Two criteria used in the design are annealing temperature and primer specificity.

6A. Optimum Annealing Temperature: User-defined ranges for annealing temperature (preferably 60-65° C.) and oligonucleotide overlap length are input. To increase temperature, the size of the oligonucleotide overlap length is increased and vice-versa. The GeMS program designs the oligonucleotides within specified annealing temperature boundaries. The criterion is an uniform (preferably, narrow range of) annealing temperature for the entire set of oligonucleotides that are to be assembled by a single PCR reaction. Annealing temperature is measured using the nearest neighbor model described by Breslauer (Breslauer et al., 1986 “Predicting DNA Duplex Stability from the Base Sequence.” Proceedings of the National Academy of Sciences USA 83:3746-3750.) and Baldino (Baldino, 1989, “High Resolution In Situ Hybridization Histochemistry” in Methods in Enzymology, (P. M. Conn, ed.), 168:761-777, Academic Press, San Diego, Calif., USA.). An additional method for narrowing the melting temperature range of designed oligonucleotide duplexes, by automatically adding or removing bases from oligonucleotide components, is also implemented.

6B. Primer Specificity:—Each of the overlapping oligonucleotide sequences generated for each synthon (or synthetic gene) is subjected to primer specificity tests against the entire synthon. In order to ensure optimal priming, each of the oligonucleotide sequences in a synthon are tested by alignment against the entire synthon sequence. Alignment is determined by comparing the numbers of matches and mismatches between the oligonucleotide sequence and the sequence of the synthon. Oligonucleotides that align with a degree of alignment higher than a predetermined value are selected for synthesis. In one embodiment, this is performed by aligning the oligonucleotide sequence against the synthon sequence starting at position 1 and sliding it across the length of the synthon sequence one base at a time.

In one embodiment, an oligonucleotide sequence is determined to be unsuitable for use according to the following series of steps:

Step I: align the last three (3) bases of both the oligonucleotide sequence and synthon reference sequence such that they are identical;

Step 2: count the number of matches and mismatches in the aligned sequences with matches being identical bases in both sequences at the same position;

Step 3: calculate the ratio of matches to the total number of bases forming the overlap or alignment.

If the ratio is greater than a user-defined threshold value of 0.7 (or 70%) the oligonucleotide is suitable for synthesis. In one embodiment, oligonucleotides whose threshold value fall lower than the user-defined value can be subjected to manual modification of its sequence to increase the extent of alignment and meet the threshold requirement.

7. Oligonucleotide Quality Testing: The software checks for any undesired degree of aberrant priming among the oligonucleotides of each synthon. If present, it repetitively redesigns synthons in which this occurs until the design is improved. In difficult cases, it reports the results and prompts user to manually repair the errors.

8. Input Validation Routines: One or more user input validation routines can be implemented to run independently in parallel with the synthon design routines. These perform validation checks on instructions input by the user. These routines validate instructions typically input by a user during a step of the GeMS process and include validation of restriction site positions based on the site prediction algorithm, frame shifts and synthon boundaries. Identification of errors at the input stage prevents the user from providing any input that results in a faulty design.

9. Output Validation Routine—A program output validation routine can be used to reduce the time to validate the designed synthons. This allows the end-to-end design process to operate in a high-throughput manner. This program reassembles the designed synthons while maintaining the correct order and recreates a synthetic gene. The new synthetic gene is then translated to its amino acid sequence and compared with the original input protein sequence for possible errors. The restriction site pattern for the assembled sequence is verified as being the one desired. The restriction site pattern for each designed synthon (including the synthon-specific primers) is verified as well. Other quality tests can be preformed, including tests for undesired mRNA secondary structure and undesired ribosome start sites.

10. User Interface. An optional web-based software implementation provides a graphical interface which minimizes the number of steps needed to complete a design. Where applicable the user is provided on-screen links to web sites and/or databases of gene sequences, gene functions, restriction sites, etc. that aid in the design process.

This concludes the pipeline and outputs a list of suitable oligonucleotides for each synthon of the synthetic gene.

5.3 Software Implementation

In one embodiment, the GeMS software is implemented to execute within a web-browser application making it a platform-neutral system. Its design is based on the client-server model and implemented using the Common Gateway Interface (CGI) standard.

All CGI scripts and the application programming interface (API) for GeMS was implemented in Python version 2.2. Development, testing and hosting of the application was performed on a 1.0 GHz Intel Pentium III based processor server running RedHat Linux version 7.3. The web interface runs on the Apache HTTP Server version 2.0.

The annealing temperature module in the GeMS API utilizes the EMBOSS software analysis package (Rice, P. Longden, I. and Bleasby, A., 2000, “EMBOSS: The European Molecular Biology Open Software Suite” Trends in Genetics 16:276-77) and implements the nearest neighbor model described by Breslauer (Breslauer et al., 1986, Proc. Nat'l Acad. Sci. USA 83:3746-50) and Baldino (Baldino Jr., 1989, In Methods in Enzymology 168:761-77).

Publicly available software such as DNA Builder (Bu et al., “DNA Builder: A Program to Design Oligonucleotides for the PCR Assembly of DNA Fragments.” Center for Biomedical Inventions, University of Texas Southwestern Medical Center), DNAWorks (David M. Hoover and Jacek Lubkowski, 2002. “DNAWorks: an automated method for designing oligonucleotides for PCR-based gene synthesis.” Nucleic Acids Research 30, No. 10, e43), and CODOP (Withers-Martinez et al., 1999. “PCR-based gene synthesis as an efficient approach for expression of the A+T-rich malaria genome.” Protein Eng 12: 1113-20) can be configured by the skilled practitioner to accomplish some (but not all), of the tasks used by GeMS for automated design of polyketide modules.

In one aspect, the invention provides a computer readable medium having computer executable instructions for performing a step or method useful for design of synthetic genes as described herein.

### 6. MULTIMODULE CONSTRUCTS AND LIBRARIES

6.1 Introduction

Synthetic genes designed and/or produced according to the methods disclosed herein can be expressed (e.g., after linkage to a promoter and/or other regulatory elements). In one aspect of the invention, a synthetic gene is linked in a single open reading frame with another synthetic gene(s) to encode a “fusion polypeptide.” It will be recognized that the DNA encoding the fusion polypeptide is itself a synthetic gene (generated from the linkage of smaller genes). In a related aspect, multiple different open reading frames can be co-expressed (or their protein products combined in vitro) to form multiprotein complexes. This is analogous to naturally occurring polyketide synthases, which are complexes of several polypeptides, each containing two or more modules and/or accessory units.

Thus, in the context of production of polyketides, the present invention contemplates

(A) producing synthetic genes that encode polypeptides comprising combinations of PKS modules and/or accessory units;

(B) expressing two or more different polypeptides of (A) which associate with each other to form a multipolypeptide complex.

Methods for producing polypeptide-encoding synthetic genes comprising combinations of PKS modules and/or accessory units include by designing and stitching together synthons that together encode a gene-encoding the combination, using methods discussed above, (e.g., in Section 4). Alternatively, two or more synthetic genes that can encode different portions of the single polypeptide may be joined by conventional recombinant techniques (including ligation independent methods and linker-mediated methods, and other methods) using sites or sequence motifs located (e.g., engineered) at particular locations in the gene sequences (e.g., in regions encoding termini of modules, domains, accessory units, and the like). One important new benefit of the design and synthetic methods of the present invention is the ability to control gene sequences to facilitate the cloning of modules, domains, etc. A particularly useful ramification of these methods is the ability to make multiple large libraries of genes encoding structurally or functionally similar units (for example modules, accessory units, linkers, other functional polypeptide sequences), in which restriction sites or other sequence motifs are located an analogous positions of all members of the library. For example, a PKS module gene can be synthesized with unique restriction sites at the termini (e.g., Xba I and Spe I sites) facilitating cloning into the same sites in a vector.

In a related aspect, the invention provides multiple large libraries genes encoding polypeptides comprising regions (linkers) that allow the polypeptides to associate with other polypeptides encoded by members of the library or by members other libraries.

In a related aspect, the invention provides, for example, vectors and vector sets that can be used for manipulation, expression and analysis of numerous different polypeptide segment-encoding genes. For example, the invention provides useful vectors (referred to as ORF vectors) that facilitate preparation of libraries of genes encoding multimodule constructs.

The following sections describe exemplary methods for making and using vectors and vector libraries comprising ORFs encoding PKS modules and accessory units. Section 6.2, below describes how libraries can be used to analyse interactions between modules and other polypeptide units. This section is intended to illustrate how libraries can be used, and make the description of library construction more clear. Section 6.3 discusses module and linker combinations. Section 6.4 describes certain ORF vectors and methods for constructing them.

6.2. Exemplary Uses of ORF Vector Libraries

In one aspect, the invention provides methods for expression of PKS module-encoding genes in combinations not found in nature. Such novel module architecture enables production of novel polyketides, more efficient production of known polyketides, and further understanding of the “rules” governing interactions of PKS modules, domains and linkers. Combinations of “heterologous” modules (i.e. modules that do not naturally interact) may not be productive or efficient. For example, at a heterologous module interface, the product of the first module may not be the natural substrate for the second or subsequent modules and the accepting module(s) may not accept the foreign substrate efficiently. In addition, inter-module transfer of the polyketide chain (from the ACP thiol ester of one module to the KS thiol ester of the next) may not occur efficiently. See U.S. Patent Publication No. US20030068676A1: Methods to mediate polyketide synthase module effectiveness. The present invention provides methods for vectors, libraries, and methods for evaluating the ability of modules, domains, linker and other polypeptide segments to function productively.

In one aspect of the invention, libraries of vectors are prepared in which different members of the library comprise different extension modules. In one aspect of the invention, libraries of vectors are prepared in which the members of the library comprise the same extension module(s) but comprise different accessory units (e.g., different loading modules and/or different linker domains and/or different thioesterase domains). Thus, the invention provides methods for synthesizing an expression library of PKS module-encoding genes by: making a plurality of different synthetic PKS module-encoding genes (e.g., as described herein) and cloning each gene into an expression vector. In one embodiment, the library includes at least about 50 or at least about 100 different module-encoding genes. In one aspect of the invention, such libraries are used in pairs to identify productive interactions between pairs or combinations of PKS modules.

For illustration, one application of libraries of the present technology can be illustrated by describing two (of many possible) ORF vector libraries. The skilled practitioner, guided by this disclosure, will recognize a variety of comparable or analogous libraries that can be made and used. A first ORF library comprises vectors comprising an open reading frame encoding a loading domain (LD), a PKS module (Mod), and a left linker (LL) and where different members of the library encode the same LD and LL, but different modules, i.e.:

[LD-Mod-LL]ₙ [Exemplary Library I]

where n is usually >20. A second ORF library comprises vectors comprising an open reading frame encoding a right linker (RL), a module (Mod), and a thioesterase domain (TE), where different members of the library encode different modules, i.e.:

[RL-Mod-TE]ₙ [Exemplary Library II]

The terms “right linker” (RL) and “left linker” (LL) refer to interpolypeptide linkers that allow two polypeptides to associate. For construction of polyketide synthases which contain more than one polypeptide, the appropriate sequence of transfers can be accomplished by matching the appropriate C-terminal amino acid sequence of the donating module with the appropriate N-terminal amino acid sequence of the interpolypeptide linker of the accepting module. This can be done, for example, by selecting such pairs as they occur in native PKS. For example, two arbitrarily selected modules could be coupled using the C-terminal portion of module 4 of DEBS and the N-terminal of portion of the linking sequence for module 5 of DEBS. Alternatively, novel combinations of linkers or artificial linkers can be used.

In one embodiment, for illustration, each of the two libraries shown contains four members, each member containing a gene encoding a different module, i.e., module A, B, C or D (“ModA,” “ModB,” “ModC,” “ModD”). Using a library of the 8 exemplary vectors shown below, all possible combinations of Modules A, B, C and D (“ModA,” “ModB,” “ModC,” “ModD”) can be tested for functionality after transfer to appropriate expression vectors.

| | |
|---|---|
| LD-ModA-LL | RL-ModA-TE |
| LD-ModB-LL | RL-ModB-TE |
| LD-ModC-LL | RL-ModC-TE |
| LD-ModD-LL | RL-ModD-TE |

To test for functionality of combinations of modules (e.g., pairwise combinations) from Library I and Library II can be co-transfected into a suitable host (e.g., E. coli engineered to support PKS post-translational modification and substrate Co-A thioester production) and product triketides may be analyzed by appropriate methods, such as TLC, HPLC, LC-MS, GC-MS, or biological activity. Alternatively the library members may be expressed individually and Library I-Library II combinations can be made in vitro. Affinity and/or labelling tags may be affixed to one or both termini of the module constructs to facilitate protein isolation and testing for activity and physical interaction of the module combinations.

When productive combinations are identified, the productive pair can be combined and tested in new pairwise combinations. For example, if LD-ModA-LL+RL-ModD-TE was productive, the construct LD-ModA-ModD-LL could be synthesized and tested in combination with members of Library II. Similarly, a third library, containing [LL-Mod-RL]ₙ constructs, can be used. A number of other useful libraries made available by the methods of the present invention will be apparent to the practitioner guided by this disclosure. In a complementary strategy, the interactions of accessory units and modules can be assessed by keeping the module gene constant and varying the accessory units (e.g., using a library in which different members encode the same extension module(s) but different loading modules or linkers).

It will be apparent that gene libraries can be used for uses other than identification of production protein-protein interactions. For example, members of the ORF libraries described herein can be used for production, as intermediates for construction of other libraries, and other uses.

6.3 Module and Linker Combinations

This section describes in more detail how module genes can be expressed with native or heterologous linker sequences. As is described below, useful fusion proteins of the invention can include a number of elements. Examples include:

| construct # | structure |
|---|---|
| 1. | LD-Mod1-LL |
| 2. | LD-Mod2-LL_{H} |
| 3. | RL-Mod3-TE |
| 4. | RL_{H}-Mod4-TE |
| 5. | RL-Mod5-Mod6-LL |
| 6. | LD-Mod7-*-Mod8-LL |

where, “LD” refers to a PKS loading module, “TE” refers to a thioesterase domain; “RL” and “LL” refer to PKS interpolypeptide linkers, subscript “H”_{H} means a “heterologous” linker, “*” indicates that a heterologous AKL (ACP-KS Linker, see definitions, Section 1) is present, and “Mod” refers to various PKS modules. The modules can differ not only with respect to sequence and domain content, but also with regard to the nature of the interpolypeptide and intermodular linkers. A general discussion of PKS linkers is provided in Section 1, above, and the references cited there. Briefly, PKS extension modules in different polypeptides can be linked by “interpolypeptide” linkers (i.e., RL and LL) found (or placed) and multiple PKS extension modules in the same polypeptide can be linked by AKLs.

Extension modules used in the constructs can correspond to naturally occurring modules located at the amino terminus of a naturally occurring polypeptide or other than the amino-terminus, and be placed at the amino terminus of a polypeptide encoded by a synthetic gene (e.g., Mod3) or other than the amino-terminus (e.g., Mod 6).

It will be apparent to one of ordinary skill in the art that in an ORF comprising a synthetic gene encoding a module, the module can be joined to a variety of different linkers. For example, a module corresponding to a naturally occurring module can be associated with a sequence encoding an interpolypeptide or other intermodular linker sequence associated with the naturally occurring module, or can be associated with a sequence encoding an interpolypeptide or other intermodular linker sequence not associated with the naturally occurring module (e.g., a heterologous, artificial, or hybrid linker sequence). It will be apparent that depending on the final construct desired, a synthetic module may or may not include the AKL of the corresponding naturally occurring module. Conveniently, Spe I and Mfe I sites optionally placed in a synthetic module-encoding gene or library of genes of the invention can be used to add, remove or swap AKLs for replacement with different AKLs.

6.4 Exemplary Orf Vector Constructs

As noted above, modules may be cloned into “ORF (open reading frame) vectors,” for construction of complex polypeptides. Although a number of alternative strategies will be apparent, it is generally convenient to have specialized vectors serve different roles in the synthesis and expression of synthetic genes. For example, in one embodiment of the invention, synthon stitching is carried out in one vector set (e.g., assembly vectors), genes encoding modules and/or accessory units are combined in a different set of vectors (e.g., ORF vectors), polypeptides are expressed in a third set of vectors (expression vectors). However, a other strategies will be apparent to the reader guided by this disclosure. For example, ORF vectors of the invention can be configured to also serve as expression vectors.

It is often convenient, when cloning from assembly vectors to ORF vectors to use assembly vectors that include useful restriction sites flanking the multisynthon of the assembly vector. Accordingly, useful assembly vectors may contain restriction sites in addition to those described in Section 4 positioned on either side of the SIS (and thus on either side of the module contained in the occupied assembly vectors). Since these flanking restriction sites (“FRSs”) are usually absent from the sequences synthetic module genes (i.e., “removed” during gene design) it is generally advantageous to use rare sites (e.g., 8-bp recognition sites).

In the descriptions of the methods described below, the following abbreviations are used for illustration only: 1=Nde I site, 2=Xba I site, 3=Pac I site, 4=Not I site, 5=Spe I site, 6=Eco RI site, 7=Bbs I site, 8=Bsa I site, *=a common sequence motif. When considering the illustrations below it is important to keep in mind that useful vectors are not limited to those with the specific restriction sites shown. For example, any of the sites shown can be substituted for by using a different site (able to function in the same manner). For example, any of a large numbers of sites recognized by Type IIS enzymes can be used for sites 7 and 8; any of a variety of sites can be used for sites 3 and 4, although rare sites (e.g., with 7 or 8 basepair recognition sequences) are preferred. Similarly, any number of sites can be used in place of Xba I and Spe I, provided that compatible cohesive ends are generated by digestion of the sites (and preferably, neither site is not regenerated upon ligation of the cohesive ends). Further, although all of these sites are useful, not all are required for the present methods, as will be apparent to the reader of ordinary skill. In many embodiments one of more of the sites is omitted. In the discussions below, a multisynthon transferred from an assembly vector to an ORF vector is sometimes referred to as, simply, a “module.”

6.4.1 ORF Vectors Comprising Amino- and- Carboxy Terminal Accessory Units or Other Polypeptide Sequences

To synthesize a multimodule gene construct, an ORF vector having the following structure can be used for manipulation:

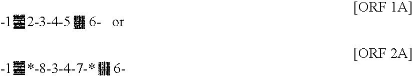

where [symbol] and [symbol] indicate a nucleotide sequence encoding a structural or functional polypeptide segment such as a non-PKS polypeptide segment (e.g., NRPS modules) or PKS accessory unit. For example, [symbol] can be a gene sequence encoding a loading module or interpolypeptide linker and [symbol] can be a gene sequence encoding a thioesterase domain, other releasing domain, interpolypeptide linker, and the like. For example, an ORF vector in which the 1-2 fragment comprises a methionine start codon and a synthetic gene sequence encoding the DEBS loading domain, the central region comprises a synthetic gene sequence encoding DEBS modules 2 and 3, and the C-terminal region comprises a synthetic gene sequence encoding a DEBS TE domain would encode a polypeptide comprising the DEBS N-LM-DEBS2-DEBS3-TE-C (all contiguous synthetic polypeptide-encoding gene sequences described herein are in-frame with each other).

Coding sequences of accessory units are known (see, e.g., GenBank) and synthetic accessory unit genes can be made by synthon stitching and other methods described herein. Exemplary methods for construction of ORF vectors with such N-terminal and C-terminal regions is described below.

6.4.2 ORF Vector Synthesis

This section describes “ORF 2” type vectors useful for construction of a gene libraries of interchangeable elements. Three general types of vectors include

| | |
|---|---|
| Internal type- | 4-[7-*]-[*-8]-3 |
| Left-edge type- | 4-[7-1]-[*-8]-3 |
| Right-edge type- | 4-[7-*]-[6-8]-3 |

The brackets are used to refer to the fact that the required distance from 7 to * is fixed once 7 is picked; similarly the required distance from * to 8 is fixed once 8 is picked; and the remaining bracketed pairs [7-1] and [6-8] optionally can be chosen to be usefully proximate to each other, as described below. To use the three vectors the enzymes whose recognition sites are 7 and 8 have mutually compatible overhang products at all locations marked [7-*] or [*-8], preferably accomplished by having a) equal overhang lengths (which may be zero); b) by having cut sites creating identical overhangs (if any) at those locations [with the identical sequences within the module or accessory gene fragment at the overhangs (if any) being labelled *]; and c) the cut sites are required to be similarly compatible with the open reading frame [so the two occurrences of * (if any) initiate at the same positions with respect to the frame; or if the enzymes whose recognition sites are 7 and 8 are blunt cutters, the cut sites must be equivalently placed with respect to the frame].

The site labelled 1 becomes the left edge of the construct, and can be chosen to be a restriction recognition site for an enzyme cutting within its site (e.g., Nde I). Similarly, the site labelled 6 becomes the right edge of the construct, and can be chosen to be a restriction recognition site for an enzyme cutting within its site (e.g., Eco RI). This pair of sites can be usefully chosen to be pairs convenient for moving the final construct into various expression vectors as desired. The construction method itself does not require either 1 or 6 to be a restriction enzyme recognition site, but simply a place at which cuts can be created with the following conditions:
a) the cut at 1 in the assembly (library) vector is compatible with a cut which can be created at site 1 in the ORF construction vector family during ORF construct creation;
b) the cut at site 6 in the assembly (library) is compatible with a cut which can be created at site 6 in the ORF construction vector family during ORF construct creation;
c) in each case, after transfer of the library ORF element to the ORF construction vector, the recognition sites for the Type IIS enzymes chosen for sites 7 & 8 are unique (if present) in the vector product.

For example, the Type IIS enzyme for 7 could be used to cut at site 1, creating an overhang at 1 which could be used for transfer.

Construction of an ORF Vector with an Initial Defined N-Terminal Region:

A library vector of left-edge type (with site pattern 4-[7-1]-[*-8]-3) is cut at 1 and at 3, and the fragment 1-[*-8]-3 is saved; an ORF vector (initially with site pattern 1-3-4-6) is cut at 1 and 3, and the fragment 3-4-6-1 is joined to the donor fragment 1-[*8]-3 to create a fragment with pattern 1-[*-8]-3-4-6.

Construction of an ORF Vector with an Initial Defined C-Terminal Region:

A library vector of right-edge type (with site pattern 4-[7-*]-[6-8]-3) is cut at 4 and at 6, and the fragment 4-[7-*]-6 is saved; an ORF vector (initially with site pattern 1-3-4-6) is cut at 4 and 6, and the fragment 6-1-3-4 is joined to the donor fragment 4-[7-*]-6 to create a fragment with pattern 1-3-4-[7-*]-6.

The construction of a left edge by an equivalent method can be done in the presence of a previously constructed right edge. In this case, the donor is again a library vector of left-edge type (with site pattern 4-[7-1]-[*-8]-3); and the acceptor now an ORF vector with site pattern 1-3-4-[7-*]-6; once again, the donor fragment 1-[*-8]-3 replaces the acceptor fragment 1-3.

Similarly, the construction of a right edge by an equivalent method can be done in the presence of a previously constructed left edge. In this case, the donor is again a library vector of right-edge type (with site pattern 4-[7-*]-[6-8]-3); and the acceptor now an ORF vector with site pattern 1-[*-8]-3-4-6; once again, the donor fragment 4-[7-*]-6 replaces the acceptor fragment 4-6.

Once either a left or a right edge has been added, that edge can be extended arbitrarily many times by the standard internal extension procedure without interfering with the potential for extension at the other edge. At any time after a left and right edge have been added, together with arbitrarily many extensions at the left and/or right by library gene fragments of internal type, the procedure can be terminated by cleaving the ORF construction vector at [*-8] and [7-*], and joining the overhangs (or blunt ends, in the blunt-end type IIS case) created at the two * sites.

It will be apparent from the foregoing that Internal type, Left-edge type, and Right-edge type-constructs can also be made in “ORF 1” type vectors described in the next section, using modifications of the method above that account for the differences in the restriction sites in the ORF1 and ORF2 vectors.

6.4.3 Exemplary ORF Vector Construction Methods

This section described three exemplary methods for constructing multimodule genes. The examples given show construction in ORF vectors such as those described above, but it will be apparent to the practitioner that many variations of each approach are possible and that the cloning strategies shown can be used in other contexts. For simplicity, the methods below are shown without the presence of sequences encoding the amino and carboxy-terminal regions (e.g., accessory units) discussed above in Section 6.4.3. However, the possible inclusion of such regions will be apparent to the reader.

### Exemplary Construction Method 1

In this exemplary method, assembly vectors are used in which a unique Not I site (4) and a unique Eco R1 site (6) flank the synthon insertion site. Accordingly, the module genes, each of which is designed so that (a) the module gene contains no Not I or Eco RI sites. In addition, it is assumed for this example that each module gene in the library is designed with unique Spe I (5) site at the 5′/amino-terminal edge of the module and a unique Xba I site (2) at the 3′/carboxyterminal edge of the module (see FIG. 6). The structure of the module-containing assembly vector can be described as:

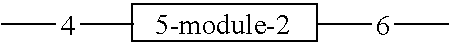

where “module” refers to a module gene and the boxed region indicates the module boundary (i.e., in this example, sites 5 and 2 are within the module gene). A library of such module-containing assembly vectors (containing different modules A, B, C, . . . ) can be described as:

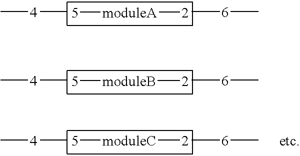

A module-containing assembly vector in a library can be called an “assembly vector” or a “library vector.”

To synthesize a multimodule gene construct, an ORF (“open reading frame”) vector is used for manipulation. In this example, the ORF vector can have the following structure:

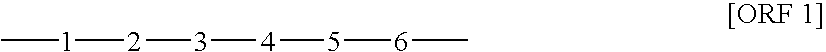

The Nde I site (1), which contains a methionine start codon is convenient because, as will be seen, it can be used to delimit the amino terminus of the open reading frame; however, it is not required in all embodiments (for example, the methionine start codon can be designed in the module rather than provided by the ORF vector). The Pac I site (3) in this construct is useful for restriction analysis but also is not required. (The absence of the Pac I site in the final ORF construct indicates that the region delimited by 3-4 has been successfully removed during the production process; see below.)

To insert a first module gene (e.g., a module A gene) into the ORF vector, the ORF vector is digested with Not I (4) and Spe I (5), the library vector is digested with Not I (4) and Xba I (2), and the 4-2 fragment of the library vector is cloned into the ORF vector, producing:

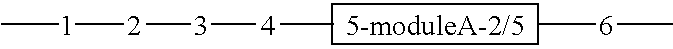

Restriction sites 2 and 5 have compatible cohesive ends that when ligated destroy both sites (2/5). To insert a second module, the process is repeated; the ORF vector containing module A is digested with Not I (4) and Spe I (5), and the 4-2 fragment of a second library vector is cloned into the ORF vector, producing:

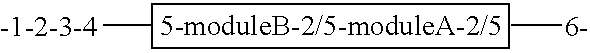

Additional modules, accessory units, or other sequences can be added in a similar manner.

### Exemplary Construction Method 2

In a second exemplary method, Type IIS restriction enzymes are used (as described above in Section 4). In this case, the structure of the module gene-containing assembly vectors in the library can be described as:

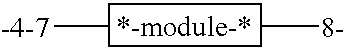

for example,

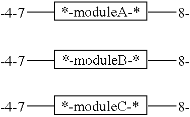

where 7 and 8 are recognition sites for Type IIS enzymes which can form a cohesive and compatible ends (e.g., having the same length and orientation overhang) and * is a common sequence motif as described below. For the sake of clarity, in the discussion below 7 will be Bbs 1 and 8 will be Bsa I. In this case, the modules are designed so that (a) the module gene contains no Bbs I (7) sites or Bsa I (8) sites as well as being free of Not I (4) sites.

The generation of cohesive and compatible ends by action of the Type IIS enzymes 7 and 8 requires that a common sequence motif be present at each end of a module and the Type IIS recognition sites be positioned to produce overhangs having the sequence of the common sequence motif. In one embodiment, restriction sites for Xba I and Spe I, positioned at different ends of the module (e.g., as in FIG. 6) are used for convenience. In this embodiment, the common sequence motif is 5′-C T A G-3′, the central region of both the Xba I (5′-T̂C T A G A-3′/3′-A G A T ĈT-5′) and Spe I sites (5′-ÂC T A G T-3′/3′-T G A T ĈA-5′). Cleavage by Bbs I and Bsa I produces compatible cohesive ends (5′-N N N N C T A G-3′). Importantly, it will be recognized that the common sequence motif need not be a restriction site (or any particular restriction site) and any number of motifs can be used. It will also be recognized that the introduction of the common sequence motif into the module sequence should not disrupt the function (e.g., biological activity) of the polypeptides encoded by the library. As discussed elsewhere herein, introduction of the Spe I and Xba I sites is expected to fulfill this requirement; an alternative would be, for example, motifs encoding (in combination with the surrounding gene sequence) Ala-Ala.

To synthesize a multimodule construct, an ORF vector with the following structure can be used:

-1-*-8-3-4-7-*-6- [ORF 2]

To insert a first module (e.g., module A) into the ORF vector, the ORF vector is digested with Not I (4) and Bbs I (7), and the library vector is digested with Not I (4) and Bsa I (8). The module containing fragment (with a Not I cohesive end and a second cohesive end compatible with Spe I) is cloned into the ORF vector, producing:

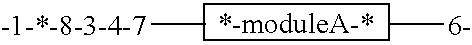

To insert a second module, the assembly vector is digested as for the first module (resulting in e.g.,

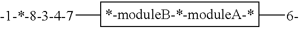

and the ORF vector containing module A is digested with Not I (4) and Bbs I (7), producing

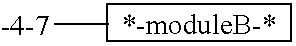

This construct can be cut with both Bbs I (7) and Bsa I (8) to produce:

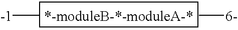

### Exemplary Construction Method 3

In this exemplary method, assembly vectors in which a unique Not I site (4) and a unique Pac I site (3) flank the synthon insertion site are used to make a library of PKS module genes, each of which is designed so that (a) the module gene contains no Not I or Pac I sites. Further, the module gene has a unique Spe I (5) site at the 5′-edge of the module gene and an Xba I site (2) at the 3′-edge of the module gene.

The structure of the module gene-containing assembly vectors in the library can be described as:

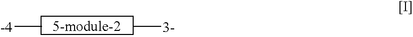

A library of such assembly vectors can be described as:

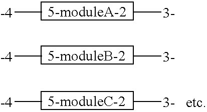

Using Exemplary Method 3, module genes can be assembled bidirectionally in a vector. For example, to generate a vector containing genes for modules A-B-C-D-E, the module genes could be individually added to the vector in the order A, B, C, D, E; E, D, C, B, A; C, B, D, E, A; etc.

Using an ORF vector having the sites

-1-2-3-4-5-6- [ORF 1]

the first module gene (A) can be introduced by cutting with Not I (4) and Xba I (2) in the module, and digesting the ORF vector with Not I (4) and Spe I (5) resulting in

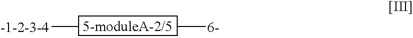

or cutting with Spe I (5) and Pac I (3) in the assembly vector and Xba I (2) and Pac I (3) in the ORF vector to obtain the resulting construct

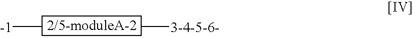

To add a second module gene, the module B gene, to the left of the module A gene in construct III, the assembly vector containing module B is digested with Spe I (5) and Pac I (3), and the ORF vector containing the module A gene is digested with Xba I (2) and Pac I (3), resulting in

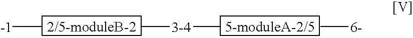

Additional modules can then be added to construct (V), either next to the module B gene or module A gene. For example, the constructs

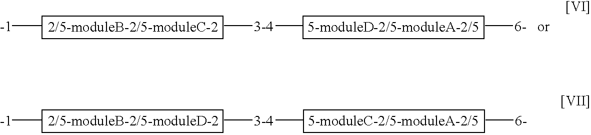

can be made. Constructs (V)-(VIII) can be digested with Spe I (5) and Xba I (2) to remove the 2-5 fragment, producing a gene encoding a polypeptide containing contiguous modules in a single open-reading frame.

The module-containing open reading frames made using these methods can be excised from the ORF vector and inserted into an expression vector. For example, in the example shown above, the open reading frame can be excised using the Nde I (1) and Eco RI (6) sites.

It will be appreciated that the examples shown above are merely to illustrate the ability to use libraries of assembly modules for production of multimodule constructs. It will be recognized that a variety of other combinations of restriction sites, enzymes, common sequence motifs and cleavage sites can be used to accomplish the results illustrated in the preceding paragraphs. For example, a library (or toolbox) can contain incomplete ORFs comprising various combinations of four modules plus accessory units (for example, constructs such as [VI] and [VII] above

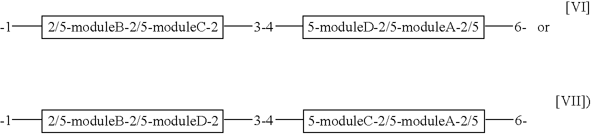

Such libraries could contain, for example, combinations of modules known or believed likely to be productive. Using such a library, the activity of a PKS or NRPS module, or other polypeptide segment, can be tested in a variety of environments. It will be clear from the discussion above that a number of useful libraries are made possible by the methods disclosed herein.

### 7. MULTIMODULE DESIGN BASED ON NATURALLY OCCURRING COMBINATIONS

An alternative, or complementary, strategy for design of synthetic genes encoding polyketide synthases is based on that described in Khosla et al., WO 01/92991 (“Design of Polyketide Synthase Genes”) in which the starting point is a desired polyketide (e.g., a naturally occurring polyketide or a novel analog of a naturally occurring polyketide). In one strategy, the structure of a desired polyketide is assigned a polyketide code (string) by converting the polyketide into a “sawtooth” format (i.e., it is linearized and any post-synthetic modifications are removed) and assigning a one-letter code corresponding to each of the possible 2-carbon ketide units found in polyketides to create a string that describes the polyketide. The ketide units of desired polyketide are converted to a module code by determining possible modules that could produce the polyketide. The module code is then aligned with those corresponding to known polyketide synthases (preferably by computer implemented scanning of a database of such structures) to identify combinations of modules that function in nature.

In one embodiment of the present invention, potential sources of module sequences are selected based on the alignment of conceptual modules that could produce the desired polyketide with known PKS modules. Alignments can be ranked by, for example, minimizing non-native inter-module and/or inter-protein interfaces. For example, to synthesize a gene with the structure LD-A-B-C-D-E-F, where LD is a loading domain, and A-E are PKS modules, the alignment might produce in the output shown in Table 6.

TABLE 6
HYPOTHETICAL ALIGNMENT OF PKS MODULES

| Target | LD | A | B | C | D | E | F |
|---|---|---|---|---|---|---|---|
| PKS 1 | LD | A | C | D | A | | |
| PKS 2 | D | A | B | C | | | |
| PKS 3 | | | B | C | | | |
| PKS 4 | | | | | D | E | F |
| PKS 5 | | | D | E | D | E | F |

In this example several sources are identified for each of the following module sequences: LD A, B-C, D-E-F. The junctions A-B and C-D are connected to form a functional PKS. Some module sequences may serve the purpose better than others. For example, sequences #2 and #3 may both serve as sources of B-C; however, in sequence #2 the native substrate of B is the product of A, and may therefore be more likely to be productive.

### 8. DOMAIN SUBSTITUTION

In some embodiments, the invention provides libraries of synthetic module genes that contain useful restriction sites at the boundaries of functional domains (see, e.g., FIG. 4). Because these sites are common to the entire library, “domain swaps” can be easily accomplished. For example, in module genes having a unique Pst I site at the C-terminus of the KS domain and a unique Kpn I at the C-terminus of the AT domain (see, e.g., FIG. 4), the AT domains of these modules can be removed and replaced by different AT domain encoding genes bounded by these sites can be exchanged.

For example, using the methods of the invention, a library of 150 synthetic module genes, each corresponding to a different naturally occurring module gene, can be synthesized, in which each synthetic gene has a unique Spe I restriction site at the 5′ end of the gene, an Xba I site at the 3′ end of the gene, a Kpn I site at the 3′ boundary of each KS domain encoding region, and a Pst I site at the 3′ boundary of each AT domain. Any of the 150 modules could then be cloned into a common vector, or set of vectors, for analysis, manipulation and expression and, in addition, the presence of common restriction sites allows exchange or substitution of domains or combinations of domains. For example, in the example above, the Kpn I and Pst I sites could be used to exchange domains in any modules having a KS domain followed by an AT domain.

### 9. EXEMPLARY PRODUCTS

9.1 Synthetic PKS Module Genes

In one aspect, the invention provides a synthetic gene encoding a polypeptide segment that corresponds to a reference polypeptide segment, where the coding sequence of the synthetic gene is different from that of a naturally occurring gene encoding the reference polypeptide segment. For example, in one embodiment, the invention provides a synthetic gene encoding a PKS domain that corresponds to a domain of a naturally occurring PKS, where the coding sequence of the synthetic gene is different from that of the gene encoding the naturally occurring PKS. Exemplary domains include AT, ACP, KS, KR, DH, ER, MT, and TE. In a related embodiment, the invention provides a synthetic gene encoding at least a portion of a PKS module that corresponds to a portion of a PKS module of a naturally occurring PKS, where the coding sequence of the synthetic gene is different from that of the gene encoding the naturally occurring PKS, and where the portion of a PKS module includes at least two, sometimes at least three, and sometimes at least four PKS domains. In a related embodiment, the invention provides a synthetic gene encoding a PKS module that corresponds to a PKS module of a naturally occurring PKS, where the coding sequence of the synthetic gene is different from that of the gene encoding the naturally occurring PKS. In one embodiment, the polypeptide segment encoded by the synthetic gene corresponds to at least about 20, at least about 30, at least about 50 or at least about 100 contiguous amino acid residues encoded by the naturally occurring gene

Differences between the synthetic coding sequence and the naturally occurring coding sequence can include (a) the nucleotide sequence of the synthetic gene is less than about 90% identical to that of the naturally occurring gene, sometimes less than about 85% identical, and sometimes less than about 80% identical; and/or (b) the nucleotide sequence of the synthetic gene comprises at least one unique restriction site that is not present or is not unique in the polypeptide segment-encoding sequence of the naturally occurring gene; and/or (c) the codon usage distribution in the synthetic gene is substantially different from that of the naturally occurring gene (e.g., for each amino acid that is identical in the polypeptide encoded by the synthetic and naturally occurring genes, the same codon is used less than about 90% of the instances, sometimes less than 80%, sometimes less than 70%); and/or (d) the GC content of the synthetic gene is substantially different from that of the naturally occurring gene (e.g., % GC differs by more than about 5%, usually more than about 10%).

In the above-described approaches, the amino acid sequences of individual domains, linkers, combinations of domains, and entire modules can be based on (i.e., “correspond to”) the sequences of known (e.g., naturally occurring) domains, combinations of domains, and modules. As used herein, a first amino acid sequence (e.g., encoding at least one, at least two, at least three, at least four, at least five or at least six PKS domains selected from AT, ACP, KS, KR, DH, and ER) corresponds to a second amino acid sequence when the sequences are substantially the same. In various embodiments of the invention, the naturally occurring domains, linkers, combinations of domains, and modules are from one of erythromycin PKS, megalomicin PKS, oleandomycin PKS, pikromycin PKS, niddamycin PKS, spiramycin PKS, tylosin PKS, geldanamycin PKS, pimaricin PKS, pte PKS, avermectin PKS, oligomycin PSK, nystatin PKS, or amphotericin PKS.

In this context, two amino acids sequences are substantially the same when they are at least about 90% identical, preferably at least about 95% identical, even more preferably at least about 97% identical. Sequence identity between two amino acid sequences can be determined by optimizing residue matches by introducing gaps if necessary. One of several useful comparison algorithms is BLAST; see Altschul et al., 1990, “Basic local alignment search tool.” J. Mol. Biol. 215:403-410; Gish et al., 1993, “Identification of protein coding regions by database similarity search.” Nature Genet. 3:266-272; Altschul et al., 1997, “Gapped BLAST and PSI-BLAST: a new generation of protein database search programs.” Nucleic Acids Res. 25:3389-3402. Also see Thompson et al., 1994, “CLUSTAL W: improving the sensitivity of progressive multiple sequence alignment through sequence weighting, position-specific gap penalties and weight matrix choice,” Nucleic Acids Res. 22:4673-80. (When using BLAST and CLUSTAL W or other programs, default parameters are used.)

In one aspect, the invention provides a synthetic gene that encodes one or more PKS modules (e.g., a sequence encoding an AT, ACP and KS activity, and optionally one or more of a KR, DH and ER activity). In some embodiments, the synthetic gene has at most one copy per module-encoding sequence of a restriction enzyme recognition site such as Spe I, Mfe I, Afi II, Bsi WI, Sac II, Ngo MIV, Nhe I, Kpn I, Msc I, Bgl II, Bss HII, Sac II, Age I, Pst I, Kas I, Mlu I, Xba I, Sph I, Bsp E, and Ngo MIV recognition sites. In an embodiment, the invention provides a synthetic gene encoding a PKS module having a Spe I site near the sequence encoding the amino-terminus of the module-encoding sequence; and/or b) a Mfe I site near the sequence encoding the amino-terminus of a KS domain; and/or c) a Kpn I site near the sequence encoding the carboxy-terminus of a KS domain; and/or d) a Msc I site near the sequence encoding the amino-terminus of an AT domain; and/or e) a Pst I site near the sequence encoding the carboxy-terminus of an AT domain; and/or f) a BsrB I site near the sequence encoding the amino-terminus of an ER domain; and/or g) an Age I site near the sequence encoding the amino-terminus of a KR domain; and/or h) an Xba I site near the sequence encoding the amino-terminus of an ACP domain. A synthetic gene of the invention can contain at least one, at least two, at least three, at least four, at least five, at least six, at least seven, or at least eight of (a)-(h), above.

In a related aspect, the invention provides a vector (e.g., an expression vector) comprising a synthetic gene of the invention. In one embodiment, the invention provides a vector that comprises sequence encoding a first PKS module and one or more of (a) a PKS extension module; (b) a PKS loading module; (c) a thioesterase domain; and (d) an interpolypeptide linker. Exemplary vectors are described in Section 7, above.

In an aspect, the invention provides a cell comprising a synthetic gene or vector of the invention, or comprising a polypeptide encoded by such a vector. In a related aspect, the invention provides a cell containing a functional polyketide synthase at least a portion of which is encoded by the synthetic gene. Such cells can be used, for example, to produce a polyketide by culture or fermentation. Exemplary useful expression systems (e.g., bacterial and fungal cells) are described in Section 3, above.

9.2 Vectors

The invention provides a large variety of vectors useful for the methods of the invention (including, for example, stitching methods described in Section 4 and analysis using multimodule constructs as described in Section 7).

Thus, in one aspect the invention provides a cloning vector comprising, in the order shown, (a) SM4-SIS-SM2-R₁ or (b) L-SIS-SM2-R₁ (where SIS is a synthon insertion site, SM2 is a sequence encoding a first selectable marker, SM4 is a sequence encoding a second selectable marker different from the first, R₁ is a recognition site for a restriction enzyme, and L is a recognition site for a different restriction enzyme). In one embodiment, the SIS comprises —N₁—R₂—N₂— (where N₁ and N₂ are recognition sites for nicking enzymes, and may be the same or different, and R₂ is a recognition site for a restriction enzyme that is different from R₁ or L). The invention also provides composition containing such vectors and a restriction enzyme(s) that recognizes R₁ and/or a nicking enzyme (e.g., N. BbvC IA).

In one aspect, the invention provides a vector comprising SM4-2S₁-Sy₁-2S₂-SM2-R₁, where 2S₁ is a recognition sites for first Type IIS restriction enzyme, 2S₂ is a recognition sites for a different Type IIS restriction enzyme, and Sy is synthon coding region. In one aspect, the invention provides a vector comprising L-2S₁-Sy₂-2S₂-SM2-R₁. In an embodiment, Sy encodes a polypeptide segment of a polyketide synthase. In one embodiment, Bbs I and/or Bsa I are used as the Type IIS restriction enzymes. In an embodiment, the invention provides a composition containing such a vector and a Type IIS restriction enzyme that recognizes either 2S, or 2S₂.

In a related aspect, the invention provides a kit containing a vector and a type IIS restriction enzyme that recognizes 2S, or 2S₂, (or a first type IIS restriction enzyme that recognizes 2S, and a second type IIS restriction enzyme that recognizes 2S₂).

In one embodiment, the invention provides a composition containing a cognate pair of vectors. As used herein, a “cognate pair” means a pair of vectors that can be used in combination to practice a stitching method of the invention. In one embodiment the composition contains a vector comprising SM4-2S₁-Sy₁-2S₂-SM2-R₁, digested with a Type IIS restriction enzyme that recognizes 2S₂, and a vector comprising SM5-2S₃-Sy₂-2S₄-SM3-R₁ digested with a Type IIS restriction enzyme that recognizes 2S₁. In another embodiment the composition contains a vector comprising L-2S₁-Sy₁-2S₂-SM2-R, digested with a Type IIS restriction enzyme that recognizes 2S₂, and a vector comprising L′-2S₁-Sy₂-2S₂-SM3-R₁ digested with a Type IIS restriction enzyme that recognizes 2S₁. (SM1, SM2, SM3, SM4 are sequences encoding different selection markers, R₁ is a recognition site for a restriction enzyme, L and L′ are recognition sites for two different restriction enzymes, each different from R₁, 2S₁ and 2S₂ are recognition sites for two different Type US restriction enzymes, and Sy₁ and Sy₂ adjacent synthons which, in some embodiments, can encode polypeptide segments of a polyketide synthase.)

In a related embodiment, the invention provides a vector containing a first selectable marker, a restriction site (R₁) recognized by a first restriction enzyme, a synthon coding region flanked by a restriction site recognized by a first Type IIS restriction enzyme and a restriction site recognized by a second Type IIS restriction enzyme, where digestion of the vector with the first restriction enzyme and the first Type IIS restriction enzyme produces a fragment containing the first selectable marker and the synthon coding region, and digestion of the vector with the first restriction enzyme and the second Type IIS restriction enzyme produces a fragment containing the synthon coding region and not comprising the first selectable marker. In one embodiment, the vector has a second selectable marker and digestion of the vector with the first restriction enzyme and the first Type IIS restriction enzyme produces a fragment containing the first selectable marker and the synthon coding region, and not containing the second selectable marker, and digestion of the vector with the first restriction enzyme and the second Type IIS restriction enzyme produces a fragment comprising the second selectable marker and the synthon coding region, and not containing the first selectable marker. In an embodiment, the vector can contain a third selectable marker.

In a related aspect, the invention provides vectors, vector pairs, primers and/or enzymes useful for the methods disclosed herein, in kit form. In one embodiment, the kit includes a vector pair described above, and optionally restriction enzymes (e.g., Type IIS enzymes) for use in a stitching method.

9.3 Libraries

In an aspect, the invention provides useful libraries of synthetic genes described herein (“gene libraries”). In one example, a library contains a plurality of genes (e.g., at least about 10, more often at least about 100, preferably at least about 500, and even more preferably at least about 1000) encoding modules that correspond to modules of naturally occurring PKSs, where the modules are from more than one naturally occurring PKS, usually three or more, often ten or more, and sometimes 15 or more. In one example, a library contains genes encoding domains that correspond to domains from more than one polyketide synthase protein, usually three or more, often ten or more, and sometimes 15 or more. In one example, a library contains genes encoding domains that correspond to domains from more than one polyketide synthase module, usually fifty or more, and sometimes 100 or more.

In some aspects of the invention, the members of the library have shared characteristics, e.g., shared structural or functional characteristics. In an embodiment, the shared structural characteristics are shared restriction sites, e.g., shared restriction sites that are rare or unique in genes or in designated functional domains of genes. For example, in one embodiment a library of the invention contains genes each of which encodes a PKS module, where the module-encoding regions of the genes share at least three unique restriction sites (for example, Spe I, Mfe I, Afi II, Bsi WI, Sac II, Ngo MIV, Nhe I, Kpn I, Msc I, Bgl II, Bss HII, Sac II, Age I, Pst I, Bsr BI, Kas I, Mlu I, Xba I, Sph I, Bsp E, and Ngo MIV recognition sites). In one embodiment, a library of the invention contains genes that encode more than one PKS module each, where each module-encoding region shares at least three unique restriction sites. In some embodiments, the number of shared restriction sites is more than 4, more than 5 or more than 6. Exemplary sites and locations of shared restriction sites include a) a Spe I site near the sequence encoding the amino-terminus of the module-encoding sequence; and/or b) a Mfe I site near the sequence encoding the amino-terminus of a KS domain; and/or c) a Kpn I site near the sequence encoding the carboxy-terminus of a KS domain; and/or d) a Msc I site near the sequence encoding the amino-terminus of an AT domain; and/or e) a Pst I site near the sequence encoding the carboxy-terminus of an AT domain; and/or f) a BsrB I site near the sequence encoding the amino-terminus of an ER domain; and/or g) an Age I site near the sequence encoding the amino-terminus of a KR domain; and/or h) an Xba I site near the sequence encoding the amino-terminus of an ACP domain.

In one aspect, genes of the library are contained in cloning or expression vectors. In one aspect, the PKS module-encoding genes in a library also have in-frame coding sequence for an additional functional domain, such as one or more PKS extension modules, a PKS loading module, a thioesterase domain, or an interpolypeptide linker.

9.4 Databases

In one aspect, the invention provides a computer readable medium having stored sequence information. The computer readable medium may include, for example, a floppy disc, a hard drive, random access memory (RAM), read only memory (ROM), CD-ROM, magnetic tape, and the like. Additionally, a data signal embodied in a carrier wave (e.g., in a network including the Internet) may be the computer readable storage medium. The stored sequence information may be, for example, (a) DNA sequences of synthetic genes of the invention or encoded polynucleotides, (b) sequences of oligonucleotides useful for assembly of polynucleotides of the invention, (c) restriction maps for synthetic genes of the invention. In an embodiment, the synthetic genes encode PKS domains or modules.

### 10. HIGH THROUGHPUT SYNTHON SYNTHESIS AND ANALYSIS

10.1 Automation of Synthesis

The gene synthesis methods described herein can be automated, using, for example, computer-directed robotic systems for high-throughput gene synthesis and analysis. Steps that can be automated include synthon synthesis, synthon cloning, transformation, clone picking, and sequencing. The following discussion of particular embodiments is for illustration and not intended to limit the invention.

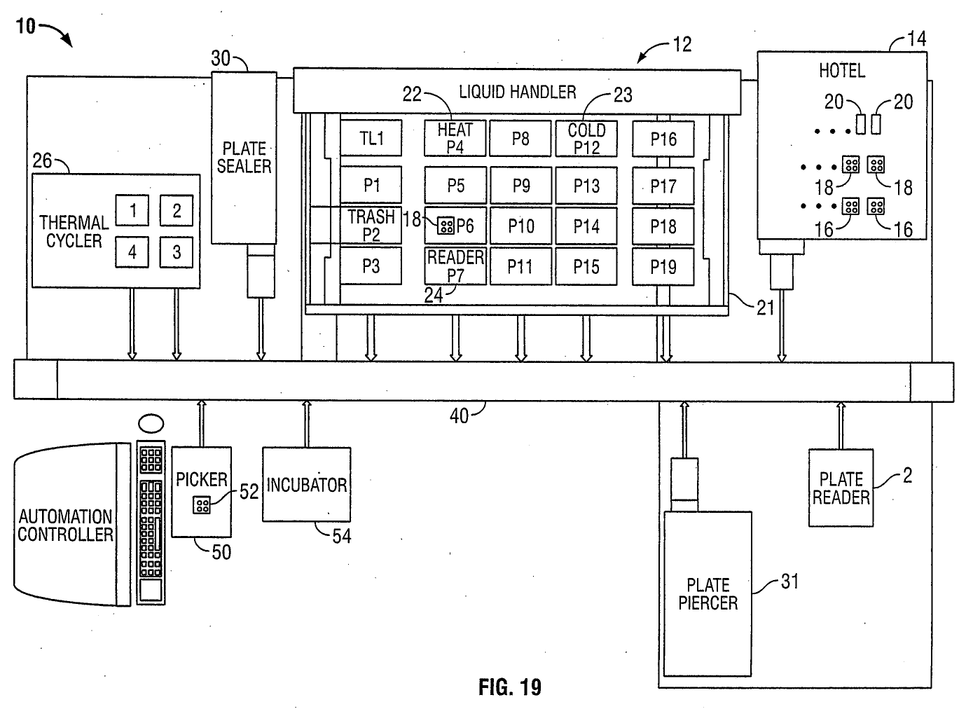

As illustrated in FIG. 19, the invention provides an automated system 10 comprising a liquid handler 12 (e.g., Biomek FX liquid handler; Beckman-Coulter), and a random access hotel 14 (e.g., Cytomat™ Hotel; Kendro) coupled to the liquid handler 12. Liquid handler 12 includes a plurality of positions P1 through P19 which can accept microplates and other vessels used in system 10. As discussed below and as shown in FIG. 19, a number of the positions include additional functionality. The random access hotel 14 is capable of storage of one or more source microplates 16 each carrying oligonucleotide solutions one or more PCR plates 18 comprising synthon assembly wells, and one or more (optional) sources 20 of LIC extension primers (e.g., uracil-containing oligonucleotides), and is capable of delivery of plates and pipette tips to liquid handler 12. In some embodiments, the hotel contains >5, >10, or >20 microplates (and, for example >50, >100, or >200 different oligonucleotide solutions). In the example of FIG. 19, source 20 includes a micro-centrifuge tube. Source 20 could also be a vial or any other suitable vessel. Random access hotel 14 is used for primer mixing, PCR-related procedures, sequencing and other procedures. In one embodiment, liquid handler 12 comprises a deck 21 with heating element 22 at position P4 and cooling element 23 at position P12. Deck 21 can also include an automatic reading device 24, such as a bar code reader, located at position P7 in the example of FIG. 19. System 10 also includes a thermal cycler 26, a plate reader 28, a plate sealer 31 and a plate piercer 30. The reading device 24 is capable of tracking data, and enables hit picking for library compression and expansion as discussed in section 6 above. Hit picking can be useful, for example, for rearranging clones from a library according to user input.

Random access hotel 32 provides plate storage needed for high-throughput primer (oligonucleotide) mixing, and decreases user intervention during plasmid preparations and sequencing. Plate reader 28 includes a spectrophotometer for measuring DNA concentration of samples. Data taken from plate reader 28 is used to normalize DNA concentrations prior to sequencing. Thermal cycler 26 serves as a variable temperature incubator for the PCR-steps necessary for gene synthesis. The reading device 24 is integrated for sample tracking. System 10 also includes robotic arm 40 for transporting sample and plates between different elements in system 10 such as between liquid handler 12 and random access hotel 14.

For illustration and not as any limitation, synthesis can be automated in the following fashion:

Primer Mixing. Robotic arm 40 is coupled to the liquid handler 12 and transports one or more source microplates and PCR plates from random access hotel 14 to liquid handler 12. Liquid handler 12 dispenses appropriate amounts of each of about 25 oligonucleotides from source microplates 16 into a “synthon assembly” well of a PCR plate 18 such that each well contains equimolar amounts of the primers necessary to make a synthon. Since each primer mix contains a different primers (oligonucleotides), as described above, a spreadsheet program is optionally utilized to identify the primer and automatically extract the data necessary for liquid handler 12 to determine which primers correspond to which synthon assembly well. In one embodiment, data from the GEMS output identifying oligonucleotide primer locations and destinations is used to generate corresponding transfer data for the liquid handler 12. Creation of such transfer data from location and destination data is well understood in the art. In embodiments, the hotel 14 carries at least about 50, at least about 100, at least about 150, at least about 200, or at least about 1000, oligonucleotide mixes in different wells of microwell-type plates).

Synthon Synthesis by PCR. Once the PCR plate 18 is loaded with primer mixes, the liquid handler 12 delivers the assembly PCR amplification mixture (including polymerase, buffer, dNTPs, and other components needed for “synthon assembly”) to each well, and PCR is performed therein. Robotic arm 40 moves PCR plate 18 to plate sealer 31 to seal the PCR plate 18. After sealing, PCR plate 18 is moved by robotic arm 40 to thermal cycler 26.

LIC extensions containing uracil are added by liquid handler 12 to the PCR products (amplicons) by a second PCR step. In the second PCR step, the primers containing LIC extensions are added (LIC extension mixture) to each well to prepare the “linkered-synthon.”

A synthon cloning mixture is prepared by combining the Tinkered synthon and a synthon assembly vector in liquid handler 12. Each synthon cloning mixture is then transferred to a sister plate containing competent E. coli cells for transformation, which are positioned at cooling element 12. After transformation, cells in each well are spread on petri dishes, which are incubated to form isolated colonies.

Following incubation of the bacterial cell culture, the plates are transferred by robot arm 40 from an incubator 54 to an automated colony picker 50 (e.g., Mantis; Gene Machines). Automated colony picker 50 identifies 5 to 10 isolated colonies on a plate, picks them, and deposits them in individual wells of a deep-well titer plate 52 containing liquid growth medium.

Liquid growth medium is used to prepare DNA for sequencing, e.g., as described above. The liquid handler 12 then sets up sequencing reactions using primers in both directions. Sequencing is carried out using an automated sequencer (e.g., ABI 3730 DNA sequencer).

The sequence is analysed as described below.

10.2 Rapid Analysis of Chromatograms (RACOON)

A bottleneck in the gene synthesis efforts can be the analysis of DNA sequencing data from synthons. For example, sequence analysis of a single synthon may require sequencing 5 clones in both directions. In one embodiment, a typical PKS gene might involve analysis of 100 synthons, with 5-forward and 5-reverse sequences each (1000 total sequences).

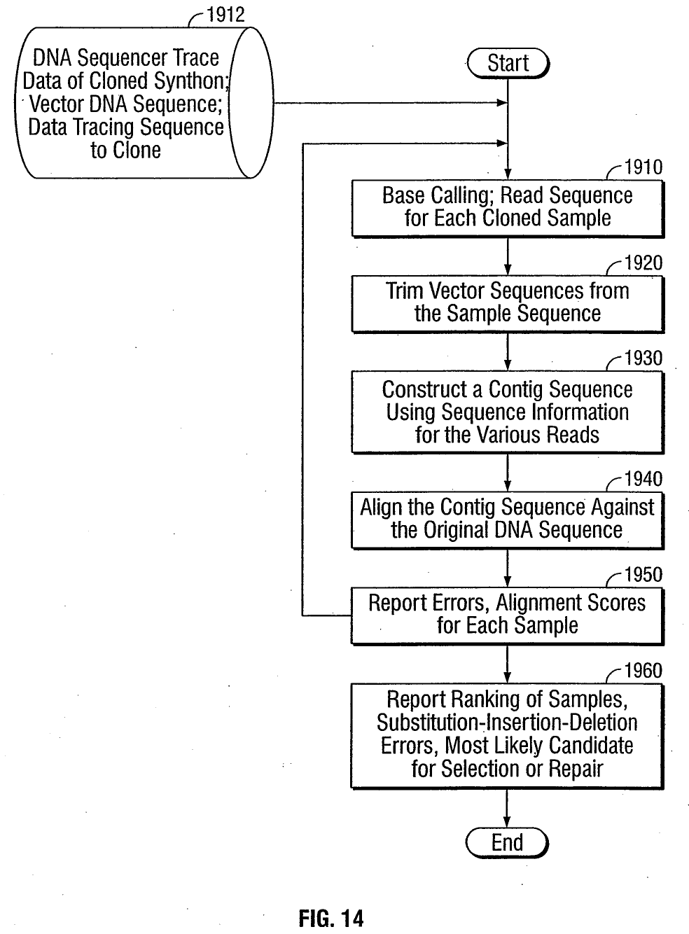

To ensure accuracy in synthesis of large genes, a rapid analysis of the results is performed by a RACOON program as shown in the schematic of FIG. 14. A sequence of a synthetic gene, wherein the synthetic gene is divided into a plurality of synthons, sequences of synthon clones wherein each synthon of the plurality of synthons is cloned in a vector, a sequence of the vector without an insert is entered in the program 1912. In addition, DNA sequencer trace data tracing each synthon sequence to a particular clone are also provided 1912. For all reads, the nucleotide sequence is analyzed (by base calling) 1910 for each cloned sample and vector sequences that occur in the sample sequence are eliminated 1920. To improve accuracy of data processing software in high-throughput sequencing and reliably measuring that accuracy, a base-calling program such as PHRED is used to estimate a probability of error for each base-call, as a function of certain parameters computed from the trace data. A map depicting the relative order of a linked library of overlapping synthon clones representing a complete synthetic gene segment is constructed (“contig map”) 1930 and the contig sequences are aligned against the reference sequence of the synthetic gene 1940. The program identifies errors and alignment scores for each sample 1950 and generates a comprehensive report indicating ranking of samples, substitution-insertion-deletion errors, most likely candidate for selection or repair 1960.

Preparation of a single synthon might entail sequencing five clones in both directions. The sequences are called and vector sequence is stripped by PHRED/CROSS_MATCH. Next, the sequences are sent to PHRAP for alignment, and the user analyzes the data: the correct (if any) sequence is chosen by comparison to the desired one, and errors in others are captured and analyzed for future statistical comparisons.

The Racoon algorithm has been developed to automate tedious manual parts of this process. PHRED reads DNA sequencer trace data, calls bases, assigns quality values to the bases, and writes the base calls and quality values to output files. PHRED can read trace data from SCF files and ABI model 373 and 377 DNA sequencer files, automatically detecting the file format. After calling bases, PHRED writes the sequences to files in either FASTA format, the format suitable for XBAP, PHD format, or the SCF format. Quality values for the bases are written to FASTA format files or PHD files, which can be used by the PHRAP sequence assembly program in order to increase the accuracy of the assembled sequence. After processing sequences by PHRED, Racoon consolidates the forward and reverse sequences of each clone, and sends the composite to PHRAP for alignment with others from the same synthon. The software calls out the correct sequences, and identifies and tabulates the position, type (insertion, deletion, substitution) and number of errors in all clones. It also detects silent mutations, amino acid changes, unwanted restriction sites and other parameters that can disqualify the sample. The user then decides how to use the data (error analysis, statistics, etc.).

The features of Racoon include: (i) reading multiple data formats (SCF, ABI, ESD); (ii) performing base calling, alignments, vector sequence removal and assemblies; (iii) high throughput capability for analysis for multiple 96 well plate samples; (iv) detecting insertions, deletions and substitutions per sample, and silent mutations; (v) detecting unwanted restriction sites created by silent mutations; (vi) generating statistical reports for sample sets which results can be downloaded or stored to a database for further analysis.

The Racoon system is implemented using the following software components: Phred, Phrap, Cross_Match (Ewing B, Hillier L, Wendl M, Green P: Base calling of automated sequencer traces using phred. I. Accuracy assessment. Genome Research 8, 175-185 (1998); Ewing B, Green P: Basecalling of automated sequencer traces using phred. II. Error probabilities. Genome Research 8, 186-194 (1998); Gordon, D., C. Desmarais, and P. Green. 2001. Automated Finishing with Autofinish. Genome Research. 11(4):614-625); Python 2.2 as integration and scripting language (Python Essential Reference, Second Edition by David M. Beazley); GeMS Application Programming Interface (Kosan proprietary software); Apache Web Server version 2.0.44 (http://httpd.apache.org); and Red Hat Linux Operating System version 8.0 (http://www.redhat.com).

### RACOON Algorithm

Step I: Data population. The user inputs into the Racoon program raw sequencing data, vector sequence, and a look-up table that maps the sample to a specific synthon. The program creates run folders for each sample and correctly puts the sequencing files (forward and reverse directions) in its folder, along with the desired synthon sequence. The program uses the look-up table to find the related synthon sequence from a database containing the synthetic gene design data.

Step II. Base calling, vector screening and sequence assembly. Multiple reads can be analyzed using base-calling software such as PHRED and PHRAP (see, e.g., Ewing and Green (1998) Genome Research 8:175-185; Ewing and Green (1998) Genome Research 8:186-194; and Gordon et al. (1998) Genome Research. 8:195-202) to obtain a certainty value for each sequenced nucleotide. A python script is executed on each sample folder containing the chromatogram files for a particular synthon. This script in turn executes the following programs in succession:

PHRED: a base calling software to determine the nucleotide sequence on the basis of multi-color peaks in the sequence trace. PHRED is a publicly available computer program that reads DNA sequencer trace data, calls bases, assigns quality values to the bases, and writes the base calls and quality values to output files (see, for example, Ewing and Green, Genome Research 8:186-194 (1998). After calling bases, PHRED writes the sequences to files in either FASTA format, the format suitable for XBAP, PHD format, or the SCF format. Those skilled in the art will be able to select a nucleotide sequence characterization program compatible with the output of a particular sequencing machine, and will be able to adapt an output of a sequencing machine for analysis with a variety of base-calling programs.

CROSS_MATCH: an implementation of the Smith-Waterman sequence alignment algorithm. It is used in this step to remove the vector sequence from each sample.

PHRAP: a package of programs for assembling shotgun DNA sequence data. It is used to construct a contig sequence as a mosaic of the highest quality parts of reads. The resulting assembly files are candidates for comparison and analysis.

Step III. Error detection, ranking of samples. A python script reruns CROSS_MATCH with the purpose of determining variation between the original synthon sequence and the resulting assembly files for each sample.

Each synthon folder has a collection of sample folders and the associated files generated by PHRED, PHRAP and CROSS_MATCH. A python program detects each of the related samples and associates them with a synthon. It looks for the required information from the output files and ranks the samples. The program looks for silent mutations; checks freshly introduced restriction sites; and generates a report that can be used for further analysis.

Racoon is capable of processing large datasets rapidly. About 200 samples can be analyzed in less than 2 minutes. This included the base calling, vector screening, detection of errors and generation of reports. The results can be saved as HTML files or the individual sample runs can be downloaded to the desktop for further analysis.

### 11. EXAMPLES

### Example 1

### Gene Assembly and Amplification Protocols

This example describes protocols for gene assembly and amplification.

### Assembly

The assembly of synthetic DNA fragments is adapted from a previously developed procedure (Stemmer et al., 1995, Gene 164:49-53; Hoover and Lubkowski, 2002, Nucleic Acids Res. 30:43). The gene synthesis method uses 40-mer oligonucleotides for both strands of the entire fragment that overlap each other by 20 nucleotides.

Equal volumes of overlapping oligonucleotides for a synthon are added together and diluted with water to a final concentration of 25 μM (total). The oligo mix is assembled by PCR. The PCR mix for assembly is 0.5 μl Expand High Fidelity Polymerase (5 units/L, Roche), 1.0 μl 10 mM dNTPs, 5.0 μl 10×PCR buffer, 3.0 μl 25 mM MgCl₂, 2.0 μl 25 μM Oligo mix, 38.5 μl water. The PCR conditions for assembly begins with a 5 minute denaturing step at 95° C., followed by 20-25 cycles of denaturing 95° C. at 30 seconds, annealing at 50 or 58° C. for 30 seconds, and extension temperature 72° C. for 90 seconds.

### Amplification

Aliquots of the assembly reaction are taken and used as the template for the amplification PCR. In the amplification PCR, regions of the primers used contain uracil residues, for use in LIC-UDG cloning. The primers are: 316-4-For_Morph_dU:

316-For_Morph_dU:

[SEQ ID NO:1]

5′GCUAUAUCGCUAUCGAUGAGCUGCCACTGAGCACCAACTACG 3′
and
316-4-Rev_Morph_dU:

[SEQ ID NO:2]

5′GCUAGUGAUCGAUGCAUUGAGCUGGCACTTCGCTCACTACACC 3′.

Uracil-containing regions are underlined. As noted, a common pair of linkers can be used for many different synthons, by design of common sequences at synthon edges.

The reaction mix for the amplification PCR is 0.5 μl Expand High Fidelity Polymerase, 1.0 μl 10 mM dNTPs, 5.0 μl 10×PCR buffer, 3.0 μl 25 mM MgCl2 (1.5 mM), 1.0 μl 50 μM stock of forward Oligo, 1.0 μl 50 μM stock of reverse Oligo, 1.25 μl of assembly round PCR sample (template), and 37.25 μl water The program for amplification includes an initial denaturing step of 5 minutes at 95° C. Twenty-five cycles of 30 seconds of denaturing at 95° C., annealing at 62° C. for 30 seconds, and extension at 72° C. of 60 seconds, with a final extension of 10 minutes.

The amplification of samples is verified by gel electrophoresis. If the desired size is produced, the sample is cloned into a UDG cloning vector. When amplification does not work, a second round of assembly is performed using a PCR mix for assembly of 16 μL first round assembly 0.5 μL Expand High Fidelity polymerase, 1.0 μL 10 mM dNTPs, 3.3 μL 10×PCR buffer, 2.0 μL 25 mm MgCl₂, 2.0 μL oligo mix, and 35.2 μL water. The PCR conditions for the second assembly are the same as the first assembly described above. After the second assembly an amplification PCR is performed.

### Example 2

### Ligation Independent Cloning Methods

Protocols for cloning of synthons into a stitching vector are described below with reference to vectors pKos293-172-2 or pKos293-172-A76. The reader with knowledge of the art will easily identify those changes used to accommodate vectors with different restriction sites, different synthon insertion sites, or different selection markers.

### Exonuclease III Method

Vector preparation: To prepare vectors for UDG-LIC, 10 μL of vector (1-2 μg) is digested with 1 μL Sac I (20 units/μL) at 37° C. for 2 h. 1 μL of nicking endonuclease N. BbvC IA (10 units/μL) is added and the sample is incubated an additional two hours at 37° C. The enzymes are heat inactivated by incubation at 65° C. for 20 minutes, and then a MicroSpin G-25 Sephadex column (Amersham Biosciences) is used to exchange the digestion buffer for water. The samples are treated with 200 units of Exonuclease III (Trevigen) for 10 minutes at 30° C. and purified on a Qiagen quik column, eluting to a final volume of 30 μL. Samples are checked for degradation by gel electrophoresis and used for test UDG-cloning reaction to determine efficiency of cloning.

UDG cloning of fragments: To clone the synthetic gene fragments, they are treated with UDG in the presence of the LIC vector. 2 μL of PCR product (10 ng) is digested for 30 minutes at 37° C. with 1 μL (2 units) of UDG (NEB) in the presence of 4 μL of pre-treated dU vector (50 ng) in a final reaction volume of 10 μL.

The resulting mixtures are placed on ice for 2 minutes, and the entire reaction volume (10 μL) is transformed into DH5α E. coli cells, and selected on LB plates with 100 μg/mL carbenicillin (i.e., SM1). The plasmids are purified for characterization and subsequent cloning steps.

### Endonuclease VIII Method

Vector Preparation: The vector is linearized by digestion with Sac I. Nicking endonuclease (100 units N. BbvC IA) is added and the mixture incubated at 37° C. for 2 h. DNA is isolated from the reaction mixture by phenol/chloroform extraction followed by ethanol precipitation.

UDG Cloning: 20 ng linearized vector, 10 ng PCR product, and 1 unit USER enzyme (a mixture of endonuclease VIII and UDG available as a kit from New England Biolabs) are combined and incubated 15 m at 37° C., 15 m at room temperature, and 2 m on ice, and used to transform E coli DH5α. Endonuclease VIII is described in Melamede et al., 1994, Biochemistry 33:1255-64.

### Example 3

### Characterization and Correction of Cloned Synthons

Identification of clones: To identify clones containing the correct PCR product (e.g. not having sequence errors), plasmid DNA is isolated from several (typically five or more) clones and sequenced. Any suitable sequencing method can be used. In one embodiment, sequencing is carried out using DNA obtained by rolling circle amplification (RCA), using phi29 DNA polymerase (e.g., Templicase; Amersham Biosciences). See, Nelson et al., 2002, “TempliPhi, phi29 DNA polymerase based rolling circle amplification of templates for DNA sequencing” Biotechniques Suppl:44-7. In one embodiment, each colony containing a plasmid to be sequenced is suspended in 1.4 mL LB medium and 1 μl is used in the amplification/sequencing reaction.

Sequence analysis: After sequencing, the results can be aligned and compared to the intended sequence. Preferably this process is automated using a RACOON program (described below) to identify the correct sequences after aligning the sequences corresponding to each synthon.

Storage of clones: Clones of interest can be stored in a variety of ways for retrieval and use, including the Storage IsoCode® ID™ DNA library card (Schleicher & Schuell BioScience).

Site-Directed Mutagenesis to Correct Sequence Errors: Synthon samples can be sequenced until a clone with the desired sequence is found. Alternatively, clones with only 1 or 2 point mutations can be corrected using site-directed mutagenesis (SDM). One method for SDM is PCR-based site-directed mutagenesis using the 40-mer oligonucleotides used in the original gene synthesis. For example, a sample with only one point mutation from the desired target sequence was corrected as follows: The overlapping oligonucleotides from the assembly of the synthons that corresponded to that part of the synthon were identified and used for the correction of the synthon. The error-containing sample DNA was amplified using a Pfu based PCR method using overlapping oligonucleotides (nos. 1 and 2) that cover the area of the mutation (see Fischer and Pei, 1997, “Modification of a PCR-based site directed mutagenesis method” Biotechniques 23:570-74). The reaction mixture included DNA template [5-20 ng], 5.0 μL; 10×Pfu buffer, 0.5 μL; Oligo #1 [25 μM], 0.5 μL; Oligo #2 [25 μM], 1.0 μL; 10 mM dNTPs, 1.0 μL; Pfu DNA polymerase, and sterile water to 50 μL. PCR conditions were as follows: 95° C. 30 seconds (2 minutes if using Pfu with heat sensitive ligand), 12-18 cycles of: 95° C. 30 seconds, 55° C. 1 minutes, 68° C. 2 minutes/kb plasmid length (1 min/kb if Pfu Turbo). Next, the methylated (parental) DNA was degraded by adding 1 μL Dpn I (10 units) to the PCR reaction and incubating 1 hr at 37° C. The resulting sample was transformed into competent DH5α cells. Plasmid DNA from four clones was isolated and sequenced to identify desired clones.

### Example 4

### Identification of Useful Restriction Sites in PKS Modules

To identify useful sites in PKS modules, the amino acid sequences of 140 modules from PKS genes were analysed. A strategy was developed for identifying theoretical restriction sites (i.e., that could be place in a gene encoding the module without resulting in a disruptive change in the module sequence) that fulfill some or all of the following criteria:
1. Sites were about 500 bp apart in the gene and/or are at domain or module edges,
2. Compatible with high-throughput assembly of modules from synthons (often by virtue of being unique within a module),
3. Similarly placed among different modules, and
4. Do not disrupt the function, (activity) of the PKS.

Two types of restriction sites were identified. The first set of sites are those located at the edge of domains (including the Xba I and Spe I sites at the edges of modules). The second set of sites could be located at synthon edges, but were not generally found at domain edges.

It will be understood that the restriction sites described in this example are exemplary only, and that additional and different sites can be identified by the methods of disclosed herein, and used in the synthetic methods of the invention.

The amino acid sequences of selected regions of 140 modules taken from some 14 PKS gene clusters were aligned (see Table 9). Then, regions of high homology near edges of domains that, when reverse translated to all possible DNA sequences, revealed a 6-base or greater restriction site were identified. In specified cases, a conservative change of the amino acid in order to place the restriction site was allowed, provided that change was found in many of the PKS modules. In a few cases, restriction sites were placed in putative inter-domain sequences that required change of amino acids. In such cases there was experimental evidence that the modified amino acid sequence did not disturb functionality in some PKSs.

The results of the gene design for the four common variants ([KS+AT+ACP]; [KS+AT+ACP+KS]; [KS+AT+ACP+KS+DH]; [KS+AT+ACP+KS+DH+ER] of PKS modules are shown in FIG. 4 and Tables 7-11. The positions of the restriction sites are referenced to the homologous amino acid target sites within a domain where possible, and to module 4 of the 6-DEBS gene or protein (which contains all six of the common domains). For the latter, numbering of the amino acid and nucleotide sequence used for reference begins at the first residue of the EPIAIV found on the N-terminal edge of the KS domain; homologous motifs are found at the N-terminal edges of all 140 KS domains in the sample.

TABLE 7
RESTRICTION SITES NEAR DOMAIN EDGES

| | Domain/ | Nucleotide | AA Sequence | Amino acid |
|---|---|---|---|---|
| Restriction | Terminal | Position of site | near site in | motif in |
| Enzyme | Orientation | in ery mod4* | ery mod4 | ery mod4 |
| Spe I | ACP (C) | 54 bp before KS | | VG-not conserved |
| Mfe I | KS (N) | 5-10 | PIAIVG | PIA |
| Kpn I | KS (C) | 1243-1248 | GTNAHV | GT |
| Msc I | AT (N) | 1590-1595 | PGQGAQ | GQ |
| Pst I | AT (C) | 2611-2616 | PRPHRP | PR-not conserved |
| BsrB I | ER ( N) | 4075-4080 | PLRAGE | PL |
| Age I | KR (N) | 5029-5034 | TGGTGT | TG (initial TG) |
| Xba I | ACP (C) | 6001-6006 | FADSAP | FA (not conserved) |
| | | | | from DEBS2 near |
| | | | | terminus |

* Numbering for each module begins at the N-terminus of the KS domain taken to be the amino acid at the site homologous to that of the glutamate (E) of the E-P-I-A-I-V of module 4 of erythromycin.

An Mfe I site is incorporated near the left edge of the KS coding sequence using bases 2-7 of the 9 bases coding for the tripeptides homologous to the PIV of the initial motif of the KS. 70% of the 140 KSs need no change in amino acids; the remaining 30% require only conservative changes [81% V->I, 17% L->I and 2% M to I]. On the right edge of 100% of the 140 KS domains, there is a conserved GT (nt 1267-1272) that can be encoded by the sequence for a Kpn I restriction site.

An Msc I site is incorporated near the left edge of the AT coding sequence (nt 1590-1595) at the site of the GQ dipeptide found in 100% of the sampled ATs. A Pst I site was placed at the right side of the AT (nt 2611-2617) at a position where Pst I and Xho I had been previously placed without loss of functionality after domain swaps. This variable sequence region is identified in many modules by a Y-x-F-x-x-x-R-x-W motif where “x” is any amino acid; in others, alignments always produce a well-defined equivalent position. The two amino acids to the immediate right (C-terminal to W) of this motif are modified to introduce the Pst I site.

For modules containing a KR, an Age I site was placed at the TG dipeptide (nt 4894-5542) found in 100% of the 136 KRs in the test sequences. When an ER domain is present in the module, a Bsr BI site is placed at its left edge, which codes for the conserved PL dipeptide (nt 4072-4929) found in all but one of the 17 ERs in the test sequences (the remaining ER is the only ER domain in the sample without activity). Since the ER and KS domains are separated by only 4 to 6 amino acids, the Age I site of the KR serves as the other excision site for the ER.

At the carboxy end of the module, a Xba I site was placed at a well-defined position adjacent to the carboxy side of the ACP of the module. There are two leucines (L) at positions 36 and 40 to the right of the active site serine (S) of all ACPs. The codons of the two amino acids following the leucine at position 40 (normally positions 41 and 42 after the active site serine) were changed to the recognition sequences for Xba I (C-terminal end).

In modules that naturally followed another, a Spe I cloning site was incorporated as the amino terminus site. This site is analogous to that described for the Xba I, above (normally positions 41 and 42 after the active site serine), and is followed by the intermodular linker to the MfeI site in the KS. In modules that exist at the N-terminus of proteins (i.e. no ACP to the left), the Spe I to MfeI linker sequence is not needed, and the segment of the module synthesized consists of only the MfeI-Xba I body.

It will be appreciated by the reader that the present invention provides, inter alia, a method for identifying restriction enzyme recognition sites useful for design of synthetic genes by (i) obtaining amino acid sequences for a plurality of functionally related polypeptide segments; (ii) reverse-translating said amino acid sequences to produce multiple polypeptide segment-encoding nucleic acid sequences for each polypeptide segment; (iii) identifying restriction enzyme recognition sites that are found in at least one polypeptide segment-encoding nucleic acid sequence of at least about 50% of the polypeptide segments. Preferred restriction enzyme recognition sites are found in at least one polypeptide segment-encoding nucleic acid sequence of at least about 75% of the polypeptide segments, even more preferably at least about 80%, even more preferably at least about 85%, even more preferably at least about 90%, even more preferably at least about 95%, and sometimes about 100%. Examples of functionally related polypeptide segments include polyketide synthase and NRPS modules, domains, and linkers. In one embodiment, the functionally related polypeptide segments are regions of high homology in PKS modules or domains (i.e., rather than the entire extent of a module or domain).

The invention also provides a method of making a synthetic gene encoding a polypeptide segment by (i) identifying one, two three or more than three restriction sites as described above, and (ii) producing a synthetic gene encoding the polypeptide segment that differs from the naturally occurring gene by the presence of the restriction site(s) and (iii) optionally differs from the naturally occurring gene by the removal of the restriction site(s) from other regions of the polypeptide segment encoding sequence.

TABLE 8
RESTRICTION SITES BY MODULE TYPE

| | | # modules of | sites required |
|---|---|---|---|
| module type | # synthons | this type in list | (see list below) |
| DH/KR/ER | 14 | 17 | 1-11, DH1&2, ER1&2 |
| DH/KR | 12 | 48 | 1-11, DH1&2 |
| KR only | 10 | 72 | 1-11 |
| no KR | 7 | 3 | 1-7&11 |

| | |
|---|---|
| total modules in list: | 140 |

TABLE 9
PATTERN OF RESTRICTION SITES USED FOR MODULE DESIGN

| | | | | | | # | | | |
|---|---|---|---|---|---|---|---|---|---|
| | | | | | | Currently | % Currently | | |
| | | | | | # | designed | designed | | |
| | | RestriCtion | | | required | from | from | | |
| | synthon | Site (or set of | | | in set of | database | database | domain | |
| site | edge | alternates) | frame | overhang | 140 | sequenCe | sequenCe | edge | use |

| | | | | | | | | | | |
|---|---|---|---|---|---|---|---|---|---|---|
| 1 | yes | SpeI | ACTAGT | 1 | −4 | 140 | 140* | 100.0% | yes | ACP Cter |
| 1a | | MfeI | CAATTG | 3 | −4 | 140 | 140 | 100.0% | yes | KS nter |
| 2 | yes | set#1 | see Table 7 | 1 or 2 | −4 or 2 | 140 | 140 | 100.0% | | |
| 3 | yes | NheI | GCTAGC | 1 | −4 | 140 | 140 | 100.0% | | |
| 4 | yes | KpnI | GGTACC | 1 | 4 | 140 | 140 | 100.0% | yes | KS Cter |
| 4a | | MsCI | TGGCCA | 2 | blunt | 140 | 139 | 99.3% | yes | AT nter |
| 5 | yes | set#2 | see Table 7 | 1 or 2 | −4 or 2 | 140 | 140 | 100.0% | | |
| 6 | yes | AgeI* | see Table 4 | 1 | −4 | 140 | 98 | 70.0% | | |
| 7 | yes | PstI | CTGCAG | 1 | 4 | 140 | 140 | 100.0% | yes | AT Cter |
| 8 | yes | KasI or MluI | see below | 1 | −4 | 137 | 121 | 88.3% | pre- | |
| | | or both | | | | | | | reduCtive | |
| | | | | | | | | | | region |
| | | | | | | | | | | nter |
| 9 | yes | AgeI | ACCGGT | 1 | −4 | 137 | 132 | 96.4% | yes | KR nter |
| 10 | yes | set#2 | see Table 7 | 1 or 2 | −4 or 2 | 137 | 109 | 79.6% | | |
| 11 | yes | XbaI | TCTAGA | 1 | −4 | 140 | 140* | 100.0% | yes | ACP Cter |
| DH1 | yes | SphI | GCATGC | 2 | 4 | 65 | 54 | 83.1% | | |
| DH2 | yes | set#3 | see Table 7 | 1 or 2 | −4 | 65 | 65 | 100.0% | | |
| ER1 | yes | NgoMIV or | see Table 7 | 1 | −4 | 17 | 17 | 100.0% | | |
| | | BspEI | | | | | | | | |
| ER2 | yes | XbaI* | see Table 8 | 1 | −4 | 17 | 17 | 100.0% | | |

In one embodiment, each site #1 can be joined to site #11 of a second module (or an equivalent Xba I from another upstream unit); and each #11 to an Spe I. Thus #1/#11 in the final construct is only a single location, coding for the dipeptide SerSer (this location has previously been successfully used in cases where the native amino acids were replaced with the homologous dipeptide ThrSer). No amino acid changes are required in sites other than #1a, #7 and #1/#11. At each of these three sites, a history of previous successful exchanges is available.

In site #7, any native dipeptide is replaced with LeuGln. In reported sequences this site is not well conserved, except that the first amino acid is often of large hydrophobic type (as is Leu). [L->I, V->I, M->I]

In one aspect, the invention provides a PKS polypeptide having a non-natural amino sequence, comprising a KS domain comprising the dipeptide Leu-Gln at the carboxy-terminal edge of the domain; and/or an ACP domain comprising the dipeptide Ser-Ser at the carboxy-terminal edge of the domain.

Restriction sites used for synthon edges, but not domain edges, do not require that the restriction site be compatible between modules. At certain sites in Table 10 a list of restriction enzymes is provided, such that the stated number of cases for each site (see Table 9) one of the list is compatible with the amino acid sequence.

TABLE 10

LISTS OF RESTRICTION SITES FOR
CERTAIN SYNTHON EDGE LOCATIONS

| | |
|---|---|
| frame | overhang |

set #1 (at site #2):

| | | | |
|---|---|---|---|
| AflII | CTTAAG | 2 | −4 |
| BsiWI | CGTACG | 2 | −4 |
| SacII | CCGCGG | 1 | 2 |
| NgoMIV | GCCGGC | 1 | −4 |

set #2 (at sites #5 and #10):

| | | | |
|---|---|---|---|
| BglII | AGATCT | 1 | −4 |
| BssHII | GCGCGC | 2 | −4 |
| SacII | CCGCGG | 2 | 2 |

set #3 (at site #DH2):

| | | | |
|---|---|---|---|
| AgeI | ACCGGT | 2 | −4 |
| AflII | CTTAAG | 2 | −4 |
| BspEI | TCCGGA | 1 | −4 |
| NgoMIV | GCCGGC | 1 | −4 |
| site #8: | | | |

| | | | |
|---|---|---|---|
| KasI | GGCGCC | 1 | −4 |
| MluI | ACGCGT | 1 | −4 |
| site #ER1: | | | |

| | | | |
|---|---|---|---|
| NgoMIV | GCCGGC | 1 | −4 |
| BspEI | TCCGGA | 1 | −4 |

TABLE 11

SITES USING PAIRS OF
COMPATIBLE RESTRICTION ENZYMES.

| | |
|---|---|
| frame | overhang |

| | | | | |
|---|---|---|---|---|
| | site #6 (″AgeI*): | | | |
| 5′synthon | AgeI | ACCGGT | 1 | −4 |
| 3′synthon | NgoMIV | GCCGGC | 1 | −4 |

(alternates to NgoMIV: XmaI or BspEI)

| | | | | |
|---|---|---|---|---|
| | site #ER2 (″XbaI*): | | | |
| 5′synthon | XbaI | TCTAGA | 1 | −4 |
| 3′synthon | AvrII | CCTAGG | 1 | −4 |

In certain cases (see sites #6 and #ER2) the constructs are designed by using one restriction site for the 5′ synthon, and a second with compatible overhang for the 3′ synthon. This allows use of certain restriction sites for the synthons that are not desired in the final product (e.g., the Xba I at site #ER2 would interfere with the use of the 3′ Xba I site at #11 for gene construction).

TABLE 12
SOURCES OF 140 MODULES IN INITIAL ANALYZED SET

| | | | source | # extension |
|---|---|---|---|---|
| cluster | accession # | source (genus) | (species) | modules |
| erythromycin | M63676/M63677 | Saccharopolyspora | erythraea | 6 |
| megalomicin | AF263245 | Micromonospora | megalomicea | 6 |
| oleandomycin | AF220951/L09654 | Streptomyces | antibioticus | 6 |
| pikromycin | AF079138 | Streptomyces | venezuelae | 6 |
| niddamycin | AF016585 | Streptomyces | caelestis | 7 |
| spiramycin | | Streptomyces | ambofaciens | 7 |
| tylosin | AF055922 | Streptomyces | fradiae | 7 |
| geldanamycin | | Streptomyces | hygroscopicus | 7 |
| pimaricin | AJ278573 | Streptomyces | natalensis | 12 |
| pte | AB070949 | Streptomyces | avermitilis | 12 |
| avermectin | AB032367 | Streptomyces | avermitilis | 12 |
| oligomycin | AB070940 | Streptomyces | avermitilis | 16 |
| nystatin | AF263912 | Streptomyces | nodosus | 18 |
| amphotericin | AF357202 | Streptomyces | noursei | 18 |
| | | | total: | 140 |

Other sequences of domains, modules and ORFs of PKSs and PKS-like polypeptides can be obtained from public databases (e.g., GenBank) and include, for illustration and not limitation, accession numbers sp|Q03131|ERY1_SACER; gb|AAG13917.1|AF263245_{—}13; gb|AAA26495.1; pir∥S13595; prf∥1702361A; sp|Q03133|ERY3_SACER; gb|AAG13919.1|AF263245_{—}15; ref|NP_{—}851457.1; dbj|BAA87896.1; ref|NP_{—}851455.1; gb|AAF82409.1|AF220951_{—}2; gb|AAF82408.1|AF220951_{—}1; ref|NP_{—}824071.1; ref|NP 822118.1; gb|AAG23266.1; ref|NP_{—}821591.1; sp|Q07017|OL56 STRAT; pir∥T17428; gb|AAF86393.11|AF235504_{—}14; gb|AAF71766.1|AF263912.5; ref|NP_{—}821593.1; dbj|BAB69304.1; ref|NP_{—}824075.1; gb|AAB66507.1; ref|NP_{—}824068.1; ref|NP_{—}821594.1; dbj|BAB69303.1; gb|AAF86396.1|AF235504_{—}17; ref|NP_{—}823544.1; ref|NP_{—}822117.1; pir|T17463; gb|AAK73501.1|AF357202_{—}4; dbj|BAC57030.1; emb|CAB41041.1; ref|NP_{—}336573.1; emb|CAC20920.1; ref|NP_{—}822114.1; gb|AAC46028.1; emb|CAC20921.1; ref|NP_{—}855724.1; dbj|BAC57031.1; ref|NP_{—}216564.1; gb|AAB66504.1; ref|NP_{—}824073.1; gb|AAG23262.1; gb|AAG23263.1; ref|NP_{—}824072.1; gb|AAO06916.1; gb|AAG23264.1; gb|AAF86392.1|AF235504_{—}13; gb|AAP42855.1; ref|NP_{—}630373.1; gb|AAB66508.1; pir|T30226; gb|AAK73514.1|AF357202_{—}17; gb|AAB66506.1; pir|T17410; pir|T30283; gb|AAP42874.1; pir∥T17464; ref|NP_{—}822113.1; gb|AAC0711.1; gb|AAG09812.1|AF275943_{—}1; ref|NP_{—}733695.1; pir∥T30225; ref|NP_{—}824074.1; gb|AAO06918.1; pir∥T03221; gb|AAM81586.1; pir∥T30228; pir∥T17409; gb|AAC46026.1; gb|AAC46024.1; gb|AAO65800.1|AF440781_{—}19; gb|AAK73513.1|AF357202_{—}16; gb|AAM54078.1|AF453501_{—}4; gb|AAK73502.1|AF357202_{—}5; gb|AAP42858.1; pir∥T03223; gb|AAM81585.1; gb|AAF71775.1|AF263912_{—}14; gb|AAG23265.1; gb|AAP42856.1; emb|CAC20919.1; pir∥T17412; pir|T17467; gb|AAF71776.1|AF263912_{—}15; pir∥T17411; gb|AAO65799.1|AF440781_{—}18; ref|NP_{—}821590.1; dbj|BAC54914.1; gb|AAF71768.1|AF263912_{—}7; gb|AAO65796.1|AF440781_{—}15; ref|NP_{—}824069.1; gb|AAO61200.1; gb|AAP42859.1; gb|AAO65806.1|AF440781_{—}25; gb|AAF71774.1|AF263912_{—}13; gb|AAL07759.1; ref|NP_{—}851456.1; ref|NP_{—}821592.1; pir∥T03224; gb|AAO06917.1; gb|AAO65797.1|AF440781_{—}16; gb|AAK73512.1|AF357202_{—}15; ref|NP_{—}301229.1; gb|AAC46025.1; ref|NP_{—}856616.1; emb|CAB41040.1; gb|AAC01712.1; pir∥T17465; gb|AAP42857.1; gb|AAK73503.1|AF357202_{—}6; gb|AAO65801.1|AF440781_{—}20; gb|AAO65798.1|AF440781_{—}17; pir∥T17466; pir∥S23070; sp|Q03132|ERY2_SACER; gb|AAG13918.1|AF263245_{—}14; emb|CAA44448.1; ref|NP_{—}794435.1 gb|AAM54075.1|AF453501_{—}1; gb|AAA50929.1; gb|AAP42860.1; dbj|BAC57032.1; dbj|BAC57028.1; dbj|BAA76543.1; gb|AAP42873.1; ref|NP_{—}855341.1; ref|NP_{—}216177.1; gb|AAM54076.1|AF453501_{—}2; gb|AAP40326.1; gb|AAC46027.1; gb|AAM54077.1|AF453501_{—}3; gb|AAN63813.1; emb|CAD43451.1; gb|AAK19883.1; ref|NP_{—}630372.1; gb|AAO65807.1|AF440781_{—}26; gb|AAA79984.2; gb|AAF26921.1|AF210843_{—}18; emb|CAD43448.1; ref|NP_{—}794436.1; gb|AAB66505.1; gb|AAF43113.1; gb|AAF62883.1|AF217189_{—}6; dbj|BAC57029.1; pir∥T03222; gb|AAP42867.1; ref|NP_{—}822727.1; emb|CAD43450.1; gb|AAD03048.1; gb|AAP45192.1; gb|AAO61221.1; gb|AAF82077.1|AF232752_{—}2; ref|NP_{—}486720.1; gb|AAO65790.1|AF440781_{—}9; ref|NP_{—}485688.1; gb|AAM81584.1; emb|CAD43449.1; ref|ZP_{—}00108795.1; ref|NP_{—}302534.1; gb|AAP42872.1; pir|T28658; ref|ZP_{—}00105790.1; ref|NP_{—}217447.1; ref|NP_{—}337514.1; emb|CAD19091.1; ref|NP_{—}856601.1; gb|AAF19810.1|AF188287_{—2}; ref|ZP_{—}00110107.1; ref|ZP_{—}00110105.1; ref|NP_{—}217449.1; ref|NP_{—}337516.1; gb|AAF62880.1|AF217189_{—}3; gb|AAK57188.1|AF319998_{—}7; ref|ZP_{—}00108802.1; ref|ZP_{—}00110106.1; ref|NP_{—}217450.1; ref|NP_{—}856604.1; pir∥T30871; gb|AAF26919.1|AF210843_{—}16; ref|ZP_{—}00107887.1; ref|NP_{—}856602.1; ref|NP_{—}217448.1; emb|CAD19092.1; ref|NP 336931.1; ref|NP_{—}216898.1; gb|AAO62584.1; ref|ZP_{—}00108796.1; pir∥S73013; ref|NP_{—}302535.1; gb|AAM70355.1|AF505622_{—}27; gb|AAF26922.1|AF210843_{—}19; gb|AAK57186.1|AF319998_{—}5; gb|AAK57187.1|AF319998_{—}6; emb|CAD19190.1; ref|NP_{—}302536.1; ref|ZP_{—}00108803.1; emb|CAD19087.1; gb|AAF62884.1|AF217189_{—}7; pir∥T17421; ref|NP_{—}302533.1; pir∥S73021; gb|AAO64405.1; gb|AAF19813.1|AF188287_{—}5; ref|NP_{—}602063.1; emb|CAD19088.1; gb|AAO64407.1; gb|AAF00959.1|AF183408_{—}7; gb|AAF26923.1|AF210843_{—}20; emb|CAD29794.1; gb|AAF19814.1|AF188287_{—}6; emb|CAD29793.1; ref|ZP_{—}00108797.1; gb|AAF62885.1|AF217189_{—}8; dbj|BAB12210.1; ref|ZP_{—}00074381.1; gb|AAO62582.1; ref|NP_{—}214919.1; ref|NP_{—}630013.1; ref|NP_{—}334828.1; gb|AAK57189.1|AF319998_{—}8; ref|ZP_{—}00110108.1; ref|NP_{—}739315.1; gb|AAM33470.1|AF395828_{—}3; emb|CAD19086.1; emb|CAD19089.1; ref|NP_{—}217456.1; ref|NP_{—}486719.1; ref|NP_{—}856610.1; pir∥B44110; ref|ZP_{—}00107886.1; ref|NP_{—}485689.1; gb|AAF00958.1|AF183408_{—}6; ref|NP_{—}301233.1; ref|NP_{—}854867.1; ref|NP_{—}215696.1; ref|NP_{—}335661.1; ref|NP_{—}218317.1; ref|ZP_{—}00107888.1; emb|CAD19085.1; ref|NP_{—}857467.1; ref|NP_{—}301199.1; pir∥T17420; ref|NP_{—}218342.1; gb|AAK57190.1|AF319998_{—}9; dbj|BAB12211.1; gb|AAM77986.1; gb|AAC49814.1; ref|NP_{—}522202.1; ref|NP_{—}870253.1; ref|NP_{—}301890.1; ref|NP 216043.1; ref|NP_{—}855206.1; dbj|BAA20102.1; emb|CAD19093.1; ref|ZP_{—}00130214.1; gb|AAK26474.1|AF285636_{—}26; gb|AAK48943.1|AF360398_{—}1; ref|NP_{—}867299.1; ref|NP_{—}828360.1; dbj|BAB69235.1; ref|NP_{—}349947.1; ref|NP_{—}519927.1; gb|AAC23536.1; ref|XP_{—}324222.1; ref|NP_{—}841435.1; ref|ZP_{—}00107678.1; sp|P22367|MSAS_PENPA; ref|NP_{—}854075.1; ref|NP_{—}630898.1; gb|AAN85523.1|AF484556_{—}45; ref|NP_{—}389599.1; emb|CAB13589.2; gb|AAB49684.1; ref|NP_{—}389603.1; emb|CAB13604.2; gb|AAN85522.1|AF484556_{—44}; ref|ZP_{—}00102851.1; gb|AAO62426.1; gb|AAM12913.1; dbj|BAC20566.1; gb|AAN17453.1; ref|ZP_{—}00126161.1; ref|ZP_{—}00065888.1; ref|XP_{—}325868.1; ref|NP_{—}216180.1; ref|NP_{—}855344.1; gb|AAD34559.1; ref|ZP_{—}00050081.1; ref|ZP_{—}00074378.1; ref|ZP_{—}00126160.1; gb|AAL27851.1; dbj|BAB69698.1; gb|AAB08104.1; pir∥T44806; dbj|BAC20564.1; pir∥T31307; ref|XP_{—}330288.1; ref|NP_{—}851435.1; gb|AAN60755.1|AF405554_{—}3; ref|ZP_{—}00103294.1; gb|AAD39830.1|AF151722_{—}1; ref|XP_{—}330106.1; gb|AAF19812.1|AF188287_{—}4; ref|NP_{—}085630.1; ref|XP_{—}329445.1; gb|AAF26920.1|AF210843_{—}17; emb|CAB13603.2; ref|NP_{—}534177.1; ref|NP_{—}356936.1; gb|AAM12909.1; ref|NP 792409.1; gb|AAG02357.1|AF210249_{—}16; ref|NP 384683.1; gb|AAF62882.1|AF217189_{—}5; emb|CAB13602.2; ref|NP_{—}389600.1; ref|NP_{—}822424.1; gb|AAK15074.1; ref|NP_{—}356944.1; ref|NP_{—}754352.1; gb|AAO52333.1; ref|NP_{—}851438.1; ref|ZP_{—}00130212.1; ref|ZP_{—}00110270.1; ref|NP_{—}389601.1; ref|NP_{—}721710.1; gb|AAM33468.1|AF395828_{—}1; emb|CAC94008.1; ref|XP_{—}324368.1; gb|AAO52327.1; ref|NP_{—}486686.1; ref|ZP_{—}00111186.1; ref|NP_{—}851434.1; ref|ZP_{—}00110255.1; emb|CAD70195.1; ref|ZP_{—}00124542.1; ref|ZP_{—}00110274.1; ref|NP_{—}856605.1; ref|NP_{—}217451.1; ref|ZP_{—}00108701.1; ref|ZP_{—}00126162.1; gb|AAD43562.1|AF155773_{—}1; ref|NP_{—}519931.1; ref|NP_{—}754319.1; pir∥T30342; ref|NP_{—}405471.1; gb|AAM12911.1; ref|ZP_{—}00012847.1; gb|AAN74983.1; ref|ZP_{—}00110275.1; ref|ZP_{—}00108808.1; ref|ZP_{—}00110898.1; ref|NP_{—}486675.1; dbj|BAB88752.1; ref|NP_{—}302532.1; ref|ZP_{—}00074380.1; gb|AAF15892.2|AF204805_{—}2; ref|NP_{—}492417.1; ref|ZP_{—}00106167.1; emb|CAA84505.1; emb|CAC44633.1; sp|P12276|FAS_CHICK; ref|ZP_{—}00110267.1; gb|AAO62585.1; ref|NP_{—}823457.1; ref|XP_{—}322886.1; gb|AAN32979.1; sp|P12785|FAS_RAT; ref|NP_{—}059028.1; emb|CAA46695.2; sp|Q03149|WA_EMENI; emb|CAB92399.1; ref|NP_{—}821274.1; gb|AAA41145.1; ref|NP 851440.1; dbj|BAB12213.1; ref|NP_{—}754362.1; gb|AAF00957.1|AF183408_{—}5; gb|AAM93545.1|AF395534_{—}1; ref|NP_{—}828538.1; ref|NP_{—}004095.3; pir∥G01880; emb|CAB38084.1; pir∥S18953; emb|CAD19100.1; pir∥S60224; ref|ZP_{—}00083375.1; ref|XP_{—}126624.1; sp|Q12053|PKS1_ASPPA; ref|NP_{—}608748.1; emb|CAC88775.1; ref|NP_{—}822020.1; dbj|BAC45240.1; gb|AAO64404.1; gb|AAD38786.1|AF151533_{—}1; emb|CAA76740.1; gb|AAC39471.1; ref|NP_{—}754360.1; sp|Q12397|STCA_EMENI; ref|NP_{—}670704.1; ref|NP_{—}819808.1; ref|XP_{—}319941.1; sp|P36189|FAS_ANSAN; gb|AAN59953.1; dbj|BAB88688.1; gb|AAO25864.1; emb|CAD29795.1; gb|AAO51709.1; gb|AAM12934.1; gb|AAO51707.1; sp|P49327|FAS_HUMAN; pir∥T18201; ref|ZP_{—}00102377.1; ref|NP_{—}624465.1; ref|NP_{—}828537.1; ref|ZP_{—}00124458.1; ref|NP_{—}647613.1; dbj|BAB88689.1; ref|ZP_{—}00089514.1; ref|NP_{—}624466.1; gb|AAO52142.1; ref|NP_{—}754345.1; gb|AAD31436.3|AF130309_{—}1; gb|AAM12925.1; gb|AAO51578.1; emb|CAA31780.1; ref|XP_{—}316979.1; ref|XP_{—}321166.1; gb|AAG10057.1; ref|ZP_{—}00052686.1; gb|AAO51589.1; gb|AAA48767.1; ref|NP_{—}754350.1; ref|NP_{—}389604.1; gb|AAF31495.1|AF071523_{—}1; gb|AAK16098.1|AF288085_{—}2; gb|AAN75188.1; ref|NP_{—}508923.1; gb|AAO25858.1; emb|CAA65133.1; gb|AAO25899.1; gb|AAN79725.1; pir∥T30183; gb|AAO39786.1; gb|AAO50749.1; ref|ZP_{—}00109665.1; gb|AAO25874.1; gb|AAO25848.1; gb|AAK72879.1|AF378327_{—}1; ref|NP_{—}489391.1; gb|AAO25869.1; gb|AAM94794.1; dbj|BAA89382.1; gb|AAD43312.1|AF144052_{—}1; gb|AAL01060.1|AF409100_{—}7; emb|CAA84504.1; gb|AAD43307.1|AF144047_{—}1; gb|AAO25844.1; gb|AAO25836.1; ref|ZP_{—}00108217.1; gb|AAD43310.1|AF144050_{—}1; gb|AAO25852.1; ref|NP_{—}717214.1; ref|ZP_{—}00068117.1; gb|AAO39778.1; gb|AAO39788.1; gb|AAO25904.1; gb|AAL06699.1; gb|AAO25889.1; gb|AAO25884.1; gb|AAD43309.1|AF144049_{—}1; ref|NP_{—}485686.1; pir|T30937; gb|AAO39787.1; gb|AAO39780.1; gb|AAF76933.1; gb|AAO25879.1; ref|NP_{—}851482.1; gb|AAO39781.1; gb|AAO39790.1; ref|NP_{—}630000.1; gb|EAA46042.1; gb|AAO51629.1; gb|AAO25894.1; gb|AAL01062.1|AF409100_{—}9; 181 2e-44; gb|AAN28672.1; gb|AAD43308.1|AF144048_{—}1; and gb|AAO39107.1.

### Example 5

### Synthesis of DEBS Module 2

DEBS Module 2 is a 4344 bp module. The module was designed to give 10 synthons of varying length (range, 350-700 bp). Each of the synthons was prepared, and the composite results are provided in Table 13. The ten synthons of DEBS Module2 were assembled by conventional methods (e.g., 3-way ligations) into a single module and secondary sequencing was performed to verify the presence of the desired sequence. Synthons for which the correct sequence was not obtained the first attempt were used for optimization and error determination and the numbers in parenthesis in Table 13 represent the second set of results.

TABLE 13
SUMMARY OF SYNTHESIS OF MODULE 001 (DEBS MODULE 2)

| | | | Total | Percent | |
|---|---|---|---|---|---|
| Synthon | Fragment Size | Correct | Sequenced | Correct | Errors/kb |
| 001-01 | 419 | 0 (31) | 26 (85) | 0 (36) | 8.4 |
| 001-02 | 527 | 1 | 12 | 8 | 4.8 |
| 001-03 | 485 | 1 | 19 | 5 | 6.6 |
| 001-04 | 739 | 3^{a} | 12 | 25 | 1.9 |
| 001-05 | 383 | 0^{b} | 24 | 0 | 8.5 |
| 001-06 | 404 | 1 | 14 | 7 | 6.8 |
| 001-07 | 392 | 0 (15) | 19 (95) | 0 (16) | 6.3 |
| 001-08 | 326 | 0^{b} | 24 | 0 | 5.9 |
| 001-09 | 517 | 1 | 45 | 2 | 6.7 |
| 001-10 | 617 | 0 (6) | 12 (17) | 0 (35) | 8.1 |

^{a}Oligos used in the assembly of synthon 001-04 were partially purified by HPLC. Different polymerase was also used for the assembly of this synthon.
^{b}Correct amino acid sequences were obtained for synthons 001-05 and 001-08 using samples that contained only silent mutations that had acceptable codon usage.

### Example 6

### Expression of Synthetic DEBS Mod2 in E. coli

The DEBS Mod2 gene in an E. coli strain having high 15-Me-6dEB production was replaced with a synthetic version (Example 5) and protein expression and polyketide titer were compared. The strain employed expresses a DEBS Mod2 derivative (with the KS5 N-terminal linker) from a stable RSF1010-based vector and DEBS2&3 from a single pET vector. The background strain (K207-3) has genes required for pantetheinylation and CoA thioester synthesis integrated on the chromosome. T7 promoters control Mod2 and DEBS 2&3 expression. Induced cultures are fed with propyl diketide to yield 15-Me-6dEB.

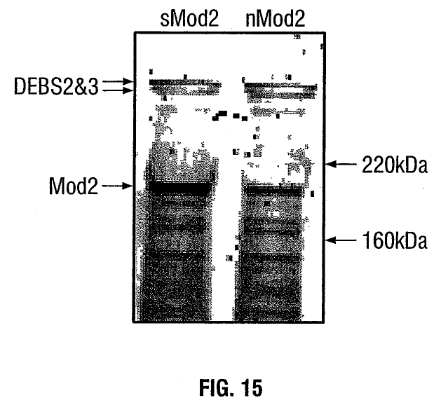

Synthetic (2) and natural (1) sequence Mod2 expressing strains produced indistinguishable levels of 15-Me-6dEB after 25 h (8 mg/L) and 42 h (25 mg/L) of expression. Quantitative PAGE analysis of the soluble protein fraction showed considerably higher protein expression from the synthetic Mod2 gene versus the natural sequence gene (FIG. 15). Approximately 3.2-fold more Mod2 protein was observed from the synthetic gene after 42 h of expression at 22° C. Equivalent titer despite higher expression level suggests that Mod2 is not production limiting in the strain used, as expected from previous work (unpublished).

Methods: Expression strain construction The ORF for synthetic DEBS Mod2 was assembled in the following way. The Spe I-Eco RI fragment of MPG011 (LLK1) was ligated into the ORF assembly vector (pKOS337-159-1). The NotI-Xba I fragment MPG001 (DEBS Mod2) was then ligated into this vector at the NotI-Spe I site. The AatII-MfeI fragment of the resulting plasmid was replaced with that from MPG009 (DEBS Mod5) to add the KS5 N-terminal linker sequence. The NdeI-EcoRI fragment of this plasmid (pKOS378-014) containing the Mod2 ORF was inserted into an pRSF1010 backbone to create the expression vector pKOS378-030. The E. coli host strain used was K207-3, which has sfp, prpE, pccB, and accA1 genes for ACP pantetheinylation and CO-A thioester synthesis integrated on its chromosome. K207-3 harboring the pET vector pBP130 [Pheifer et al., 2001, Science 291:1790-92], which expresses genes for DEBS2&3 under T7 promoter control, was transformed with pKOS378-030 and pKOS207-142a (WT Mod2 in pRSF1010; from J. Kennedy) to create synthetic (2) and WT (1) Mod2 strains, respectively. The protein sequences of the synthetic and WT Mod2 constructions are identical except for 4 substitutions in the synthetic gene required for restriction site engineering (L914Q, G1467S, T1468S, and P1551G)

PKS expression and polyketide analysis For the expression of Mod2+DEBS2&3 genes, strains grown at 37° C. to mid-log phase. Expression was induced with the addition of IPTG to 0.5 mM and fed with the addition of 500 mg/L 2-methyl-3-hydroxyhexanoyl-N-acetylcysteamine thioester (propyl diketide), 5 mM propionate, 50 mM succinate, and 50 mM glutamate. Induced cultures were incubated at 22° C. for the time indicated. At each sampling, culture supernatants were extracted with ethyl acetate and 15-Me-6dEB titer was quantitated by LC/MS (Ref). Cells were harvested, lysed with BPERII reagent (Pierce), and soluble protein was quantitated (Coomassie Plus; Pierce) and analyzed by SDS-PAGE. Gels were stained with Sypro Red (Molecular Probes) and quantitatively imaged with a Typhoon imager (Molecular Devices).

### Example 7

### Synthetic DEBS Gene Expression in E. coli

The complete 30,852 bp of the DEBS PKS gene cluster (loading di-domain, 6 elongation modules, and thioesterase releasing domain) was successfully synthesized. Using the GeMS software developed in this laboratory, the component oligonucleotides for each module and TE were designed; in total, approximately 1600 ˜40mer oligonucleotides were designed and prepared. The design utilized codons optimal for high E. coli expression and incorporated restriction sites to facilitate assembly and module interchange. Sixty-seven synthons ranging from 238 to 754 bp were prepared and cloned as described above. We observed >90 success rate in UDG cloning, and error rate of gene assembly was 3 in 1000. An average of 22% of clones sequenced were correct. Synthons were assembled into modules using the stitching sewing method, with approximately 75% of clones containing the desired vector. Module 001 (DEBSmodule2) was used for initial testing of gene synthesis and therefore the error rate (avg of ˜6.5 errors/kb) was higher for these synthons.

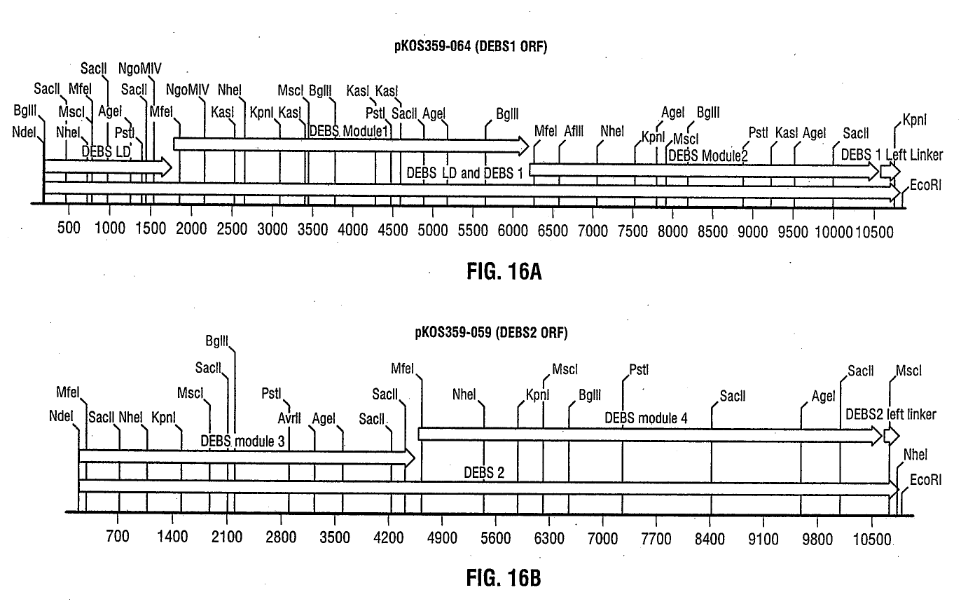

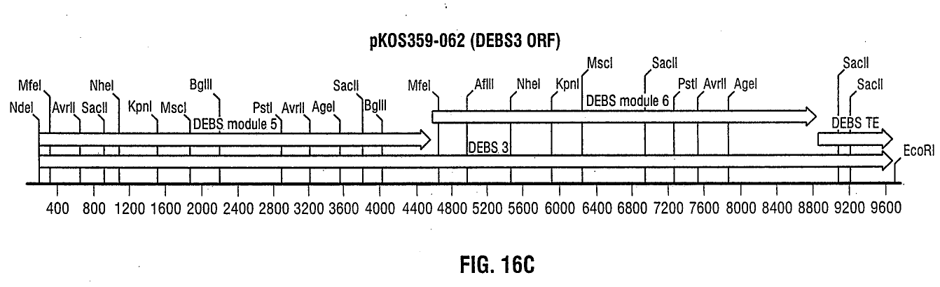

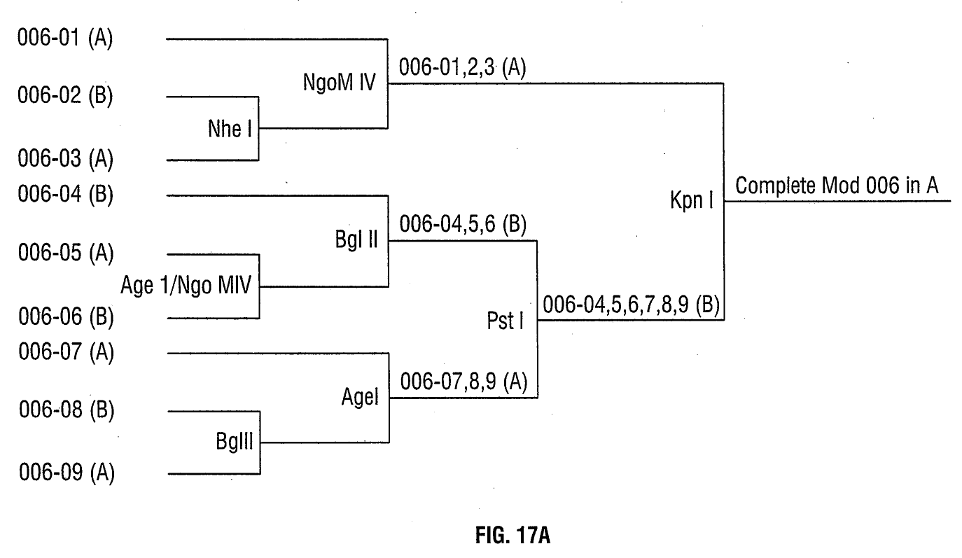

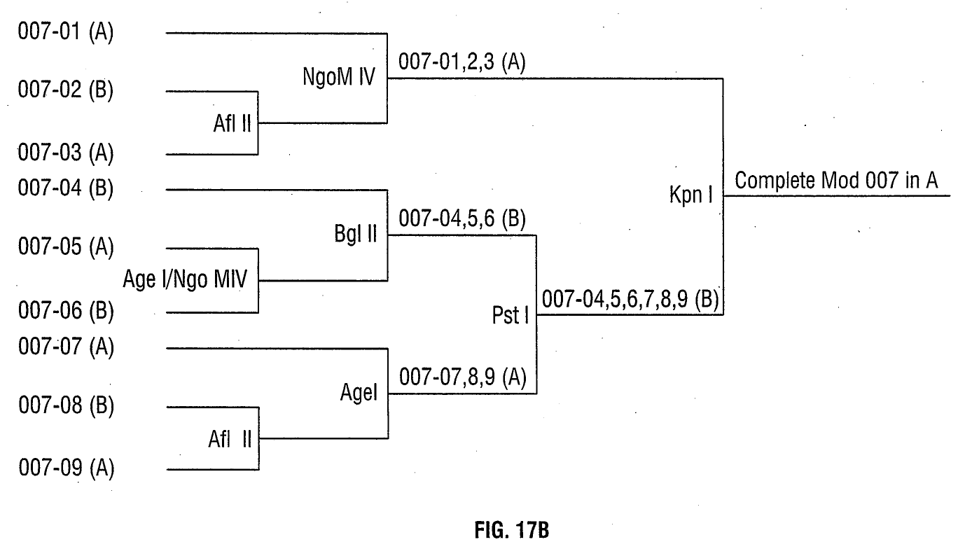

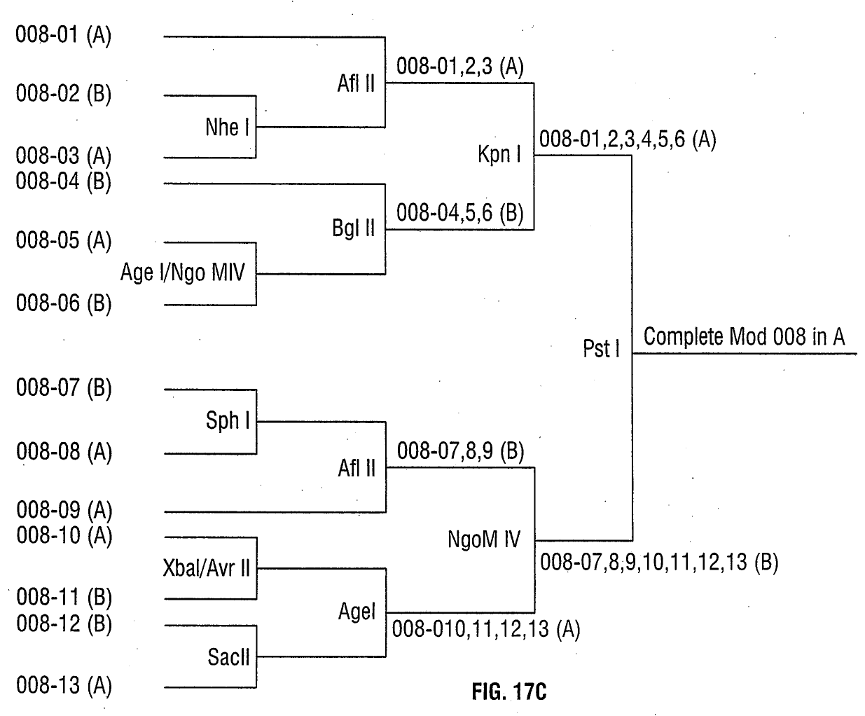

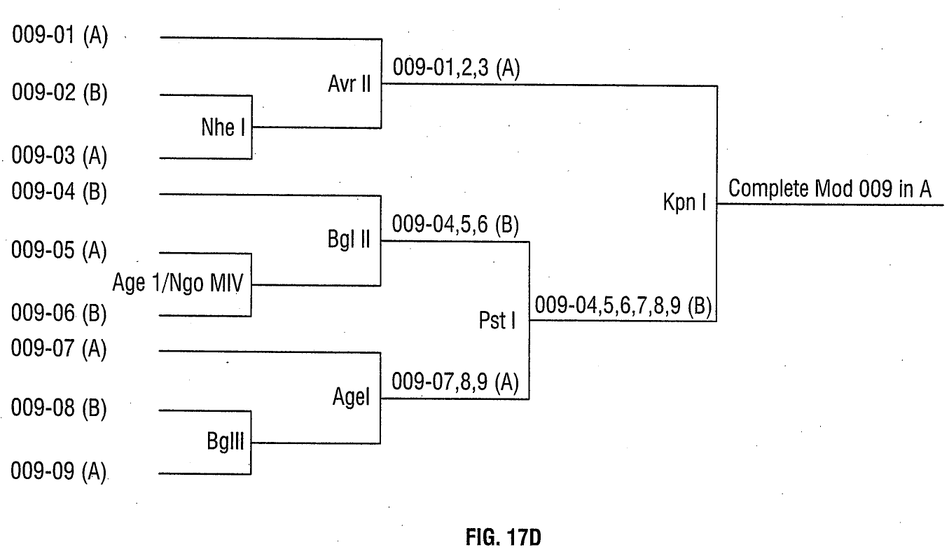

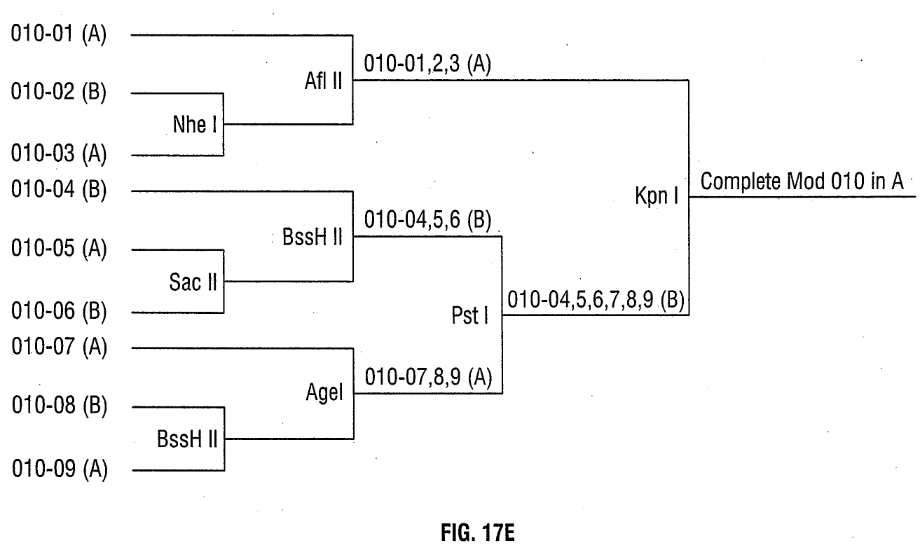

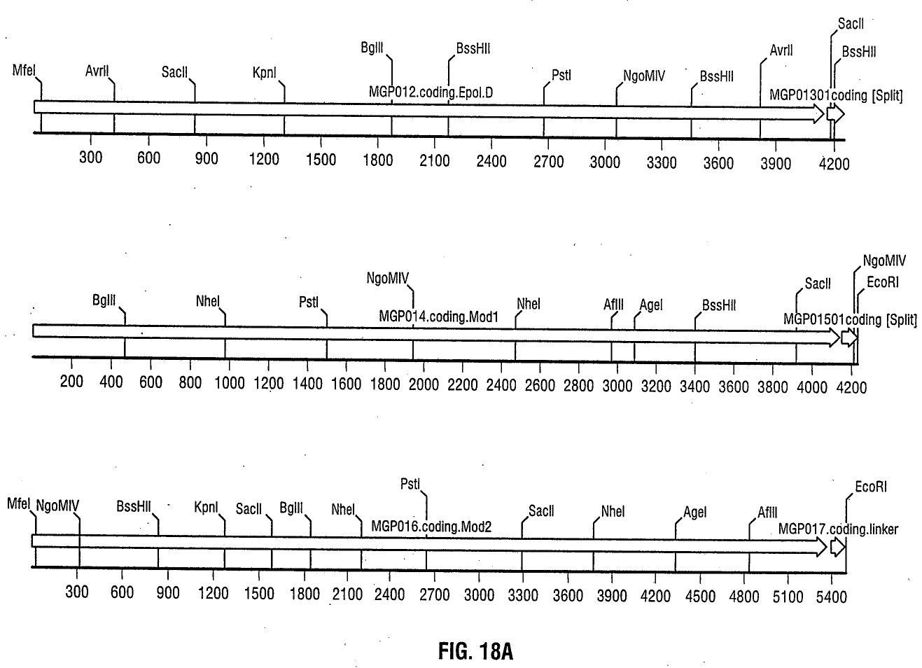

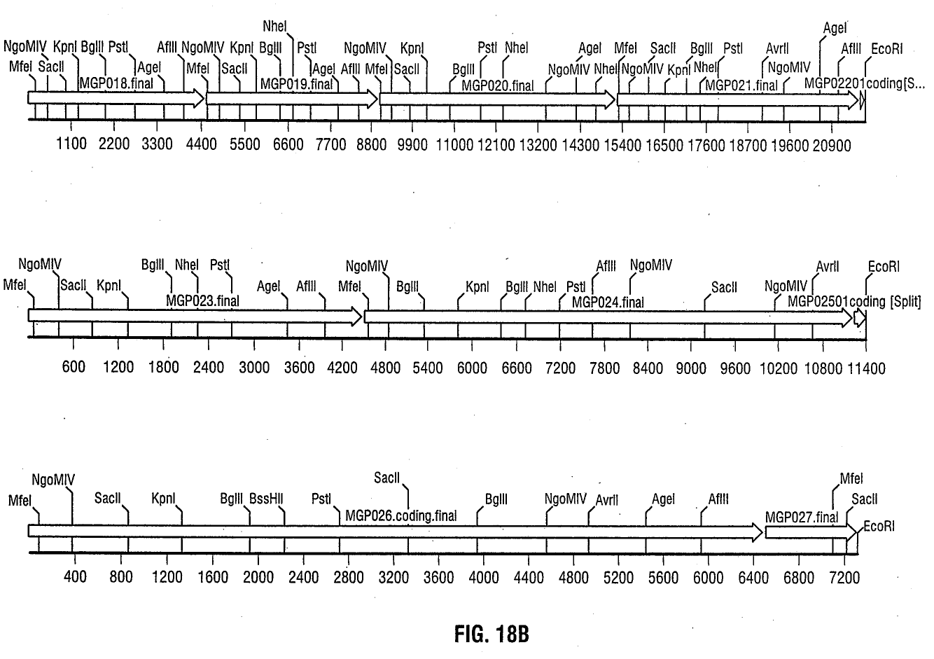

Module 2 was prepared as described in Example 5. The multi-synthon components of the remaining modules were then stitched together and selected according to the strategy shown in FIG. 16 and FIG. 17.

In an example experimental set of 10 ligations with the DEBS gene, seven gave 7/8 or 8/8 correct ligants, one gave 6/8, and two gave 3/8 and 1/8 correct; the incorrect samples were all that of the donor vector, which must have survived uncut.

All DEBS subunit genes have been fully synthesized and assembled into complete ORFs. These genes are transformed into an E. coli host strain for activity and expression testing. Synthetic and natural DEBS components are co-expressed in various combinations to determine the effects of gene synthesis codon usage and amino acid substitutions on individual subunit activities (FIG. 4-2). Synthetic DEBS1 has been successfully expressed in active form in E. coli. Total DEBS1 expression is >3-fold higher for the synthetic codon-optimized subunit than the natural sequence subunit. Synthetic DEBS1 co-expressed with natural DEBS 2 & 3 subunits supports similar levels of 6-dEB product as the natural DEBS1 construct.

The sequence of the three DEBS open reading frames of the synthetic genes are shown below in Table 14B. (Each of the sequences includes a 3′ Eco RI site which was included to facilitate addition of tags.) Table 14A shows the overall sequence similarity for the synthetic sequence and the reported sequences of DEBS2 and 3, and a corrected sequence for DEBS1.

TABLE 14A
COMPARISON OF SYNTHETIC AND NATURALLY OCCURRING SEQUENCES

| | NATURALLY OCCURRING | | | | SYNTHETIC | | |
|---|---|---|---|---|---|---|---|
| | GENE SEQUENCE¹ | | | | GENE SEQUENCE | | |
| | | Naturally | | | # | | |
| | Naturally | Occurring | | | aa | | |
| | Occurring DNA | Polypeptide | | | changes | | |
| | Sequence | Sequence | | | compared | % identity | % identity |
| | (accession #) | (accession #) | | | to | vs nat. | vs nat. |
| | Corrected | Corrected | #bp | #aa | nat. seq. | seq. | seq. |
| DEBS1 | M63676² | AAA26493¹ | 10632 | 3544 | 9 | 99.75% | 76% |
| DEBS2 | M63677 | AAA26494 | 10701 | 3567 | 9 | 99.75% | 76% |
| DEBS3 | M63677 | AAA26495 | 9510 | 3170 | 5 | 99.84% | 76% |

¹As reported in GenBank accession nos., except as noted
²DEBS1 was resequenced and the following changes relative to M63676 were used in the design of the synthetic DEBS1 gene: An early frameshift has the effect of replacing the initial 18 aa of AAA26493 with an alternate 71-aa N-terminal sequence; there are changes in an approximately 100-bp region include complementing frameshifts, which have the effect of replacing 32 aa in the reported sequence with a different 33 aa segment.

TABLE 14B
SEQUENCE OF SYNTHETIC DEBS1-3 (SEQ ID NO: 3)

DEBS1
ATGGCAGATCTGAGCAAACTCTCCGATTCTCGCACCGCCCAGCCGGCCCG
CATCGTCCGCCCATGGCCGCTGTCTGGCTGCAATGAATCCGCATTGCGTG
CTCGCGCCCGGCAGCTTCGGGCACACCTGGACCGTTTTCCGGACGCGGGC
GTGGAGGGCGTGGGTGCGGCATTGGCCCACGACGAGCAGGCGGACGCAGG
TCCGCATCGTGCGGTGGTTGTTGCTTCATCGACCTCAGAATTACTGGATG
GTCTGGCCGCGGTGGCCGATGGTCGCCCGCATGCGAGCGTCGTACGCGGC
GTTGCCCGTCCTTCTGCCCCGGTAGTGTTTGTGTTTCCTGGGCAGGGGGC
ACAGTGGGCAGGTATGGCGGGCGAGCTGCTTGGCGAGTCGCGCGTGTTCG
CTGCCGCCATGGACGCCTGTGCTCGCGCGTTCGAACCTGTGACAGACTGG
ACGCTTGCACAGGTCCTGGATAGCCCTGAACAAAGCCGCCGCGTTGAAGT
GGTCCAGCCAGCGTTATTCGCCGTGCAAACTTCGCTAGCGGCGCTCTGGC
GTTCCTTTGGCGTGACCCCAGATGCTGTGGTTGGCCATTCAATTGGTGAA
TTAGCAGCGGCGCATGTTTGCGGTGCCGCAGGTGCGGCGGATGCAGCGCG
CGCAGCGGCACTCTGGAGTCGCGAGATGATTCCGTTGGTGGGCAACGGCG
ACATGGCCGCTGTCGCTCTGTCGGCAGATGAAATTGAACCACGTATCGCG
CGCTGGGACGATGACGTAGTGCTGGCGGGCGTCAACGGTCCGCGGTCCGT
CCTGTTGACAGGGTCACCTGAACCCGTAGCTCGTCGTGTGCAGGAACTGA
GCGCCGAGGGCGTACGCGCCCAGGTAATCAATGTTAGCATGGCTGCGCAT
AGCGCTCAGGTTGATGACATCGCTGAGGGTATGCGTAGTGCCCTGGCGTG
GTTTGCCCCAGGCGGCTCCGAAGTTCCGTTCTACGCCTCACTGACCGGCG
GTGCGGTTGATACCCGTGAGTTAGTAGCCGATTACTGGCGTCGTTCTTTT
CGGCTACCGGTACGGTTTGATGAAGCGATCCGCAGTGCCTTGGAAGTAGG
CCCGGGTACGTTTGTCGAAGCGAGCCCGCATCCTGTGTTGGCGGCGGCGC
TGCAACAGACCCTGGATGCCGAAGGTTCAAGCGCGGCTGTTGTACCTACA
CTGCAGCGTGGTCAAGGGGGCATGCGTCGCTTCCTGTTGGCCGCGGCCCA
GGCTTTCACTGGCGGCGTCGCGGTTGACTGGACGGCCGCTTACGATGATG
TTGGTGCCGAACCAGGTTCGCTGCCTGAGTTCGCTCCGGCCGAAGAAGAG
GACGAGCCGGCAGAGTCCGGGGTTGATTGGAACGCACCGCCACACGTGCT
CCGCGAACGTCTGCTGGCTGTGGTGAACGGGGAGACCGCAGCTCTTGCAG
GCCGCGAAGCTGACGCAGAGGCGACCTTTCGCGAATTAGGTCTCGATTCT
GTGTTAGCAGCCCAGCTGCGCGCGAAAGTCAGCGCGGCCATTGGCCGTGA
AGTGAATATTGCGCTGTTATATGACCATCCAACCCCGCGTGCACTTGCGG
AGGCACTGTCTAGTGGGACGGAAGTAGCCCAACGCGAGACTCGCGCCCGT
ACAAACGAAGCTGCACCTGGCGAACCAATTGCGGTAGTAGCGATGGCATG
TCGTTTACCGGGCGGTGTATCGACCCCTGAAGAGTTCTGGGAGCTGTTGT
CAGAAGGCCGGGATGCGGTGGCGGGGCTTCCGACTGACAGAGGGTGGGAC
CTGGATAGCCTGTTCCACCCGGATCCAACTCGTTCGGGCACCGCCCATCA
GCGGGGCGGTGGGTTTCTGACCGAGGCGACGGCTTTTGATCCGGCCTTCT
TTGGTATGAGCCCGCGCGAGGCGTTAGCCGTGGATCCTCAGCAGCGCTTG
ATGCTGGAACTTTCTTGGGAAGTCTTAGAACGTGCCGGCATCCCGCCGAC
TTCCCTACAGGCAAGTCCGACGGGTGTTTTCGTCGGGCTGATTCCGCAGG
AGTACGGCCCACGTCTGGCGGAAGGCGGCGAAGGGGTGGAAGGCTACCTG
ATGACGGGCACGACTACATCGGTAGCGTCCGGTCGTATCGCGTACACCTT
AGGTTTGGAGGGCCCAGCTATCAGTGTCGATACGGCGTGTTCTTCGTCAC
TGGTAGCCGTACATCTCGCGTGCCAGAGCCTGCGCCGTGGCGAAAGCTCT
CTCGCCATGGCGGGCGGTGTTACCGTGATGCCGACACCGGGGATGCTGGT
TGATTTTTCGCGCATGAACAGCTTGGCGCCAGATGGTCGCTGCAAAGCGT
TCTCGGCTGGTGCGAACGGTTTCGGCATGGCTGAAGGCGCGGGCATGCTG
CTGCTGGAACCCTTATCTGACGCCCGTCGTAATGGGCACCCAGTGCTGGC
AGTGCTGCGTGGCACCGCTGTGAATAGCGATGGCGCTAGCAACGGGCTGT
CCGCTCCAAATGGTCGGGCCCAAGTCCGTGTGATCCAGCAGGCGTTAGCG
GAATCAGGTTTGGGTCCGGCGGACATTGATGCCGTTGAAGCGCATGGGAC
TGGAACCCGTCTGGGTGATCCGATTGAGGCCCGTGCACTGTTTGAAGCTT
ACGGCCGCGACCGTGAGCAGCCACTGCATCTTGGCAGTGTCAAAAGTAAC
TTAGGGCACACCCAGGCAGCCGCTGGCGTAGCAGGAGTAATCAAAATGGT
GCTTGCGATGCGCGCGGGCACCTTACCGCGCACTCTCCATGCAAGCGAGC
GTAGCAAAGAAATCGACTGGAGCAGCGGTGCTATTTCGCTGCTTGACGAA
CCTGAGCCTTGGCCTGCTGGTGCCCGGCCGCGCCGTGCCGCGGTGAGCAG
CTTTGGCATCAGCGGTACCAATGCCCATGCCATTATCGAGGAAGCCCCAC
AGGTTGTAGAAGGGGAACGTGTTGAGGCTGGCGATGTAGTTGCACCGTGG
GTGTTATCAGCCTCCTCAGCGGAAGGTCTTCGCGCACAGGCGGCGCGTTT
GGCAGCGCACCTGCGCGAACACCCTGGGCAGGACCCACGTGACATCGCGT
ACAGCCTGGCTACAGGCCGCGCGGCGCTGCCACACCGTGCGGCTTTTGCG
CCGGTGGACGAATCCGCAGCGCTGCGCGTTCTGGATGGCCTGGCGACCGG
CAATGCGGACGGCGCCGCCGTGGGTACAAGCCGGGCTCAACAGCGTGCTG
TCTTCGTGTTCCCTGGCCAGGGTTGGCAGTGGGCGGGCATGGCGGTCGAC
CTCCTGGACACAAGTCCGGTGTTCGCAGCCGCGCTCCGTGAGTGTGCAGA
TGCCCTGGAACCACATCTGGATTTTGAAGTCATTCCGTTTTTACGTGCCG
AGGCCGCGCGGCGCGAGCAGGACGCGGCTTTGAGTACGGAACGTGTGGAT
GTTGTCCAACCTGTGATGTTTGCAGTGATGGTTTCTCTGGCATCCATGTG
GCGCGCGCACGGCGTCGAACCGGCAGCGGTGATTGGGCACAGCCAAGGCG
AAATTGCTGCCGCATGCGTTGCAGGGGCACTGTCCCTGGATGATGCGGCG
CGCGTAGTGGCCCTGAGATCTCGCGTGATTGCTACTATGCCAGGCAACAA
AGGGATGGCGTCAATCGCGGCACCAGCCGGGGAAGTGCGTGCACGTATTG
GCGATCGTGTGGAGATTGCCGCTGTTAATGGCCCACGCTCGGTGGTAGTG
GCCGGTGACAGCGATGAATTAGATCGTCTCGTCGCATCTTGTACTACCGA
ATGTATTCGCGCGAAACGTCTCGCCGTAGATTATGCGAGCCATTCATCTC
ACGTAGAAACGATCCGTGACGCGCTCCATGCCGAATTAGGTGAAGATTTC
CATCCACTGCCTGGCTTTGTCCCTTTTTTTTCGACCGTGACCGGCCGTTG
GACCCAACCAGACGAACTGGACGCTGGTTATTGGTATCGTAATCTCCGTC
GCACGGTGCGCTTTGCAGATGCAGTACGGGCCCTGGCAGAACAGGGCTAT
CGCACGTTTCTGGAGGTGAGTGCGCATCCAATCCTGACAGCCGCGATTGA
GGAGATTGGTGATGGCAGTGGCGCCGACCTGTCCGCAATCCATAGCCTGC
GTCGCGGCGACGGCAGCCTGGCGGATTTTGGTGAAGCTCTGAGTCGTGCA
TTCGCGGCTGGCGTGGCAGTCGATTGGGAGTCTGTACACCTGGGCACTGG
TGCCCGCCGCGTACCGCTGCCGACCTATCCGTTTCAGCGCGAACGCGTGT
GGCTGCAGCCGAAACCTGTGGCTCGCCGGTCTACCGAGGTTGATGAAGTC
TCTGCGCTGCGCTACCGTATCGAGTGGCGTCCAACTGGCGCCGGTGAACC
GGCACGCTTGGATGGTACGTGGCTTGTAGCTAAATATGCGGGCACAGCCG
ATGAAACGAGCACTGCGGCACGCGAAGCGCTGGAATCCGCTGGGGCCCGT
GTGCGCGAACTTGTCGTCGATGCCCGTTGTGGCCGGGATGAATTAGCAGA
ACGTCTGCGTTCGGTCGGCGAAGTCGCCGGTGTTCTGAGCTTACTCGCCG
TCGATGAAGCGGAACCAGAGGAAGCGCCGCTGGCACTGGCAAGCTTAGCA
GATACGCTGAGCCTGGTTCAGGCTATGGTATCCGCGGAACTGGGGTGCCC
GCTGTGGACAGTGACCGAATCAGCAGTGGCTACGGGCCCGTTCGAACGTG
TTCGTAATGCCGCACACGGTGCGCTGTGGGGGGTAGGTCGTGTTATCGCG
CTTGAGAACCCGGCGGTCTGGGGCGGTCTCGTTGACGTACCTGCCGGTAG
CGTGGCGGAGCTTGCGCGCCACTTAGCCGCCGTGGTTTCGGGGGGCGCAG
GCGAAGATCAACTGGCGTTGCGTGCTGATGGGGTTTACGGTCGTCGTTGG
GTGCGCGCAGCAGCGCCCGCAACAGATGATGAATGGAAACCGACGGGGAC
CGTTCTGGTGACCGGTGGCACTGGTGGTGTAGGCGGCCAAATCGCCCGCT
GGTTAGCACGTCGGGGTGCTCCTCACCTTCTCCTGGTTAGCCGTAGCGGC
CCGGATGCTGATGGTGCGGGCGAACTGGTTGCAGAACTTGAAGCCCTGGG
GGCGCGTACCACGGTTGCGGCATGTGACGTGACGGACCGCGAGTCTGTGC
GCGAGCTGTTGGGCGGTATTCGCGATGACGTACCGTTATCAGCCGTCTTC
CATGCGGCGGCAACCTTGGATGACGGCACCGTCGATACTCTGACAGGTGA
ACGGATTGAACGCGCAAGCCGCGCCAAAGTGTTAGGGGCGCGCAATCTGC
ATGAGCTGACACGTGAGCTGGATCTGACCGCGTTCGTGCTGTTTTCCAGT
TTTGCGTCGGCCTTTGGTGCACCGGGTCTCGGCGGGTATGCGCCAGGCAA
CGCTTACCTGGATGGTTTGGCCCAGCAGCGTAGATCTGATGGTCTGCCTG
CTACCGCCGTGGCATGGGGGACGTGGGCGGGCTCAGGTATGGCCGAAGGG
GCCGTAGCCGATCGCTTTCGGCGTCACGGTGTTATTGAAATGCCGCCTGA
AACCGCCTGTCGTGCCTTACAGAATGCTCTGGATCGCGCAGAAGTCTGCC
CGATTGTTATCGATGTTCGTTGGGACCGCTTTTTATTAGCGTACACCGCG
CAGCGTCCAACACGCCTGTTTGATGAAATTGACGATGCCCGCCGGGCGGC
CCCGCAGGCCCCTGCTGAGCCACGCGTAGGTGCCCTGGCCTCCCTCCCGG
CTCCAGAGCGGGAAGAAGCGCTGTTCGAACTGGTGCGCTCACATGCGGCG
GCAGTGCTGGGCCATGCGTCTGCGGAACGCGTCCCTGCTGACCAAGCTTT
CGCGGAGTTGGGTGTGGATTCTCTTTCAGCGCTGGAACTGCGTAACCGCT
TAGGCGCGGCGACGGGTGTGCGTCTTCCAACCACGACAGTGTTCGATCAC
CCAGATGTTCGTACGTTGGCCGCCCATCTCGCGGCGGAATTGTCTAGTGC
AACCGGCGCGGAACAAGCGGCACCTGCGACGACTGCGCCGGTCGATGAAC
CAATTGCTATCGTCGGTATGGCTTGTCGCCTGCCGGGTGAGGTGGACTCA
CCGGAACGTCTTTGGGAATTAATTACCTCTGGCCGGGACTCTGCGGCGGA
GGTTCCAGACGATCGCGGTTGGGTGCCTGATGAGCTGATGGCTAGTGACG
CTGCGGGGACCCGTGCACATGGGAACTTCATGGCAGGTGCCGGTGACTTC
GATGCGGCTTTTTTCGGCATTAGCCCGCGTGAAGCACTGGCGATGGATCC
GCAGCAGCGCCAGGCGCTGGAAACGACCTGGGAAGCGTTGGAAAGTGCAG
GCATTCCTCCGGAAACCTTAAGGGGTAGTGACACGGGTGTTTTTGTGGGT
ATGTCTCACCAGGGCTACGCAACGGGGCGTCCACGTCCGGAAGACGGCGT
CGACGGTTATCTTTTAACCGGCAACACCGCAAGTGTCGCGAGTCGGCGTA
TCGCCTATGTCCTGGGGTTGGAGGGCCCGGCACTTACTGTGGACACGGCA
TGTTCCAGCAGTCTGGTGGCCTTGCACACCGCGTGTGGGAGTTTACGGGA
CGGTGATTGCGGCCTGGCTGTTGCGGGTGGCGTCTCAGTAATGGCGGGCC
CGGAAGTATTTACCGAGTTCTCGCGTCAGGGTGCGCTGTCCCCGGATGGC
CGCTGTAAACCGTTTTCCGATGAAGCTGATGGCTTCGGGCTGGGCGAAGG
TAGCGCGTTCGTTGTTTTACAACGTCTGTCGGATGCGCGCCGTGAAGGTC
GCCGCGTTTTAGGTGTGGTCGCAGGTTCGGCCGTGAACCAGGATGGCGCT
AGCAACGGTCTGTCGGCTCCTTCCGGTGTAGCTCAGCAGCGCGTGATCCG
TCGCGCCTGGGCTCGTGCGGGTATTACGGGAGCCGATGTAGCGGTGGTGG
AAGCGCACGGAACTGGTACTCGTCTGGGCGATCCAGTTGAGGCATCGGCC
CTGCTGGCTACTTACGGCAAATCACGCGGCAGCAGTGGTCCGGTGCTGCT
GGGGTCGGTCAAATCCAATATTGGTCATGCCCAAGCCGCCGCTGGCGTGG
CGGGCGTGATCAAAGTGCTGCTTGGTCTTGAACGGGGCGTGGTTCCGCCT
ATGCTGTGCCGTGGGGAGCGGTCAGGGCTGATTGACTGGAGTTCTGGGGA
GATCGAACTCGCCGACGGGGTGCGCGAATGGTCCCCGGCAGCAGATGGCG
TACGTCGTGCGGGCGTTTCAGCCTTTGGTGTGAGCGGTACCAATGCCCAC
GTGATTATTGCGGAACCGCCGGAACCGGAGCCGGTGCCGCAGCCTCGTCG
TATGCTGCCTGCCACGGGTGTAGTTCCGGTTGTGTTGTCAGCTCGTACGG
GTGCTGCGCTGCGTGCGCAGGCTGGCCGTCTGGCGGATCATTTAGCGGCG
CACCCGGGCATTGCTCCGGCCGACGTGTCCTGGACGATGGCGCGCGCCCG
CCAACACTTTGAAGAACGTGCTGCTGTGCTTGCAGCCGATACCGCCGAAG
CAGTTCACCGGTTGCGTGCTGTCGCAGACGGCGCTGTGGTCCCTGGTGTT
GTGACTGGTAGCGCGAGTGATGGTGGGAGCGTTTTCGTTTTCCCTGGCCA
GGGGGCCCAATGGGAGGGCATGGCCCGCGAACTGCTGCCTGTTCCGGTTT
TCGCCGAATCTATTGCCGAATGCGATGCTGTTCTCAGTGAGGTGGCCGGT
TTTAGCGTGTCGGAAGTTTTAGAGCCGCGCCCGGATGCACCGTCCCTGGA
GCGGGTGGATGTGGTGCAACCAGTGCTGTTTGCGGTGATGGTGTCTTTGG
CGCGCTTATGGCGTGCGTGTGGCGCGGTTCCATCGGCTGTTATTGGACAT
AGCCAGGGCGAAATTGCGGCGGCGGTAGTTGCAGGTGCGCTGTCACTTGA
AGATGGCATGCGCGTCGTTGCTCGTAGATCTCGCGCCGTCCGTGCAGTTG
CGGGGCGTGGGAGTATGCTGTCGGTACGTGGTGGTCGCAGCGATGTCGAG
AAACTGCTGGCGGATGACAGCTGGACCGGGCGACTTGAAGTAGCGGCCGT
AAATGGTCCTGACGCCGTCGTCGTCGCTGGTGACGCGCAGGCGGCACGTG
AGTTCTTAGAATATTGTGAAGGCGTTGGCATCCGTGCCCGCGCGATTCCT
GTGGATTACGCCAGTCATACCGCCCATGTGGAACCAGTGCGCGATGAACT
TGTGCAGGCTCTGGCGGGTATCACGCCGCGCCGGGCGGAAGTCCCATTCT
TTTCCACTCTGACCGGCGATTTTTTGGATGGTACGGAATTAGATGCAGGC
TATTGGTATCGCAACTTACGTCACCCGGTCGAATTTCATTCAGCGGTACA
GGCGCTGACGGATCAGGGTTACGCAACTTTTATTGAAGTAAGCCCGCATC
CTGTGCTGGCATCGTCAGTACAGGAAACCCTGGATGACGCTGAATCTGAT
GCTGCCGTCTTGGGCACTCTGGAACGCGATGCGGGCGATGCGGACCGTTT
TCTGACTGCCCTTGCTGATGCCCATACGCGTGGCGTAGCAGTCGATTGGG
AGGCCGTTCTGGGCCGGGCGGGCCTTGTTGATCTTCCGGGTTACCCGTTC
CAGGGCAAACGCTTCTGGCTGCAGCCTGATCGGACCACTCCGCGTGACGA
ACTGGATGGTTGGTTCTATCGCGTCGACTGGACGGAGGTGCCGCGTTCTG
AACCGGCAGCACTTCGGGGCCGCTGGCTGGTGGTTGTCCCGGAAGGTCAT
GACGAAGACGGCTGGACCGTGGAGGTCCGTTCCGCTCTGGCCGAAGCGGG
GGCCGAACCCGAGGTGACCCGTGGCGTGGGCGGCCTCGTCGGCGATTGCG
CGGGCGTAGTCAGCTTACTGGCATTGGAGGGCGACGGTGCTGTTCAGACC
TTGGTCCTCGTCCGTGAATTGGACGCTGAGGGCATTGATGCCCCGTTATG
GACGGTCACTTTCGGCGCCGTGGATGCTGGTTCCCCAGTCGCCCGGCCTG
ATCAGGCGAAACTGTGGGGTCTCGGGCAAGTAGCATCGTTGGAACGTGGG
CCACGCTGGACTGGTCTGGTGGACTTGCCGCACATGCCGGATCCAGAGCT
GCGCGGACGCCTGACGGCAGTTCTTGCGGGCTCTGAGGATCAGGTCGCTG
TTCGTGCGGATGCCGTCCCGGCCCGCCGTCTGAGCCCTGCGCATGTCACC
GCGACCTCCGAATACGCCGTGCCGGGCGGCACGATTTTGGTTACCGGTGG
GACCGCAGGGCTGGGTGCGGAAGTCGCCCGCTGGCTGGCAGGCCGTGGCG
CTGAACATCTGGCACTGGTGAGTCGCCGGGGTCCTGACACCGAAGGGGTC
GGCGATCTGACCGCCGAACTGACCCGCTTGGGTGCCCGCGTTAGCGTGCA
CGCGTGCGATGTATCTTCACGTGAACCAGTGCGTGAACTGGTGCACGGCC
TGATTGAACAAGGCGATGTGGTACGTGGCGTGGTCCATGCTGCGGGCTTG
CCGCAGCAGGTGGCGATCAATGACATGGATGAGGCGGCGTTTGACGAAGT
CGTCGCGGCTAAAGCTGGTGGCGCGGTTCATCTGGACGAACTTTGCAGCG
ATGCCGAACTTTTCCTGTTATTTAGCAGCGGTGCTGGCGTCTGGGGGAGC
GCGCGCCAAGGTGCCTATGCAGCGGGTAACGCCTTCCTTGACGCCTTCGC
TCGTCACCGCCGCGGTCGCGGTTTACCGGCTACCAGTGTTGCATGGGGCC
TGTGGGCCGCAGGTGGGATGACGGGGGATGAAGAGGCCGTAAGCTTTCTG
CGTGAACGTGGCGTACGCGCCATGCCAGTACCGCGTGCGCTGGCTGCTTT
AGATCGCGTGTTGGCATCCGGGGAGACCGCCGTCGTAGTTACCGATGTGG
ACTGGCCTGCGTTTGCCGAATCTTACACCGCCGCCCGTCCGCGCCCATTG
CTGGACCGTATCGTTACCACGGCACCGAGCGAGCGCGCTGGCGAGCCGGA
AACCGAATCCCTGCGCGATCGCTTGGCCGGGCTCCCTCGTGCGGAACGGA
CGGCGGAGCTCGTTCGTTTGGTGCGCACGTCGACGGCAACCGTTCTGGGT
CACGACGATCCGAAAGCCGTGCGGGCCACCACCCCATTTAAAGAATTGGG
TTTCGACTCTCTTGCTGCCGTGCGCCTCCGTAATCTGCTCAATGCGGCAA
CTGGCCTGCGCCTGCCGTCCACGCTTGTTTTCGATCATCCGAACGCCAGT
GCTGTCGCCGGTTTCTTGGATGCTGAGCTGTCTAGTGAAGTGCGTGGCGA
AGCTCCGTCCGCCCTGGCTGGTCTGGATGCATTGGAGGGCGCGCTGCCGG
AAGTGCCTGCGACGGAACGTGAGGAGCTGGTCCAGCGTCTGGAACGCATG
CTCGCGGCACTGCGGCCGGTAGCCCAAGCAGCTGACGCGAGTGGTACCGG
CGCGAACCCAACCGGTGACGATCTTGGTGAAGCCGGTGTTGATGAACTGT
TGGAGGCTTTAGGGCGCGAATTAGATGGGGACGGGAATTCT
DEBS2 (SEQ ID NO: 4)
ATGACAGACAGTGAGAAAGTTGCTGAGTATCTGCGCCGCGCCACCCTGGA
TCTTCGTGCGGCACGCCAGCGCATCCGTGAACTGGAAAGTGATCCAATTG
CTATTGTCAGCATGGCGTGTCGCCTGCCAGGGGGTGTTAATACGCCACAG
CGCTTGTGGGAGTTACTGCGTGAGGGTGGCGAAACTCTGTCGGGCTTTCC
TACTGACCGTGGCTGGGACCTGGCACGTCTGCACCACCCGGATCCAGACA
ATCCGGGGACGTCATACGTGGATAAAGGCGGTTTCTTGGACGACGCCGCA
GGCTTCGACGCCGAGTTTTTTGGTGTGAGCCCGCGTGAGGCTGCGGCGAT
GGATCCTCAGCAACGCTTGTTACTGGAAACCTCCTGGGAACTGGTGGAAA
ACGCAGGTATCGACCCGCACAGCTTAAGAGGTACGGCGACGGGTGTCTTC
CTGGGTGTTGCTAAATTTGGCTATGGTGAAGATACCGCCGCTGCGGAGGA
CGTAGAAGGGTACTCGGTGACCGGGGTGGCGCCCGCGGTGGCGTCCGGCC
GTATTTCCTACACTATGGGCCTGGAGGGGCCGTCGATTAGCGTCGATACC
GCTTGCTCCTCCTCATTAGTTGCGTTACACCTTGCCGTTGAGTCTCTGCG
TAAAGGGGAGAGCAGCATGGCGGTTGTCGGTGGCGCGGCCGTCATGGCAA
CACCTGGCGTTTTCGTCGATTTTTCTCGCCAACGTGCACTCGCAGCGGAT
GGTCGGAGCAAAGCCTTTGGCGCGGGCGCCGATGGTTTCGGCTTTAGCGA
ACGTGTAACCTTGGTTCTGCTGGAGCGTCTGTCCGAAGCGCGGCGCAACG
GCCATGAAGTGCTGGCTGTCGTTCGTGGGAGCGCACTGAACCAAGATGGC
GCTAGCAATGGCTTGAGCGCTCCTTCCGGGCCAGCACAGCGCCGTGTAAT
TCGCCAAGCGCTGGAAAGCTGCGGTCTCGAACCAGGCGATGTGGACGCGG
TAGAAGCACACGGCACGGGCACGGCTCTGGGTGATCCGATTGAGGCAAAC
GCTTTGCTGGATACCTATGGCCGTGATCGTGATGCAGACCGCCCACTTTG
GCTGGGCTCTGTTAAATCAAACATCGGCCATACCCAGGCCGCGGCAGGCG
TGACTGGCTTACTGAAAGTGGTTCTGGCGTTACGCAACGGCGAGCTGCCC
GCGACCCTGCATGTTGAAGAACCGACACCTCACGTGGATTGGAGTTCGGG
CGGCGTCGCGCTTCTGGCCGGGAACCAGCCATGGCGCCGTGGCGAACGGA
CGCGCCGGGCCCGTGTTTCCGCATTTGGCATTTCTGGTACCAACGCACAT
GTGATTGTGGAAGAAGCACCGGAGCGTGAACATCGTGAAACCACCGCTCA
CGACGGCAGACCTGTCCCGCTGGTTGTCAGCGCCCGGACTACAGCGGCTC
TTCGCGCACAGGCCGCTCAGATCGCTGACCTGTTAGAGCGTCCGGACGCC
GATTTAGCCGGGGTGGGCCTGGGTTTGGCGACCACACGCGCCCGGCACGA
GCATCGCGCCGCCGTGGTGGCCTCCACCCGGGAAGAGGCGGTGCGTGGGC
TGCGCGAAATTGCTGCTGGGGCCGCGACTGCCGATGCAGTGGTCGAGGGG
GTTACTGAAGTAGACGGTCGCAATGTAGTCTTTTTATTCCCTGGCCACGG
CTCCCAGTGGGCGGGTATGGGCGCGGAATTGCTGTCCAGTTCACCCGTCT
TCGCAGGTAAAATTCGCGCCTGTGACGAAAGCATGGCGCCAATGCAGGAT
TGGAAAGTTTCAGATGTGCTGCGTCAGGCTCCAGGGGCGCCAGGTCTGGA
TCGTGTTGATGTTGTACAACCAGTTCTGTTTGCCGTAATGGTTAGCTTAG
CCGAGCTGTGGCGCAGCTATGGCGTGGAACCGGCCGCGGTGGTGGGTCAT
TCGCAGGGCGAGATTGCGGCAGCACATGTCGCTGGGGCTCTCACCCTCGA
AGATGCTGCCAAATTAGTAGTGGGTAGATCTCGTTTGATGCGCTCTTTAT
CTGGGGAAGGGGGGATGGCTGCCGTGGCATTAGGCGAGGCAGCAGTTCGC
GAGCGTCTGCGTCCGTGGCAGGATCGCCTTTCTGTTGCGGCAGTGAATGG
CCCGCGTAGCGTTGTGGTATCAGGCGAGCCAGGTGCTCTGCGTGCGTTCT
CAGAAGATTGCGCGGCCGAGGGTATTCGCGTGCGTGACATCGATGTAGAT
TATGCAAGCCATTCTCCGCAGATCGAACGCGTTCGCGAAGAGCTGCTGGA
GACAGCCGGCGATATTGCTCCGCGTCCGGCGCGTGTGACCTTCCACAGTA
CCGTTGAATCGCGTTCGATGGATGGCACCGAACTTGATGCCCGGTATTGG
TATCGCAATTTGCGGGAAACGGTCCGCTTTGCGGATGCGGTCACACGTCT
GGCAGAATCTGGTTATGATGCCTTCATTGAGGTTAGTCCTCATCCGGTGG
TGGTTCAGGCAGTGGAAGAGGCCGTGGAGGAAGCTGACGGCGCTGAAGAC
GCGGTGGTTGTCGGTAGTCTTCACCGCGACGGTGGCGACCTGAGCGCGTT
CCTTCGTTCGATGGCAACGGCACACGTAAGCGGTGTGGACATCCGTTGGG
ATGTAGCGCTTCCGGGGGCTGCCCCATTTGCTTTACCTACGTACCCTTTT
CAACGCAAACGCTACTGGCTGCAGCCAGCGGCACCTGCTGCCGCGAGCGA
TGAACTGGCGTACCGCGTTTCATGGACACCTATTGAAAAACCAGAGAGCG
GTAATCTGGATGGTGATTGGTTGGTTGTGACCCCGCTGATCTCACCGGAA
TGGACTGAGATGCTGTGTGAAGCAATCAACGCTAACGGTGGCCGCGCCCT
GCGTTGCGAAGTCGACACAAGCGCGTCTCGGACGGAGATGGCTCAAGCGG
TTGCGCAGGCTGGCACGGGTTTTCGCGGCGTGCTGAGCCTTTTATCCTCC
GATGAAAGTGCCTGTCGCCCGGGCGTCCCTGCCGGTGCCGTTGGGTTGCT
GACGCTTGTCCAGGCCCTAGGCGACGCAGGTGTAGACGCGCCGGTGTGGT
GCCTGACTCAAGGTGCGGTGCGCACCCCGGCGGACGATGATTTAGCACGT
CCGGCGCAGACCACCGCCCATGCTTTTGCCCAAGTGGCGGGCCTGGAATT
GCCAGGGCGGTGGGGGGGTGTAGTTGATCTGCCAGAGTCTGTAGATGACG
CAGCACTGCGTCTTCTGGTGGCAGTCTTGCGGGGTGGCGGTCGTGCGGAG
GATCATCTGGCCGTCCGTGATGGTCGTCTCCATGGTCGCCGCGTAGTGAG
AGCTAGTCTCCCACAATCGGGTAGTCGCAGCTGGACCCCTCACGGCACAG
TGTTGGTTACCGGTGCGGCAAGCCCGGTCGGCGATCAACTGGTCCGTTGG
CTGGCCGACCGTGGCGCTGAACGTCTGGTTCTGGCAGGCGCATGCCCGGG
GGATGATCTGCTTGCGGCCGTTGAAGAAGCTGGCGCGTCAGCGGTCGTCT
GTGCGCAAGACGCCGCCGCGCTGCGTGAAGCTTTAGGCGACGAACCCGTG
ACTGCTTTAGTGCACGCTGGCACTCTGACGAACTTTGGCTCTATTTCCGA
GGTAGCTCCGGAGGAATTTGCAGAAACCATCGCGGCGAAAACTGCGCTCC
TGGCCGTCCTGGATGAGGTTCTGGGTGATCGCGCCGTGGAACGCGAAGTA
TATTGCTCGTCTGTGGCCGGTATTTGGGGCGGTGCGGGGATGGCAGCTTA
TGCAGCGGGTTCGGCATATTTGGACGCGCTGGCTGAACACCATCGGGCAC
GCGGTCGTTCATGCACCTCCGTTGCTTGGACGCCATGGGCGTTGCCGGGC
GGTGCCGTTGATGATGGCTACTTAAGAGAACGCGGTTTGCGTTCACTGTC
GGCTGACCGCGCGATGCGTACCTGGGAACGTGTTCTGGCAGCAGGCCCGG
TGTCCGTCGCCGTCGCCGACGTAGATTGGCCGGTGCTGTCAGAAGGTTTC
GCGGCGACCCGTCCTACTGCCCTCTTCGCAGAACTGGCGGGCCGCGGGGG
TCAGGCAGAAGCCGAACCGGACAGTGGTCCGACGGGCGAGCCTGCTCAGC
GCTTGGCTGGGTTGTCGCCGGACGAACAGCAGGAAAACCTGCTGGAATTA
GTTGCCAATGCGGTTGCCGAAGTTTTAGGCCATGAGTCCGCGGCCGAGAT
CAACGTGCGCCGGGCATTTAGCGAGCTGGGTTTAGACAGTTTAAATGCAA
TGGCCCTCCGCAAACGCCTCAGCGCCAGCACCGGCCTGCGCTTACCGGCG
TCGCTCGTGTTCGATCATCCGACTGTCACGGCATTAGCCCAACACCTTCG
CGCTCGTCTCTCTAGTGACGCCGATCAGGCGGCGGTTCGCGTTGTGGGCG
CAGCGGATGAAAGCGAGCCAATTGCCATTGTCGGCATCGGCTGCCGTTTC
CCGGGTGGCATCGGCTCTCCTGAACAGCTGTGGCGCGTTCTTGCAGAAGG
GGCCAATCTGACGACCGGCTTTCCGGCAGATCGCGGCTGGGACATCGGCC
GTCTGTACCATCCAGACCCGGATAATCCGGGCACGTCCTATGTCGACAAA
GGTGGCTTTCTCACCGACGCAGCGGATTTTGATCCGGGTTTTTTTGGTAT
TACACCGCGCGAAGCTTTGGCAATGGACCCGCAGCAGCGCTTAATGCTTG
AAACAGCATGGGAGGCAGTCGAACGTGCGGGCATTGACCCGGATGCCTTA
AGAGGCACCGACACAGGCGTTTTCGTAGGCATGAACGGTCAAAGTTACAT
GCAGTTACTGGCAGGTGAAGCGGAGCGTGTAGATGGTTACCAAGGCTTAG
GCAACAGCGCATTCGTTTTGAGTGGTCGTATCGCTTATACGTTTGGTTGG
GAAGGCCCGGCGCTGACTGTTGATACCGCGTGTTCGTCTTCGTTGGTTGG
TATTCATCTGGCAATGCAAGCGCTCCGTCGTGGGGAATGCTCTCTCGCCC
TGGCTGGTGGTGTTACCGTCATGTCAGACCCGTATACCTTCGTCGACTTC
TCGACCCAGCGTGGTCTGGCTAGTGATGGTCGCTGTAAAGCGTTCTCAGC
GCGGGCTGATGGTTTCGCGCTTTCGGAAGGCGTGGCCGCCCTCGTGCTGG
AACCGCTTAGCCGTGCGCGTGCCAACGGGCACCAAGTGCTGGCGGTGCTG
CGTGGTTCTGCCGTTAACCACGATGGGGCTAGCAATGGCCTGGCCGCCCC
AAACGGTCCATCGCAGGAACGTGTCATCCGTCAGGCGCTCGCCGCCAGCC
GGGTGCCTGCTGCTGACGTGGATGTCGTGGAAGCGCACGGCACTGGTACA
GAATTGGGCGACCCAATCGAGGCGGGTGCTCTGATCGCAACGTACGGGCA
GGATCGTGACCGCCCGCTGCGTTTGGGGAGCGTGAAAACCAACATTGGTC
ATACCCAAGCAGCAGCGGGGGCCGCAGGGGTAATTAAAGTAGTGCTGGCG
ATGCGTCATGGTATGCTGCCGCGTAGCCTGCACGCTGACGAACTGTCTCC
TCATATCGATTGGGAGTCAGGCGCTGTGGAGGTCCTGCGTGAAGAAGTAC
CGTGGCCCGCAGGCGAACGCCCGCGCCGCGCGGGTGTTTCCTCCTTCGGC
GTTTCAGGTACCAACGCGCACGTTATTGTGGAAGAGGCACCGGCCGAACA
GGAAGCGGCTCGTACCGAACGCGGCCCGCTGCCGTTCGTTCTGTCTGGGC
GCTCCGAAGCTGTGGTAGCCGCGCAGGCCCGCGCACTTGCTGAGCACTTA
CGCGACACCCCAGAGCTGGGGCTGACCGATGCTGCGTGGACTCTGGCGAC
CGGCCGTGCACGTTTCGACGTGCGCGCCGCCGTATTGGGCGATGATCGCG
CTGGTGTATGCGCGGAACTGGATGCCTTAGCGGAAGGTCGCCCGTCTGCG
GATGCGGTGGCACCAGTCACCTCCGCGCCACGTAAACCAGTCCTGGTTTT
CCCTGGCCAGGGGGCCCAGTGGGTTGGTATGGCCCGCGACTTACTGGAAA
GTTCTGAGGTCTTTGCCGAGTCGATGAGCCGCTCCGCGGAAGCGCTGTCG
CCTCACACTGATTGGAAACTTCTTGACGTTGTGCGTGGTGATGGTGGTCC
AGATCCGCACGAGCGTGTAGACGTCTTACAGCCGGTCCTGTTTTCCATTA
TGGTCTCTCTCGCGGAACTGTGGCGTGCCCACGGTGTGACTCCGGCCGCT
GTTGTAGGTCACTCTCAAGGCGAAATTGCAGCCGCACACGTGGCGGGTGC
GTTAAGCTTGGAAGCCGCAGCTAAAGTGGTGGCCTTGAGATCTCAAGTAC
TGCGTGAGCTTGATGATCAGGGCGGGATGGTTTCAGTAGGGGCATCTCGG
GATGAACTGCAAACGGTGCTGGCACGCTGGGACGGCCGCGTAGCACTGGC
CGCTGTGAATGGTCCAGGGACCTCAGTTGTCGCAGGCCCTACTGCCGAAT
TGGATGAGTTCTTTGCCGAAGCCGAAGCCCGTGAAATCAAACCACGCCCT
ATCGCAGTTCGTTATGCGAGCCATTCCCCGGAAGTCGCACGTATTGAAGA
TCGTCTGGCAGCCGAACTCGGTACAATTACCGCCGTTCGCGGCAGCGTAC
CTCTGCATAGCACGGTTGCCGGCGAAGTAATTGATACCAGCGCGATGGAC
CCGTCTTATTGGTATCGTAACTTGCGCCGTCCGGTTTTGTTTGAACAAGC
CGTGCGTGGTCTCGTCGAACAGGGGTTTGACACATTTGTCGAGGTTTCCC
CACATCCGGTTCTGCTGATGGCAGTGGAGGAGACAGCAGAACATCCAGGG
GCGGAAGTCACCTGTGTTCCTACGCTTCGTCGCGAGCAGTCCGGCCCGCA
TGAGTTTCTGCGGAACCTGCTGCGCGCCCATGTCCACGGCGTTGGCGCCG
ATCTGCGTCCTGCCGTTGCTGGCGGCCGTCCGGCTGAATTACCAACTTAC
CCGTTCGAACATCAACGTTTTTGGCTGCAGCCGCACCGCCCAGCAGATGT
TAGCGCCTTAGGCGTACGCGGGGCAGAGCACCCTCTGCTCCTGGCAGCCG
TTGACGTTCCGGGTCACGGTGGTGCCGTTTTCACCGGGCGTCTGTCTACG
GACGAGCAGCCGTGGCTGGCCGAACATGTCGTGGGCGGTCGTACCTTGGT
GCCGGGTTCCGTGCTGCTGGACCTGGCGCTGGCGGCCGGTGAAGATGTAG
GGCTGCCGGTATTGGAAGAATTGGTTTTACAACGCCCACTGGTACTGGCA
GGTGCGGGCGCTCTCCTGCGTATGTCGGTCGGCGCTCCGGATGAATCAGG
CCGCCGTACTATTGATGTCCACGCGGCAGAAGATGTAGCGGACCTCGCGG
ACGCCCAGTGGTCGCAGCATGCGACAGGTACATTGGCGCAAGGCGTCGCC
GCTGGCCCTCGGGATACCGAACAGTGGCCGCCTGAAGATGCGGTTCGCAT
CCCGCTTGATGACCATTATGACGGCCTGGCAGAACAGGGCTACGAGTATG
GTCCGTCTTTCCAGGCGTTACGTGCGGCCTGGCGCAAAGATGACTCTGTC
TACGCAGAAGTTTCAATCGCGGCGGACGAAGAGGGCTACGCGTTTCACCC
GGTGCTGCTGGACGCGGTAGCTCAAACGCTGAGCTTAGGGGCACTCGGTG
AACCGGGTGGCGGGAAACTTCCATTTGCATGGAATACGGTGACCCTTCAC
GCGAGTGGCGCGACTTCGGTTCGTGTAGTGGCGACCCCAGCTGGTGCCGA
TGCCATGGCCCTGCGTGTGACGGATCCGGCAGGTCATTTAGTGGCTACCG
TTGATTCTCTTGTGGTCCGCTCAACTGGTGAGAAATGGGAACAACCGGAA
CCGCGCGGGGGCGAAGGGGAGCTTCATGCACTGGACTGGGGCCGCTTGGC
GGAACCAGGCTCTACTGGTCGTGTTGTAGCAGCTGACGCCAGCGATTTAG
ACGCCGTCTTAAGGTCTGGTGAACCGGAGCCAGATGCCGTTTTAGTTCGT
TACGAGCCGGAGGGTGATGATCCTCGCGCTGCGGCACGCCACGGTGTGCT
GTGGGCTGCGGCGCTGGTTCGCCGCTGGCTGGAACAGGAGGAACTGCCGG
GCGCCACGCTGGTGATCGCAACGTCAGGGGCCGTCACTGTGAGTGATGAC
GATTCTGTTCCGGAGCCGGGCGCCGCGGCCATGTGGGGCGTCATTCGCTG
CGCGCAAGCGGAATCCCCGGATCGTTTCGTATTGTTAGATACTGATGCCG
AGCCTGGTATGCTGCCTGCGGTGCCAGACAATCCGCAACTTGCGCTTCGG
GGTGACGACGTGTTTGTGCCTCGTCTGAGCCCGCTCGCGCCGAGTGCCCT
GACGCTGCCAGCAGGCACCCAACGCCTTGTCCCGGGCGATGGCGCTATTG
ATTCTGTGGCATTCGAACCTGCGCCGGACGTTGAGCAGCCTCTGCGCGCG
GGTGAGGTACGGGTTGATGTGCGTGCGACCGGCGTAATTTTTCGTGATGT
TTTGTTAGCCCTGGGCATGTATCCGCAAAAAGCCGATATGGGTACGGAAG
CAGCCGGCGTAGTGACTGCCGTAGGCCCAGATGTTGATGCCTTCGCCCCT
GGTGATCGGGTGCTTGGCCTGTTCCAAGGCGCGTTCGCGCCAATCGCTGT
TACAGACCATCGCTTGTTAGCACGTGTTCCTGATGGTTGGTCGGATGCCG
ACGCTGCGGCCGTTCCTATCGCCTATACAACTGCACATTATGCCCTGCAT
GATCTGGCGGGCTTGCGCGCCCGTCAGAGTGTCCTTATTCACGCTGCCGC
TGGTGGTGTCGGTATGGCAGCTGTAGCTCTGGCACGTCGGGCTGGCGCCG
AGGTGTTAGCTACCGCTGGTCCGGCTAAACACGGCACTCTGCGTGCGCTC
GGTCTGGATGATGAGCATATTGCGAGTTCTAGGGAGACTGGTTTCGCCCG
TAAATTTCGTGAACGCACAGGCGGGCGTGGGGTTGACGTTGTGCTCAACT
CCTTGACTGGCGAACTCCTGGATGAGTCAGCAGACCTCCTTGCTGAAGAT
GGCGTGTTTGTAGAGATGGGCAAAACCGATCTGCGTGATGCCGGGGACTT
TCGTGGGCGCTACGCGCCATTTGATCTGGGGGAGGCAGGGGATGATCGTC
TGGGTGAAATTCTCCGTGAAGTAGTGGGCTTACTTGGCGCAGGCGAATTG
GATCGCCTGCCGGTAAGTGCATGGGAATTGGGGTCCGCGCCTGCCGCGCT
CCAGCACATGAGTCGCGGTCGTCACGTAGGTAAACTTGTACTGACCCAGC
CTGCGCCGGTCGACCCTGACGGCACTGTGTTAATCACCGGTGGTACAGGC
ACCCTGGGGCGTTTGTTAGCACGCCATCTGGTGACGGAACATGGTGTGCG
GCATCTGTTGCTGGTTAGTCGTCGTGGTGCTCACGCGCCGGGCTCCGATG
AACTGCGCGCAGAAATTGAGGATTTGGGTGCAAGCGCGGAAATTGCGGCG
TGCGACACAGCGGATCGCGACGCCCTGAGTGCCCTGCTGGATGGTTTGCC
CCGGCCTCTGACCGGGGTTGTGCACGCAGCCGGTGTGCTGGCCGATGGCT
TGGTGACAAGCATCGACGAACCGGCGGTGGAACAGGTTCTGCGTGCCAAA
GTCGATGCCGCGTGGAACCTCCATGAACTGACCGCAAATACCGGCTTGAG
CTTCTTTGTCCTGTTCAGTTCTGCGGCAAGCGTGTTAGCAGGCCCTGGGC
AAGGTGTGTATGCGGCGGCGAATGAAAGTCTGAATGCATTAGCGGCTCTG
CGTCGCACCCGCGGTTTGCCTGCCAAAGCGCTGGGTTGGGGCCTCTGGGC
CCAAGCGTCCGAAATGACTAGCGGTCTGGGTGACCGCATTGCGCGTACAG
GTGTTGCCGCGTTGCCGACCGAACGTGCTCTGGCCCTGTTCGACAGCGCA
TTGCGTCGCGGGGGTGAGGTGGTTTTTCCGCTGTCAATCAACCGCTCAGC
GCTGCGCCGCGCTGAATTTGTACCAGAGGTTCTGCGTGGCATGGTACGTG
CAAAACTTCGGGCTGCTGGGCAGGCTGAAGCTGCGGGCCCAAACGTAGTT
GACCGCTTAGCCCGTCGTAGCGAATCGGATCAGGTGGCGGGCCTCGCGGA
ACTGGTGCGTAGCCATGCAGCCGCCGTGAGTGGTTACGGCAGCGCCGATC
AGTTGCCGGAACGCAAAGCGTTTAAAGACTTGGGCTTCGATAGCCTGGCC
GCCGTCGAGCTCCCCAACCGCCTGGGCACAGCCACAGGCGTGCGGCTTCC
AAGCACGCTGGTGTTTGATCATCCGACGCCGTTGGCGGTAGCGGAGCATC
TGCGGGACCGGCTGTCTAGTGCCTCGCCGGCTGTTGACATCGGGGATCGG
CTGGATGAATTGGAAAAAGCACTGGAAGCCCTGTCAGCCGAGGATGGCCA
TGATGATGTGGGCCAGCGTCTGGAGAGCCTGCTTCGCCGCTGGAACAGTC
GTCGTGCGGACGCGCCGTCCACCTCTGCGATTTCTGAAGACGCTAGCGAT
GATGAATTATTTAGCATGCTCGACCAACGCTTTGGTGGTGGCGAGGACCT
GGGGAATTCG
DEBS3 (SEQ ID NO: 5)
ATGTCTGGTGATAATGGCATGACGGAAGAAAAATTACGTCGCTACTTGAA
ACGCACCGTTACCGAGCTCGATTCCGTTACCGCCCGTTTGCGCGAAGTCG
AACACCGCGCAGGTGAGCCAATTGCGATCGTAGGTATGGCCTGTCGCTTT
CCGGGCGATGTGGACTCTCCAGAATCTTTTTGGGAATTTGTTTCTGGCGG
GGGCGATGCGATTGCAGAAGCGCCAGCGGATCGTGGCTGGGAGCCTGATC
CAGATGCGCGTTTAGGCGGTATGTTAGCTGCGGCGGGCGATTTTGATGCA
GGTTTTTTCGGCATTTCGCCGCGTGAAGCCCTTGCGATGGATCCACAACA
GCGGATTATGCTGGAAATTTCATGGGAAGCCCTGGAACGGGCCGGTCACG
ATCCGGTGTCGCTGCGTGGCTCCGCCACAGGCGTATTCACTGGGGTTGGT
ACAGTCGATTATGGCCCTAGGCCAGATGACGCCCCTGATGAAGTCCTTGG
TTACGTTGGCACGGGCACCGCATCATCGGTCGCCAGTGGTCGTGTAGCCT
ACTGCCTTGGCCTTGAGGGGCCCGCCATGACCGTGGATACGGCATGCTCA
TCCGGCCTCACCGCCCTGCATTTGGCTATGGAATCCCTGCGCCGGGACGA
ATGTGGTTTAGCGCTGGCGGGCGGGGTTACCGTTATGAGCTCTCCTGGCG
CGTTCACAGAATTTCGCTCGCAGGGGGGTTTGGCCGCGGATGGTCGTTGT
AAACCGTTCAGTAAAGCGGCAGACGGCTTCGGGCTTGCAGAGGGGGCGGG
TGTCTTGGTGTTACAGCGTCTGTCAGCTGCTCGCCGTGAGGGGCGCCCGG
TACTGGCCGTCCTGCGCGGCAGTGCCGTAAATCAGGATGGTGCTAGCAAC
GGCTTAACGGCACCAAGCGGCCCAGCCCAACAACGTGTAATTCGTCGTGC
ACTGGAGAACGCGGGCGTTCGGGCGGGGGATGTAGATTACGTAGAAGCGC
ACGGCACAGGCACTCGTTTAGGCGACCCAATCGAAGTCCACGCTCTGCTG
TCGACGTATGGTGCTGAACGTGATCCTGATGACCCGTTATGGATTGGTTC
GGTTAAATCCAACATCGGCCATACCCAAGCTGCCGCTGGCGTCGCGGGCG
TTATGAAAGCGGTACTGGCCTTACGGCACGGCGAGATGCCACGCACCCTG
CATTTCGACGAACCAAGTCCTCAGATTGAATGGGACCTTGGGGCAGTTAG
CGTAGTTTCTCAGGCACGTTCGTCGCCCGCAGGCGAGCGTCCGCGCCGTG
CAGGCGTTAGTTCTTTTGGCATTAGCGGTACCAACGCGCATGTGATTGTT
GAGGAAGCCCCTGAAGCCGACGAACCGGAGCCCGCGCCGGATTCGGGTCC
GGTCCCTCTGGTGCTTAGCGGTCGCGATGAACAGGCCATGCGGGCACAGG
CGGGTCGCTTAGCCGATCACCTGGCTCGGGAACCACGGAACTCTCTGCGT
GACACAGGTTTTACCTTGGCTACGCGCCGCAGCGCCTGGGAACATCGCGC
TGTTGTGGTGGGCGATCGTGATGATGCGCTGGCCGGTCTGCGCGCCGTGG
CGGACGGTCGTATTGCGGATCGTACTGCGACTGGTCACGCGCGCACGCGT
CGCGGTGTGGCTATGGTGTTCCCTGGCCAGGGTGCGCAATGGCAGGGCAT
GGCGCGTGACCTGCTTCGTGAAAGCCAGGTTTTTGCCGATAGTATTCGCG
ACTGCGAACGTGCCTTGGCACCGCACGTAGATTCGAGTCTGACTGATCTG
CTGTCTGGGGCTCGTCCGCTGGATCGTGTTGACGTGGTGCAGCCTGCCCT
GTTTGCCGTTATGGTGTCCTTAGCCGCGCTGTGGCGTTCACATGGGGTAG
AGCCCGCAGCGGTCGTAGGCCACAGTCAAGGCGAAATTGCAGCCGCGCAT
GTTGCGGGGGCTCTGACGTTAGAGGATGCAGCTAAATTGGTTGCAGTAAG
ATCTCGTGTTTTAGCCCGTTTGGGCGGCCAGGGCGGCATGGCGTCGTTCG
GCCTGGGTACGGAACAGGCTGCGGAACGGATTGGCCGTTTCGCGGGCGCC
CTGTCAATCGCGAGCGTTAACGGCCCACGTTCTGTCGTGGTAGCAGGGGA
ATCTGGCCCTCTGGATGAACTGATCGCCGAGTGCGAAGCGGAAGGTATTA
CCGCACGCCGTATCCCAGTGGATTATGCGAGTCACTCCCCTCAGGTTGAA
TCTCTGCGCGAAGAACTTCTGACTGAGCTGGCGCGCATTAGCCCTGTGAG
CGCAGATGTCGCCCTGTATTCCACGACGACCGGCCAGCCGATCGACACGG
CAACCATGGATACCGCGTATTGGTATGCAAATCTCCGTGAGCAGGTGCGC
TTCCAAGACGCTACGCGTCAACTGGCCGAAGCCGGTTTTGATGCTTTCGT
GGAAGTATCTCCACATCCGGTCCTGACTGTGGGTATTGAGGCCACTCTTG
ATAGTGCATTGCCAGCAGATGCAGGCGCATGCGTTGTTGGTACGTTACGC
CGTGATCGTGGCGGCCTGGCAGACTTTCATACCGCATTAGGCGAAGCCTA
TGCCCAGGGCGTGGAGGTGGATTGGTCACCTGCTTTTGCGGATGCCCGCC
CAGTGGAATTACCAGTGTATCCGTTTCAGCGTCAGCGTTACTGGCTGCAG
ATTCCGACAGGTCGGCGGGCTCGTGACGAACATGATGATTGGCGTTATCA
GGTCGTTTGGCGTGAAGCGGAATGGCAGTCTGCGTCCCTCGCCGGTCGCC
TGCTGCTGGTAACCGGCCCGGGTGTACCATCTGAGCTGTCCGATGCCATC
CGGTCAGGGCTGGAGCAGTCGGGGGCAACGGTTTTGACATGCGACGTCGA
AAGCCGTTCCACGATCGGCACGGCGTTGGAAGCTGCTGATACTGATGCGC
TGAGCACCGTAGTATCGCTGTTAAGCCGTGATGGCGAGGCTGTCGATCCG
AGTCTCGATGCTCTGGCTTTGGTGCAGGCCCTAGGTGCTGCTGGCGTCGA
AGCACCGCTGTGGGTCCTGACCCGTAATGCTGTCCAGGTTGCTGATGGTG
AGCTGGTGGATCCTGCCCAAGCCATGGTGGGCGGGCTGGGCCGCGTCGTT
GGTATCGAACAACCGGGTCGCTGGGGCGGCTTGGTCGACCTGGTTGACGC
CGACGCAGCTTCCATCCGTAGTCTTGCTGCGGTGCTCGCGGATCCGCGTG
GTGAGGAACAAGTTGCCATCCGTGCAGATGGTATCAAAGTGGCGCGCCTG
GTTCCAGCACCGGCTCGCGCGGCACGTACCCGGTGGAGCCCTCGCGGTAC
GGTGCTGGTAACCGGTGGGACAGGTGGCATCGGGGCACACGTTGCACGTT
GGCTGGCGCGCAGTGGTGCGGAACATCTGGTTCTTCTGGGCCGCCGTGGC
GCCGACGCGCCAGGCGCCAGCGAACTCCGCGAAGAACTGACCGCGCTGGG
CACCGGCGTGACTATTGCAGCTTGCGACGTTGCGGATCGCGCTCGGTTAG
AAGCAGTATTGGCAGCGGAACGCGCGGAAGGTCGTACCGTCTCTGCCGTT
ATGCATGCCGCGGGTGTGTCAACCAGCACCCCGCTGGATGATTTAACCGA
AGCCGAGTTCACGGAGATCGCTGACGTGAAAGTCCGGGGCACCGTTAACC
TGGACGAGCTGTGTCCGGACCTGGATGCGTTCGTTCTCTTTTCGTCAAAT
GCTGGCGTTTGGGGGTCTCCGGGTCTGGCGTCCTACGCCGCTGCGAACGC
GTTTCTTGATGGTTTCGCACGCCGCCGCAGATCTGAAGGCGCACCCGTCA
CGAGTATCGCATGGGGGTTGTGGGCCGGTCAGAACATGGCCGGTGATGAA
GGCGGTGAGTATCTGCGTAGCCAGGGCCTGCGCGCAATGGACCCAGATCG
TGCGGTGGAAGAACTGCATATCACGCTGGATCACGGTCAGACCTCCGTCT
CAGTGGTCGATATGGACCGTCGCCGTTTTGTGGAGTTGTTCACGGCTGCC
CGTCACCGCCCTTTGTTTGATGAAATCGCGGGTGCACGGGCGGAAGCTCG
CCAGAGTGAAGAGGGGCCTGCGCTGGCGCAGCGTCTGGCCGCACTGTCTA
CCGCCGAGCGCCGCGAGCACCTGGCACACCTGATCCGTGCCGAAGTGGCA
GCGGTTCTTGGTCACGGCGACGATGCGGCGATTGACCGCGATCGTGCATT
CCGCGATCTGGGGTTTGACTCCATGACTGCCGTTGACCTGCGCAACCGTC
TCGCAGCCGTCACGGGGGTACGTGAGGCTGCCACAGTTGTATTTGACCAT
CCAACGATCACGCGCTTGGCGGATCATTATTTGGAGCGTCTCTCTAGTGC
CGCTGAAGCGGAACAGGCCCCAGCCCTGGTTCGCGAAGTTCCAAAAGATG
CCGATGACCCAATTGCGATCGTGGGCATGGCGTGCCGTTTTCCGGGCGGG
GTTCACAACCCGGGCGAGCTGTGGGAGTTCATCGTAGGCCGTGGCGATGC
CGTGACGGAAATGCCTACGGACCGGGGGTGGGATTTAGATGCACTGTTCG
ATCCAGATCCGCAGCGTCACGGAACCTCCTATTCTCGCCATGGTGCCTTC
TTAGATCGTGCCGCAGATTTTGACGCGGCTTTTTTTGGCATTTCACCTCG
TGAGGCGTTGGCAATGGATCCACAGCAGCGTCAGGTGCTGGAAACCACCT
GGGAGTTATTCGAAAACGCCGGTATCGATCCGCACAGCTTAAGAGGTTCA
GATACGGGTGTGTTTTTGGGCGCTGCCTATCAAGGTTACGGTCAGGATGC
GGTGGTCCCAGAGGATAGCCAGGGGTATCTGCTGACGGGGAACTCGTCTG
CCGTCGTGTCGGGCCGCGTCGCGTACGTGCTTGGCTTAGAAGGTCCGGCG
GTAACCGTGGACACGGCATGCTCTTCCAGCCTGGTGGCCTTACACTCCGC
TTGTGGCTCCCTGCGCGACGGTGATTGCGGGTTAGCGGTCGCCGGTGGCG
TCTCCGTGATGGCAGGGCCTGAAGTCTTCACTGAGTTCAGCCGCCAGGGT
GGCCTGGCGGTGGATGGCCGTTGTAAAGCGTTCTCTGCCGAGGCCGATGG
TTTCGGTTTTGCCGAGGGCGTGGCAGTGGTACTGCTTCAGCGTCTGAGCG
ATGCACGCCGGGCGGGCCGCCAAGTCCTGGGTGTGGTGGCCGGTTCCGCC
ATTAATCAGGACGGTGCTAGCAACGGTCTGGCGGCGCCAAGCGGTGTGGC
CCAACAACGTGTGATTCGTAAAGCATGGGCTCGCGCCGGTATTACTGGTG
CAGACGTCGCGGTGGTTGAAGCGCATGGGACTGGGACCCGCCTTGGTGAT
CCAGTTGAAGCGTCTGCGCTGCTGGCTACCTACGGGAAATCCCGTGGCAG
CTCAGGTCCGGTACTGCTGGGCTCTGTGAAAAGCAATATCGGGCACGCCC
AGGCGGCGGCTGGCGTTGCTGGGGTTATCAAAGTAGTGTTAGGTCTGAAC
CGGGGCCTCGTTCCGCCGATGCTGTGCCGAGGCGAACGTTCCCCGCTGAT
CGAATGGAGCAGTGGTGGCGTGGAGCTCGCCGAAGCTGTCAGCCCGTGGC
CGCCGGCAGCAGACGGCGTTCGGAGGGCAGGCGTGTCTGCGTTCGGCGTG
AGCGGTACCAACGCTCATGTCATTATTGCCGAGCCGCCAGAGCCTGAGCC
GCTGCCAGAACCGGGGCCGGTCCGTGTACTCGCCGCTGCGAATAGTGTTC
CGGTTCTCCTTAGCGCCCGCACCGAAACCGCGCTGGCTGCACAAGCACGC
CTGCTGGAAAGCGCCGTTGACGATTCGGTTCCACTGACGGCGTTGGCTTC
CGCTCTGGCTACCGGCCGCGCCCACCTTCCGCGTCGCGCGGCTCTGTTAG
CAGGTGACCACGAACAACTGCGGGGTCAGCTGCGTGCAGTGGCCGAAGGT
GTTGCAGCACCGGGCGCGACGACAGGTACGGCGTCCGCAGGTGGTGTGGT
CTTTGTCTTTCCTGGCCAGGGCGCCCAATGGGAAGGTATGGCTCGGGGGT
TGCTGAGTGTGCCAGTTTTCGCCGAATCGATCGCCGAATGTGACGCCGTT
CTGAGTGAAGTTGCAGGTTTTTCAGCTTCAGAAGTTCTGGAACAGCGCCC
TGATGCACCGTCACTCGAACGCGTGGACGTTGTGCAACCAGTGCTGTTCT
CTGTTATGGTTAGTTTAGCCCGTTTATGGGGCGCGTGTGGGGTGAGCCCG
TCAGCCGTTATCGGTCATAGTCAGGGCGAAATTGCGGCGGCCGTCGTGGC
CGGCGTTCTGAGTTTGGAGGATGGCGTTCGTGTGGTCGCGTTGCGCGCGA
AAGCCCTCCGTGCACTCGCGGGCAAAGGCGGCATGGTCTCCTTGGCGGCC
CCTGGCGAACGCGCCCGTGCGTTGATTGCCCCGTGGGAAGACCGCATCAG
TGTGGCGGCCGTAAACAGTCCTAGCAGCGTTGTAGTTAGCGGTGATCCTG
AAGCACTTGCGGAGCTGGTAGCGCGTTGCGAAGATGAAGGCGTTCGCGCC
AAAACGCTCCCAGTGGACTATGCGAGCCATTCTCGGCACGTGGAAGAGAT
TCGCGAAACAATCTTGGCGGACCTGGATGGTATCTCTGCACGTCGTGCGG
CGATCCCGCTGTACAGCACCCTTCATGGCGAGCGTCGCGACGGGGCGGAT
ATGGGGCCGCGGTATTGGTATGACAATTTGCGCAGTCAGGTCCGGTTCGA
TGAAGCGGTTTCAGCGGCCGTTGCCGATGGTCATGCCACCTTTGTGGAAA
TGAGCCCGCACCCGGTTCTGACCGCCGCCGTGCAGGAGATCGCGGCCGAT
GCCGTGGCGATCGGTTCTCTGCACCGTGATACGGCTGAGGAGCATTTAAT
TGCCGAATTAGCACCCGCTCATGTACACGGCGTCGCTGTCGATTGGCGCA
ACGTGTTTCCAGCGGCACCACCCGTGGCTCTGCCGAACTACCCGTTCGAG
CCGCAGCGCTACTGGCTGCAGCCGGAGGTGTCTGACCAGCTGGCGGACTC
CCGGTATCGCGTGGATTGGCGTCCACTGGCGACAACGCCGGTGGATCTGG
AAGGCGGTTTTCTGGTGCACGGCTCAGCGCCTGAATCACTCACCTCCGCA
GTAGAGAAAGCAGGCGGGCGCGTAGTTCCAGTGGCGAGCGCCGATCGGGA
AGCCTCTGCTGCCTTGCGTGAGGTTCCGGGCGAAGTGGCTGGCGTGCTGT
CGGTGCACACTGGCGCCGCTACTCACCTGGCGCTGCACCAGTCCCTAGGC
GAAGCAGGTGTGCGCGCCCCGTTATCGTTAGTGACCAGCCGTGCCGTGGC
GCTCGGTGAATCCGAACCAGTTGATCCGGAACAAGCGATGGTGTGGGGCC
TGGGCCGCGTTATGGGGCTGGAAACCCCGGAGCGTTGGGGCGGCTTAGTA
GATTTGCCGGCCGAACCTGCCCCTGGGGATGGCGAAGCCTTCGTCGCATG
TCTTGGCGCGGATGGTCACGAAGATCAAGTCGCGATTCGTGATCACGCGC
GTTATGGGCGCCGTCTGGTGAGGGCTCCGCTGGGTACTCGGGAGAGCAGC
TGGGAACCGGCGGGTACTGCATTGGTGACCGGTGGCACGGGGGCGTTGGG
CGGTCACGTGGCTCGCCATCTGGCCCGCTGCGGCGTCGAGGACCTGGTGC
TGGTCAGCCGCCGTGGTGTAGACGCCCCGGGCGCGGCGGAGCTGGAAGCT
GAGCTTGTGGCGCTGGGCGCCAAAACGACAATTACGGCATGCGATGTAGC
GGATCGTGAACAGCTGTCGAAACTTTTAGAAGAATTACGTGGGCAGGGTC
GTCCGGTGCGCACAGTCGTTCATACTGCGGGCGTCCCGGAATCACGCCCG
CTGCATGAGATTGGGGAATTGGAATCTGTGTGCGCCGCCAAAGTTACCGG
CGCCCGCCTGCTTGACGAACTGTGTCCTGATGCGGAGACTTTTGTGTTGT
TTAGCTCCGGGGCGGGCGTGTGGGGCTCCGCAAATTTAGGCGCATATTCG
GCGGCAAACGCCTACCTCGATGCTCTGGCTCATCGTCGGCGCGCAGAAGG
CCGCGCAGCCACCAGTGTTGCCTGGGGGGCGTGGGCCGGCGAAGGCATGG
CAACGGGCGACTTAGAAGGGCTGACGCGCCGTGGCTTGCGCCCGATGGCG
CCGGAGCGGGCAATTCGGGCGCTCCACCAAGCTCTGGACAATGGTGACAC
TTGCGTCTCTATTGCCGACGTCGACTGGGAGGCGTTCGCTGTGGGGTTTA
CCGCCGCACGTCCGCGTCCACTGCTCGATGAACTGGTCACGCCGGCGGTG
GGTGCAGTACCAGCTGTTCAGGCGGCTCCAGCCCGTGAAATGACTAGCCA
AGAACTGCTGGAGTTCACACACTCGCATGTTGCCGCAATCTTGGGTCATA
GCAGTCCGGATGCCGTCGGCCAAGACCAGCCGTTTACGGAACTGGGTTTC
GATAGTCTGACTGCCGTTGGCCTGCCGAACCAGCTACAGCAAGCAACTGG
TCTGGCGTTACCGGCAACTTTAGTCTTCGAACATCCGACAGTACGCCGCT
TGGCCGATCACATCGGGCAACAACTGTCTAGTGGCACCCCGGCGCGGGAA
GCGTCTAGTGCTCTGCGCGACGGGTATCGTCAGGCTGGCGTGTCGGGGCG
CGTACGCAGTTACTTGGATCTCCTGGCAGGTCTTTCCGACTTCCGCGAGC
ATTTCGATGGTTCTGATGGCTTTAGCCTTGACCTGGTGGATATGGCCGAT
CGTCCAGGCGAAGTGACGGTCATCTGCTGTGCGGGGACCGCGGCCATTTC
AGGCCCGCACGAGTTTACTCGTCTCGCTGGCGCATTGCGCGGCATTGCTC
CTGTGCGTGCAGTTCCGCAACCAGGCTATGAGGAAGGCGAACCACTGCCG
AGCAGCATGGCCGCCGTGGCCGCGGTGCAGGCTGATGCAGTCATTCGCAC
CCAAGGTGACAAACCTTTCGTGGTAGCAGGCCACAGCGCCGGCGCACTCA
TGGCCTATGCACTCGCGACCGAGCTGTTGGATCGTGGTCACCCGCCACGC
GGGGTTGTCCTGATTGATGTATACCCGCCGGGCCACCAAGACGCTATGAA
CGCCTGGCTCGAAGAATTGACCGCCACGTTATTTGACCGTGAGACCGTAC
GCATGGACGACACTCGCTTGACCGCGCTGGGTGCGTACGACCGCCTGACA
GGTCAGTGGCGTCCGCGCGAAACGGGTCTGCCGACACTTCTGGTGTCTGC
GGGCGAACCTATGGGCCCATGGCCGGATGATTCGTGGAAACCGACCTGGC
CGTTTGAGCATGACACAGTGCCTGTCCCAGGCGACCATTTCACGATGGTT
CAGGAACACGCCGATGCGATTGCTCGTCATATCGACGCCTGGCTTGGAGG
CGGGAATTCG

### Example 8

### Method for Quantitative Determination of Relative Amounts of Two Proteins

A double-mab technique was developed to quantitatively determine the relative amounts of two or more PKS proteins expressed in the same cell. According to this method, different epitope tags are used for each PKS protein, and they are quantitated simultaneously by Western blot using a mixture of two differently labelled antibodies (e.g. labelled with CY3 and CY5). The ratio of dyes provides an assessment of the relative stoichiometry of the two proteins expressed.

As a model system to develop this technology, we used a protein that was labelled with two different epitope tags (cmyc-AtoC-FLAG-BRS-His) on either end (the 55 kDa AtoC). This provided a protein in which the two tags are present in a known ratio.

In our initial experiments, we had difficulties obtaining reproducible ratios of two Mab's bound to the protein after Western blot, especially with sub-microgram quantities. We therefore made the effort to develop the methods of analysis needed using dot-blots of cmyc-AtoC-FLAG. In the data shown below, two fluorescently labelled antibodies (cymc-AlexaFluor488 and FLAG-Cy5) were used simultaneously to quantitate a dot-blot of the AtoC construct mentioned above. The blot was scanned using a Typhoon 9410 Fluorescent Imager, and analysis was performed using ImageQuant software. Results are shown in Table 15.

TABLE 15
RESIDUAL ANALYSIS OF DOT-BLOT DATA

| | cmyc-AlexaFluor488 | | FLAG-Cy5 | | |
|---|---|---|---|---|---|
| | predicted | | predicted | | ratio of areas |
| ng on blot | ng | % error | ng | % error | (AF488/Cy5) |
| 10 | 5.80 | 42.02 | −4.17 | 58.34 | 0.151 |
| 50 | 48.28 | 3.44 | 41.97 | 16.06 | 0.139 |
| 100 | 109.01 | 9.01 | 119.99 | 19.99 | 0.125 |
| 250 | 243.78 | 2.49 | 260.24 | 4.09 | 0.132 |
| 500 | 504.70 | 0.94 | 491.97 | 1.61 | 0.146 |
| 1000 | 998.43 | 0.16 | 495.34 | 50.47 | 0.284 |

The cmyc-AlexaFluor488 antibody provides a very accurate range of quantitation in the 50-1000 ng range. The FLAG-Cy5 antibody is accurate across a range of 50-500 ng, and clearly suffers from signal saturation at the 1000 ng level. The ratios of the peak areas are also stable across the 10-500 ng range, allowing for detection of N-terminal or C-terminal degradation, as well as stoichiometric analysis of protein levels.

Epitope-tagged DEBS proteins have now been expressed and purified for use as epitope tagged standards for quantitative Western analysis.

TABLE 16

| Protein | Epitope Tags | Configuration of tags |
|---|---|---|
| DEBS module 2 | HA, flag, brs, his | HA-mod2-flag-brs-his |
| DEBS module 2 | c-myc, flag, brs, his | cmyc-mod2-flag-brs-his |
| DEBS module 2 | HA, his | mod2-HA-his |
| DEBS2 | c-myc, his | DEBS2-c-myc-his |

A synthetic DEBS module 2 protein (mod2) was expressed in E. coli K-207-3 as a fusion protein (c-myc-mod2-flag-brs-his). Cloning of the module 2 gene into an expression vector in frame with genes encoding the tag sequences was facilitated by inclusion of an Eco RI site in the synthetic gene. DEBS module2 with N- and C-terminal epitope tags was co-expressed with DEBS2 and DEBS3 in an E. coli k-207-3. At 20 and 40 hours, samples from production cultures were subjected to SDS-PAGE (two colonies of each strain were tested). Gels were either stained with sypro red or subjected to Western blotting, using fluorescently-labeled antibodies directed against the epitope tags, c-myc, flag and biotin. Monoclonal antibodies were labeled with fluorescent dyes (alexa 488 and alexa 647) such that two fluorescent signals could be monitored simultaneously.

### Example 9

### Epothilone PKS Gene Synthesis

The complete 54,489 bp epothilone synthase gene (loading didomain, 9 elongation modules, and thioesterase of the DEBS gene) was synthesized, and assembled.

The gene was designed by using a version of GeMS software developed. Modules were synthesized using Method R and Type II vectors. To synthesize the approximately 55 kb of DNA, the gene cluster was broken down into 118 synthon fragments ranging in size from 156 to 781 bp. The 3000 oligonucleotides were pooled into oligonucleotide mixtures using the Biomek FX and the assembly and amplification were performed using the conditions described in Example 1. They were cloned into a UDG-LIC vector (Method R and Type II vectors were used) and a >90 success rate in UDG cloning. Eight colonies for each synthon were picked into 1.5 mL LB/carb and aliquots were taken for use as template for the RCA reaction to provide samples for sequencing. Clones were obtained that contained the correct sequence for all 118 synthons that make up the Epo gene cluster. The average error rates for the 118 synthons was 2.4/1000 and on average 32% of the samples sequenced were correct. This was an improvement from the DEBS gene cluster numbers of 3 errors per kb and only 22% correct. Correct samples for 104 of 118 (88%) were obtained from this first round of sequencing eight samples; for the remaining 12 synthons, correct sequences were found after sequencing additional clones. After the correct clone was identified through sequencing, the plasmid DNA was isolated from stored cultures and the assembling the synthons into modules was performed using the stitching strategy aforementioned.

The sequences of synthetic ORFs encoding epothilone synthase polypeptides EpoA—are shown below in Table 17B. (Each of the sequences includes a 3′ Eco RI site which was included to facilitate addition of tags.) Table 17A shows the overall sequence identity between the DNA sequences of the synthetic genes and the reported epothilone synthase sequences.

TABLE 17A
SIMILARITY OF SYNTHETIC AND NATURALLY OCCURRING SEQUENCES

| | NATURALLY OCCURRING | | | | | | |
|---|---|---|---|---|---|---|---|
| | GENE SEQUENCE¹ | | | | SYNTHETIC GENE SEQUENCE | | |
| | | Naturally | | | # aa | | |
| | | Occurring | | | changes | | % identity |
| | Naturally Occurring | Polypeptide | | | compared | % identity | vs nat. |
| epothilone | DNA Sequence | Sequence | | | to | vs nat. | seq. |
| PKS | (accession #) | (accession #) | #bp | #aa | nat. seq. | seq. (aa) | (dna) |
| EpoA | AF217189 | AAF62880 | 4263 | 1421 | 4 | 99.72% | 75% |
| EpoB | AF217189 | AAF62881 | 4230 | 1410 | 2 | 99.86% | 75% |
| EpoC | AF217189 | AAF62882 | 5496 | 1832 | 4 | 99.78% | 75% |
| EpoD | AF217189 | AAF62883 | 21771 | 7257 | 15 | 99.79% | 75% |
| EpoE | AF217189 | AAF62884 | 11394 | 3798 | 8 | 99.79% | 74% |
| EpoF | AF217189 | AAF62885 | 7317 | 2439 | 5 | 99.79% | 75% |

¹As reported in GenBank accession nos. shown.

TABLE 17B
SEQUENCE OF SYNTHETIC EPOTHILONE SYNTHASE

EpoA (SEQ ID NO: 6)
ATGGCCGACCGCCCGATCGAACGTGCAGCGGAGGATCCAATTGCGATTGT
AGGCGCGGGCTGCCGCCTGCCGGGCGGCGTGATTGACCTCTCGGGCTTCT
GGACGCTGTTAGAAGGCTCCCGCGACACCGTCGGTCAAGTGCCAGCGGAG
CGGTGGGATGCTGCGGCGTGGTTCGATCCGGATCTGGATGCACCTGGCAA
AACACCAGTGACCCGCGCCAGCTTTTTAAGCGATGTCGCCTGCTTCGATG
CCTCTTTTTTCGGGATCAGTCCGCGCGAAGCCCTTCGCATGGATCCGGCC
CACCGGCTGCTGCTGGAAGTGTGCTGGGAAGCATTGGAAAACGCAGCTAT
TGCCCCGTCGGCCCTGGTTGGCACGGAAACTGGCGTCTTTATTGGCATCG
GTCCAAGCGAATATGAAGCGGCACTGCCTAGGGCTACTGCCAGCGCAGAA
ATTGATGCTCACGGCGGCCTGGGCACGATGCCTTCAGTTGGTGCAGGTCG
TATTTCATACGTCCTGGGCCTTCGTGGTCCGTGTGTGGCGGTGGACACCG
CATATAGTTCTAGCTTAGTCGCAGTACACCTGGCGTGTCAGTCGTTACGT
TCCGGCGAATGCTCGACCGCGCTTGCAGGTGGGGTCAGCCTTATGCTGTC
CCCGAGCACTTTAGTCTGGTTGAGCAAGACACGTGCGTTGGCAACCGACG
GTCGCTGCAAAGCCTTCAGCGCGGAGGCCGATGGGTTTGGTCGTGGCGAA
GGTTGCGCAGTGGTCGTGCTGAAGCGTTTGTCCGGCGCACGTGCGGATGG
GGACCGCATCCTCGCAGTTATCCGCGGCTCGGCCATCAACCATGATGGTG
CCAGCTCCGGTCTCACTGTTCCGAACGGTTCTTCACAGGAAATTGTACTG
AAACGCGCCTTAGCCGATGCTGGTTGCGCCGCATCTTCCGTGGGGTACGT
CGAAGCTCATGGGACGGGTACTACCTTAGGCGATCCGATTGAAATTCAGG
CGCTCAATGCCGTCTACGGCCTGGGTCGGGATGTCGCGACCCCTTTGCTG
ATCGGGTCGGTCAAGACTAACCTCGGCCATCCAGAGTATGCCTCCGGGAT
CACTGGTCTGCTGAAGGTTGTGTTGTCCTTGCAGCACGGTCAAATTCCGG
CGCACCTCCATGCTCAGGCGTTAAATCCGCGCATTAGCTGGGGCGATCTG
CGTCTGACCGTTACCCGTGCTCGGACCCCGTGGCCTGACTGGAACACGCC
TCGCCGCGCGGGCGTCTCCTCGTTTGGCATGAGTGGTACCAATGCCCACG
TTGTTCTGGAGGAAGCCCCAGCAGCAACGTGCACCCCGCCAGCCCCAGAA
CGTCCAGCCGAATTGTTAGTGCTGTCTGCGCGTACCGCTGCCGCTCTGGA
CGCACATGCGGCCCGTTTGCGCGACCATTTAGAAACATACCCGTCACAAT
GTTTAGGTGACGTTGCCTTCTCGCTGGCGACTACCCGTAGTGCGATGGAA
CATCGCCTGGCGGTGGCCGCTACGTCCTCGGAGGGTCTGCGTGCGGCCTT
AGACGCCGCAGCTCAGGGTCAGACCCCGCCGGGTGTTGTCCGTGGTATCG
CAGACTCGTCTCGCGGCAAACTGGCTTTTCTGTTTACTGGCCAGGGTGCC
CAGACGCTCGGCATGGGCCGGGGCCTGTACGATGTTTGGCCTCCTTTTCG
CGAAGCGTTTGATTTGTGTGTGCCCCTGTTTAACCAAGAACTGGATCGTC
CGCTGCGTGAAGTAATGTGGGCAGAACCAGCATCAGTAGATGCCGCACTT
TTAGACCAGACAGCTTTTACACAGCCAGCGCTTTTTACGTTTGAGTATGC
TCTGGCTGCACTGTGGAGATCTTGGGGCGTAGAACCAGAACTGGTGGCCG
GTCACTCGATTGGCGAACTGGTGGCGGCGTGCGTTGCGGGTGTGTTCAGT
TTGGAGCACGCCGTGTTCCTGGTCGCGGCACGCGGTCGTCTCATGCAGGC
GCTGCCTGCTGGTGGTGCAATGGTGTCTATTGCGGCGCCAGAAGCGGACG
TCGCGGCGGCGCTCGCGCCTCATGCCGCATCAGTAAGTATCGCGGCTGTT
AATGGCCCAGACCAAGTGGTAATCGCGGGCGCAGGGCAGCCGGTGCATGC
GATCGCCGCTGCAATGGCGGCGCGCGGTGCCCGGACCAAAGCGCTTCACG
TGAGCCACGCGTTCCACAGTCCACTGATGGCACCGATGTTAGAAGCGTTT
GGCCGCGTTGCTGAATCCGTAAGTTATCGTCGTCCGAGCATCGTACTCGT
TAGTAATCTGAGCGGCAAAGCAGGGACAGATGAAGTATCCAGCCCTGGCT
ATTGGGTGCGTCATGCTCGGGAGGTTGTGCGTTTCGCAGATGGCGTGAAA
GCGCTCCATGCCGCAGGTGCAGGCACGTTTGTTGAAGTGGGTCCGAAGTC
TACTCTTTTGGGTTTAGTTCCGGCGTGTTTGCCAGACGCTCGTCCGGCGC
TTCTGGCAAGTTCTCGTGCCCGGCGCGATGAACCAGCCACTGTTCTGGAA
GCTCTGGGGGGTCTGTGGGCCGTTGGTGGTCTTGTATCGTGCGCAGGTCT
GTTTCCGAGTGGCGGTCCCCGCGTGCCTCTGCCGACGTATCCGTGGCAAC
GTGAGCGTTACTGGCTGCAGACCAAGGCGGATGACGCAGCGCGTGGTGAT
CGGCGAGCACCGGGTGCGGGCCATGACGAAGTCGAAAAAGGCGGGGCGGT
CAGAGGTGGGGATCGCCGCAGCGCCCGTTTGGATCATCCACCGCCAGAGA
GCGGACGCCGTGAAAAGGTGGAGGCAGCGGGCGACCGTCCGTTTCGTTTG
GAGATTGATGAGCCTGGCGTGCTGGACCGGCTCGTTCTGCGTGTTACGGA
GCGTCGCGCACCGGGCTTAGGTGAGGTGGCGGTTGCTGTAGATGCGGCAG
GTCTGAGTTTTAACGACGTGCAGCTGGCTCTGGGTATGGTTCCGGATGAT
CTGCCGGGTAAACCGAATCCGCCGCTGCTGTTAGGCGGGGAATGTGCCGG
CCGCATTGTGGCGGTTGGGGAAGGCGTAAATGGTCTGGTTGTAGGTCAGC
CGGTGATTGCACTGAGCGCTGGTGCTTTCGCAACCCATGTCACCACGTCA
GCCGCCCTGGTGCTGCCACGCCCTCAGGCGCTGTCCGCGACCGAGGCCGC
AGCTATGCCAGTGGCATATCTCACCGCGTGGTATGCTCTGGATGGCATTG
CCCGCCTTCAACCTGGCGAGCGCGTGCTGATCGCTGCGGCCACGGGTGGC
GTTGGCCTGGCGGCAGTACAGTGGGCCCAGCACGTCGGGGCCGAAGTTCA
CGCTACTGCGGGTACGCCAGAGAAACGCGCTTACCTTGAAAGCCTCGGGG
TTCGTTACGTTTCAGATTCTCGCAGCGACCGCTTTGTAGCAGATGTGCGC
GCCTGGACCGGCGGCGAAGGCGTTGATGTCGTTCTGAACTCTCTGTCAGG
TGAACTGATTGATAAGTCATTCAACTTACTGCGGTCTCATGGTCGTTTTG
TCGAACTCGGCAAACGCGATTGTTATGCTGATAATCAGCTCGGCCTTCGC
CCTTTCCTGCGTAACCTTTCATTTTCTTTGGTTGATCTGCGCGGCATGAT
GCTGGAACGCCCGGCACGTGTGCGTGCCTTGTTTGAGGAGCTGCTGGGTT
TAATTGCCGCTGGTGTGTTCACCCCGCCGCCGATCGCCACGCTTCCTATT
GCTCGCGTGGCGGACGCCTTCCGTTCGATGGCGCAAGCACAGCATTTAGG
CAAACTCGTACTGACCCTAGGGGATCCGGAGGTCCAAATCCGTATTCCGA
CACACGCGGCGGCCGGTCCGTCTACCGGCGACCGGGACCTGCTGGATCGT
CTTGCGAGTGCTGCACCGGCGGCTCGTGCGGCGGCCTTAGAAGCTTTTTT
GCGCACCCAGGTGTCGCAAGTGCTGCGCACACCTGAAATTAAAGTAGGGG
CTGAAGCTTTGTTCACACGGCTGGGTATGGATTCCCTGATGGCAGTGGAA
CTTCGTAATCGTATTGAGGCGAGCTTGAAGCTGAAATTATCTACAACCTT
CCTTAGCACGAGCCCGAACATCGCCCTGCTGACCCAAAACTTGTTGGATG
CACTCTCTAGTGCATTAAGTTTGGAACGTGTTGCCGCGGAGAACCTGCGC
GCGGGCGTCCAATCCGACTTTGTGTCGTCAGGGGCCGATCAGGATTGGGA
AATCATTGCTCTGGG
EpoB (SEQ ID NO: 7)
ATGACCATTAATCAGTTACTGAATGAATTAGAACACCAGGGCGTTAAATT
AGCCGCAGATGGGGAGCGCCTCCAGATTCAGGCACCAAAAAATGCCCTGA
ACCCGAACTTGTTAGCACGCATTTCTGAACATAAATCCACGATCTTAACC
ATGCTGCGCCAGCGCCTTCCGGCGGAGTCTATTGTCCCAGCCCCAGCGGA
ACGGCATGTGCCGTTCCCTCTGACCGACATCCAGGGCTCTTATTGGCTCG
GTCGTACTGGTGCCTTTACGGTTCCGTCGGGCATCCATGCCTACCGTGAA
TATGATTGCACGGATCTGGACGTGGCCCGGCTTAGTCGTGCATTCCGTAA
AGTCGTTGCACCGCATGATATGCTGAGGGCTCATACCCTGCCGGATATGA
TGCAGGTGATCGAACCTAAAGTAGATGCGGACATCGAAATCATTGACCTG
CGTGGCCTCGATAGATCTACACGCGAAGCTCGGTTGGTGTCCCTGCGTGA
CGCCATGTCTCACCGGATTTATGATACGGAACGCCCGCCGCTGTATCACG
TTGTGGCCGTTCGCTTAGATGAACAACAGACCCGCCTGGTGCTGAGCATT
GATCTGATTAACGTTGACCTGGGCAGTCTGAGCATTATCTTTAAAGATTG
GTTGAGCTTTTACGAAGATCCTGAAACCTCGCTGCCAGTGCTGGAACTGA
GTTACCGCGACTACGTCCTGGCGTTGGAATCGCGTAAAAAATCGGAAGCC
CACCAGCGCTCAATGGACTACTGGAAACGCCGTGTTGCTGAACTCCCACC
ACCGCCAATGCTGCCAATGAAAGCGGATCCGTCGACGTTGCGTGAAATTC
GCTTCCGTCATACCGAACAGTGGCTCCCGTCTGATAGTTGGTCGCGTTTA
AAACAACGTGTAGGCGAACGGGGTCTGACCCCAACGGGTGTAATCCTCGC
AGCTTTCTCTGAGGTGATCGGCCGCTGGTCCGCTAGCCCGCGCTTTACCC
TCAACATCACTTTATTCAACCGTCTCCCTGTGCATCCCCGGGTCAATGAT
ATTACTGGTGATTTTACAAGCATGGTGCTGTTGGACATTGATACGACGCG
CGACAAATCATTCGAACAGCGTGCTAAACGCATTCAGGAACAGCTGTGGG
AAGCCATGGACCACTGCGATGTTTCTGGGATTGAAGTACAGCGCGAAGCG
GCACGTGTGCTGGGCATTCAACGCGGCGCACTGTTCCCGGTAGTACTGAC
CTCAGCCCTCAATCAACAGGTGGTTGGGGTTACGTCTCTGCAACGTCTGG
GCACCCCGGTTTACACGAGCACTCAGACTCCGCAGCTCCTGCTCGATCAT
CAGCTGTACGAACATGACGGTGACCTGGTCCTGGCGTGGGATATTGTGGA
TGGCGTGTTTCCGCCGGATCTGCTGGATGATATGTTAGAAGCCTATGTCG
CCTTTTTACGTCGCCTGACGGAGGAACCGTGGTCTGAACAAATGCGCTGC
AGCCTGCCGCCCGCTCAGTTAGAGGCACGTGCATCCGCCAATGAAACTAA
CTCACTGCTGTCTGAACATACTCTGCATGGTCTGTTTGCCGCTCGGGTGG
AGCAGTTACCGATGCAGCTTGCAGTGGTTAGCGCTCGTAAAACCCTGACG
TATGAGGAATTGTCTCGCCGCTCCCGGCGGCTGGGTGCCCGCCTGCGGGA
ACAAGGCGCACGCCCGAATACCTTGGTCGCCGTCGTTATGGAGAAAGGTT
GGGAACAAGTGGTTGCGGTCCTTGCCGTGCTGGAAAGCGGCGCGGCTTAT
GTTCCGATTGATGCCGACCTGCCAGCAGAACGTATTCATTACCTGCTTGA
TCACGGTGAGGTTAAATTGGTGCTGACTCAACCGTGGCTGGATGGCAAAC
TTAGCTGGCCGCCAGGGATCCAGCGTCTGCTGGTAAGCGACGCCGGCGTC
GAAGGGGACGGCGACCAACTGCCGATGATGCCGATTCAGACCCCATCGGA
CTTAGCATACGTCATCTACACCAGTGGTTCGACTGGTTTGCCGAAAGGTG
TTATGATTGATCACCGTGGCGCTGTCAATACAATTTTGGACATCAACGAG
CGCTTTGAGATTGGTCCTGGGGATCGCGTGCTGGCCCTGTCCTCACTTTC
TTTTGATCTGTCGGTTTATGACGTTTTCGGTATCCTCGCGGCGGGCGGGA
CCATTGTGGTGCCAGATGCGTCAAAACTGCGTGACCCAGCCCACTGGGCT
GCACTTATTGAACGCGAAAAAGTCACTGTGTGGAATAGTGTACCGGCACT
GATGCGTATGCTGGTCGAACACTCTGAAGGGCGCCCTGATTCGCTGGCAC
GTAGCCTGCGCCTCAGCCTGCTGAGTGGTGATTGGATCCCTGTGGGGCTC
CCGGGTGAACTTCAGGCTATCCGTCCGGGCGTCAGTGTTATTAGCCTGGG
GGGTGCCACAGAGGCTAGCATCTGGAGCATTGGCTATCCTGTTCGCAACG
TGGACCCGTCCTGGGCATCAATTCCGTATGGCCGCCCGCTTCGCAATCAG
ACGTTCCACGTGCTTGACGAGGCGCTGGAGCCACGGCCGGTATGGGTGCC
AGGCCAACTGTATATCGGTGGCGTTGGCCTGGCACTGGGCTATTGGCGTG
ACGAGGAAAAAACTCGTAACTCTTTTCTCGTCCATCCGGAAACGGGGGAA
CGCCTGTATAAAACCGGGGATCTCGGGCGCTACCTTCCGGATGGCAATAT
TGAATTTATCGGCCCCGAGGATAACCAAATTAAACTGCGGGGCTATCGCG
TGGAATTGGGTGAAATCGAAGAAACCCTGAAAAGCCATCCTAACGTGCGC
GATGCGGTCATCGTGCCGGTTGGCAATGATGCCGCAAATAAATTACTGCT
TGCGTATGTGGTACCGGAGGGCACCCGCCGCCGTGCGGCGGAACAGGACG
CATCACTTAAGACGGAACGTGTTGATGCGCGTGCGCATGCAGCCAAAGCG
GACGGCCTGAGCGACGGTGAGCGCGTCCAGTTCAAACTGGCACGTCATGG
CCTGCGTCGCGATCTGGATGGCAAACCGGTGGTAGACCTGACGGGTCTGG
TACCGCGCGAAGCGGGGCTGGATGTATATGCTCGTCGTCGTTCGGTCCGC
ACTTTCTTAGAGGCACCGATCCCGTTCGTAGAATTTGGTCGCTTTCTGTC
TTGTCTTAGCTCAGTGGAGCCTGATGGCGCAGCTCTCCCTAAATTCCGTT
ACCCTTCGGCGGGTAGTACCTACCCGGTCCAAACATACGCCTATGCGAAA
AGCGGCCGTATCGAGGGTGTAGACGAAGGCTTCTATTACTATCATCCATT
CGAGCATCGTCTGCTGAAAGTTAGTGATCACGGTATTGAACGTGGCGCGC
ACGTGCCGCAGAACTTCGACGTGTTTGACGAAGCTGCCTTTGGTTTACTC
TTTGTTGGCCGTATCGATGCGATCGAGAGCCTGTACGGGTCATTGAGCCG
CGAATTTTGTCTGTTGGAAGCTGGTTATATGGCCCAACTGCTCATGGAGC
AAGCGCCGTCGTGCAACATTGGGGTCTGCCCTGTAGGGCAGTTTGATTTT
GAACAGGTACGCCCAGTTCTTGATTTACGCCATTCCGATGTTTACGTACA
CGGTATGCTGGGCGGTCGCGTGGATCCTCGCCAGTTTCAGGTCTGTACCC
TCGGCCAGGATTCCAGCCCACGTCGTGCTACGACGCGCGGTGCCCCACCG
GGTCGCGACCAACATTTTGCTGACATCCTTCGGGACTTTCTTCGCACTAA
ACTGCCGGAATATATGGTACCGACCGTTTTCGTCGAGTTGGACGCGTTAC
CGCTCACTTCTAACGGCAAAGTGGATCGCAAAGCGCTGCGGGAACGCAAA
GATACATCATCCCCGCGGCACTCCGGTCACACCGCCCCGCGTGATGCTCT
GGAAGAGATTCTGGTCGCCGTTGTTCGTGAAGTTCTCGGTCTGGAAGTGG
TCGGGCTGCAACAGTCTTTTGTAGACCTGGGTGCTACTTCCATCCATATC
GTTCGTATGCGCAGCCTGTTGCAGAAACGCCTGGACCGCGAAATTGCCAT
TACAGAACTTTTCCAGTACCCAAATCTGGGTTCGTTAGCCAGCGGTCTTT
CTAGTGATAGTAAAGATTTAGAACAACGTCCGAATATGCAGGACCGCGTC
GAGGCTCGCCGCAAAGGCCGGCGTCGTTCAGGGAATTC
Epoc (SEQ ID NO: 8)
ATGGAAGAACAAGAATCCAGTGCAATTGCCGTGATTGGCATGTCAGGTCG
GTTTCCAGGGGCCCGCGATCTGGATGAGTTCTGGCGCAATCTGCGCGACG
GCACCGAGGCCGTCCAGCGCTTTAGTGAGCAGGAACTGGCGGCGTCCGGC
GTTGATCCGGCTCTTGTGTTAGATCCGAACTATGTGCGGGCAGGTAGCGT
TCTGGAAGATGTCGATCGTTTTGATGCCGCTTTCTTTGGTATCTCCCCGC
GTGAAGCGGAACTGATGGACCCGCAGCACCGGATCTTTATGGAATGCGCG
TGGGAAGCACTCGAAAACGCCGGCTATGACCCGACTGCATACGAGGGTAG
CATCGGCGTGTATGCGGGGGCCAACATGAGCAGTTATTTAACCTCAAATT
TACATGAACATCCGGCGATGATGCGTTGGCCGGGTTGGTTCCAGACGCTG
ATCGGGAACGATAAAGATTACTTGGCAACGCACGTGTCTTACCGTCTGAA
CTTGCGTGGCCCGAGTATCTCCGTCCAAACTGCGTGCTCAACCTCGCTTG
TCGCTGTTCATTTAGCTTGTATGAGCCTCCTGGACCGGGAATGCGACATG
GCACTGGCAGGGGGCATCACCGTCCGCATCCCGCACCGTGCTGGTTATGT
GTACGCGGAAGGCGGTATTTTCTCACCAGATGGTCATTGTCGCGCATTCG
ATGCCAAGGCTAATGGAACCATTATGGGCAATGGCTGCGGCGTTGTGCTG
CTGAAGCCGTTAGATCGTGCGCTGTCCGACGGCGACCCTGTTCGCGCCGT
AATTCTGGGCAGCGCGACCAATAATGACGGTGCGCGCAAGATTGGGTTTA
CCGCGCCTTCAGAGGTGGGTCAGGCGCAAGCGATCATGGAGGCGCTGGCG
CTGGCGGGTGTTGAGGCGCGTAGTATCCAGTACATTGAAACACATGGCAC
CGGCACACTGCTCGGGGACGCAATCGAAACGGCAGCCTTACGCCGCGTTT
TCGATCGCGACGCGTCGACTCGCCGCTCTTGCGCCATCGGCTCTGTAAAA
ACCGGCATCGGTCATCTGGAATCTGCCGCTGGCATTGCTGGTTTGATTAA
GACCGTACTGGCGCTTGAACATCGTCAGCTGCCGCCTTCCCTCAACTTCG
AAAGCCCAAATCCGTCGATCGATTTTGCCTCATCTCCATTCTACGTGAAC
ACGTCACTGAAAGACTGGAACACTGGTAGCACACCACGCCGCGCCGGGGT
ATCAAGCTTTGGTATTGGCGGTACCAACGCCCATGTGGTGCTGGAAGAAG
CTCCGGCAGCCAAATTGCCAGCTGCCGCTCCAGCCCGTAGCGCCGAACTG
TTCGTTGTGTCAGCTAAATCAGCAGCAGCGTTGGATGCAGCGGCGGCTCG
TCTGCGCGATCACCTGCAAGCTCACCAGGGTTTGTCCCTGGGCGATGTCG
CCTTTAGTCTGGCTACTACACGCTCCCCTATGGAACATCGTTTGGCAATG
GCGGCCCCGAGTCGGGAAGCACTGCGCGAGGGTTTGGATGCGGCAGCCCG
TGGACAAACGCCTCCTGGCGCGGTCCGCGGTCGTTGTTCCCCTGGCAACG
TCCCGAAAGTCGTCTTCGTCTTTCCTGGCCAGGGTAGCCAGTGGGTGGGT
ATGGGTCGTCAGTTGTTGGCCGAAGAACCAGTTTTTCATGCCGCGCTTTC
CGCCTGCGATCGTGCAATCCAAGCTGAAGCTGGTTGGAGTTTATTGGCCG
AACTGGCTGCCGATGAAGGTTCTAGCCAGATCGAACGTATTGACGTGGTG
CAACCAGTTCTGTTCGCCTTAGCAGTAGCATTCGCTGCCCTGTGGAGATC
TTGGGGCGTTGGTCCTGACGTCGTAATCGGCCATAGCATGGGTGAGGTTG
CAGCTGCTCACGTTGCAGGCGCTCTGTCCCTCGAAGACGCGGTGGCAATC
ATTTGTCGCCGCAGCCGTCTGCTGCGGCGTATTTCGGGTCAGGGCGAGAT
GGCTGTTACTGAACTGAGCCTCGCGGAAGCAGAAGCCGCGCTGCGTGGCT
ATGAAGACCGTGTCTCGGTCGCGGTGAGCAATAGCCCGCGCTCTACCGTG
CTGTCGGGTGAACCTGCCGCAATCGGGGAGGTTTTGTCCAGCTTAAACGC
GAAGGGGGTATTTTGTCGTCGCGTGAAAGTAGATGTGGCTAGCCACTCAC
CACAGGTAGATCCATTACGTGAAGACCTGCTGGCAGCGCTGGGTGGCTTA
CGCCCGCGTGCGGCGGCCGTGCCGATGCGGTCAACTGTCACTGGTGCGAT
GGTGGCAGGCCCGGAACTGGGCGCTAACTACTGGATGAATAATCTGCGCC
AACCAGTTCGCTTCGCGGAAGTTGTTCAAGCGCAGCTCCAGGGCGGTCAC
GGTCTGTTTGTCGAAATGTCTCCGCATCCGATTCTGACCACCTCGGTCGA
GGAAATGCGTCGGGCGGCGCAACGCGCAGGCGCGGCAGTTGGTAGCTTAC
GTCGCGGCCAGGATGAACGGCCCGCCATGCTGGAGGCGTTAGGGGCGCTG
TGGGCCCAAGGTTATCCAGTTCCGTGGGGGCGCCTTTTTCCGGCAGGCGG
GCGCCGCGTTCCGTTGCCGACTTACCCTTGGCAGCGTGAACGCTACTGGC
TGCAGGCGCCAGCCAAAAGCGCCGCAGGCGATCGTCGCGGTGTTCGTGCA
GGCGGCCATCCGCTCTTGGGCGAAATGCAAACCTTATCAACGCAAACGTC
TACCCGCCTGTGGGAAACCACCTTGGATTTGAAGCGCCTGCCATGGCTGG
GTGATCATCGCGTCCAGGGCGCAGTGGTGTTTCCGGGTGCGGCCTATCTG
GAGATGGCTATTTCCTCGGGTGCTGAAGCCCTGGGCGATGGTCCGCTACA
GATTACGGACGTTGTTCTGGCGGAGGCACTTGCGTTCGCGGGCGACGCTG
CGGTACTGGTTCAGGTGGTGACGACAGAACAGCCGAGCGGGCGTTTACAG
TTTCAGATTGCAAGCCGTGCGCCGGGTGCGGGCCACGCGAGTTTTCGTGT
TCACGCACGCGGCGCTTTATTACGTGTAGAGCGCACTGAGGTGCCTGCGG
GGCTTACGCTTTCTGCGGTCCGGGCTCGCTTACAGGCGTCTATGCCAGCC
GCAGCGACGTATGCGGAACTTACGGAGATGGGGCTCCAGTACGGTCCGGC
ATTTCAGGGCATTGCCGAACTGTGGCGCGGCGAGGGGGAGGCATTGGGCC
GCGTACGTTTGCCGGACGCAGCGGGGAGCGCCGCGGAATATCGGCTCCAT
CCAGCGCTGCTGGATGCTTGCTTTCAAGTGGTGGGTTCTTTATTTGCTGG
CGGTGGGGAGGCTACCCCGTGGGTGCCGGTGGAAGTTGCTTCTCTGCGTC
TGCTGCAACGTCCTTCTGGGGAATTATGGTGTCACGCACGCGTAGTTAAC
CATGGCCGTCAGACTCCGGACCGTCAGGGTGCCGATTTCTGGGTAGTCGA
CAGCAGTGGCGCGGTGGTAGCGGAAGTGAGTGGCCTGGTGGCACAGCGTT
TGCCTGGCGGTGTCCGCCGTCGCGAAGAAGATGACTGGTTTCTTGAGCTT
GAGTGGGAGCCAGCCGCCGTCGGGACGGCTAAGGTTAATGCGGGTCGGTG
GTTGCTCCTGGGTGGCGGTGGCGGGCTGGGTGCTGCACTTCGTTCGATGC
TGGAAGCTGGCGGTCACGCGGTTGTGCATGCGGCCGAGAGCAATACATCT
GCGGCGGGCGTCCGGGCCCTGCTAGCGAAGGCGTTCGATGGGCAAGCTCC
TACAGCCGTGGTTCACCTGGGCTCGCTGGATGGCGGTGGCGAACTTGACC
CGGGCCTGGGGGCACAGGGGGCGCTGGATGCTCCTCGTAGTGCAGATGTG
TCGCCAGATGCACTGGATCCGGCCCTGGTGCGCGGCTGCGATAGTGTACT
GTGGACGGTCCAAGCGCTGGCAGGTATGGGCTTTCGCGACGCCCCGCGTC
TGTGGTTGCTGACTCGGGGTGCCCAGGCGGTAGGCGCCGGTGACGTGAGT
GTGACCCAGGCACCGCTGCTCGGTTTGGGTCGTGTTATTGCCATGGAACA
CGCTGACCTCCGTTGTGCTCGCGTGGATCTGGATCCTACCCGTCCGGATG
GTGAACTGGGTGCGCTGCTTGCGGAACTCCTTGCTGATGATGCCGAAGCC
GAAGTTGCCTTACGTGGCGGCGAGCGCTGTGTGGCTCGCATTGTTCGCCG
TCAGCCGGAAACCCGCCCTCGCGGTCGCATCGAAAGCTGCGTCCCAACTG
ATGTGACAATCCGTGCAGATAGCACCTATCTGGTCACCGGTGGTCTTGGC
GGCTTAGGCTTGTCGGTTGCGGGTTGGCTCGCGGAGCGCGGTGCAGGTCA
TCTGGTCCTGGTAGGCCGTAGCGGTGCCGCCTCTGTGGAGCAGAGGGCTG
CGGTGGCAGCTTTGGAAGCACGCGGGGCGCGTGTGACCGTGGCTAAAGCT
GACGTAGCTGATCGCGCCCAGTTAGAACGCATTTTACGGGAAGTGACGAC
CTCGGGCATGCCGTTACGCGGCGTCGTTCATGCCGCCGGGATTCTGGATG
ACGGGTTACTGATGCAGCAAACGCCCGCACGCTTTCGTAAAGTGATGGCG
CCAAAAGTTCAAGGCGCACTCCATCTTCATGCACTCACGCGCGAGGCACC
GCTGAGTTTTTTTGTCCTCTACGCCTCCGGCGTCGGCCTGTTGGGTTCTC
CGGGTCAGGGGAATTATGCGGCGGCCAATACCTTCTTGGATGCGCTGGCG
CACCACCGTCGTGCTCAGGGGTTACCAGCCTTAAGTGTGGATTGGGGCCT
GTTCGCGGAGGTTGGTATGGCTGCCGCACAAGAAGACCGGGGTGCACGTC
TGGTATCGCGCGGCATGCGCTCGCTGACCCCGGACGAAGGTCTGAGCGCT
CTGGCTCGTCTTCTTGAATCGGGCCGTGTTCAAGTGGGGGTCATGCCAGT
GAACCCTCGCCTGTGGGTGGAGTTGTATCCGGCGGCTGCGAGTTCACGCA
TGCTGTCTCGTCTCGTAACAGCACATCGTGCATCCGCTGGCGGCCCTGCG
GGCGACGGCGATCTTCTGCGTCGTCTGGCTGCGGCGGAGCCTTCCGCACG
TTCGGGTTTACTGGAACCGCTCCTTCGCGCCCAGATTTCACAGGTGCTGC
GGCTCCCAGAGGGCAAAATTGAGGTAGATGCGCCACTGACATCCCTGGGC
ATGAACAGTCTCATGGGTCTGGAGCTGCGGAACCGTATTGAAGCCATGTT
GGGCATTACGGTTCCGGCGACTCTTCTTTGGACGTATCCGACCGTAGCAG
CACTTTCGGGGCACTTAGCGCGTGAAGCATCTAGTGCTGCGCCGGTGGAG
AGTCCGCATACAACCGCAGATAGCGCAGTTGAAATCGAAGAAATGTCCCA
GGATGACCTGACTCAACTGATTGCCGCGAAATTTAAAGCCCTGACGGGGA
ATTC
EpoD (SEQ ID NO: 9)
ATGACCACACGTGGCCCGACCGCTCAACAAAATCCACTGAAACAAGCAGC
AATTATCATTCAGCGCCTTGAAGAACGCCTTGCAGGTCTGGCACAAGCGG
AACTGGAGCGTACTGAGCCAATTGCGATCGTAGGCATCGGGTGTCGTTTT
CCGGGTGGCGCAGACGCGCCGGAAGCATTCTGGGAACTGCTCGATGCTGA
GCGCGATGCCGTTCAGCCTTTGGACCGTCGCTGGGCACTGGTCGGGGTAG
CGCCAGTGGAAGCGGTCCCTCATTGGGCGGGTTTATTGACCGAACCGATT
GACTGTTTCGATGCGGCCTTTTTTGGTATTTCGCCGCGTGAAGCACGTAG
CTTGGATCCGCAGCACCGTCTGCTCCTTGAAGTAGCATGGGAGGGGCTGG
AAGACGCCGGCATCCCACCGCGTAGCATTGACGGCTCTCGCACTCGTGTC
TTTGTGGGTGCGTTCACCGCCGATTATGCCCGTACTGTTGCTCGCCTGCC
TCGTGAAGAACGCGACGCGTACAGCGCGACAGGTAACATGTTATCCATCG
CGGCTGGGCGTTTGTCGTATACGTTGGGCCTCCAGGGCCCGTGTTTGACC
GTTGATACCGCATGCTCGTCCTCTCTTGTTGCTATTCATCTGGCGTGCCG
CTCCTTGCGGGCTGGCGAAAGTGACCTGGCCCTTGCAGGCGGCGTCTCGA
CGTTGTTATCACCTGATATGATGGAAGCGGCGGCACGCACCCAGGCCCTG
TCCCCGGATGGCCGCTGTCGTACTTTCGATGCGTCGGCGAATGGCTTTGT
ACGTGGTGAGGGTTGTGGTCTGGTCGTTCTCAAACGTTTATCCGACGCAC
AGCGTGACGGCGACCGTATTTGGGCGTTAATCCGCGGCTCAGCGATTAAT
CATGACGGTCGCTCCACGGGCCTGACAGCGCCGAACGTCCTTGCGCAGCA
AACGGTGCTGCGCGAAGCACTGCGTAGTGCGCACGTTGAAGCAGGGGCCG
TGGATTACGTGGAGACTCATGGCACCGGCACCAGCCTGGGCGATCCGATC
GAAGTGGAGGCCCTGAGAGCCACCGTCGGCCCAGCCCCGAGCCACGGTAC
TCGCTGTGTGTTAGGCGCGGTAAAAACGAACATTGGACACCTGGAGGCAG
CCGCTGGTGTAGCTGGGCTGATTAAAGCTGCGCTGTCCTTAACGCACGAA
CGCATCCCGCGTAACCTGAACTTTCGTACCTTGAACCCGCGTATCCGTCT
TGAAGGCTCTGCATTGGCGCTCGCAACCGAGCCAGTTCCTTGGCCGCGCA
CAGATCGCCCACGCTTTGCCGGTCTGAGTTCATTTGGCATGTCGGGTACC
AATGCTCACGTGGTACTGGAGGAGGCTCCGGCCGTGGAACTGTGGCCTGC
GGCGCCGGAACGTTCCGCTGAACTGCTGGTGCTGAGCGGCAAATCTGAAG
GTGCCCTGGATCCTCAAGCTGCCCGTCTGCGTGAACATTTGGACATGCAC
CCGGAACTGGGGTTAGGCGATGTGGCTTTCTCCCTGGCAACGACCCGCTC
TGCGATGACACATCGGTTGGCTGTTGCGGTAACCTCCCGCGAAGGTCTGT
TGGCCGCCTTGTCAGCGGTTGCACAGGGCCAAACGCCAGCAGGCGCTGCA
CGGTGCATTGCGAGCTCTAGTCGCGGTAAGCTGGCTCTGCTGTTTACTGG
CCAGGGCGCCCAAACTCCGGGTATGGGTCGCGGCTTATGTGCCGCCTGGC
CCGCTTTTCGTGAAGCCTTTGATCGCTGTGTAACGTTATTTGACCGTGAG
CTGGATCGGCCACTGCGGGAGGTTATGTGGGCGGAAGCTGGGTCCGCCGA
ATCATTACTGTTAGACCAGACCGCGTTCACGCAGCCCGCGCTGTTCGCTG
TCGAATATGCCCTGACGGCGCTCTGGAGATCTTGGGGTGTCGAACCAGAA
CTGCTGGTTGGACACTCTATTGGCGAACTGGTCGCGGCGTGCGTGGCTGG
CGTTTTCTCTCTTGAAGACGGTGTGCGCCTCGTGGCGGCTCGGGGTCGCC
TCATGCAGGGGCTGAGCGCTGGCGGCGCCATGGTGTCACTGGGTGCTCCA
GAGGCAGAAGTAGCAGCAGCCGTCGCACCACATGCGGCATGGGTTTCAAT
CGCCGCCGTAAATGGCCCAGAGCAGGTAGTTATTGCAGGCGTCGAACAAG
CGGTGCAGGCAATCGCCGCAGGGTTTGCGGCGCGCGGCGTGCGCACTAAA
CGCCTCCACGTCTCTCATGCCTTTCACTCCCCGCTGATGGAACCAATGCT
GGAAGAGTTCGGTCGCGTGGCAGCGTCTGTTACCTACCGTCGTCCTAGCG
TCTCGCTCGTTTCCAACCTGAGTGGTAAAGTGGTTACTGACGAGCTGAGC
GCCCCAGGCTACTGGGTTCGTCATGTGCGCGAAGCCGTCCGTTTTGCTGA
TGGTGTGAAAGCCCTGCACGAAGCGGGCGCGGGCACCTTTCTGGAAGTCG
GTCCGAAACCAACCCTGCTGGGCCTGCTCCCGGCGTGCCTGCCAGAAGCA
GAACCTACGTTATTAGCGAGCTTGCGGGCGGGCCGTGAAGAAGCAGCGGG
TGTTCTGGAGGCCCTTGGGCGTTTGTGGGCGGCAGGCGGTTCCGTTTCTT
GGCCTGGCGTTTTTCCAACCGCTGGTCGCCGTGTGCCGCTTCCGACCTAT
CCGTGGCAACGTCAGCGCTATTGGCTGCAGGCACCGGCGGAAGGGCTGGG
TGCGACTGCGGCAGATGCGTTAGCCCAGTGGTTTTATCGCGTGGATTGGC
CGGAAATGCCACGGAGTAGCGTTGATTCTCGCCGTGCGCGTTCGGGCGGC
TGGCTTGTCCTGGCGGACCGTGGCGGGGTGGGCGAAGCAGCCGCAGCGGC
ACTGAGTAGTCAAGGCTGCTCATGTGCGGTGTTACATGCTCCGGCGGAGG
CGTCCGCCGTCGCCGAACAGGTGACCCAGGCCCTGGGCGGGCGCAATGAT
TGGCAGGGCGTTCTGTACTTGTGGGGTCTGGATGCAGTCGTCGAGGCGGG
CGCATCCGCAGAGGAGGTGGGTAAAGTGACACACCTGGCGACCGCTCCGG
TGTTAGCACTGATTCAGGCCGTCGGGACTGGCCCGCGCAGCCCTCGCCTG
TGGATTGTAACGCGTGGGGCTTGTACGGTCGGTGGCGAGCCGGATGCTGC
CCCGTGTCAGGCTGCACTGTGGGGGATGGGTCGTGTGGCAGCCTTGGAAC
ATCCGGGCTCCTGGGGTGGTCTGGTTGATCTGGATCCGGAAGAATCTCCA
ACGGAAGTAGAAGCGCTGGTGGCTGAACTGCTGTCTCCGGATGCCGAAGA
TCAGCTCGCATTTCGTCAAGGCCGTCGTCGTGCCGCCCGCTTGGTCGCCG
CGCCACCGGAGGGCAACGCAGCGCCGGTGTCGTTAAGCGCGGAAGGTTCA
TATTTGGTTACCGGTGGTCTGGGCGCTCTGGGTCTGCTGGTGGCTCGCTG
GCTGGTGGAACGTGGTGCGGGTCATCTGGTTTTAATCTCTCGGCACGGGC
TTCCTGATCGCGAAGAATGGGGCCGTGATCAACCACCTGAGGTACGGGCC
CGTATCGCAGCGATTGAGGCCCTCGAAGCTCAAGGCGCACGCGTAACGGT
TGCCGCCGTGGATGTTGCAGACGCTGAGGGGATGGCCGCTCTTTTAGCAG
CCGTGGAGCCGCCACTGCGCGGCGTGGTCCATGCCGCTGGCCTGCTGGAC
GACGGTCTGTTAGCGCACCAGGATGCAGGTCGCCTGGCTCGGGTGTTACG
TCCGAAAGTTGAAGGTGCTTGGGTTCTGCATACCCTGACCCGCGAGCAGC
CTCTTGATCTGTTTGTTCTGTTTAGCTCCGCAAGTGGTGTTTTCGGTTCC
ATCGGCCAGGGCTCTTATGCGGCAGGGAACGCATTTTTGGATGCTCTGGC
GGATCTGCGTCGTACACAAGGCTTGGCGGCCTTAAGCATTGCATGGGGCC
TGTGGGCGGAAGGGGGTATGGGCTCACAAGCCCAGCGCCGCGAGCATGAG
GCATCCGGTATCTGGGCGATGCCGACGTCTCGCGCCCTGGCGGCAATGGA
ATGGCTCCTGGGCACCCGCGCCACGCAGCGTGTGGTAATTCAGATGGACT
GGGCTCACGCGGGTGCAGCACCACGGGATGCTTCCAGAGGGCGTTTCTGG
GATCGTCTCGTAACCGTCACCAAAGCAGCTAGTAGCAGTGCTGTGCCCGC
AGTTGAACGCTGGCGTAATGCAAGCGTGGTCGAAACCCGTTCGGCTCTGT
ATGAGCTGGTGCGCGGCGTGGTAGCAGGTGTGATGGGTTTTACTGATCAA
GGCACATTAGATGTCCGGCGCGGCTTTGCAGAGCAGGGTTTAGATAGCCT
CATGGCGGTTGAAATTCGTAAACGTCTGCAAGGCGAGCTGGGTATGCCGT
TGTCTGCCACATTGGCGTTCGATCATCCGACCGTAGAACGTTTGGTGGAA
TATTTACTTAGCCAAGCGTCTAGTTTACAGGACCGTACGGATGTCCGCTC
CGTGCGTCTGCCAGCAACGGAAGATCCAATTGCGATTGTTGGGGCGGCAT
GCCGTTTTCCGGGTGGCGTCGAGGACCTGGAATCTTACTGGCAGTTGCTG
ACGGAAGGTGTGGTCGTTTCTACCGAAGTACCGGCAGACCGTTGGAACGG
GGCGGACGGCCGTGGCCCTGGCAGCGGTGAAGCACCGCGCCAGACCTATG
TCCCCCGCGGTGGCTTTCTCCGCGAAGTCGAAACTTTTGACGCGGCCTTC
TTTCACATCTCTCCGCGTGAAGCTATGTCCCTGGACCCGCAGCAACGCCT
GTTGTTAGAAGTCTCGTGGGAAGCAATCGAACGTGCCGGCCAGGATCCCA
GTGCCCTGCGTGAATCTCCTACTGGAGTGTTTGTGGGTGCGGGCCCGAAT
GAGTATGCAGAACGTGTTCAGGACTTAGCTGATGAAGCAGCAGGGCTCTA
CTCCGGAACTGGCAATATGCTGAGCGTCGCGGCAGGGCGTCTTTCCTTTT
TTTTGGGGTTACACGGCCCGACCCTGGCAGTCGACACTGCCTGTAGTAGC
AGTCTGGTCGCGTTGCACCTTGGCTGTCAATCACTGCGCCGTGGCGAGTG
TGACCAAGCTTTGGTGGGGGGCGTTAATATGTTACTGTCCCCAAAAACGT
TTGCCCTGCTTTCACGCATGCATGCGCTGTCACCTGGTGGACGTTGTAAG
ACTTTCTCGCCTGACGCTGACGGGTATGCCCGCGCCGAAGGCTGTGCCGT
TGTCGTCCTGAAGCGGCTGTCTGATGCACAACGGGATCGCGATCCGATCC
TGGCAGTAATCCGCGGTACAGCAATTAACCATGATGGTCCGAGCAGTGGC
TTGACAGTGCCCTCGGGTCCGGCACAGGAAGCCTTACTTCGTCAAGCGCT
GGCACATGCGGGCGTAGTGCCTGCTGATGTGGACTTCGTTGAATGCCATG
GCACGGGGACCGCTTTAGGTGATCCGATTGAGGTTCGCGCACTGTCCGAC
GTATACGGTCAGGCCCGCCCGGCGGATCGTCCGCTCATTCTGGGCGCGGC
CAAAGCGAATCTCGGGCACATGGAACCGGCAGCAGGCTTAGCTGGGCTGT
TGAAGGCCGTGCTGGCGCTGGGCCAGGAACAAATTCCGGCTCAGCCTGAA
CTGGGTGAACTGAACCCGCTGCTGCCATGGGAAGCCCTGCCCGTGGCGGT
GGCACGTGCGGCGGTCCCGTGGCCGCGCACGGATCGTCCGCGTTTTGCAG
GTGTGAGTTCGTTCGGTATGAGCGGTACCAACGCGCATGTTGTCCTTGAA
GAAGCGCCCGCCGTAGAATTATGGCCTGCGGCGCCGGAACGCTCGGCGGA
ATTGCTGGTTCTTTCTGGCAAGAGCGAGGGCGCACTGGACGCGCAGGCCG
CACGCCTGCGTGAACACTTAGACATGCATCCGGAACTGGGCCTGGGCGAT
GTAGCCTTCTCCCTGGCAACAACGCGCAGCGCGATGAACCATCGTCTGGC
CGTCGCTGTGACGAGTCGCGAAGGCTTATTAGCAGCTCTGAGCGCCGTTG
CGCAGGGTCAAACCCCGCCGGGTGCGGCTCGTTGCATTGCGAGCTCAAGC
CGTGGTAAGCTGGCCTTTCTGTTCACTGGCCAGGGGGCGCAGACCCCGGG
TATGGGCCGTGGGCTGTGCGCAGCATGGCCTGCTTTCCGCGAAGCATTTG
ATCGCTGCGTCGCCTTGTTTGATCGCGAACTGGACCGCCCGCTGTGTGAG
GTTATGTGGGCCGAGCCGGGTTCGGCGGAATCTCTGTTACTCGATQAAAC
AGCATTTACTCAGCCAGCCCTGTTTACGGTAGAATATGCCCTGACCGCGC
TGTGGAGATCTTGGGGCGTCGAACCTGAACTGGTGGCGGGGCACTCAGCG
GGCGAACTGGTGGCAGCCTGTGTAGCTGGTGTGTTCTCTCTGGAAGATGG
TGTCCGCCTTGTCGCGGCGCGTGGCCGCCTGATGCAGGGTCTGTCCGCTG
GTGGCGCGATGGTTAGTCTGGGTGCTCCGGAGGCGGAAGTTGCTGCCGCC
GTAGCTCCACATGCGGCTTGGGTATCAATCGCAGCGGTAAATGGTCCGGA
ACAAGTTGTCATTGCAGGCGTGGAACAGGCAGTTCAGGCAATCGCGGCGG
GTTTCGCAGCACGCGGGGTCCGTACGAAACGGCTGCACGTTAGTCATGCT
AGCCACTCTCCTCTGATGGAACCCATGCTGGAGGAGTTCGGCCGCGTTGC
TGCTTCTGTTACCTACCGCCGCCCATCTGTGTCGCTGGTTAGCAACCTGA
GTGGTAAGGTTGTCACCGATGAACTTTCTGCCCCGGGTTACTGGGTCCGT
CACGTGCGTGAAGCGGTCCGCTTTGCGGATGGTGTGAAAGCGTTACATGA
GGCTGGGGCTGGTACGTTTCTGGAGGTAGGGCCTAAACCGACCCTCCTGG
GCCTTCTGCCAGCATGCCTGCCGGAAGCGGAGCCGACGCTGTTGGCGAGC
CTTCGCGCAGGACGTGAGGAAGCAGCAGGCGTCTTAGAGGCCCTGGGTCG
TCTTTGGGCCGCCGGAGGAAGCGTCTCGTGGCCCGGTGTGTTTCCGACCG
CTGGCCGCCGTGTCCCCCTTCCAACCTATCCTTGGCAACGCCAGCGCTAC
TGGCTGCAGATCGAACCTGATAGTCGTCGCCACGCGGCGGCGGATCCGAC
ACAAGGTTGGTTTTACCGCGTGGATTGGCCGGAAATTCCTCGGAGTCTCC
AGAAGTCAGAGGAGGCTTCACGTGGGAGCTGGCTGGTTCTGGCCGATAAA
GGCGGTGTAGGCGAAGCGGTTGCGGCGGCTCTGTCTACACGCGGGTTACC
GTGCGTTGTCCTGCATGCCCCAGCCGAAACGTCAGCGACTGCGGAGCTGG
TGACGGAGGCTGCGGGCGGTCGCAGCGATTGGCAGGTTGTGCTGTATTTA
TGGGGGCTTGATGCGGTCGTCGGTGCTGAAGCAAGTATCGATGAAATTGG
GGATGCTACTCGTCGCGCGACCGCCCCGGTTCTGGGTCTCGCGCGCTTCC
TGTCGACCGTTAGTTGTAGCCCTCGGCTGTGGGTTGTTACACGCGGCGCG
TGCATCGTTGGTGATGAGCCCGCCATCGCGCCGTGCCAGGCAGCACTGTG
GGGGATGGGTCGCGTTGCCGCACTTGAACACCCTGGCGCATGGGGGGGCC
TCGTGGATTTGGATCCGCGAGCGTCTCCGCCTCAGGCTTCACCAATCGAC
GGTGAAATGTTAGTTACTGAACTGCTTAGTCAAGAAACCGAAGATCAGCT
TGCGTTCCGCCACGGCCGCCGCCATGCCGCTCGCCTCGTAGCCGCGCCAC
CGCGTGGGGAGGCAGCGCCTGCGTCCTTGAGCGCCGAAGCAAGTTACCTG
GTGACCGGTGGCCTGGGTGGCCTTGGCTTGATTGTCGCGCAGTGGCTGGT
GGAATTAGGCGCCCGTCATCTCGTGCTGACTTCACGTCGCCGGTTGCCGG
ATCGTCAGGCTTGGCGCGAACAGCAACCACCAGAAATCCGCGCTCGTATC
GCCGCTGTGGAAGCACTGGAAGCTCGTGGTGCCCGCGTTACTGTAGCAGC
CGTGGATGTCGCAGATGTCGAACCTATGACCGCCCTCGTGTCTTCAGTGG
AACCGCCGCTGCGCGGTGTTGTCCACGCTGCGGGCGTCTCGGTTATGCGT
CCGCTGGCTGAAACAGATGAGACGCTGTTAGAGTCTGTGCTGCGTCCTAA
GGTGGCGGGGAGCTGGTTATTGCATCGCCTGCTGCACGGCCGTCCGTTGG
ACCTGTTTGTGCTGTTCTCAAGCGGTGCCGCCGTTTGGGGCAGTCACAGC
CAGGGTGCGTATGCTGCTGCAAACGCGTTTTTGGATGGTCTGGCACATCT
GCGTCGCTCTCAGTCACTGCCCGCCTTAAGCGTAGCCTGGGGTCTCTGGG
CCGAAGGTGGCATGGCGGATGCTGAGGCGCATGCCCGCTTATCAGATATT
GGTGTGCTTCCAATGTCGACCTCTGCTGCCTTATCCGCATTGCAGCGTCT
GGTGGAAACCGGCGCAGCACAACGTACTGTCACGCGGATGGACTGGGCCC
GCTTTGCGCCAGTGTACACGGCACGTGGCCGTCGTAACCTGCTGAGCGCT
TTAGTGGCTGGTCGCGATATTATTGCGCCTAGCCCTCCGGCAGCTGCTAC
ACGTAATTGGCGGGGCCTCAGTGTCGCGGAGGCCCGCATGGCGCTGCATG
AAGTGGTCCATGGTGCAGTTGCGCGTGTTTTAGGCTTTTTGGACCCTTCT
GCACTGGATCCGGGCATGGGCTTTAACGAACAAGGTTTGGACTCTCTGAT
GGCCGTGGAGATTCGGAACCTTTTGCAGGCAGAACTGGACGTGCGTCTCT
CAACGACATTAGCGTTCGATCACCCTACTGTGCAGCGCCTGGTGGAGCAT
CTGCTCGTGGATGTGTCTAGTTTAGAAGACCGCTCTGATACGCAGCATGT
GCGCTCGCTGGCCTCCGACGAGCCAATTGCAATCGTGGGCGCTGCCTGCC
GTTTTCCGGGCGGCGTGGAAGACCTGGAAAGCTACTGGCAGTTACTGGCA
GAAGGGGTAGTGGTTTCGGCCGAAGTCCCTGCGGACCGCTGGGACGCGGC
CGATTGGTACGATCCGGATCCGGAAATCCCAGGGCGGACCTATGTTACCA
AAGGCGCGTTTTTGCGCGATCTTCAACGCCTGGATGCCACGTTCTTCCGC
ATTAGCCCGCGTGAGGCTATGAGCCTCGACCCGCAACAGCGCCTGCTTTT
GGAAGTGTCCTGGGAAGCGCTGGAGAGCGCCGGCATCGCCCCGGACACCT
TGCGTGACAGTCCGACTGGTGTCTTCGTAGGTGCGGGCCCAAACGAGTAT
TACACGCAGCGGTTACGGGGTTTTACTGACGGCGCCGCTGGTCTCTATGG
TCGCACTGGCAACATGCTCTCTGTGGCAGCAGGGCGCCTTTCGTTTTTTT
TAGGCTTGCACGGGCCGACATTGGCGATGGACACGGCGTGTTCGAGCTCG
TTAGTAGCGCTTCATCTGGCTTGTCAGTCGCTGCGTCTGGGTGAATGCGA
TCAGGCATTGGTTGGCGGCGTGAATGTCCTTTTAGCGCCGGAAACCTTTG
TCCTGCTGTCACGTATGCGTGCCTTGTCACCAGATGGTCGTTGTAAAACA
TTCAGCGCCGATGCAGATGGCTACGCACGTGGTGAAGGCTGTGCAGTGGT
GGTTCTGAAACGCCTCCGTGATGCGCAGAGGGCCGGTGACTCGATTCTGG
CGCTGATCCGCGGTAGTGCTGTAAACCATGATGGTCCGTCCTCGGGTCTG
ACCGTACCTAATGGTCCGGCGCAACAGGCACTCTTGCGTCAGGCTCTGAG
CCAAGCAGGTGTGTCCCCTGTGGATGTTGATTTCGTCGAATGCCATGGCA
CTGGTACGGCTCTGGGTGACCCGATTGAAGTTCAAGCTCTGAGTGAAGTA
TACGGTCCGGGTCGTAGCGAGGATCGCCCTCTCGTATTAGGCGCCGTTAA
AGCCAATGTTGCCCACTTGGAAGCAGCGAGCGGCCTGGCATCATTACTGA
AAGCGGTGCTTGCGTTACGCCACGAACAGATTCCAGCGCAGCCAGAGCTC
GGGGAGCTGAACCCGCACTTGCCGTGGAATACTCTCCCAGTGGCGGTTCC
ACGTAAAGCCGTGCCATGGGGCCGTGGCGCTCGTCCGCGCCGTGCGGGCG
TGAGTGCCTTTGGTTTATCGGGTACCAACGTTCATGTGGTGTTAGAAGAA
GCGCCGGAGGTAGAGTTAGTGCCAGCTGCACCTGCGCGTCCGGTCGAACT
GGTGGTGTTGAGTGCGAAAAGCGCTGCGGCTCTGGACGCTGCGGCAGAAC
GCCTGAGCGCCCATCTGAGCGCACATCCGGAGCTGTCGTTGGGCGATGTA
GCCTTTAGTCTGGCTACTACTCGGAGCCCGATGGAACACCGCCTGGCGAT
TGCGACCACCAGTCGCGAAGCCTTACGTGGTGCCCTGGATGCCGCAGCCC
AGCGCCAGACCCCGCAAGGCGCAGTGCGCGGCAAAGCCGTATCCAGCCGA
GGCAAATTAGCCTTCCTGTTTACTGGCCAGGGGGCCCAGATGCCGGGTAT
GGGGCGCGGCCTGTACGAAGCTTGGCCTGCCTTCCGCGAGGCGTTTGACC
GCTGCGTAGCGCTGTTTGACCGTGAACTGGATCAGCCGTTGCGTGAAGTT
ATGTGGGCGGCGCCAGGTTTGGCGCAAGCTGCGCGTTTAGATCAAACTGC
CTACGCGCAGCCAGCCCTGTTTGCACTTGAATACGCACTGGCTGCGCTGT
GGAGATCTTGGGGTGTCGAACCTCACGTTCTTCTGGGTCATTCGATTGGT
GAACTCGTTGCGGCGTGCGTGGCTGGTGTATTTAGCTTAGAGGACGCTGT
GCGCCTTGTGGCCGCACGCGGGCGTCTGATGCAGGCGTTGCCCGCTGGTG
GCGCCATGGTGGCTATCGCAGCGAGTGAAGCGGACGTAGCGGCGAGTGTC
GCTCCACACGCAGCCACCGTGAGTATCGCAGCCGTTAATGGTCCGGATGC
CGTGGTGATCGCAGGCGCGGAAGTTCAGGTTCTGGCGTTGGGTGCTACCT
TCGCGGCGCGCGGGATCCGTACGAAACGTCTGGCCGTATCTCACGCCTTT
CATTCACCGTTGATGGATCCTATGCTGGAGGATTTTCAACGTGTCGCGGC
GACCATTGCCTATCGTGCACCGGATCGTCCGGTAGTGTCGAACGTTACTG
GTCACGTGGCAGGTCCGGAGATCGCGACACCTGAATATTGGGTTCGTCAT
GTGCGTAGCGCGGTTCGCTTTGGCGATGGTGCTAAAGCCCTTCACGCTGC
GGGCGCAGCGACGTTTGTAGAAATTGGGCCGAAACCTGTATTGCTGGGTC
TGCTGCCAGCTTGCCTGGGCGAAGCGGACGCGGTACTTGTGCCAAGTTTA
CGCGCTGATCGCTCAGAGTGCGAAGTGGTGCTGGCAGCATTAGGCACATG
GTACGCCTGGGGTGGCGCACTGGACTGGAAAGGCGTATTTCCGGATGGGG
CCCGCCGCGTCGCGCTGCCGATGTATCCGTGGCAGCGCGAACGTCATTGG
CTGCAGCTGACACCTCGTTCTGCGGCTCCAGCGGGCATTGCGGGTCGTTG
GCCGCTGGCGGGCGTGGGTCTTTGCATGCCAGGCGCGGTGCTCCATCACG
TGCTGTCAATAGGGCCACGTCATCAGCCATTCCTGGGTGACCATCTGGTG
TTTGGTAAAGTCGTGGTGCCGGGTCCATTCCATGTGGCGGTGATTCTGAG
TATCGCAGCGGAACGCTGGCCTGAACGTGCAATCGAACTGACAGGCGTTG
AATTTCTGAAAGCCATCGCTATGGAGCCGGATCAGGAAGTGGAACTGCAT
GCTGTCCTGACGCCGGAGGCGGCAGGGGACGGGTATCTGTTCGAACTGGC
AACCTTGGCGGCACCAGAAACTGAGCGTCGTTGGACGACCCATGCTCGCG
GCCGTGTGCAACCGACAGATGGGGCACCGGGGGCCTTACCGCGTTTAGAG
GTGTTAGAAGATCGCGCCATTCAACCTTTGGACTTTGCGGGCTTCCTGGA
TCGCCTCTCAGCAGTCCGCATTGGCTGGGGCCCGTTGTGGCGGTGGCTTC
AGGATGGTCGTGTGGGTGACGAAGCTAGCCTGGCGACGCTGGTGCCGACC
TATCCAAACGCCCATGACGTGGCGCCGCTGCACCCGATTTTGTTAGATAA
CGGTTTCGCGGTGTCACTGTTGGCGACCCGGTCGGAACCAGAAGACGATG
GTACTCCACCGCTGCCGTTTGCTGTTGAACGCGTGCGCTGGTGGCGTGCA
CCTGTTGGTCGTGTCCGCTGTGGGGGCGTTCCGCGCTCACAGGCATTCGG
CGTCTCTTCGTTCGTACTTGTGGACGAAACTGGTGAAGTTGTCGCTGAGG
TGGAAGGCTTTGTGTGTCGCCGCGCTCCTCGCGAAGTCTTTCTGCGTCAG
GAATCAGGGGCGTCTACCGCTGCCCTGTATCGCCTGGATTGGCCTGAGGC
GCCGCTGCCGGATGCGCCAGCTGAGCGGATGGAAGAATCATGGGTGGTCG
TTGCAGCTCCGGGGTCCGAAATGGCAGCCGCACTGGCTACGCGCCTCAAC
CGCTGCGTGCTCGCCGAACCTAAAGGTCTGGAGGCGGCACTGGCAGGCGT
TAGCCCTGCCGGTGTGATTTGCCTGTGGGAACCTGGCGCGCATGAAGAAG
CACCTGCGGCAGCGCAGCGTGTCGCCACGGAAGGTCTGTCCGTCGTGCAG
GCACTTCGTGATCGCGCCGTACGCCTGTGGTGGGTAACCACAGGGGCTGT
GGCGGTGGAAGCTGGTGAGCGCGTGCAGGTTGCAACTGCCCCGGTCTGGG
GGCTCGGCCGCACCGTGATGCAAGAGCGTCCGGAACTGTCTTGTACGTTA
GTGGATCTGGAACCGGAAGTCGATGCAGCCCGTAGCGCCGACGTTCTGCT
CCGGGAATTAGGCCGTGCGGATGATGAAACGCAGGTCGTCTTCCGTTCCG
GCGAACGCCGTGTCGCTCGCCTGGTCAAAGCGACCACACCGGAAGGTCTT
CTTGTGCCGGACGCCGAATCTTATCGTCTCGAAGCAGGTCAGAAAGGCAC
CCTGGATCAGCTGCGGTTGGCACCAGCCCAACGGCGGGCTCCGGGCCCAG
GCGAAGTGGAAATCAAAGTAACCGCGAGCGGCCTGAATTTCCGTACTGTT
CTCGCTGTTCTGGGGATGTATCCTGGTGACGCAGGCCCGATCGGCGGGGA
TTGTGCCGGCATCGTCACCGCCGTGGGCCAGGGTGTCCATCACCTGAGCG
TAGGTGACGCGGTGATGACGTTAGGCACATTACACCGTTTTGTGACGGTG
GATGCTCGGCTGGTGGTTCGTCAACCGGCTGGCTTGACTCCTGCCCAAGC
TGCGACCGTCCCGGTTGCATTTCTGACTGCGTGGCTGGCACTGCATGATC
TGGGTAACCTCCGTCGTGGTGAACGCGTGCTGATTCATGCCGCCGCAGGT
GGCGTCGGCATGGCGGCCGTCCAAATCGCACGGTGGATCGGCGCCGAAGT
TTTTGCCACCGCCTCTCCGTCCAAATGGGCCGCTGTTCAGGCGATGGGTG
TGCCGCGTACGCACATTGCCAGTTCTAGGACTCTGGAGTTCGCTGAAACC
TTCCGCCAAGTTACGGGTGGCCGTGGTGTCGATGTTGTACTTAATGCTTT
GGCGGGCGAGTTTGTGGATGCATCTCTGAGCCTCTTGACCACTGGTGGTC
GTTTTCTGGAGATGGGCAAAACGGACATTCGCGATCGCGCCGCCGTCGCT
GCCGCCCACCCAGGGGTGCGCTACCGCGTATTTGACATCTTAGAGCTGGC
GCCAGATCGGACCCGTGAGATCCTGGAACGCGTCGTTGAAGGTTTCGCAG
CGGGCCATCTCCGCGCTTTGCCGGTGCATGCGTTTGCCATTACCAAAGCC
GAAGCGGCGTTCCGTTTCATGGCGCAGGCTCGGCACCAAGGCAAAGTCGT
CCTGCTCCCTGCGCCAAGCGCGGCCCCACTGGCCCCAACGGGGACGGTTC
TGCTGACCGGTGGCTTAGGGGCGCTCGGGTTGCATGTGGCACGCTGGTTG
GCTCAGCAGGGCGCTCCACACATGGTCCTGACGGGTCGCCGTGGTTTGGA
TACCCCAGGGGCGGCCAAAGCGGTTGCCGAAATTGAGGCTCTTGGTGCGC
GTGTCACTATTGCCGCATCTGATGTGGCTGATCGCAACGCTCTGGAGGCC
GTTTTACAAGCAATCCCAGCGGAATGGCCGCTCCAAGGCGTGATTCATGC
GGCTGGCGCACTTGATGATGGTGTCCTGGATGAACAGACCACGGACCGTT
TCAGCCGTGTATTAGCCCCGAAAGTAACTGGCGCCTGGAACCTGCACGAG
TTAACTGCGGGGAATGATCTGGCTTTTTTTGTGTTGTTTAGCTCAATGAG
TGGTCTGCTCGGTTCAGCTGGTCAGTCGAACTATGCCGCCGCCAACACCT
TTCTGGATGCGCTGGCGGCTCACCGCCGCGCAGAAGGGCTGGCAGCTCAG
TCGCTAGCTTGGGGTCCGTGGAGTGATGGCGGTATGGCGGCGGGTCTTTC
AGCCGCCCTTCAAGCACGTCTTGCACGCCACGGTATGGGCGCCCTTTCCC
CGGCGCAGGGCACCGCCCTGCTCGGTCAAGCGCTGGCACGCCCGGAAACT
CAGCTGGGTGCTATGTCCCTTGATGTGAGAGCGGCCTCCCAGGCGTCCGG
CGCCGCAGTTCCTCCAGTTTGGCGTGCCCTGGTGCGTGCAGAGGCTCGCC
ATGCCGCCGCAGGCGCCCAGGGTGCCTTAGCGGCACGCCTCGGGGCTTTG
CCTGAAGCCCGCCGCGCGGACGAAGTGCGGAAAGTTGTTCAAGCCGAAAT
TGCACGCGTGCTCAGCTGGGGGGCCGCCAGCGCCGTACCCGTTGATCGCC
CGCTGTCTGATCTGGGTTTAGATTCACTTACAGCTGTCGAATTACGCAAT
GTTCTCGGCCAGCGTGTTGGTGCAACCCTGCCAGCGACCCTTGCGTTTGA
TCACCCAACTGTAGACGCACTGACCCGTTGGCTCCTGGACAAAGTTTCTA
GTGTGGCAGAACCTTCCGTCTCCCCAGCCAAAAGCTCTCCGCAGGTTGCG
CTCGATGAACCAATTGCGGTTATTGGGATCGGTTGCCGCTTTCCGGGTGG
TGTTACCGATCCGGAAAGCTTCTGGCGCCTGCTGGAAGAAGGTAGCGATG
CGGTCGTTGAGGTCCCGCATGAGCGCTGGGACATCGATGCCTTCTATGAC
CCAGATCCGGATGTGCGTGGGAAAATGACTACGCGGTTTGGCGGGTTTTT
GTCGGATATTGACCGCTTCGAACCTGCATTTTTCGGCATTTCCCCGCGCG
AAGCTACGACCATGGATCCGCAGCAGCGCCTGCTGCTGGAAACGAGCTGG
GAAGCGTTTGAGCGTGCCGGCATTCTCCCAGAGCGTCTTATGGGTTCGGA
TACGGGTGTCTTTGTGGGTCTTTTCTATCAGGAATATGCGGCCCTGGCTG
GTGGTATTGAAGCATTTGACGGTTATCTGGGGACCGGCACCACGGCATCC
GTCGCGAGCGGCCGTATCTCGTATGTTCTGGGCTTAAAAGGTCCGTCGTT
GACTGTTGATACGGCGTGTAGTTCGTCGCTGGTGGCCGTACATCTGGCAT
GCCAAGCGCTCCGGCGGGGCGAATGCAGTGTCGCCTTAGCAGGTGGGGTG
GCTTTGATGTTGACCCCAGCTACATTTGTTGAGTTCAGTCGTCTGCGCGG
CTTGGCGCCGGACGGTCGTTGCAAATCATTCAGCGCTGCCGCAGATGGTG
TTGGTTGGTCCGAAGGCTGTGCGATGCTGCTCCTCAAACCGCTGCGCGAT
GCCCAACGCGACGGCGATCCGATCTTAGCGGTGATCCGCGGGACCGCCGT
AAACCAAGATGGCCGTAGCAACGGTTTAACGGCGCCTAATGGCTCCAGCC
AGCAGGAAGTCATCCGTCGCGCATTACAGCAGGCAGGCTTAGCGCCAGCC
GACGTGAGTTATGTCGAGTGTCATGGTACGGGAACCACCCTCGGTGATCC
GATCGAAGTGCAGGCGTTGGGTGCCGTATTAGCACAGGGCCGCCCGAGTG
ATCGTCCGCTGGTAATTGGTAGCGTCAAAAGCAACATTGGGCATACCCAG
GCTGCGGCAGGCGTGGCGGGTGTGATCAAAGTAGCTCTGGCTCTCGAACG
GGGCCTGATTCCGCGCTCCTTGCATTTTGATGCCCCGAACCCGCACATTC
CGTGGTCCGAACTGGCCGTGCAGGTCGCGGCCAAACCTGTGGAGTGGACA
CGCAACGGCGCACCGCGTCGCGCAGGCGTATCGAGTTTTGGTGTCAGCGG
TACCAATGCCCACGTCGTGTTAGAAGAAGCCCCAGCAGCGGCCTTCGCAC
CGGCCGCCGCCCGGTCAGCCGAGTTGTTTGTGCTGTCGGCGAAATCTGCG
GCGGCCCTGGATGCCCAGGCGGCACGTCTTTCTGCGCATGTCGTTGCACA
TCCTGAATTGGGCTTAGGCGATCTGGCCTTTAGTCTGGCGACTACCCGCT
CACCAATGACGTATCGCTTAGCAGTAGCTGCGACCAGCCGCGAGGCGTTG
TCTGCGGCCCTGGATACCGCCGCACAAGGGCAAGCACCTCCAGCTGCTGC
GCGTGGTCACGCGAGTACTGGCTCGGCGCCGAAAGTTGTATTTGTGTTCC
CTGGCCAAGGGAGCCAATGGTTAGGTATGGGGCAGAAACTGCTGTCCGAA
GAACCTGTATTCCGTGACGCTCTGTCAGCTTGCGATCGTGCGATTCAAGC
GGAGGCTGGGTGGTCCTTACTGGCAGAACTGGCAGCAGATGAAACCACCT
CACAGTTGGGTCGCATTGATGTGGTGCAGCCTGCGCTTTTTGCCATCGAA
GTGGCACTGAGCGCGCTGTGGAGATCTTGGGGTGTGGAACCGGATGCCGT
GGTTGGTCATTCTATGGGCGAAGTGGCGGCGGCCCACGTAGCAGGCGCCC
TTAGTCTGGAAGACGCGGTAGCGATCATTTGCAGGCGCAGCCTTTTGCTG
CGCCGTATTAGCGGGCAAGGCGAAATGGCAGTGGTCGAACTGTCCCTGGC
TGAAGCGGAAGCCGCGCTGCTGGGTTATGAAGACCGTCTTAGCGTTGCTG
TTTCGAACTCGCCACGCTCAACCGTGCTTGCGGGCGAGCCCGCTGCGCTG
GCCGAAGTTTTAGCGATCCTGGCAGCAAAAGGCGTCTTCTGTCGTCGCGT
GAAAGTAGATGTACCTAGCCACAGCCCTCAGATTGATCCATTACGTGACG
AACTGTTAGCGGCGCTGGGCGAACTGGAACCACGTCAGGCCACGGTCTCT
ATGCGGTCCACAGTAACAAGCACGATTGTGGCGGGCCCGGAACTGGTGGC
GAGCTATTGGGCAGATAATGTGCGCCAACCCGTCCGCTTCGCGGAAGCGG
TGCAATCTCTCATGGAAGGCGGGCATGGGCTGTTTGTCGAAATGTCGCCG
CACCCTATTTTGACCACCAGCGTCGAAGAAATCCGTCGGGCTACTAAACG
TGAAGGCGTTGCGGTAGGGTCGCTGCGTCGCGGCCAAGATGAACGGTTGT
CTATGCTGGAAGCGCTGGGCGCACTGTGGGTGCATGGGCAGGCTGTAGGT
TGGGAACGCCTGTTTAGTGCGGGCGGCGCAGGGCTGCGCCGTGTTCCATT
ACCAACGTACCCGTGGCAGCGCGAACGCTATTGGCTGCAGGCACCAACAG
GTGGTGCGGCGAGCGGCAGCCGTTTTGCGCATGCTGGGTCGCATCCGCTG
CTGGGTGAAATGCAGACCCTTAGTACCCAGCGTAGCACCCGCGTCTGGGA
GACCACACTCGATCTGAAACGGCTGCCGTGGCTGGGTGATCACCGTGTAC
AGGGGGCTGTAGTTTTCCCGGGTGCTGCCTATCTCGAAATGGCGCTGAGT
TCCGGTGCGGAGGCTCTGGGGGATGGTCCTCTCCAGGTTAGTGATGTGGT
CCTGGCGGAAGCCCTCGCTTTCGCGGACGACACCCCGGTGGCTGTGCAGG
TAATGGCTACGGAAGAGCGTCCGGGCCGTTTACAATTTCATGTGGCGTCA
CGTGTTCCGGGCCACGGCCGCGCTGCTTTTCGCTCTCACGCACGCGGCGT
CCTTCGTCAGACCGAGCGCGCAGAGGTGCCAGCACGCCTGGACCTGGCCG
CGCTGCGCGCACGCCTTCAGGCCAGTGCCCCAGCTGCCGCCACCTACGCA
GCCCTGGCCGAAATGGGTTTAGAATACGGCCCTGCCTTTCAAGGTTTAGT
TGAACTGTGGCGGGGTGAGGGCGAGGCGCTGCGTCGCGTACGTCTTCCGG
AGGCCGCTGGCAGCCCGGCCGCTTGTCGTCTGCATCCAGCACTGCTGGAC
GCCTCCTTTCACGTTTCTTCTGCGTTTGCTGATCGCGGGGAGGCCACACC
TTGGGTGCCGGTAGAAATCGGTTCTCTGCGCTGGTTTCAGCGGCCGTCAG
GCGAGCTTTGGTGTCATGCCCGTAGCGTATCCCATGGCAAACCTACGCCT
GATCGCCGCTCAACAGACTTTTGGGTGGTTGACTCGACTGGCGCGATCGT
GGCCGAGATTTCCGGGTTGGTTGCACAGCGTTTGGCAGGCGGCGTTCGTC
GCCGGGAAGAGGACGATTGGTTCATGGAACCTGCTTGGGAGCCGACAGCT
GTGCCTGGCTCTGAAGTTACTGCGGGCCGTTGGCTGTTGATTGGGTCGGG
TGGTGGGCTGGGTGCAGCCCTGTATAGTGCTCTGACGGAAGCAGGCCACA
GCGTGGTCCACGCCACCGGCCACGGCACCAGCGCGGCGGGCTTGCAGGCT
CTGCTGACGGCATCGTTTGACGGTCAGGCTCCGACTAGCGTCGTTCACCT
AGGTTCACTGGATGAACGCGGTGTTCTTGATGCCGACGCACCGTTTGATG
CTGACGCCCTGGAAGAGTCGCTGGTGCGCGGCTGCGATTCCGTACTGTGG
ACCGTCCAGGCGGTTGCAGGTGCGGGGTTCCGTGATCCGCCACGTCTTTG
GTTAGTGACGCGTGGGGCGCAGGCCATTGGCGCCGGTGATGTCTCTGTGG
CGCAAGCCCCACTGCTGGGTCTCGGCCGTGTGATCGCATTGGAGCACGCC
GAACTGCGTTGCGCCCGCATCGACCTGGATCCGGCGCGTCGCGACGGCGA
AGTCGATGAGCTTCTTGCAGAGCTGTTGGCTGACGATGCCGAGGAAGAAG
TTGCGTTTCGCGGCGGCGAACGCCGGGTGGCCCGCCTCGTGCGTCGTTTA
CCGGAGACAGATTGTCGTGAAAAAATCGAACCAGCTGAAGGCCGCCCTTT
TCGTCTGGAGATTGACGGTTCAGGTGTCCTGGACGATTTGGTTCTGCGTG
CCACGGAACGTCGTCCTCCGGGCCCGGGGGAAGTTGAAATCGCCGTGGAA
GCCGCCGGCCTGAATTTTTTGGATGTGATGCGTGCAATGGGCATTTACCC
TGGTCCGGGCGACGGTCCAGTAGCACTGGGCGCCGAATGTAGTGGTCGTA
TTGTTGCTATGGGCGAAGGCGTCGAAAGCCTTCGGATCGGCCAAGATGTC
GTCGCGGTCGCACCTTTCTCTTTTGGTACTCATGTGACAATCGATGCCCG
TATGGTCGCCCCGCGTCCAGCGGCGCTGACCGCAGCGCAGGCGGCTGCCC
TGCCTGTGGCCTTCATGACGGCATGGTATGGTTTAGTGCATCTGGGTCGT
CTGCGTGCGGGCGAACGTGTTTTGATTCATAGCGCCACTGGCGGCACTGG
CCTTGCGGCAGTACAAATCGCGCGCCATCTCGGGGCGGAGATATTTGCGA
CAGCAGGCACCCCGGAAAAACGCGCATGGCTCCGCGAACAAGGTATTGCG
CATGTAATGGATTCTAGGTCATTAGACTTTGCTGAACAGGTCCTGGCCGC
GACCAAAGGTGAAGGCGTGGATGTGGTTTTAAACTCCCTGTCCGGTGCGG
CAATCGATGCTTCATTAGCCACTTTAGTTCCAGACGGCCGTTTCATCGAA
CTGGGTAAAACGGACATTTACGCCGATCGCAGCCTGGGGCTGGCCCACTT
CCGCAAAAGCCTTTCCTACAGCGCAGTCGATCTGGCTGGTTTAGCGGTTC
GGCGCCCGGAGCGTGTTGCGGCTCTGCTTGCTGAGGTGGTAGACCTGCTG
GCACGTGGTGCGCTTCAGCCGTTGCCGGTAGAAATCTTTCCTTTGAGCCG
CGCGGCCGACGCGTTTCCCAAAATGGCACAAGCTCAACATCTGGGTAAAT
TGGTCCTGGCATTAGAGGATCCGGATGTGCGCATTCGCGTCCCAGGCGAG
AGTGGGGTAGCAATTCGCGCAGACGGCACGTACCTGGTGACCGGTGGGTT
AGGTGGGCTGGGTCTTAGCGTAGCGGGTTGGTTGGCCGAACAGGGCGCGG
GCCATCTGGTTCTGGTTGGTCGCTCGGGTGCCGTCAGTGCAGAACAACAG
ACCGCCGTAGCGGCCCTGGAAGCACACGGGGCTCGCGTTACAGTTGCTCG
TGCCGACGTTGCGGATCGTGCACAGATCGAACGTATCCTTCGCGAAGTGA
CCGCGTCGGGCATGCCGCTTCGTGGTGTGGTGCATGCAGCTGGCATCCTG
GATGACGGCCTGCTGATGCAGCAGACCCCGGCACGTTTTCGCGCAGTTAT
GGCTCCGAAAGTCAGAGGTGCCCTTCACTTGCATGCGCTGACCCGTGAAG
CGCCACTGAGTTTTTTCGTGTTATATGCGAGTGGTGCGGGCCTTTTGGGT
AGTCCAGGGCAGGGCAACTATGCCGCCGCGAACACTTTCTTAGATGCATT
AGCACACCACCGGCGCGCGCAGGGCCTCCCAGCCTTAAGTATTGACTGGG
GTCTGTTCGCTGATGTGGGGTTGGCCGCTGGACAGCAGAATCGCGGCGCG
CGCCTGGTAACACGTGGGACTCGCAGTCTGACCCCGGATGAAGGTCTGTG
GGCACTTGAACGTCTCCTGGATGGCGATCGGACTCAGGCAGGGGTGATGC
CGTTCGACGTGCGCCAATGGGTGGAGTTCTATCCGGCCGCTGCTTCTTCA
CGTCGCCTGAGTCGCTTGGTTACCGCCCGCCGTGTGGCGAGCGGCCGTCT
GGCAGGCGATCGCGATCTCTTAGAGCGCCTCGCTACGGCAGAAGCGGGTG
CCCGTGCAGGTATGCTCCAGGAAGTTGTTCGCGCACAAGTGTCTCAAGTG
CTTCGTCTCCCGGAAGGGAAACTTGACGTTGACGCTCCGCTGACCTCCCT
GGGCATGGATAGCTTGATGGGTCTTGAATTGCGTAACCGCATTGAAGCTG
TTTTGGGGATCACCATGCCTGCGACCCTGCTGTGGACTTATCCTACCGTC
GCGGCCCTGAGTGCGCACCTGGCGTCCCATGTGTCTAGTACTGGTGATGG
CGAGTCTGCCCGTCCACCGGACACAGGTAATGTTGCCCCTATGACCCATG
AAGTGGCGTCATTAGATGAAGATGGGTTGTTTGCTCTGATCGACGAATCC
CTGGCGCGCGCAGGCAAACGCGGGAATTC
EpoE (SEQ ID NO: 10)
ATGACCGACCGTGAAGGCCAGCTTTTGGAACCCCTGCGTGAAGTGACGTT
GGCCCTGCGGAAAACTCTGAACGAGCGCGATACCTTAGAGTTAGAAAAAA
CGGAACCAATTGCCATTGTCGGCATTGGCTGCCGTTTTCCAGGCGGTCCG
GGGACTCCGGAAGCTTTTTGGGAGCTGCTGGATGATGGTCGTGATGCGAT
CCGGCCACTTGAGGAGCGGTGGGCGCTGGTCGGGGTCGATCCTGGTGATG
ACGTCCCACGCTGGGCTGGCCTTCTGACTGAAGCGATTGACGGCTTTGAC
GCGGCCTTCTTTGGCATTGCGCCGCGCGAAGCCCGCTCTCTCGATCCTCA
GCACCGGCTGCTGCTGGAAGTTGCATGGGAAGGGTTTGAAGACGCCGGCA
TCCCGCCGCGTAGCCTGGTCGGGAGTCGCACGCGTGTCTTCGTAGGCGTA
TGTGCAACAGAATATTTACATGCGGCGGTGGCTCACCAGCCGCGCGAGGA
ACGCGATGCTTATAGCACAACGGGTAACATGTTGTCTATTGCCGCTGGCC
GCTTGTCATACACGCTTGGCCTTCAGGGCCCTTGCTTGACAGTTGACACA
GCCTGCTCTTCGAGTCTGGTGGCGATCCACCTGGCGTGTCGCTCACTCCG
TGCGCGTGAATCCGACTTAGCGCTGGCGGGTGGCGTCAATATGCTGTTAT
CTCCTGACACCATGCGCGCCCTTGCTCGTACCCAGGCATTGTCCCCGAAC
GGTCGTTGTCAAACCTTCGATGCAAGCGCGAACGGTTTTGTCCGGGGCGA
GGGTTGTGGCCTGATCGTGCTTAAACGTCTCTCCGATGCGCGTCGGGACG
GCGACCGTATTTGGGCCCTGATCCGCGGCAGCGCTATTAACCAGGATGGT
CGCTCCACAGGTCTGACCGCACCGAATGTACTGGCTCAGGGCGCACTGCT
GCGTGAAGCTTTACGTAATGCAGGGGTGGAAGCCGAAGCTATTGGCTACA
TCGAGACTCATGGCGCCGCGACTTCTTTAGGGGATCCGATTGAGATCGAA
GCCCTGCGCACTGTGGTGGGCCCGGCGCGCGCTGATGGCGCCCGTTGCGT
GCTCGGCGCGGTGAAAACCAACCTGGGCCATTTGGAAGGCGCGGCCGGGG
TTGCTGGGCTGATCAAAGCAACCCTGTCTTTGCACCATGAACGTATTCCG
CGCAACCTGAATTTCCGTACACTTAATCCGCGTATCCGCATTGAAGGGAC
GGCATTAGCCCTCGCTACCGAACCAGTTCCATGGCCTCGCACCGGCCGTA
CGCGGTTCGCCGGTGTTTCAAGCTTTGGCATGTCGGGTACCAATGCGCAT
GTTGTTCTGGAGGAAGCCCCTGCTGTTGAGCCGGAGGCAGCAGCGCCGGA
ACGGGCTGCCGAGCTGTTTGTGTTAAGTGCGAAATCAGTTGCCGCCCTGG
ATGCCCAAGCAGCGCGCCTGCGTGATCACCTGGAAAAACATGTGGAACTG
GGTCTTGGTGACGTGGCATTTAGCCTGGCGACTACCCGTAGCGCAATGGA
ACATCGCCTGGCCGTGGCAGCGAGCTCTCGTGAGGCGCTGCGCGGGGCCC
TGTCGGCTGCCGCCCAAGGCCACACGCCGCCGGGCGCGGTGCGGGGCCGC
GCATCCGGTGGGTCAGCGCCAAAAGTGGTCTTCGTGTTCCCTGGCCAGGG
TTCCCAGTGGGTAGGGATGGGCCGTAAACTGATGGCGGAAGAACCTGTCT
TTCGCGCAGCGCTGGAGGGCTGCGACCGTGCCATCGAAGCAGAAGCCGGT
TGGTCCCTGTTAGGTGAGCTGTCGGCAGATGAAGCCGCAAGCCAGCTTGG
CCGTATCGACGTTGTCCAGCCGGTACTGTTTGCTATGGAAGTGGCCTTAT
CGGCCCTGTGGAGATCTTGGGGTGTGGAGCCAGAGGCCGTAGTGGGTCAC
TCAATGGGCGAGGTAGCCGCTGCGCATGTGGCAGGTGCCCTGTCTCTGGA
AGACGCGGTGGCTATTATTTGCCGTCGCTCACGCCTGCTCCGTCGGATCT
CGGGGCAAGGTGAAATGGCACTCGTGGAGCTGTCCCTGGAGGAAGCCGAA
GCAGCCCTGCGCGGCCATGAAGGTCGCCTGTCTGTTGCTGTGTCCAATAG
CCCACGCAGCACCGTACTGGCCGGTGAACCGGCCGCACTGTCGGAAGTTC
TGGCAGCGTTGACCGCGAAAGGCGTTTTCTGGCGTCAAGTTAAAGTCGAT
GTGGCTAGCCACTCGCCGCAGGTGGACCCGTTGCGTGAAGAACTCATTGC
CGCCCTGGGTGCCATCCGCCCACGCGCAGCCGCTGTTCCAATGCGTTCCA
CCGTGACCGGCGGTGTTATTGCAGGCCCGGAACTGGGCGCGTCTTATTGG
GCTGATAACTTGCGCCAACCCGTACGGTTTGCGGCTGCCGCGCAAGCACT
GCTGGAAGGTGGTCCGACGCTGTTCATCGAAATGAGTCCGCATCCGATCC
TTGTCCCGCCGTTGGATGAAATTCAGACGGCGGTCGAACAAGGTGGTGCA
GCGGTTGGGTCACTGCGCCGTGGTCAGGACGAGCGTGCAACTTTACTGGA
AGCACTGGGGACCCTCTGGGCCTCGGGCTACCCGGTATCGTGGGCTCGTC
TGTTTCCAGCGGGGGGTCGTCGCGTACCGCTTCCAACGTATCCGTGGCAA
CACGAGCGTTGTTGGCTGCAGGTTGAACCAGATGCTCGTCGTTTAGCTGC
TGCCGACCCAACGAAAGATTGGTTCTATCGCACTGACTGGCCGGAAGTTC
CTCGCGCCGCCCCGAAAAGTGAAACAGCACACGGGAGCTGGCTTCTCCTC
GCTGACCGTGGCGGCGTTGGTGAGGCGGTCGCTGCGGCACTTAGCACCCG
TGGCCTGAGTTGTACCGTGTTACATGCGTCCGCTGATGCATCGACGGTTG
CGGAGCAAGTGAGCGAAGCCGCCAGCCGTCGCAACGATTGGCAGGGGGTA
TTGTATCTCTGGGGTCTGGATGCTGTCGTTGATGCTGGCGCGAGTGCAGA
TGAAGTTTCGGAAGCGACACGCCGCGCAACCGCGCCGGTGTTAGGTTTGG
TGCGCTTCCTGTCAGCTGCGCCGCATCCTCCCCGGTTTTGGGTTGTGACC
AGAGGTGCGTGCACCGTTGGCGGGGAGCCTGAAGTTAGTCTGTGCCAGGC
CGCGTTGTGGGGTCTGGCACGTGTGGTAGCGCTTGAACATCCGGCGGCCT
GGGGTGGCCTGGTCGATCTGGATCCGCAGAAATCACCGACCGAAATTGAA
CCACTGGTGGCTGAGCTGCTGAGCCCTGATGCCGAAGACCAGTTGGCTTT
TCGTAGTGGCCGTCGTCACGCAGCGCGGCTTGTCGCAGCGCCGCCGGAAG
GTGATGTCGCGCCGATCAGTCTTAGTGCGGAAGGCTCTTACTTAGTCACC
GGTGGCTTGGGTGGTCTGGGTCTTCTGGTGGCGCGCTGGTTGGTAGAGCG
TGGGGCCCGCCACTTGGTTCTGACTTCCCGCCATGGCCTGCCTGAACGTC
AAGCATCGGGTGGTGAACAGCCGCCCGAAGCCCGCGCACGCATTGCCGCC
GTGGAAGGTCTGGAAGCTCAGGGGGCACGTGTTACCGTAGCGGCGGTGGA
CGTAGCTGAGGCGGACCCTATGACGGCCTTGTTAGCTGCTATTGAGCCTC
CATTGCGCCGTGTCGTTCACGCCGCAGGTGTGTTTCCGGTCCGTCCGCTG
GCTGAAACTGATGAGGCCCTCTTAGAAAGCGTATTACGCCCTAAAGTTGC
CGGTAGTTGGTTACTGCATCGGCTTCTGCGTGACCGTCCTCTGGATTTGT
TTGTACTCTTCAGCAGCGGGGCGGCAGTCTGGGGGGGCAAAGGCCAGGGC
GCGTATGCAGCAGCAAATGCGTTCCTGGATGGCTTGGCACATCATCGTCG
CGCACATTCTCTGCCAGCCTTAAGTCTCGCATGGGGCCTGTGGGCGGAGG
GCGGCGTGGTTGATGCCAAAGCGCATGCGCGCTTATCTGACATCGGCGTT
CTCCCAATGGCGACGGGCCCGGCTCTCAGCGCGCTCGAACGCTTAGTGAA
CACAAGTGCGGTGCAGCGCAGCGTCACACGCATGGATTGGGCCCGCTTTG
CCCCAGTCTACGCCGCTCGTGGTCGGCGTAACCTGCTTTCCGCGCTGGTT
GCGGAAGATGAGCGCACGGCAAGCCCTCCGGTTCCAACCGCGAATCGCAT
TTGGCGCGGTCTGAGCGTAGCGGAATCACGCTCCGCGCTGTATGAACTGG
TGCGTGGTATTGTTGCACGGGTGCTGGGCTTCTCCGATCCGGGGGCGCTG
GACGTGGGTCGCGGCTTCGCGGAGCAGGGCCTGGATTCACTTATGGCGTT
GGAAATCCGCAATCGCTTACAGCGTGAACTGGGTGAGCGTTTAAGCGCCA
CCTTAGCTTTTGATCATCCGACGGTGGAACGCCTTGTCGCGCACCTGTTG
ACTGATGTGTCTAGTCTTGAAGACCGTTCCGATACGCGCCATATCCGCAG
CGTGGCCGCCGATGACGACATCGCAATTGTGGGCGCCGCATGTCGTTTTC
CGGGGGGCGATGAGGGGCTGGAGACCTACTGGCGTCACTTAGCTGAGGGC
ATGGTCGTTTCAACCGAGGTGCCAGCAGACCGTTGGCGCGCTGCGGACTG
GTATGATCCGGATCCGGAAGTACCAGGTCGTACCTACGTCGCGAAAGGTG
CCTTCCTCCGTGACGTGCGTTCGTTAGATGCGGCATTTTTTTCCATCAGT
CCGCGTGAAGCTATGAGTTTGGATCCGCAGCAGCGCCTGCTGCTGGAGGT
CTCATGGGAAGCTATCGAGCGCGCCGGCCAGGACCCGATGGCCTTACGCG
AGAGCGCCACTGGCGTCTTTGTCGGTATGATCGGTAGTGAACACGCCGAA
CGGGTCCAAGGTTTAGATGACGATGCCGCACTGCTGTACGGCACCACCGG
GAATTTGCTGTCTGTGGCAGCAGGCCGCCTGAGTTTTTTCCTGGGCCTGC
ATGGCCCGACGATGACCGTGGATACCGCTTGCTCTAGCTCCCTGGTCGCC
CTGCACCTGGCTTGCCAGTCATTACGCCTGGGCGAATGCGATCAGGCGCT
GGCTGGCGGTTCCTCTGTTCTGCTTTCGCCTCGCTCATTTGTGGCGGCCT
CCCGTATGCGTTTGCTGAGCCCTGATGGTCGCTGTAAAACGTTCAGCGCA
GCCGCCGATGGGTTTGCGCGTGCCGAAGGTTGCGCCGTGGTGGTATTAAA
ACGCCTGCGTGATGCCCAACGTGACCGCGACCCGATTTTGGCGGTGGTAA
GATCTACAGCCATTAACCACGATGGGCCTAGCAGTGGTCTCACCGTCCCG
TCTGGGCCAGCCCAACAGGCACTGTTGGGTCAAGCTCTTGCTCAAGCAGG
GGTAGCGCCTGCCGAAGTTGACTTTGTTGAGTGTCACGGAACCGGGACCG
CGCTGGGTGATCCAATAGAGGTCCAGGCTTTGGGCGCAGTGTATGGCCGT
GGTCGCCCGGCGGAGCGCCCACTGTGGTTAGGGGCAGTGAAAGCGAATCT
TGGGCATCTGGAGGCAGCCGCTGGCTTGGCAGGCGTTCTGAAAGTGCTGC
TGGCATTAGAACATGAACAAATTCCTGCGCAACCGGAACTGGATGAGCTG
AACCCTCATATTCCATGGGCGGAACTGCCGGTTGCGGTTGTCCGCGCCGC
AGTGCCGTGGCCTCGTGGCGCACGGCCACGTCGCGCCGGTGTGTCGGCAT
TCGGTCTCAGCGGTACCAACGCTCACGTCGTGCTTGAGGAGGCACCTGCT
GTTGAACCGGAGGCAGCCGCACCAGAACGTGCGGCCGAACTGTTCGTTCT
GAGCGCTAAAAGTGTGGCCGCGCTGGATGCTCAGGCCGCCCGCCTGCGTG
ATCATCTGGAAAAACACGTGGAACTTGGGCTGGGCGATGTCGCTTTCTCA
TTGGCTACCACACGTTCTGCCATGGAGCATCGTCTGGCGGTTGCAGCCAG
CTCTCGTGAAGCCCTGCGTGGTGCGTTGAGTGCCGCCGCGCAGGGTCACA
CTCCGCCGGGTGCCGTTCGCGGCCGTGCTTCTGGTGGCAGCGCCCCAAAA
GTAGTGTTCGTTTTCCCTGGCCAGGGTTCGCAGTGGGTAGGCATGGGCCG
TAAACTGATGGCGGAGGAGCCTGTATTTCGTGCCGCCCTTGAAGGCTGCG
ATCGTGCCATCGAAGCCGAAGCAGGCTGGTCCCTGCTTGGGGAACTCAGT
GCGGATGAAGCCGCCTCTCAACTTGGCCGCATTGATGTGGTCCAGCCGGT
TCTGTTTGCGGTTGAAGTGGCCCTGTCTGCTCTGTGGAGATCTTGGGGCG
TTGAACCGGAAGCTGTTGTAGGTCATAGCATGGGCGAAGTCGCAGCAGCC
CATGTTGCTGGTGCCTTGTCTCTGGAGGATGCGGTGGCGATTATCTGTCG
TCGCTCTCGCCTGCTGCGCCGGATTTCAGGCCAAGGTGAAATGGCCTTAG
TGGAACTGTCGTTAGAGGAAGCGGAAGCAGCATTCCGCGGGCATGAAGGT
CGTCTGAGCGTCGCAGTCTCAAACTCGCCTCGTTCTACCGTTTTAGCAGG
TGAACCTGCTGCTTTAAGTGAAGTTCTGGCCGCGTTGACCGCCAAAGGTG
TCTTCTGGCGTCAAGTGAAAGTGGATGTTGCTAGCCACAGTCCGCAAGTG
GACCCTTTGCGCGAGGAGCTGGTAGCTGCATTAGGCGCCATCCGCCCGCG
CGCTGCGGCGGTGCCAATGCGCAGCACCGTGACCGGGGGTGTCATTGCGG
GTCCTGAACTCGGTGCGTCTTATTGGGCTGATAACTTGCGCCAGCCAGTC
CGGTTTGCCGCAGCTGCACAAGCTTTGTTAGAAGGCGGGCCGACTCTCTT
CATTGAAATGTCCCCGCATCCGATCCTGGTTCCGCCTCTCGATGAAATCC
AGACAGCTGTGGAACAAGGGGGTGCAGCGGTTGGTTCACTGCGGCGTGGT
CAAGATGAACGCGCCACGCTGCTCGAAGCCTTGGGCACTCTGTCGGCGTC
GGGCTATCCGGTGTCATGGGCACGTCTGTTTCCTGCTGGGGGCCGTCGTG
TGCCTCTGCCGACATACCCGTGGCAGCATGAGCGGTACTGGCTGCAGGAT
TCTGTACATGGCAGCAAACCGTCCCTTCGCCTGCGCCAACTCCACAATGG
TGCAACGGATCATCCGTTACTGGGTGCGCCGTTACTGGTCAGCGCGCGCC
CTGGTGCACACCTGTGGGAACAGGCTTTGAGCCACGAACGTCTGTCTTAC
CTGTCAGAGCACCGTGTGCACGGCGAAGCGGTGCTTCCAAGCGCTGCGTA
TGTTGAGATGGCCCTTGCCGCAGGCGTCGACTTGTATGGCGCGGCGACTT
TAGTCTTAGAGCAGTTGGCATTGGAACGCGCCCTGGCAGTGCCTAGCGAG
GGGGGCCGCATTGTACAGGTTGCTCTGTCTGAAGAAGGCCCGGGCCGTGC
GTCTTTTCAGGTCTCGTCCCGTGAGGAAGCCGGTCGTTCTTGGGTACGTC
ATGCGACTGGGCACGTATGCAGCGATCAGTCCAGTGCGGTTGGTGCGCTT
AAGGAGGCGCCGTGGGAGATTCAACAGCGTTGTCCTTCCGTTCTGAGCTC
GGAAGCTCTGTACCCGTTACTGAACGAACATGCTCTTGACTATGGGCCGT
GTTTTCAGGGCGTAGAACAGGTTTGGCTGGGCACTGGCGAGGTACTGGGG
CGCGTCCGTCTCCCGGAAGACATGGCTTCGTCCAGCGGTGCGTACCGGAT
CCATCCGGCCTTGTTAGACGCGTGCTTTCAAGTCCTGACCGCACTGCTTA
CAACGCCAGAAAGTATCGAAATCCGCCGTCGCCTGACCGATCTGCACGAG
CCAGACCTGCCGCGTAGCCGTGCGCCAGTAAATCAGGCAGTGAGCGATAC
CTGGCTGTGGGATGCAGCATTGGATGGTGGTCGCAGACAGTCTGCCTCTG
TACCCGTTGACTTGGTACTTGGTTCTTTTCACGCTAAATGGGAAGTAATG
GACCGTTTGGCGCAAACTTATATCATTCGGACGCTTCGCACATGGAACGT
CTTTTGCGCCGCCGGCGAACGTCACACTATCGACGAGTTATTGGTGCGTT
TACAGATTAGTGCGGTGTATCGCAAAGTTATTAAACGCTGGATGGACCAT
CTGGTCGCCATTGGCGTGCTGGTGGGCGATGGCGAACATCTCGTATCATC
GCAGCCACTGCCGGAACACGACTGGGCGGCCGTTTTGGAGGAGGCGGCCA
CCGTGTTTGCGGACTTACCAGTTTTACTGGAGTGGTGTAAATTCGCAGGT
GAACGCCTGGCTGATGTGCTGACCGGCAAAACCCTGGCGTTGGAAATTCT
GTTTCCGGGCGGTAGCTTCGACATGGCAGAACGTATTTATCAGGACTCCC
CTATTGCGCGTTATAGTAACGGTATCGTCCGTGGTGTGGTCGAATCCGCA
GCCCGCGTCGTGGCGCCTTCGGGCACCTTTTCTATCTTAGAAATTGGCGC
AGGTACAGGGGCAACGACAGCGGCCGTTCTGCCTGTTCTGCTGCCGGACC
GTACGGAGTATCACTTCACCGATGTATCGCCGCTGTTCTTACCTCGTGCG
GAACAACGCTTTCGTGATCATCCGTTCCTGAAATACGGTATTCTGGATAT
TGATCAAGAGCCAGCGGGCCAGGGGTACGCCCATCAGAAATTCGATGTGA
TTGTGGCAGCGAATGTGATTCACGCGACCCGTGACATCCGTGCCACTGCG
AAACGTTTGCTGAGCTTGCTCGCGCCAGGCGGGCTGCTGGTGCTCGTGGA
AGGGACCGGCCACCCGATCTGGTTTGACATTACGACGGGCCTGATCGAAG
GCTGGCAGAAATATGAGGATGATCTGCGCACGGATCATCCGCTGTTGCCA
GCACGTACCTGGTGTGATGTGCTTCGCCGCGTTGGCTTCGCAGATGCCGT
GAGCCTTCCGGGCGATGGGTCTCCAGCCGGGATCCTGGGGCAGCACGTAA
TCTTATCGCGCGCGCCAGGCATCGCGGGCGCTGCTTGTGACTCAAGTGGC
GAGTCGGCTACTGAGTCTCCCGCGGCCCGGGCCGTCCGTCAAGAGTGGGC
GGATGGTTCGGCTGATGGCGTTCACCGCATGGCGCTGGAACGCATGTACT
TTCATCGCCGTCCAGGCCGCCAGGTTTGGGTGCACGGTCGCCTCCGTACA
GGGGGCGGCGCCTTCACGAAAGCACTGACGGGCGACCTGCTGCTTTTCGA
AGAAACGGGCCAGGTGGTGGCTGAGGTGCAGGGCCTGCGCCTGCCGCAGC
TTGAGGCATCTGCTTTTGCTCCGCGCGACCCACGTGAAGAGTGGTTATAC
GCGCTGGAGTGGCAGCGCAAAGATCCGATCCCTGAAGCGCCTGCCGCAGC
CTCATCCAGCACGGCGGGCGCGTGGCTTGTTCTTATGGATCAGGGCGGCA
CGGGCGCGGCCTTAGTGAGCCTGTTGGAAGGCAGAGGTGAAGCCTGCGTT
CGCGTGGTTGCAGGCACAGCGTATGCATGCTTGGCGCCTGGCCTGTATCA
GGTTGATCCGGCTCAGCCAGATGGCTTTCATACTCTGCTGCGCGACGCTT
TTGGGGAAGACCGTATGTGCCGCGCGGTGGTCCACATGTGGTCACTCGAT
GCTAAAGCCGCTGGTGAGCGTACCACAGCGGAATCGCTGCAAGCTGACCA
GCTGCTTGGTAGCCTGTCGGCCCTTAGCCTGGTGCAGGCCCTGGTACGGC
GCCGTTGGCGCAATATGCCGCGTCTTTGGCTGCTGACGCGTGCAGTGCAC
GCCGTGGGTGCGGAAGACGCTGCGGCCTCTGTCGCTCAGGCACCAGTCTG
GGGTCTTGGTCGCACACTCGCACTGGAACATCCGGAATTACGGTGCACTC
TCGTAGATGTTAATCCGGCGCCGAGTCCAGAAGATGCGGCGGCGCTGGCA
GTTGAGTTGGGCGCGAGTGATCGTGAGGATCAGATTGCCCTGCGCTCCAA
CGGTCGCTACGTTGCCCGGCTGGTTCGTTCAAGTTTCTCCGGCAAGCCGG
CGACCGACTGCGGCATTCGGGCCGATGGGTCATACGTCATCACCGATGGG
ATGGGCCGCGTTGGCCTCAGCGTTGCGCAGTGGATGGTTATGCAGGGCGC
GCGGCATGTTGTTCTCGTGGACCGTGGCGGCGCCAGTGATGCCTCTCGTG
ATGCACTTCGCTCGATGGCAGAAGCTGGTGCGGAAGTACAAATCGTCGAA
GCGGACGTGGCCCGCCGTGTAGATGTAGCCCGTTTACTGTCTAAAATTGA
ACCGAGTATGCCGCCGTTGCGGGGCATTGTGTATGTGGACGGTACGTTTC
AGGGGCATTCCAGCATGTTGGAACTCGATGCCCATCGCTTCAAAGAGTGG
ATGTATCCGAAAGTTTTGGGTGCTTGGAACTTGCACGCCCTGACACGTGA
CCGTAGCTTAGATTTTTTCGTCCTGTATAGCAGCGGTACATCTTTACTGG
GCCTTCCGGGTCAAGGTAGCCGCGCCGCAGGGGATGCCTTCTTAGATGCG
ATTGCACATCATCGCTGTCGCCTAGGTCTTACCGCGATGTCAATTAATTG
GGGCCTGCTTAGTGAAGCCAGCAGTCCGGCCACGCCAAACGATGGTGGTG
CGCGTCTCCAGTACCGTGGGATGGAAGGGCTTACCTTGGAGCAAGGTGCG
GAAGCTCTGGGTCGTTTACTTGCGCAACCACGCGCGCAGGTGGGGGTTAT
GCGCCTGAATCTCCGCCAGTGGCTGGAGTTCTACCCGAATGCGGCACGCC
TGGCATTATGGGCGGAACTGCTGAAAGAACGTGATCGCACCGATCGCAGT
GCAAGTAACGCTAGTAACCTGCGGGAAGCGCTTCAATCCGCCCGCCCGGA
GGATCGGCAGCTGGTTCTCGAAAAACACCTGTCAGAACTGCTGGGCCGTG
GTCTCCGTCTGCCACCAGAACGGATTGAACGTCATGTCCCTTTTAGCAAC
CTGGGTATGGACAGTCTCATTGGTTTAGAGCTGCGTAACCGGATTGAAGC
GGCCCTGGGTATTACCGTTCCTGCCACTCTGCTGTGGACGTATCCGACCG
TTGCCGCACTGTCCGGTAATCTCCTGGACATTCTTTCTAGTAATGCTGGC
GCGACGCATGCTCCGGCGACCGAGCGCGAAAAAAGCTTTGAAAACGACGC
CGCAGATTTAGAAGCCTTGCGTGGGATGACTGATGAACAGAAAGATGCGC
TGCTTGCGGAGAAACTCCCACAACTGGCCCAGATCGTGGGCGAAGGGAAT
TC
EpoF (SEQ ID NO: 11)
ATGGCGACGACGAACGCGGGTAAACTGGAACATGCTCTTCTGTTAATGGA
TAAGCTGGCGAAGAAGAACGCAAGTTTAGAGCAGGAACGCACTGAACCAA
TTGCGATTATTGGGATCGGCTGCCGTTTTCCGGGTGGTGCGGACACCCCG
GAAGCGTTTTGGGAACTGTTGGATAGTGGCCGCGATGCTGTGCAGCCGCT
GGATCGCCGTTGGGCGCTGGTGGGCGTCCATCCTTCAGAAGAAGTCCCGC
GCTGGGCGGGGTTGCTGACCGAGGCCGTGGATGGGTTTGACGCGGCGTTC
TTTGGTACAAGTCCGCGCGAAGCGCGTAGCCTCGATCCGCAACAGCGTCT
GCTCCTGGAGGTAACCTGGGAAGGTCTGGAAGATGCCGGCATCGCACCGC
AATCGCTGGATGGTAGCCGTACAGGCGTCTTTCTTGGGGCTTGTAGCTCC
GACTATAGCCATACTGTTGCGCAGCAGCGCCGCGAAGAACAGGACGCCTA
TGACATTACGGGCAACACTCTTTCCGTCGCTGCCGGGCGTCTCAGCTATA
CCCTCGGTCTACAGGGCCCGTGCCTCACCGTAGACACTGCGTGTAGCTCA
TCGTTGGTGGCAATTCACCTGGCGTGTCGCAGCCTCCGCGCACGCGAGTC
TGATCTGGCCCTGGCTGGCGGTGTTAATATGCTGCTGTCAAGCAAAACCA
TGATCATGCTCGGTCGCATTCAAGCACTGAGCCCGGATGGACATTGCCGT
ACCTTTGATGCGTCCGCTAATGGCTTCGTACGCGGCGAAGGCTGCGGTAT
GGTGGTATTAAAACGTCTGAGCGATGCCCAGCGGCACGGCGATCGCATTT
GGGCATTGATCCGCGGTTCAGCCATGAACCAGGACGGCCGTTCCACCGGG
TTGATGGCGCCAAACGTCCTCGCCCAGGAAGCGCTGCTGCGTCAGGCGCT
ACAGAGCGCACGTGTGGATGCTGGCGCGATCGATTACGTGGAGACACATG
GCACAGGCACCTCGCTGGGCGATCCAATAGAAGTTGACGCTCTGCGTGCA
GTCATGGGTCCGGCTCGTCCGGATGCGAGCCGTTGTGTGTTGGGTGCAGT
GAAAACAAACTTAGGCCACCTGGAGGGCGCCGCTGGGGTGGCGGGTCTGA
TCAAAGCCGCACTGGCGCTTCACCACGAAAGCATTCCTCGTAATCTGCAT
TTCCACACACTCAATCCGCGTATTCGTATTGAGGGAACCGCGCTGGCCCT
GGCAACCGAACCAGTTCCGTGGCCTCGCGCGGGTCGTCCACGCTTTGCGG
GTGTGTCTGCTTTCGGCCTGAGTGGTACCAACGTGCATGTTGTGTTGGAA
GAAGCACCTGCCACCGTGTTAGCCCCGGCAACGCCGGGCCGTTCTGCTGA
ACTGCTTGTTTTAAGCGCTAAATCCACAGCCGCTCTGGACGCACAGGCGG
CGCGGTTATCGGCCCACATCGCGGCATATCCGGAGCAAGGTCTGGGTGAT
GTGGCCTTTTCCTTAGTTGCGACCCGCAGTCCGATGGAACATCGTCTCGC
CGTTGCCGCCACGTCTCGCGAAGCGCTGCGTTCTGCGTTAGAGGCGGCGG
CACAGGGCCAAACCCCGGCAGGCGCGGCTCGTGGTCGTGCGGCCTCGTCA
CCGGGTAAATTGGCATTTCTGTTCGCTGGCCAGGGCGCCCAAGTACCAGG
TATGGGCCGTGGTCTGTGGGAAGCCTGGCCTGCGTTTCGTGAAACCTTCG
ACCGCTGCGTTACTTTGTTCGACCGTGAGCTGCACCAACCTCTGTGTGAA
GTTATGTGGGCGGAACCGGGTAGTAGCCGTTCGTCGCTTTTAGACCAAAC
GGCGTTCACCCAACCAGCGCTGTTCGCGCTTGAATACGCGCTGGCTGCGC
TGTTTAGATCTTGGGGCGTGGAACCGGAACTGATCGCGGGCCATTCTTTG
GGCGAGCTGGTGGCCGCGTGCGTTGCGGGCGTGTTTTCGCTGGAAGACGC
TGTTCGCTTGGTGGTGGCACGCGGGCGCCTGATGCAGGCGCTGCCAGCTG
GCGGTGCCATGGTTAGCATTGCCGCTCCGGAAGCCGATGTCGCCGCAGCT
GTTGCACCGCACGCGGCTAGTGTCTCAATCGCCGCCGTCAATGGCCCTGA
GCAGGTTGTCATTGCTGGCGCGGAGAAATTTGTGCAACAAATTGCCGCTG
CCTTTGCTGCGCGCGGTGCTCGCACCAAACCTTTGCATGTTTCCCACGCG
TTCCACTCCCCGCTGATGGATCCAATGCTGGAAGCATTTCGCCGCGTCAC
TGAATCTGTGACCTATCGCCGCCCGTCGATGGCGTTAGTAAGCAATCTGT
CGGGTAAACCGTGTACCGATGAGGTGTGTGCGCCTGGTTATTGGGTACGC
CATGCTCGGGAAGCGGTGCGCTTCGCAGATGGCGTTAAAGCGCTGCACGC
AGCAGGCGCGGGTATTTTTGTTGAAGTTGGTCCGAAACCTGCCCTGCTGG
GTCTGCTGCCTGCATGTCTGCCGGATGCCCGTCCAGTGTTACTGCCAGCA
AGCCGCGCAGGTCGTGACGAGGCCGCGTCAGCATTAGAAGCACTGGGTGG
GTTTTGGGTGGTTGGTGGCAGCGTAACGTGGAGTGGTGTGTTCCCGTCAG
GTGGTCGCCGTGTTCCTCTCCCAACGTATCCGTGGCAACGGGAACGGTAT
TGGCTGCAGGCACCTGTAGACGGTGAAGCGGATGGTATCGGTCGCGCACA
AGCTGGCGATCATCCATTGCTGGGTGAAGCCTTCAGTGTGTCAACCCACG
CAGGTCTGCGCCTGTGGGAGACTACCCTCGATCGTAAACGTCTGCCGTGG
CTGGGTGAGCATCGGGCGCAGGGTGAAGTAGTGTTTCCGGGGGCAGGCTA
CCTGGAAATGGCCCTTTCCTCAGGCGCCGAGATATTAGGGGATGGTCCGA
TCCAGGTAACGGATGTGGTGCTGATTGAGACCCTGACTTTTGCTGGCGAT
ACGGCAGTTCCTGTGCAGGTTGTGACAACTGAAGAACGTCCGGGTCGTCT
GCGGTTCCAGGTCGCCTCCCGCGAACCAGGGGCCCGTCGTGCAAGTTTTC
GCATTCATGCCCGTGGTGTTCTGCGTCGCGTCGGTCGTGCGGAAACGCCC
GCTCGTCTTAATCTCGCCGCACTGAGAGCCCGCCTGCATGCAGCAGTCCC
AGCCGCTGCTATCTATGGCGCATTGGCAGAAATGGGGTTACAGTACGGGC
CTGCACTGCGTGGTCTGGCAGAACTGTGGCGTGGCGAGGGTGAAGCTCTG
GGTCGCGTTCGTCTGCCAGAATCCGCGGGTTCGGCGACAGCCTATCAGCT
GCACCCGGTGCTCCTTGATGCATGCGTACACATGATTGTGGGCGCGTTCG
CGGACCGTGATGAAGCTACGCCATGGGCCCCGGTGGAGGTCGGGAGCGTG
CGTCTCTTCCAACGCTCTCCTGGCGAATTGTGGTGCCATGCCCGTGTTGT
GTCAGACGGCCAACAGGCACCGAGTCGCTGGAGCGCCGACTTTGAGCTGA
TGGACGGCACAGGGGCTGTAGTTGCAGAGATTAGCCGTCTCGTGGTTGAA
CGCTTAGCGTCCGGCGTCCGCCGCCGTGACGCGGACGATTGGTTTCTGGA
GCTCGATTGGGAACCGGCAGCATTAGAGGGTCCGAAAATCACGGCCGGTC
GCTGGCTGCTGCTGGGGGAGGGTGGGGGCTTGGGCCGTTCTTTATGTAGT
GCGCTGAAAGCGGCTGGTCATGTTGTGGTACACGCCGCAGGGGATGATAC
GTCTGCGGCAGGCATGCGTGCGTTGCTGGCGAACGCGTTCGATGGTCAGG
CGCCGACGGCTGTCGTCCACCTCAGCTCTCTGGACGGCGGCGGTCAACTG
GATCCTGGCTTGGGCGCTCAAGGCGCATTGGACGCTCCGAGATCTCCAGA
CGTGGACGCAGACGCCCTTGAGTCCGCATTAATGCGCGGTTGCGATTCCG
TGCTGAGCCTGGTGCAGGCGCTCGTCGGTATGGATCTGCGGAACGCACCA
CGTCTGTGGCTGCTTACCCGTGGCGCACAGGCAGCTGCCGCAGGCGATGT
CTCGGTGGTGCAGGCTCCGCTGCTGGGGCTCGGCCGCACGATCGCGCTGG
AACATGCAGAACTTCGCTGTATCTCAGTAGATTTGGATCCGGCACAGCCG
GAAGGCGAAGCGGACGCGCTGCTGGCCGAACTGCTGGCTGACGACGCGGA
GGAAGAAGTGGCATTGCGTGGTGGTGAACGCTTTGTGGCACGTCTGGTTC
ACCGCTTGCCGGAAGCGCAACGTCGGGAAAAAATTGCGCCAGCGGGCGAC
CGCCCGTTTCGCTTGGAAATCGATGAACCGGGTGTTTTAGATCAGTTAGT
TCTTCGTGCAACGGGTCGCCGTGCGCCGGGCCCGGGCGAAGTCGAGATCG
CCGTAGAGGCTGCGGGCCTGGATTCTATTGATATTCAGCTTGCCGTCGGG
GTAGCACCGAACGACTTGCCTGGCGGGGAGATCGAGCCGTCGGTCCTGGG
TAGTGAATGCGCCGGCCGCATCGTAGCAGTAGGTGAAGGCGTGAATGGGT
TGGTAGTGGGTCAGCCGGTTATTGCCTTAGCGGCGGGTGTTTTTGCGACG
CATGTTACGACTTCTGCGACCCTGGTGCTGCCGCGTCCGCTCGGGTTGAG
CGCGACCGAAGCGGCGGCGATGCCATTGGCGTATCTTACCGCTTGGTATG
CGCTTGATAAAGTTGCTCACCTTCAGGCAGGCGAACGTGTTCTGATTCGG
GCGGAGGCCGGGGGCATTGGTCTGTGCGCCGTCCGGTGGGCGCAGCGCGT
TGGTGCTGAGGTCTATGCGACCGCCGACACGCCAGAAAAACGTGCCTACC
TTGAGTCGCTGGGTGTGCGCTACGTGAGCGATCCTAGGTCTGGTCGCTTC
GCAGCGGATGTCCATGCGTGGACCGATGGGGACGGCGTTGATGTGGTTCT
GGACTCTCTGTCCGGCGAACATATCGATAAAAGTCTGATGGTTTTACGCG
CATGTGGGCGCCTCGTTAAACTGGGTCGCCGTGACGATTGCGCTGACACC
CAACCAGGGCTGCCACCGTTGTTGCGCAACTTTTCATTTTCTCAGGTGGA
TCTGCGTGGCATGATGCTGGACCAGCCCGCGCGGATTCGTGCTCTTCTGG
ATGAATTGTTTGGCCTGGTGGCGGCCGGTGCGATTTCCCCTTTAGGGAGC
GGTCTGCGGGTTGGTGGCAGCCTGACCCCGCCACCTGTCGAAACCTTCCC
AATTAGTCGTGCCGCTGAAGCCTTCCGTCGCATGGCGCAGGGTCAGCATC
TCGGTAAACTGGTCCTGACCCTGGATGATCCAGAGGTTCGTATTCGTGCG
CCAGCCGAAAGCAGCGTGGCAGTTCGTGCAGATGGCACCTATTTAGTTAC
CGGTGGTTTAGGTGGCTTGGGCTTACGTGTTGCTGGCTGGCTGGCAGAAC
GCGGTGCTGGGCAGTTAGTGTTAGTGGGCCGTAGCGGCGCTGCCTCCGCA
GAACAGAGAGCCGCCGTGGCCGCCCTGGAGGCCCATGGCGCCCGCGTCAC
CGTAGCTAAAGCTGATGTAGCGGATCGTTCACAAATTGAACGCGTACTGC
GCGAAGTCACGGCTTCCGGCATGCCGCTGCGGGGCGTTGTCCACGCCGCT
GGTTTAGTAGACGACGGCCTGTTGATGCAACAGACCCCGGCCCGCCTTCG
TACGGTAATGGGCCCTAAAGTGCAAGGTGCCCTTCATCTGCACACTCTGA
CTCGGGAAGCACCTTTATCTTTCTTTGTTCTGTATGCAAGTGCAGCAGGT
TTATTCGGCAGCCCGGGTCAGGGTAATTACGCTGCTGCAAACGCTTTTCT
GGATGCGCTGAGTCATCACCGGCGTGCGCATGGGTTGCCAGCCTTAAGCA
TTGACTGGGGCATGTTTACCGAAGTGGGGATGGCGGTCGCACAAGAGAAC
CGTGGCGCACGCCTTATTAGTCGGGGCATGCGCGGTATTACGCCGGACGA
AGGGCTGTCAGCGTTGGCCCGCCTTCTCGAAGGTGATCGTGTTCAAACGG
GTGTGATCCCGATTACACCGCGTCAGTGGGTGGAGTTCTATCCGGCCACA
GCGGCCACTCGTCGTCTCAGCCGCCTGGTCACAACTCAGCGTGCGGTCGC
TGATCGCACCGCCGGGGATCGCGATCTCCTCGAACAGTTGGCCTCGGCGG
AACCATCCGCTCGGGCTGGCCTGTTGCAAGATGTCGTACGCGTGCAGGTG
TCGCATGTGCTCCGCCTGCCGGAGGATAAAATCGAGGTGGACGCACCGTT
ATCCAGTATGGGTATGGATAGTTTGATGTCGCTGGAATTACGCAATCGTA
TCGAAGCCGCCCTGGGCGTAGCGGCTCCGGCAGCTCTGGGTTGGACTTAC
CCGACGGTGGCAGCTATTACCCGTTGGTTACTGGATGATGCTCTTTCTAG
TCGCTTAGGCGGCGGGAGCGATACGGATGAATCCACTGCATCGGCGGGTA
GCTTTGTTCACGTCCTGCGTTTTCGCCCGGTAGTAAAACCGCGTGCACGC
CTGTTTTGTTTTCACGGTTCGGGGGGTTCTCCAGAAGGCTTCCGTAGCTG
GTCTGAAAAATCAGAGTGGAGTGACCTCGAAATTGTCGCGATGTGGCATG
ATCGTTCCTTGGCATCTGAGGATGCCCCGGGCAAAAAATATGTTCAGGAA
GCTGCCAGTCTCATCCAACATTATGCGGATGCCCCATTTGCTCTTGTGGG
TTTCTCTTTGGGTGTTCGCTTTGTAATGGGCACAGCGGTGGAGCTGGCTT
CTCGGAGTGGGGCGCCAGCACCATTGGCGGTGTTCGCACTGGGTGGCTCC
CTGATTTCCAGCAGCGAAATCACTCCGGAGATGGAGACCGATATTATCGC
GAAACTGTTTTTTCGTAACGCGGCCGGTTTCGTGCGCTCAACACAGCAAG
TCCAGGCTGACGCCCGCGCGGATAAAGTGATTACTGATACCATGGTCGCC
CCTGCGCCGGGTGATAGCAAAGAACCGCCGTCAAAAATCGCGGTGCCGAT
CGTTGCAATTGCCGGTTCGGATGACGTGATCGTCCCTCCATCGGACGTTC
AGGACTTACAGAGCCGTACCACCGAACGGTTTTACATGCATCTGCTGCCG
GGCGACCATGAGTTCCTGGTTGACCGCGGGCGTGAAATTATGCATATTGT
AGATTCACACCTTAATCCGCTGTTAGCTGCCCGCACCACGTCCAGTGGCC
CGGCCTTCGAAGCAAAAGGGAATTC

All publications and patent documents cited herein are incorporated herein by reference as if each such publication or document was specifically and individually indicated to be incorporated herein by reference.

Although the present invention has been described in detail with reference to specific embodiments, those of skill in the art will recognize that modifications and improvements are within the scope and spirit of the invention. Citation of publications and patent documents is not intended as an admission that any such document is pertinent prior art, nor does it constitute any admission as to the contents or date of the same. The invention having now been described by way of written description, those of skill in the art will recognize that the invention can be practiced in a variety of embodiments and that the foregoing description are for purposes of illustration and not limitation.

1-29. (canceled)

30. A composition comprising a cognate pair of vectors, wherein said cognate pairs are:
a) a first vector comprising SM42S₁-Sy₁-2S₂-SM2-R₁ digested with a Type IIS restriction enzyme that recognizes 2S₂, and
a second vector comprising SM5-2S₃-Sy₂-2S₄-SM3-R₁ digested with a Type IIS restriction enzyme that recognizes 2S₃;
or
b) a first vector comprising L-2S₁-Sy₁-2S₂-SM2-R₁ digested with a Type IIS restriction enzyme that recognizes 2S₂, and
a second vector comprising L′-2S₃-Sy₂-2S₄-SM3-R₁ digested with a Type IIS restriction enzyme that recognizes 2S₃;
wherein SM1, SM2, SM3, SM4 are sequences encoding different selection markers, R₁ is a recognition site for a restriction enzyme, L and L′ are recognition sites that are the same or the same or different, and each different from R₁, 2S₁, 2S₂, 2S₃, and 2S₄ are recognition sites for Type IIS restriction enzymes, wherein 2S₁, 2S₂ are not the same, 2S₃, and 2S₄ are not the same, and digestion of the first vector with 2S₂ and the second vector with 2S₃ results in compatible ends.

31. The composition of claim 30 wherein 2S₁, and 2S₃ are the same and 2S₂ and 2S₄ are the same.

32. The composition of claim 30 wherein Sy₁ and Sy₂ encode polypeptide segments of a polyketide synthase.

33. (canceled)

34. A method for joining a series of DNA units using a vector pair comprising
a) providing a first set of DNA units, each in a first-type selectable vector comprising a first selectable marker and providing a second set of DNA units, each in a second-type selectable vector comprising a second selectable marker different from the first,
wherein said first-type and second-type selectable vectors can be selected based on the different selectable markers,
b) recombinantly joining a DNA unit from the first set with an adjacent DNA unit from the second set to generate a first-type selectable vector comprising a third DNA unit, and obtaining a desired clone by selecting for the first selectable marker
c) recombinantly joining the third DNA unit with an adjacent DNA unit from the second set to generate a first-type selectable vector comprising a fourth DNA unit, and obtaining a desired clone by selecting for the first selectable marker, or
recombinantly joining the third DNA unit with an adjacent DNA unit from the second series to generate a second-type selectable vector comprising a fourth DNA unit, and obtaining a desired clone by selecting for the second selectable marker.

35. The method of claim 34 wherein step (c) comprises recombinantly joining the third DNA unit with an adjacent DNA unit from the second set to generate a first-type selectable vector comprising a fourth DNA unit, and obtaining a desired clone by selecting for the first selectable marker, said method further comprising
recombinantly combining the fourth DNA unit with an adjacent DNA unit from the second series to generate a first-type selectable vector comprising a fifth DNA unit, and obtaining a desired clone by selecting for the first selection marker, or
recombinantly combining the third DNA unit with an adjacent DNA unit from the second set to generate a second-type selectable vector comprising a fifth DNA unit, and obtaining a desired clone by selecting for the second selection marker.

36. The method of claim 34 wherein step (c) comprises recombinantly joining the third DNA unit with an adjacent DNA unit from the second series to generate a second-type selectable vector comprising a fourth DNA unit, and obtaining a desired clone by selecting for the second selectable marker, said method further comprising
recombinantly joining the fourth DNA unit with an adjacent DNA unit from the first set to generate a first-type selectable vector comprising a fifth DNA unit, and obtaining a desired clone by selecting for the first selection marker, or
recombinantly joining the third DNA unit with an adjacent DNA unit from the first set to generate a second-type selectable vector comprising a fifth DNA unit and obtaining a desired clone by selecting for the second selection marker.

37. The method of claim 34 wherein the desired clone comprises a sequence encoding a PKS domain.

38-60. (canceled)

61. A system for high through-put synthesis of synthetic genes comprising:
at least one source microwell plate containing oligonucleotides for assembly PCR
a source for an assembly PCR amplification mixture
a source for LIC extension primer mixture
at least one PCR microwell plate for amplification of oligonucleotides
a liquid handling device which
retrieves a plurality of predetermined sets of oligonucleotides from the source microwell plate(s)
combines the predetermined sets and the amplification mixture in wells of the at least one PCR microwell plate;
retrieves LIC extension primer mixture; and
combines the LIC extension primer mixture and amplicons in a well of the at least one PCR microwell plate; and
a heat source for PCR amplification configured to accept the at least one PCR microwell plate.

62. The system of claim 1 further comprising a source for at least two assembly vectors.

63-64. (canceled)


SEQUENCE LISTING (36 sequences)

SEQ ID NO: 1 - DNA, 42 bp, Artificial Sequence
  Synthetic construct - 316-4-For_Morph_dU
gcuauaucgcuaucgaugagcugccactgagcaccaactacg

SEQ ID NO: 2 - DNA, 43 bp, Artificial Sequence
  Synthetic construct - 316-4-Rev_Morph_dU
gcuagugaucgaugcauugagcuggcacttcgctcactacacc

SEQ ID NO: 3 - DNA, 10641 bp, Artificial Sequence
  Synthetic construct - DEBS1
atggcagatctgagcaaactctccgattctcgcaccgcccagccgggccgcatcgtccgcccatggccgctgtctggctgcaatgaatccgcattgcgtgctcgcgcccggcagcttcgggcacacctggaccgttttccggacgcgggcgtggagggcgtgggtgcggcattggcccacgacgagcaggcggacgcaggtccgcatcgtgcggtggttgttgcttcatcgacctcagaattactggatggtctggccgcggtggccgatggtcgcccgcatgcgagcgtcgtacgcggggttgcgcgtccttctgccccggtagtgtttgtgtttcctgggcagggggcacagtgggcaggtatggcgggcgagctgcttggcgagtcgcgcgtgttcgctgccgccatggacgcctgtgctcgcgcgttcgaacctgtgacagactggacgcttgcacaggtcctggatagccctgaacaaagccgccgcgttgaagtggtccagccagcgttattcgccgtgcaaacttcgctagcggcgctctggcgttcctttggcgtgaccccagatgctgtggttggccattcaattggtgaattagcagcggcgcatgtttgcggtgccgcaggtgcggcggatgcagcgcgcgcagcggcactgtggagtcgcgagatgattccgttggtgggcaacggcgacatggccgctgtcgctctgtcggcagatgaaattgaaccacgtatcgcgcgctgggacgatgacgtagtgctggcgggcgtcaacggtccgcggtccgtcctgttgacagggtcacctgaacccgtagctcgtcgtgtgcaggaactgagcgccgagggcgtacgcgcccaggtaatcaatgttagcatggctgcgcatagcgctcaggttgatgacatcgctgagggtatgcgtagtgccctggcgtggtttgccccaggcggctccgaagttccgttctacgcctcactgaccggcggtgcggttgatacccgtgagttagtagccgattactggcgtcgttcttttcggctaccggtacggtttgatgaagcgatccgcagtgccttggaagtaggcccgggtacgtttgtcgaagcgagcccgcatcctgtgttggcggcggcgctgcaacagaccctggatgccgaaggttcaagcgcggctgttgtacctacactgcagcgtggtcaagggggcatgcgtcgcttcctgttggccgcggcccaggctttcactggcggcgtcgcggttgactggacggccgcttacgatgatgttggtgccgaaccaggttcgctgcctgagttcgctccggccgaagaagaggacgagccggcagagtccggggttgattggaacgcaccgccacacgtgctccgcgaacgtctgctggctgtggtgaacggggagaccgcagctcttgcaggccgcgaagctgacgcagaggcgacctttcgcgaattaggtctcgattctgtgttagcagcccagctgcgcgcgaaagtcagcgcggccattggccgtgaagtgaatattgcgctgttatatgaccatccaaccccgcgtgcacttgcggaggcactgtctagtgggacggaagtagcgcaacgcgagactcgcgcccgtacaaacgaagctgcacctggcgaaccaattgcggtagtagcgatggcatgtcgtttaccgggcggtgtatcgacccctgaagagttctgggagctgttgtcagaaggccgggatgcggtggcggggcttccgactgacagagggtgggacctggatagcctgttccacccggatccaactcgttcgggcaccgcccatcagcggggcggtgggtttctgaccgaggcgacggcttttgatccggccttctttggtatgagcccgcgcgaggcgttagccgtggatcctcagcagcgcttgatgctggaactttcttgggaagtcttagaacgtgccggcatcccgccgacttccctacaggcaagtccgacgggtgttttcgtcgggctgattccgcaggagtacggcccacgtctggcggaaggcggcgaaggggtggaaggctacctgatgacgggcacgactacatcggtagcgtccggtcgtatcgcgtacaccttaggtttggagggcccagctatcagtgtcgatacggcgtgttcttcgtcactggtagccgtacatctcgcgtgccagagcctgcgccgtggcgaaagctctctcgccatggcgggcggtgttaccgtgatgccgacaccggggatgctggttgatttttcgcgcatgaacagcttggcgccagatggtcgctgcaaagcgttctcggctggtgcgaacggtttcggcatggctgaaggcgcgggcatgctgctgctggaacgcttatctgacgcccgtcgtaatgggcacccagtgctggcagtgctgcgtggcaccgctgtgaatagcgatggcgctagcaacgggctgtccgctccaaatggtcgggcccaagtccgtgtgatccagcaggcgttagcggaatcaggtttgggtccggcggacattgatgccgttgaagcgcatgggactggaacccgtctgggtgatccgattgaggcccgtgcactgtttgaagcttacggccgcgaccgtgagcagccactgcatcttggcagtgtcaaaagtaacttagggcacacccaggcagccgctggcgtagcaggagtaatcaaaatggtgcttgcgatgcgcgcgggcaccttaccgcgcactctccatgcaagcgagcgtagcaaagaaatcgactggagcagcggtgctatttcgctgcttgacgaacctgagccttggcctgctggtgcccggccgcgccgtgccggggtgagcagctttggcatcagcggtaccaatgcccatgccattatcgaggaagccccacaggttgtagaaggggaacgtgttgaggctggcgatgtagttgcaccgtgggtgttatcagcctcctcagcggaaggtcttcgcgcacaggcggcgcgtttggcagcgcacctgcgcgaacaccctgggcaggacccacgtgacatcgcgtacagcctggctacaggccgcgcggcgctgccacaccgtgcggcttttgcgccggtggacgaatccgcagcgctgcgcgttctggatggcctggcgaccggcaatgcggacggcgccgccgtgggtacaagccgggctcaacagcgtgctgtcttcgtgttccctggccagggttggcagtgggcgggcatggcggtcgacctcctggacacaagtccggtgttcgcagccgcgctccgtgagtgtgcagatgccctggaaccacatctggattttgaagtcattccgtttttacgtgccgaggccgcgcggcgcgagcaggacgcggctttgagtacggaacgtgtggatgttgtgcaacctgtgatgtttgcagtgatggtttctctggcatccatgtggcgcgcgcacggcgtcgaaccggcagcggtgattgggcacagccaaggcgaaattgctgccgcatgcgttgcaggggcactgtccctggatgatgcggcgcgcgtagtggccctgagatctcgcgtgattgctactatgccaggcaacaaagggatggcgtcaatcgcggcaccagccggggaagtgcgtgcacgtattggcgatcgtgtggagattgccgctgttaatggcccacgctcggtggtagtggccggtgacagcgatgaattagatcgtctcgtcgcatcttgtactaccgaatgtattcgcgcgaaacgtctcgccgtagattatgcgagccattcatctcacgtagaaacgatccgtgacgcgctgcatgccgaattaggtgaagatttccatccactgcctggctttgtcccttttttttcgaccgtgaccggccgttggacccaaccagacgaactggacgctggttattggtatcgtaatctccgtcgcacggtgcgctttgcagatgcagtacgggccctggcagaacagggctatcgcacgtttctggaggtgagtgcgcatccaatcctgacagccgcgattgaggagattggtgatggcagtggcgccgacctgtccgcaatccatagcctgcgtcgcggcgacggcagcctggcggattttggtgaagctctgagtcgtgcattcgcggctggcgtggcagtcgattgggagtctgtacacctgggcactggtgcccgccgcgtaccgctgccgacctatccgtttcagcgcgaacgcgtgtggctgcagccgaaacctgtggctcgccggtctaccgaggttgatgaagtctctgcgctgcgctaccgtatcgagtggcgtccaactggcgccggtgaaccggcacgcttggatggtacgtggcttgtagctaaatatgcgggcacagccgatgaaacgagcactgcggcacgcgaagcgctggaatccgctggggcccgtgtgcgcgaacttgtcgtcgatgcccgttgtggccgggatgaattagcagaacgtctgcgttcggtcggcgaagtcgccggtgttctgagcttactcgccgtcgatgaagcggaaccagaggaagcgccgctggcactggcaagcttagcagatacgctgagcctggttcaggctatggtatccgcggaactggggtgcccgctgtggacagtgaccgaatcagcagtggctacgggcccgttcgaacgtgttcgtaatgccgcacacggtgcgctgtggggggtaggtcgtgttatcgcgcttgagaacccggcggtctggggcggtctcgttgacgtacctgccggtagcgtggcggagcttgcgcgccacttagccgccgtggtttcggggggcgcaggcgaagatcaactggcgttgcgtgctgatggggtttacggtcgtcgttgggtgcgcgcagcagcgcccgcaacagatgatgaatggaaaccgacggggaccgttctggtgaccggtggcactggtggtgtaggcggccaaatcgcccgctggttagcacgtcggggtgctcctcaccttctcctggttagccgtagcggcccggatgctgatggtgcgggcgaactggttgcagaacttgaagccctgggggcgcgtaccacggttgcggcatgtgacgtgacggaccgcgagtctgtgcgcgagctgttgggcggtattggcgatgacgtaccgttatcagccgtcttccatgcggcggcaaccttggatgacggcaccgtcgatactctgacaggtgaacggattgaacgcgcaagccgcgccaaagtgttaggggcgcgcaatctgcatgagctgacacgtgagctggatctgaccgcgttcgtgctgttttccagttttgcgtcggcctttggtgcaccgggtctcggcgggtatgcgccaggcaacgcttacctggatggtttggcccagcagcgtagatctgatggtctgcctgctaccgccgtggcatgggggacgtgggcgggctcaggtatggccgaaggggccgtagccgatcgctttcggcgtcacggtgttattgaaatgccgcctgaaaccgcctgtcgtgccttacagaatgctctggatcgcgcagaagtctgcccgattgttatcgatgttcgttgggaccgctttttattagcgtacaccgcgcagcgtccaacacgcctgtttgatgaaattgacgatgcccgccgggcggccccgcaggcccctgctgagccacgcgtaggtgccctggcctccctcccggctccagagcgggaagaagcgctgttcgaactggtgcgctcacatgcggcggcagtgctgggccatgcgtctgcggaacgcgtccctgctgaccaagctttcgcggagttgggtgtggattctctttcagcgctggaactgcgtaaccgcttaggcgcggcgacgggtgtgcgtcttccaaccacgacagtgttcgatcacccagatgttcgtacgttggccgcccatctcgcggcggaattgtctagtgcaaccggcgcggaacaagcggcacctgcgacgactgcgccggtcgatgaaccaattgctatcgtcggtatggcttgtcgcctgccgggtgaggtggactcaccggaacgtctttgggaattaattacctctggccgggactctgcggcggaggttccagacgatcgcggttgggtgcctgatgagctgatggctagtgacgctgcggggacccgtgcacatgggaacttcatggcaggtgccggtgacttcgatgcggcttttttcggcattagcccgcgtgaagcactggcgatggatccgcagcagcgccaggcgctggaaacgacctgggaagcgttggaaagtgcaggcattcctccggaaaccttaaggggtagtgacacgggtgtttttgtgggtatgtctcaccagggctacgcaacggggcgtccacgtccggaagacggcgtcgacggttatcttttaaccggcaacaccgcaagtgtcgcgagtgggcgtatcgcctatgtcctggggttggagggcccggcacttactgtggacacggcatgttccagcagtctggtggccttgcacaccgcgtgtgggagtttacgggacggtgattgcggcctggctgttgcgggtggcgtctcagtaatggcgggcccggaagtatttaccgagttctcgcgtcagggtgcgctgtccccggatggccgctgtaaaccgttttccgatgaagctgatggcttcgggctgggcgaaggtagcgcgttcgttgttttacaacgtctgtcggatgcgcgccgtgaaggtcgccgcgttttaggtgtggtcgcaggttcggccgtgaaccaggatggcgctagcaacggtctgtcggctccttccggtgtagctcagcagcgcgtgatccgtcgcgcctgggctcgtgcgggtattacgggagccgatgtagcggtggtggaagcgcacggaactggtactcgtctgggcgatccagttgaggcatcggccctgctggctacttacggcaaatcacgcggcagcagtggtccggtgctgctggggtcggtcaaatccaatattggtcatgcccaagccgccgctggcgtggcgggcgtgatcaaagtgctgcttggtcttgaacggggcgtggttccgcctatgctgtgccgtggggagcggtcagggctgattgactggagttctggggagatcgaactcgccgacggggtgcgcgaatggtccccggcagcagatggcgtacgtcgtgcgggcgtttcagcctttggtgtgagcggtaccaatgcccacgtgattattgcggaaccgccggaaccggagccggtgccgcagcctcgtcgtatgctgcctgccacgggtgtagttccggttgtgttgtcagctcgtacgggtgctgcgctgcgtgcgcaggctggccgtctggcggatcatttagcggcgcacccgggcattgctccggccgacgtgtcctggacgatggcgcgcgcccgccaacactttgaagaacgtgctgctgtgcttgcagccgataccgccgaagcagttcaccggttgcgtgctgtcgcagacggcgctgtggtccctggtgttgtgactggtagcgcgagtgatggtgggagcgttttcgttttccctggccagggggcccaatgggagggcatggcccgcgaactgctgcctgttccggttttcgccgaatctattgccgaatgcgatgctgttctcagtgaggtggccggttttagcgtgtcggaagttttagagccgcgcccggatgcaccgtccctggagcgggtggatgtggtgcaaccagtgctgtttgcggtgatggtgtctttggcgcgcttatggcgtgcgtgtggcgcggttccatcggctgttattggacatagccagggcgaaattgcggcggcggtagttgcaggtgcgctgtcacttgaagatggcatgcgcgtcgttgctcgtagatctcgcgccgtccgtgcagttgcggggcgtgggagtatgctgtcggtacgtggtggtcgcagcgatgtcgagaaactgctggcggatgacagctggaccgggcgacttgaagtagcggccgtaaatggtcctgacgccgtcgtcgtcgctggtgacgcgcaggcggcacgtgagttcttagaatattgtgaaggcgttggcatccgtgcccgcgcgattcctgtggattacgccagtcataccgcccatgtggaaccagtgcgcgatgaacttgtgcaggctctggcgggtatcacgccgcgccgggcggaagtcccattcttttccactctgaccggcgattttttggatggtacggaattagatgcaggctattggtatcgcaacttacgtcacccggtcgaatttcattcagcggtacaggcgctgacggatcagggttacgcaacttttattgaagtaagcccgcatcctgtgctggcatcgtcagtacaggaaaccctggatgacgctgaatctgatgctgccgtcttgggcactctggaacgcgatgcgggcgatgcggaccgttttctgactgcccttgctgatgcccatacgcgtggcgtagcagtcgattgggaggccgttctgggccgggcgggccttgttgatcttccgggttacccgttccagggcaaacgcttctggctgcagcctgatcggaccactccgcgtgacgaactggatggttggttctatcgcgtcgactggacggaggtgccgcgttctgaaccggcagcacttcggggccgctggctggtggttgtcccggaaggtcatgaggaagacggctggaccgtggaggtccgttccgctctggccgaagcgggggccgaaccggaggtgacccgtggcgtgggcggcctcgtcggcgattgcgcgggcgtagtcagcttactggcattggagggcgacggtgctgttcagaccttggtcctcgtccgtgaattggacgctgagggcattgatgccccgttatggacggtcactttcggcgccgtggatgctggttccccagtcgcccggcctgatcaggcgaaactgtggggtctcgggcaagtagcatcgttggaacgtgggccacgctggactggtctggtggacttgccgcacatgccggatccagagctgcgcggacgcctgacggcagttcttgcgggctctgaggatcaggtcgctgttcgtgcggatgccgtccgggcccgccgtctgagccctgcgcatgtcaccgcgacctccgaatacgccgtgccgggcggcacgattttggttaccggtgggaccgcagggctgggtgcggaagtcgcccgctggctggcaggccgtggcgctgaacatctggcactggtgagtcgccggggtcctgacaccgaaggggtcggcgatctgaccgccgaactgacccgcttgggtgcccgcgttagcgtgcacgcgtgcgatgtatcttcacgtgaaccagtgcgtgaactggtgcacggcctgattgaacaaggcgatgtggtacgtggcgtggtccatgctgcgggcttgccgcagcaggtggcgatcaatgacatggatgaggcggcgtttgacgaagtcgtcgcggctaaagctggtggcgcggttcatctggacgaactttgcagcgatgccgaacttttcctgttatttagcagcggtgctggcgtctgggggagcgcgcgccaaggtgcctatgcagcgggtaacgccttccttgacgccttcgctcgtcaccgccgcggtcgcggtttaccggctaccagtgttgcatggggcctgtgggccgcaggtgggatgacgggggatgaagaggccgtaagctttctgcgtgaacgtggcgtacgcgccatgccagtaccgcgtgcgctggctgctttagatcgcgtgttggcatccggggagaccgccgtcgtagttaccgatgtggactggcctgcgtttgccgaatcttacaccgccgcccgtccgcgcccattgctggaccgtatcgttaccacggcaccgagcgagcgcgctggcgagccggaaaccgaatccctgcgcgatcgcttggccgggctccctcgtgcggaacggacggcggagctcgttcgtttggtgcgcacgtcgacggcaaccgttctgggtcacgacgatccgaaagccgtgcgggccaccaccccatttaaagaattgggtttcgactctcttgctgccgtgcgcctccgtaatctgctcaatgcggcaactggcctgcgcctgccgtccacgcttgttttcgatcatccgaacgccagtgctgtcgccggtttcttggatgctgagctgtctagtgaagtgcgtggcgaagctccgtccgccctggctggtctggatgcattggagggcgcgctgccggaagtgcctgcgacggaacgtgaggagctggtccagcgtctggaacgcatgctcgcggcactgcggccggtagcccaagcagctgacgcgagtggtaccggcgcgaacccaagcggtgacgatcttggtgaagccggtgttgatgaactgttggaggctttagggcgcgaattagatggggacgggaattct

SEQ ID NO: 4 - DNA, 10710 bp, Artificial Sequence
  Synthetic construct - DEBS2
atgacagacagtgagaaagttgctgagtatctgcgccgcgccaccctggatcttcgtgcggcacgccagcgcatccgtgaactggaaagtgatccaattgctattgtcagcatggcgtgtcgcctgccagggggtgttaatacgccacagcgcttgtgggagttactgcgtgagggtggcgaaactctgtcgggctttcctactgaccgtggctgggacctggcacgtctgcaccacccggatccagacaatccggggacgtcatacgtggataaaggcggtttcttggacgacgccgcaggcttcgacgccgagttttttggtgtgagcccgcgtgaggctgcggcgatggatcctcagcaacgcttgttactggaaacctcctgggaactggtggaaaacgcaggtatcgacccgcacagcttaagaggtacggcgacgggtgtcttcctgggtgttgctaaatttggctatggtgaagataccgccgctgcggaggacgtagaagggtactcggtgaccggggtggcgcccgcggtggcgtccggccgtatttcctacactatgggcctggaggggccgtcgattagcgtcgataccgcttgctcctcctcattagttgcgttacaccttgccgttgagtctctgcgtaaaggggagagcagcatggcggttgtcggtggcgcggccgtcatggcaacacctggcgttttcgtcgatttttctcgccaacgtgcactcgcagcggatggtcggagcaaagcctttggcgcgggcgccgatggtttcggctttagcgaaggtgtaaccttggttctgctggagcgtctgtccgaagcgcggcgcaacggccatgaagtgctggctgtcgttcgtgggagcgcactgaaccaagatggcgctagcaatggcttgagcgctccttccgggccagcacagcgccgtgtaattcgccaagcgctggaaagctgcggtctcgaaccaggcgatgtggacgcggtagaagcacacggcacgggcacggctctgggtgatccgattgaggcaaacgctttgctggatacctatggccgtgatcgtgatgcagaccgcccactttggctgggctctgttaaatcaaacatcggccatacccaggcggcggcaggcgtgactggcttactgaaagtggttctggcgttacgcaacggcgagctgcccgcgaccctgcatgttgaagaaccgacacctcacgtggattggagttcgggcggcgtcgcgcttctggccgggaaccagccatggcgccgtggcgaacggacgcgccgggcccgtgtttccgcatttggcatttctggtaccaacgcacatgtgattgtggaagaagcaccggagcgtgaacatcgtgaaaccaccgctcacgacggcagacctgtcccgctggttgtcagcgcccggactacagcggctcttcgcgcacaggccgctcagatcgctgagctgttagagcgtccggacgccgatttagccggggtgggcctgggtttggcgaccacacgcgcccggcacgagcatcgcgccgccgtggtggcctccacccgggaagaggcggtgcgtgggctgcgcgaaattgctgctggggccgcgactgcggatgcagtggtcgagggggttactgaagtagacggtcgcaatgtagtctttttattccctggccagggctcccagtgggcgggtatgggcgcggaattgctgtccagttcacccgtcttcgcaggtaaaattcgcgcctgtgacgaaagcatggcgccaatgcaggattggaaagtttcagatgtgctgcgtcaggctccaggggcgccaggtctggatcgtgttgatgttgtacaaccagttctgtttgccgtaatggttagcttagccgagctgtggcgcagctatggcgtggaaccggccgcggtggtgggtcattcgcagggcgagattgcggcagcacatgtcgctggggctctcaccctcgaagatgctgccaaattagtagtgggtagatctcgtttgatgcgctctttatctggggaaggggggatggctgccgtggcattaggcgaggcagcagttcgcgagcgtctgcgtccgtggcaggatcgcctttctgttgcggcagtgaatggcccgcgtagcgttgtggtatcaggcgagccaggtgctctgcgtgcgttctcagaagattgcgcggccgagggtattcgcgtgcgtgacatcgatgtagattatgcaagccattctccgcagatcgaacgcgttcgcgaagagctgctggagacagccggcgatattgctccgcgtccggcgcgtgtgaccttccacagtaccgttgaatcgcgttcgatggatggcaccgaacttgatgcccggtattggtatcgcaatttgcgggaaacggtccgctttgcggatgcggtcacacgtctggcagaatctggttatgatgccttcattgaggttagtcctcatccggtggtggttcaggcagtggaagaggccgtggaggaagctgacggcgctgaagacgcggtggttgtcggtagtcttcaccgcgacggtggcgacctgagcgcgttccttcgttcgatggcaacggcacacgtaagcggtgtggacatccgttgggatgtagcgcttccgggggctgccccatttgctttacctacgtacccttttcaacgcaaacgctactggctgcagccagcggcacctgctgccgcgagcgatgaactggcgtaccgcgtttcatggacacctattgaaaaaccagagagcggtaatctggatggtgattggttggttgtgaccccgctgatctcaccggaatggactgagatgctgtgtgaagcaatcaacgctaacggtggccgcgccctgcgttgcgaagtcgacacaagcgcgtctcggacggagatggctcaagcggttgcgcaggctggcacgggttttcgcggcgtgctgagccttttatcctccgatgaaagtgcctgtcgcccgggcgtccctgccggtgccgttgggttgctgacgcttgtccaggccctaggcgacgcaggtgtagacgcgccggtgtggtgcctgactcaaggtgcggtgcgcaccccggcggacgatgatttagcacgtccggcgcagaccaccgcccatggttttgcccaagtggcgggcctggaattgccagggcggtgggggggtgtagttgatctgccagagtctgtagatgacgcagcactgcgtcttctggtggcagtcttgcggggtggcggtcgtgcggaggatcatctggccgtccgtgatggtcgtctccatggtcgccgcgtagtgagagctagtctcccacaatcgggtagtcgcagctggacccctcacggcacagtgttggttaccggtgcggcaagcccggtcggcgatcaactggtccgttggctggccgaccgtggcgctgaacgtctggttctggcaggcgcatgcccgggggatgatctgcttgcggccgttgaagaagctggcgcgtcagcggtcgtctgtgcgcaagacgccgccgcgctgcgtgaagctttaggcgacgaacccgtgactgctttagtgcacgctggcactctgacgaactttggctctatttccgaggtagctccggaggaatttgcagaaaccatcgcggcgaaaactgcgctcctggccgtcctggatgaggttctgggtgatcgcgccgtggaacgcgaagtatattgctcgtctgtggccggtatttggggcggtgcggggatggcagcttatgcagcgggttcggcatatttggacgcgctggctgaacaccatcgggcacgcggtcgttcatgcacctccgttgcttggacgccatgggcgttgccgggcggtgccgttgatgatggctacttaagagaacgcggtttgcgttcactgtcggctgaccgcgcgatgcgtacctgggaacgtgttctggcagcaggcccggtgtccgtcgccgtcgccgacgtagattggccggtgctgtcagaaggtttcgcggcgacccgtcctactgccctcttcgcagaactggcgggccgcgggggtcaggcagaagccgaaccggacagtggtccgacgggcgagcctgctcagcgcttggctgggttgtcgccggacgaacagcaggaaaacctgctggaattagttgccaatgcggttgccgaagttttaggccatgagtccgcggccgagatcaacgtgcgccgggcatttagcgagctgggtttagacagtttaaatgcaatggcgctccgcaaacgcctcagcgccagcaccggcctgcgcttaccggcgtcgctcgtgttcgatcatccgactgtcacggcattagcccaacaccttcgcgctcgtctctctagtgacgccgatcaggcggcggttcgcgttgtgggcgcagcggatgaaagcgagccaattgccattgtcggcatcggctgccgtttcccgggtggcatcggctctcctgaacagctgtggcgcgttcttgcagaaggggccaatctgacgaccggctttccggcagatcgcggctgggacatcggccgtctgtaccatccagacccggataatccgggcacgtcctatgtcgacaaaggtggctttctcaccgacgcagcggattttgatccgggtttttttggtattacaccgcgcgaagctttggcaatggacccgcagcagcgcttaatgcttgaaacagcatgggaggcagtcgaacgtgcgggcattgacccggatgccttaagaggcaccgacacaggcgttttcgtaggcatgaacggtcaaagttacatgcagttactggcaggtgaagcggagcgtgtagatggttaccaaggcttaggcaacagcgcattcgttttgagtggtcgtatcgcttatacgtttggttgggaaggcccggcgctgactgttgataccgcgtgttcgtcttcgttggttggtattcatctggcaatgcaagcgctccgtcgtggggaatgctctctcgccctggctggtggtgttaccgtcatgtcagacccgtataccttcgtcgacttctcgacccagcgtggtctggctagtgatggtcgctgtaaagcgttctcagcgcgggctgatggtttcgcgctttcggaaggcgtggccgccctcgtgctggaaccgcttagccgtgcgcgtgccaacgggcaccaagtgctggcggtgctgcgtggttctgccgttaaccaggatggggctagcaatggcctggccgccccaaacggtccatcgcaggaacgtgtcatccgtcaggcgctcgccgccagcggggtgcctgctgctgacgtggatgtcgtggaagcgcacggcactggtacagaattgggcgacccaatcgaggcgggtgctctgatcgcaacgtacgggcaggatcgtgaccgcccgctgcgtttggggagcgtgaaaaccaacattggtcatacccaagcagcagcgggggccgcaggggtaattaaagtagtgctggcgatgcgtcatggtatgctgccgcgtagcctgcacgctgacgaactgtctcctcatatcgattgggagtcaggcgctgtggaggtcctgcgtgaagaagtaccgtggcccgcaggcgaacgcccgcgccgcgcgggtgtttcctccttcggcgtttcaggtaccaacgcgcacgttattgtggaagaggcaccggccgaacaggaagcggctcgtaccgaacgcggcccgctgccgttcgttctgtctgggcgctccgaagctgtggtagccgcgcaggcccgcgcacttgctgagcacttacgcgacaccccagagctggggctgaccgatgctgcgtggactctggcgaccggccgtgcacgtttcgacgtgcgcgccgccgtattgggcgatgatcgcgctggtgtatgcgcggaactggatgccttagcggaaggtcgcccgtctgcggatgcggtggcaccagtcacctccgcgccacgtaaaccagtcctggttttccctggccagggggcccagtgggttggtatggcccgcgacttactggaaagttctgaggtctttgccgagtcgatgagccgctgcgcggaagcgctgtcgcctcacactgattggaaacttcttgacgttgtgcgtggtgatggtggtccagatccgcacgagcgtgtagacgtcttacagccggtcctgttttccattatggtctctctcgcggaactgtggcgtgcccacggtgtgactccggccgctgttgtaggtcactctcaaggcgaaattgcagccgcacacgtggcgggtgcgttaagcttggaagccgcagctaaagtggtggccttgagatctcaagtactgcgtgagcttgatgatcagggcgggatggtttcagtaggggcatctcgggatgaactggaaacggtgctggcacgctgggacggccgcgtagcagtggccgctgtgaatggtccagggacctcagttgtcgcaggccctactgccgaattggatgagttctttgccgaagccgaagcccgtgaaatgaaaccacgccgtatcgcagttcgttatgcgagccattccccggaagtcgcacgtattgaagatcgtctggcagccgaactcggtacaattaccgccgttcgcggcagcgtacctctgcatagcacggttgccggcgaagtaattgataccagcgcgatggacgcgtcttattggtatcgtaacttgcgccgtccggttttgtttgaacaagccgtgcgtggtctcgtcgaacaggggtttgacacatttgtcgaggtttccccacatccggttctgctgatggcagtggaggagacagcagaacatgcaggggcggaagtcacctgtgttcctacgcttcgtcgcgagcagtccggcccgcatgagtttctgcggaacctgctgcgcgcccatgtccacggcgttggcgccgatctgcgtcctgccgttgctggcggccgtccggctgaattaccaacttacccgttcgaacatcaacgtttttggctgcagccgcaccgcccagcagatgttagcgccttaggcgtacgcggggcagagcaccctctgctcctggcagccgttgacgttccgggtcacggtggtgccgttttcaccgggcgtctgtctacggacgagcagccgtggctggccgaacatgtcgtgggcggtcgtaccttggtgccgggttccgtgctggtggacctggcgctggcggccggtgaagatgtagggctgccggtattggaagaattggttttacaacgcccactggtactggcaggtgcgggcgctctcctgcgtatgtcggtcggcgctccggatgaatcaggccgccgtactattgatgtccacgcggcagaagatgtagcggacctcgcggacgcccagtggtcgcagcatgcgacaggtacattggcgcaaggcgtcgccgctggccctcgggataccgaacagtggccgcctgaagatgcggttcgcatcccgcttgatgaccattatgacggcctggcagaacagggctacgagtatggtccgtctttccaggcgttacgtgcggcctggcgcaaagatgactctgtctacgcagaagtttcaatcgcggcggacgaagagggctacgcgtttcacccggtgctgctggacgcggtagctcaaacgctgagcttaggggcactcggtgaaccgggtggcgggaaacttccatttgcatggaatacggtgacccttcacgcgagtggcgcgacttcggttcgtgtagtggcgaccccagctggtgccgatgccatggccctgcgtgtgacggatccggcaggtcatttagtggctaccgttgattctcttgtggtccgctcaactggtgagaaatgggaacaaccggaaccgcgcgggggcgaaggggagcttcatgcactggactggggccgcttggcggaaccaggctctactggtcgtgttgtagcagctgacgccagcgatttagacgccgtcttaaggtctggtgaaccggagccagatgccgttttagttcgttacgagccggagggtgatgatcctcgcgctgcggcacgccacggtgtgctgtgggctgcggcgctggttcgccgctggctggaacaggaggaactgccgggcgccacgctggtgatcgcaacgtcaggggccgtcactgtgagtgatgacgattctgttccggagccgggcgccgcggccatgtggggcgtcattcgctgcgcgcaagcggaatccccggatcgtttcgtattgttagatactgatgccgagcctggtatgctgcctgcggtgccagacaatccgcaacttgcgcttcggggtgacgacgtgtttgtgcctcgtctgagcccgctcgcgccgagtgccctgacgctgccagcaggcacccaacgccttgtcccgggcgatggcgctattgattctgtggcattcgaacctgcgccggacgttgagcagcctctgcgcgcgggtgaggtacgggttgatgtgcgtgcgaccggcgtaaattttcgtgatgttttgttagccctgggcatgtatccgcaaaaagccgatatgggtacggaagcagccggcgtagtgactgccgtaggcccagatgttgatgccttcgcccctggtgatcgggtgcttggcctgttccaaggcgcgttcgcgccaatcgctgttacagaccatcgcttgttagcacgtgttcctgatggttggtcggatgccgacgctgcggccgttcctatcgcctatacaactgcacattatgccctgcatgatctggcgggcttgcgcgccggtcagagtgtccttattcacgctgccgctggtggtgtcggtatggcagctgtagctctggcacgtcgggctggcgccgaggtgttagctaccgctggtccggctaaacacggcactctgcgtgcgctcggtctggatgatgagcatattgcgagttctagggagactggtttcgcccgtaaatttcgtgaacgcacaggcgggcgtggggttgacgttgtgctcaactccttgactggcgaactcctggatgagtcagcagacctccttgctgaagatggcgtgtttgtagagatgggcaaaaccgatctgcgtgatgccggggactttcgtgggcgctacgcgccatttgatctgggggaggcaggggatgatcgtctgggtgaaattctccgtgaagtagtgggcttacttggcgcaggcgaattggatcgcctgccggtaagtgcatgggaattggggtccgcgcctgccgcgctccagcacatgagtcgcggtcgtcacgtaggtaaacttgtactgacccagcctgcgccggtcgaccctgacggcactgtgttaatcaccggtggtacaggcaccctggggcgtttgttagcacgccatctggtgacggaacatggtgtgcggcatctgttgctggttagtcgtcgtggtgctgacgcgccgggctccgatgaactgcgcgcagaaattgaggatttgggtgcaagcgcggaaattgcggcgtgcgacacagcggatcgcgacgccctgagtgccctgctggatggtttgccccggcctctgaccggggttgtgcacgcagccggtgtgctggccgatggcttggtgacaagcatcgacgaaccggcggtggaacaggttctgcgtgccaaagtcgatgccgcgtggaacctccatgaactgaccgcaaataccggcttgagcttctttgtcctgttcagttctgcggcaagcgtgttagcaggccctgggcaaggtgtgtatgcggcggcgaatgaaagtctgaatgcattagcggctctgcgtcgcacccgcggtttgcctgccaaagcgctgggttggggcctctgggcccaagcgtccgaaatgactagcggtctgggtgaccgcattgcgcgtacaggtgttgccgcgttgccgaccgaacgtgctctggccctgttcgacagcgcattgcgtcgcgggggtgaggtggtttttccgctgtcaatcaaccgctcagcgctgcgccgcgctgaatttgtaccagaggttctgcgtggcatggtacgtgcaaaacttcgggctgctgggcaggctgaagctgcgggcccaaacgtagttgaccgcttagccggtcgtagcgaatcggatcaggtggcgggcctcgcggaactggtgcgtagccatgcagccgccgtgagtggttacggcagcgccgatcagttgccggaacgcaaagcgtttaaagacttgggcttcgatagcctggccgccgtcgagctccgcaaccgcctgggcacagccacaggcgtgcggcttccaagcacgctggtgtttgatcatccgacgccgttggcggtagcggagcatctgcgggaccggctgtctagtgcctcgccggctgttgacatcggggatcggctggatgaattggaaaaagcactggaagccctgtcagccgaggatggccatgatgatgtgggccagcgtctggagagcctgcttcgccgctggaacagtcgtcgtgcggacgcgccgtccacttctgcgatttctgaagacgctagcgatgatgaattatttagcatgctcgaccaacgctttggtggtggcgaggacctggggaattcg

SEQ ID NO: 5 - DNA, 9510 bp, Artificial Sequence
  Synthetic construct - DEBS3
atgtctggtgataatggcatgacggaagaaaaattacgtcgctacttgaaacgcaccgttaccgagctcgattccgttaccgcccgtttgcgcgaagtcgaacaccgcgcaggtgagccaattgcgatcgtaggtatggcctgtcgctttccgggcgatgtggactctccagaatctttttgggaatttgtttctggcgggggcgatgcgattgcagaagcgccagcggatcgtggctgggagcctgatccagatgcgcgtttaggcggtatgttagctgcggcgggcgattttgatgcaggttttttcggcatttcgccgcgtgaagcccttgcgatggatccacaacagcggattatgctggaaatttcatgggaagccctggaacgggccggtcacgatccggtgtcgctgcgtggctccgccacaggcgtattcactggggttggtacagtcgattatggccctaggccagatgaggcccctgatgaagtccttggttacgttggcacgggcaccgcatcatcggtcgccagtggtcgtgtagcctactgccttggccttgaggggcccgccatgaccgtggatacggcatgctcatccggcctcaccgccctgcatttggctatggaatccctgcgccgggacgaatgtggtttagcgctggcgggcggggttaccgttatgagctctcctggcgcgttcacagaatttcgctcgcaggggggtttggccgcggatggtcgttgtaaaccgttcagtaaagcggcagacggcttcgggcttgcagagggggcgggtgtcttggtgttacagcgtctgtcagctgctcgccgtgaggggcgcccggtactggccgtcctgcgcggcagtgccgtaaatcaggatggtgctagcaacggcttaacggcaccaagcggcccagcccaacaacgtgtaattcgtcgtgcactggagaacgcgggcgttcgggcgggggatgtagattacgtagaagcgcacggcacaggcactcgtttaggcgacccaatcgaagtccacgctctgctgtcgacgtatggtgctgaacgtgatcctgatgacccgttatggattggttcggttaaatccaacatcggccatacccaagctgccgctggcgtcgcgggcgttatgaaagcggtactggccttacggcacggcgagatgccacgcaccctgcatttcgacgaaccaagtcctcagattgaatgggaccttggggcagttagcgtagtttctcaggcacgttcgtggcccgcaggcgagcgtccgcgccgtgcaggcgttagttcttttggcattagcggtaccaacgcgcatgtgattgttgaggaagcccctgaagccgacgaaccggagcccgcgccggattcgggtccggtccctctggtgcttagcggtcgcgatgaacaggccatgcgggcacaggcgggtcgcttagccgatcacctggctcgggaaccacggaactctctgcgtgacacaggttttaccttggctacgcgccgcagcgcctgggaacatcgcgctgttgtggtgggcgatcgtgatgatgcgctggccggtctgcgcgccgtggcggacggtcgtattgcggatcgtactgcgactggtcaggcgcgcacgcgtcgcggtgtggctatggtgttccctggccagggtgcgcaatggcagggcatggcgcgtgacctgcttcgtgaaagccaggtttttgccgatagtattcgcgactgcgaacgtgccttggcaccgcacgtagattggagtctgactgatctgctgtctggggctcgtccgctggatcgtgttgacgtggtgcagcctgccctgtttgccgttatggtgtccttagccgcgctgtggcgttcacatggggtagagcccgcagcggtcgtaggccacagtcaaggcgaaattgcagccgcgcatgttgcgggggctctgacgttagaggatgcagctaaattggttgcagtaagatctcgtgttttagcccgtttgggcggccagggcggcatggcgtcgttcggcctgggtacggaacaggctgcggaacggattggccgtttcgcgggcgccctgtcaatcgcgagcgttaacggcccacgttctgtcgtggtagcaggggaatctggccctctggatgaactgatcgccgagtgcgaagcggaaggtattaccgcacgccgtatcccagtggattatgcgagtcactcccctcaggttgaatctctgcgcgaagaacttctgactgagctggcgggcattagccctgtgagcgcagatgtcgccctgtattccacgacgaccggccagccgatcgacacggcaaccatggataccgcgtattggtatgcaaatctccgtgagcaggtgcgcttccaagacgctacgcgtcaactggccgaagccggttttgatgctttcgtggaagtatctccacatccggtcctgactgtgggtattgaggccactcttgatagtgcattgccagcagatgcaggcgcatgcgttgttggtacgttacgccgtgatcgtggcggcctggcagactttcataccgcattaggcgaagcctatgcccagggcgtggaggtggattggtcacctgcttttgcggatgcccgcccagtggaattaccagtgtatccgtttcagcgtcagcgttactggctgcagattccgacaggtgggcgggctcgtgacgaagatgatgattggcgttatcaggtcgtttggcgtgaagcggaatgggagtctgcgtccctcgccggtcgcgtgctgctggtaaccggcccgggtgtaccatctgagctgtccgatgccatccggtcagggctggagcagtcgggggcaacggttttgacatgcgacgtcgaaagccgttccacgatcggcacggcgttggaagctgctgatactgatgcgctgagcaccgtagtatcgctgttaagccgtgatggcgaggctgtcgatccgagtctcgatgctctggctttggtgcaggccctaggtgctgctggcgtcgaagcaccgctgtgggtcctgacccgtaatgctgtccaggttgctgatggtgagctggtggatcctgcccaagccatggtgggcgggctgggccgcgtcgttggtatcgaacaaccgggtcgctggggcggcttggtcgacctggttgacgccgacgcagcttccatccgtagtcttgctgcggtgctcgcggatccgcgtggtgaggaacaagttgccatccgtgcagatggtatcaaagtggcgcgcctggttccagcaccggctcgcgcggcacgtacccggtggagccctcgcggtacggtgctggtaaccggtgggacaggtggcatcggggcacacgttgcacgttggctggcgcgcagtggtgcggaacatctggttcttctgggccgccgtggcgccgacgcgccaggcgccagcgaactccgcgaagaactgaccgcgctgggcaccggcgtgactattgcagcttgcgacgttgcggatcgcgctcggttagaagcagtattggcagcggaacgcgcggaaggtcgtaccgtctctgccgttatgcatgccgcgggtgtgtcaaccagcaccccgctggatgatttaaccgaagccgagttcacggagatcgctgacgtgaaagtccggggcaccgttaacctggacgagctgtgtccggacctggatgcgttcgttctcttttcgtcaaatgctggcgtttgggggtctccgggtctggcgtcctacgccgctgcgaacgcgtttcttgatggtttcgcacgccgccgcagatctgaaggcgcacccgtcacgagtatcgcatgggggttgtgggccggtcagaacatggccggtgatgaaggcggtgagtatctgcgtagccagggcctgcgcgcaatggacccagatcgtgcggtggaagaactgcatatcacgctggatcacggtcagacctccgtctcagtggtcgatatggaccgtcgccgttttgtggagttgttcacggctgcccgtcaccgccctttgtttgatgaaatcgcgggtgcacgggcggaagctcgccagagtgaagaggggcctgcgctggcgcagcgtctggccgcactgtctaccgccgagcgccgcgagcacctggcacacctgatccgtgccgaagtggcagcggttcttggtcacggcgacgatgcggcgattgaccgcgatcgtgcattccgcgatctggggtttgactccatgactgccgttgacctgcgcaaccgtctcgcagccgtcacgggggtacgtgaggctgccacagttgtatttgaccatccaacgatcacgcgcttggcggatcattatttggagcgtctctctagtgccgctgaagcggaacaggccccagccctggttcgcgaagttccaaaagatgccgatgacccaattgcgatcgtgggcatggcgtgccgttttccgggcggggttcacaacccgggcgagctgtgggagttcatcgtaggccgtggcgatgccgtgacggaaatgcctacggaccgggggtgggatttagatgcactgttcgatccagatccgcagcgtcacggaacctcctattctcgccatggtgccttcttagatggtgccgcagattttgacgcggctttttttggcatttcacctcgtgaggcgttggcaatggatccacagcagcgtcaggtgctggaaaccacctgggagttattcgaaaacgccggtatcgatccgcacagcttaagaggttcagatacgggtgtgtttttgggcgctgcctatcaaggttacggtcaggatgcggtggtcccagaggatagcgaggggtatctgctgacggggaactcgtctgccgtcgtgtcgggccgcgtcgcgtacgtgcttggcttagaaggtccggcggtaaccgtggacacggcatgctcttccagcctggtggccttacactccgcttgtggctccctgcgcgacggtgattgcgggttagcggtcgccggtggcgtctccgtgatggcagggcctgaagtcttcactgagttcagccgccagggtggcctggcggtggatggccgttgtaaagcgttctctgccgaggccgatggtttcggttttgccgagggcgtggcagtggtactgcttcagcgtctgagcgatgcacgccgggcgggccgccaagtcctgggtgtggtggccggttccgccattaatcaggacggtgctagcaacggtctggcggcgccaagcggtgtggcccaacaacgtgtgattcgtaaagcatgggctcgcgccggtattactggtgcagacgtcgcggtggttgaagcgcatgggactgggacccgccttggtgatccagttgaagcgtctgcgctgctggctacctacgggaaatcccgtggcagctcaggtccggtactgctgggctctgtgaaaagcaatatcgggcacgcccaggcggcggctggcgttgctggggttatcaaagtagtgttaggtctgaaccggggcctcgttccgccgatgctgtgccgaggcgaacgttccccgctgatcgaatggagcagtggtggcgtggagctcgccgaagctgtcagcccgtggccgccggcagcagacggcgttcggagggcaggcgtgtctgcgttcggcgtgagcggtaccaacgctcatgtcattattgccgagccgccagagcctgagccgctgccagaaccggggccggtcggtgtactcgccgctgcgaatagtgttccggttctccttagcgcccgcaccgaaaccgcgctggctgcacaagcacgcctgctggaaagcgccgttgacgattcggttccactgacggcgttggcttccgctctggctaccggccgcgcccaccttccgcgtcgcgcggctctgttagcaggtgaccacgaacaactgcggggtcagctgcgtgcagtggccgaaggtgttgcagcaccgggcgcgacgacaggtacggcgtccgcaggtggtgtggtctttgtctttcctggccagggcgcccaatgggaaggtatggctcgggggttgctgagtgtgccagttttcgccgaatcgatcgccgaatgtgacgccgttctgagtgaagttgcaggtttttcagcttcagaagttctggaacagcgccctgatgcaccgtcactcgaacgcgtggacgttgtgcaaccagtgctgttctctgttatggttagtttagcccgtttatggggcgcgtgtggggtgagcccgtcagccgttatcggtcatagtcagggcgaaattgcggcggccgtcgtggccggcgttctgagtttggaggatggcgttcgtgtggtcgcgttgcgcgcgaaagccctccgtgcactcgcgggcaaaggcggcatggtctccttggcggcccctggcgaacgcgcccgtgcgttgattgccccgtgggaagaccgcatcagtgtggcggccgtaaacagtcctagcagcgttgtagttagcggtgatcctgaagcacttgcggagctggtagcgcgttgcgaagatgaaggcgttcgcgccaaaacgctcccagtggactatgcgagccattctcggcacgtggaagagattcgcgaaacaatcttggcggacctggatggtatctctgcacgtcgtgcggcgatcccgctgtacagcacccttcatggcgagcgtcgcgacggggcggatatggggccgcggtattggtatgacaatttgcgcagtcaggtccggttcgatgaagcggtttcagcggccgttgccgatggtcatgccacctttgtggaaatgagcccgcacccggttctgaccgccgccgtgcaggagatcgcggccgatgccgtggcgatcggttctctgcaccgtgatacggctgaggagcatttaattgccgaattagcacgcgctcatgtacacggcgtcgctgtcgattggcgcaacgtgtttccagcggcaccacccgtggctctgccgaactacccgttcgagccgcagcgctactggctgcagccggaggtgtctgaccagctggcggactcccggtatcgcgtggattggcgtccactggcgacaacgccggtggatctggaaggcggttttctggtgcacggctcagcgcctgaatcactcacctccgcagtagagaaagcaggcgggcgcgtagttccagtggcgagcgccgatcgggaagcctctgctgccttgcgtgaggttccgggcgaagtggctggcgtgctgtcggtgcacactggcgccgctactcacctggcgctgcaccagtccctaggcgaagcaggtgtgcgcgccccgttatggttagtgaccagccgtgccgtggcgctcggtgaatccgaaccagttgatccggaacaagcgatggtgtggggcctgggccgcgttatggggctggaaaccccggagcgttggggcggcttagtagatttgccggccgaacctgcccctggggatggcgaagccttcgtcgcatgtcttggcgcggatggtcacgaagatcaagtcgcgattcgtgatcacgcgcgttatgggcgccgtctggtgagggctccgctgggtactcgggagagcagctgggaaccggcgggtactgcattggtgaccggtggcacgggggcgttgggcggtcacgtggctcgccatctggcccgctgcggcgtcgaggacctggtgctggtcagccgccgtggtgtagacgccccgggcgcggcggagctggaagctgagcttgtggcgctgggcgccaaaacgacaattacggcatgcgatgtagcggatcgtgaacagctgtcgaaacttttagaagaattacgtgggcagggtcgtccggtgcgcacagtcgttcatactgcgggcgtcccggaatcacgcccgctgcatgagattggggaattggaatctgtgtgcgccgccaaagttaccggcgcccgcctgcttgacgaactgtgtcctgatgcggagacttttgtgttgtttagctccggggcgggcgtgtggggctccgcaaatttaggcgcatattcggcggcaaacgcctacctcgatgctctggctcatcgtcggcgcgcagaaggccgcgcagccaccagtgttgcctggggggcgtgggccggcgaaggcatggcaacgggcgacttagaagggctgacgcgccgtggcttgcgcccgatggcgccggagcgggcaattcgggcgctccaccaagctctggacaatggtgacacttgcgtctctattgccgacgtcgactgggaggcgttcgctgtggggtttaccgccgcacgtccgcgtccactgctcgatgaactggtcacgccggcggtgggtgcagtaccagctgttcaggcggctccagcccgtgaaatgactagccaagaactgctggagttcacacactcgcatgttgccgcaatcttgggtcatagcagtccggatgccgtcggccaagaccagccgtttacggaactgggtttcgatagtctgactgccgttggcctgcggaaccagctacagcaagcaactggtctggcgttaccggcaactttagtcttcgaacatccgacagtacgccgcttggccgatcacatcgggcaacaactgtctagtggcaccccggcgcgggaagcgtctagtgctctgcgcgacgggtatcgtcaggctggcgtgtcggggcgcgtacgcagttacttggatctcctggcaggtctttccgacttccgcgagcatttcgatggttctgatggctttagccttgacctggtggatatggccgatggtccaggcgaagtgacggtcatctgctgtgcggggaccgcggccatttcaggcccgcacgagtttactcgtctcgctggcgcattgcgcggcattgctcctgtgcgtgcagttccgcaaccaggctatgaggaaggcgaaccactgccgagcagcatggccgccgtggccgcggtgcaggctgatgcagtcattcgcacccaaggtgacaaacctttcgtggtagcaggccacagcgccggcgcactcatggcctatgcactcgcgaccgagctgttggatcgtggtcacccgccacgcggggttgtcctgattgatgtatacccgccgggccaccaagacgctatgaacgcctggctcgaagaattgaccgccacgttatttgaccgtgagaccgtacgcatggacgacactcgcttgaccgcgctgggtgcgtacgaccgcctgacaggtcagtggcgtccgcgcgaaacgggtctgccgacacttctggtgtctgcgggcgaacctatgggcccatggccggatgattcgtggaaaccgacctggccgtttgagcatgacacagtggctgtcccaggcgaccatttcacgatggttcaggaacacgccgatgcgattgctcgtcatatcgacgcctggcttggaggcgggaattcg

SEQ ID NO: 6 - DNA, 4265 bp, Artificial Sequence
  Synthetic construct - EpoA
atggccgaccgcccgatcgaacgtgcagcggaggatccaattgcgattgtaggcgcgggctgccgcctgccgggcggcgtgattgacctctcgggcttctggacgctgttagaaggctcccgcgacaccgtcggtcaagtgccagcggagcggtgggatgctgcggcgtggttcgatccggatctggatgcacctggcaaaacaccagtgacccgcgccagctttttaagcgatgtcgcctgcttcgatgcctcttttttcgggatcagtccgcgcgaagcccttcgcatggatccggcccaccggctgctgctggaagtgtgctgggaagcattggaaaacgcagctattgccccgtcggccctggttggcacggaaactggcgtctttattggcatcggtccaagcgaatatgaagcggcactgcctagggctactgccagcgcagaaattgatgctcacggcggcctgggcacgatgccttcagttggtgcaggtcgtatttcatacgtcctgggccttcgtggtccgtgtgtggcggtggacaccgcatatagttctagcttagtcgcagtacacctggcgtgtcagtcgttacgttccggcgaatgctcgaccgcgcttgcaggtggggtcagccttatgctgtccccgagcactttagtctggttgagcaagacacgtgcgttggcaaccgacggtcgctgcaaagccttcagcgcggaggccgatgggtttggtcgtggcgaaggttgcgcagtggtcgtgctgaagcgtttgtccggcgcacgtgcggatggggaccgcatcctcgcagttatccgcggctcggccatcaaccatgatggtgccagctccggtctcactgttccgaacggttcttcacaggaaattgtactgaaacgcgccttagccgatgctggttgcgccgcatcttccgtggggtacgtcgaagctcatgggacgggtactaccttaggcgatccgattgaaattcaggcgctcaatgccgtctacggcctgggtcgggatgtcgcgacccctttgctgatcgggtcggtcaagactaacctcggccatccagagtatgcctccgggatcactggtctgctgaaggttgtgttgtccttgcagcacggtcaaattccggcgcacctccatgctcaggcgttaaatccgcgcattagctggggcgatctgcgtctgaccgttacccgtgctcggaccccgtggcctgactggaacacgcctcgccgcgcgggcgtctcctcgtttggcatgagtggtaccaatgcccacgttgttctggaggaagccccagcagcaacgtgcaccccgccagccccagaacgtccagccgaattgttagtgctgtctgcgcgtaccgctgccgctctggacgcacatgcggcccgtttgcgcgaccatttagaaacatacccgtcacaatgtttaggtgacgttgccttctcgctggcgactacccgtagtgcgatggaacatcgcctggcggtggccgctacgtcctcggagggtctgcgtgcggccttagacgccgcagctcagggtcagaccccgccgggtgttgtccgtggtatcgcagactcgtctcgcggcaaactggcttttctgtttactggccagggtgcccagacgctcggcatgggccggggcctgtacgatgtttggcctgcttttcgcgaagcgtttgatttgtgtgtgcgcctgtttaaccaagaactggatcgtccgctgcgtgaagtaatgtgggcagaaccagcatcagtagatgccgcacttttagaccagacagcttttacacagccagcgctttttacgtttgagtatgctctggctgcactgtggagatcttggggcgtagaaccagaactggtggccggtcactcgattggcgaactggtggcggcgtgcgttgcgggtgtgttcagtttggaggacgccgtgttcctggtcgcggcacgcggtcgtctcatgcaggcgctgcctgctggtggtgcaatggtgtctattgcggcgccagaagcggacgtcgcggcggcggtcgcgcctcatgccgcatcagtaagtatcgcggctgttaatggcccagaccaagtggtaatcgcgggcgcagggcagccggtgcatgcgatcgccgctgcaatggcggcgcgcggtgcccggaccaaagcgcttcacgtgagccacgcgttccacagtccactgatggcaccgatgttagaagcgtttggccgcgttgctgaatccgtaagttatcgtcgtccgagcatcgtactcgttagtaatctgagcggcaaagcagggacagatgaagtatccagccctggctattgggtgcgtcatgctcgggaggttgtgcgtttcgcagatggcgtgaaagcgctccatgccgcaggtgcaggcacgtttgttgaagtgggtccgaagtctactcttttgggtttagttccggcgtgtttgccagacgctcgtccggcgcttctggcaagttctcgtgccgggcgcgatgaaccagccactgttctggaagctctggggggtctgtgggccgttggtggtcttgtatcgtgggcaggtctgtttccgagtggcggtcgccgcgtgcctctgccgacgtatccgtggcaacgtgagcgttactggctgcagaccaaggcggatgacgcagcgcgtggtgatcggcgagcaccgggtgcgggccatgacgaagtcgaaaaaggcggggcggtcagaggtggggatcgccgcagcgcccgtttggatcatccaccgccagagagcggacgccgtgaaaaggtggaggcagcgggcgaccgtccgtttcgtttggagattgatgagcctggcgtgctggaccggctcgttctgcgtgttacggagcgtcgcgcaccgggcttaggtgaggtggaaattgctgtagatgcggcaggtctgagttttaacgacgtgcagctggctctgggtatggttccggatgatctgccgggtaaaccgaatccgccgctgctgttaggcggggaatgtgccggccgcattgtggcggttggggaaggcgtaaatggtctggttgtaggtcagccggtgattgcactgagcgctggtgctttcgcaacccatgtcaccacgtcagccgccctggtgctgccacgccctcaggcgctgtccgcgaccgaggccgcagctatgccagtggcatatctcaccgcgtggtatgctctggatggcattgcccgccttcaacctggcgagcgcgtgctgatccatgcggccacgggtggcgttggcctggcggcagtacagtgggcccagcacgtcggggccgaagttcacgctactgcgggtacgccagagaaacgcgcttaccttgaaagcctcggggttcgttacgtttcagattctcgcagcgaccgctttgtagcagatgtgcgcgcctggaccggcggcgaaggcgttgatgtcgttctgaactctctgtcaggtgaactgattgataagtcattcaacttactgcggtctcatggtcgttttgtcgaactcggcaaacgcgattgttatgctgataatcagctcggccttcgccctttcctgcgtaacctttcattttctttggttgatctgcgcggcatgatgctggaacgcccggcacgtgtgcgtgccttgtttgaggagctgctgggtttaattgccgctggtgtgttcaccccgccgccgatcgccacgcttcctattgctcgcgtggcggacgccttccgttcgatggcgcaagcacagcatttaggcaaactcgtactgaccctaggggatccggaggtccaaatccgtattccgacacacgcgggggccggtccgtctaccggcgaccgggacctgctggatcgtcttgcgagtgctgcaccggcggctcgtgcggcggccttagaagcttttttgcgcacccaggtgtcgcaagtgctgcgcacacctgaaattaaagtaggggctgaagctttgttcacacggctgggtatggattccctgatggcagtggaacttcgtaatcgtattgaggcgagcttgaagctgaaattatctacaaccttccttagcacgagcccgaacatcgccctgctgacccaaaacttgttggatgcactctctagtgcattaagtttggaacgtgttgccgcggagaacctgcgcgcgggcgtccaatccgactttgtgtcgtcaggggccgatcaggattgggaaatcattgctctggg

SEQ ID NO: 7 - DNA, 4238 bp, Artificial Sequence
  Synthetic construct - EpoB
atgaccattaatcagttactgaatgaattagaacaccagggcgttaaattagccgcagatggggagcgcctccagattcaggcaccaaaaaatgccctgaacccgaacttgttagcacgcatttctgaacataaatccacgatcttaaccatgctgcgccagcgccttccggcggagtctattgtcccagccccagcggaacggcatgtgccgttccctctgaccgacatccagggctcttattggctcggtcgtactggtgcctttacggttccgtcgggcatccatgcctaccgtgaatatgattgcacggatctggacgtggcccggcttagtcgtgcattccgtaaagtcgttgcacggcatgatatgctgagggctcataccctgccggatatgatgcaggtgatcgaacctaaagtagatgcggacatcgaaatcattgacctgcgtggcctcgatagatctacacgcgaagctcggttggtgtccctgcgtgacgccatgtctcaccggatttatgatacggaacgcccgccgctgtatcacgttgtggccgttcgcttagatgaacaacagacccgcctggtgctgagcattgatctgattaacgttgacctgggcagtctgagcattatctttaaagattggttgagcttttacgaagatcctgaaacctcgctgccagtgctggaactgagttaccgcgactacgtcctggcgttggaatcgcgtaaaaaatcggaagcccaccagcgctcaatggactactggaaacgccgtgttgctgaactcccaccaccgccaatgctgccaatgaaagcggatccgtcgacgttgcgtgaaattcgcttccgtcataccgaacagtggctcccgtctgatagttggtcgcgtttaaaacaacgtgtaggcgaacggggtctgaccccaacgggtgtaatcctcgcagctttctctgaggtgatcggccgctggtccgctagcccgcgctttaccctcaacatcactttattcaaccgtctccctgtgcatccccgggtcaatgatattactggtgattttacaagcatggtgctgttggacattgatacgacgcgcgacaaatcattcgaacagcgtgctaaacgcattcaggaacagctgtgggaagccatggaccactgcgatgtttctgggattgaagtacagcgcgaagcggcacgtgtgctgggcattcaacgcggcgcactgttcccggtagtactgacctcagccctcaatcaacaggtggttggggttacgtctctgcaacgtctgggcaccccggtttacacgagcactcagactccgcagctcctgctcgatcatcagctgtacgaacatgacggtgacctggtcctggcgtgggatattgtggatggcgtgtttccgccggatctgctggatgatatgttagaagcctatgtcgcctttttacgtcgcctgacggaggaaccgtggtctgaacaaatgcgctgcagcctgccgcccgctcagttagaggcacgtgcatccgccaatgaaactaactcactgctgtctgaacatactctgcatggtctgtttgccgctcgggtggagcagttaccgatgcagcttgcagtggttagcgctcgtaaaaccctgacgtatgaggaattgtctcgccgctcccggcggctgggtgcccgcctgcgggaacaaggcgcacgcccgaataccttggtcgccgtcgttatggagaaaggttgggaacaagtggttgcggtccttgccgtgctggaaagcggcgcggcttatgttccgattgatgccgacctgccagcagaacgtattcattacctgcttgatcacggtgaggttaaattggtgctgactcaaccgtggctggatggcaaacttagctggccgccagggatccagcgtctgctggtaagcgacgccggcgtcgaaggggacggcgaccaactgccgatgatgccgattcagaccccatcggacttagcatacgtcatctacaccagtggttcgactggtttgccgaaaggtgttatgattgatcaccgtggcgctgtcaatacaattttggacatcaacgagcgctttgagattggtcctggggatcgcgtgctggccctgtcctcactttcttttgatctgtcggtttatgacgttttcggtatcctcgcggcgggcgggaccattgtggtgccagatgcgtcaaaactgcgtgacccagcccactgggctgcacttattgaacgcgaaaaagtcactgtgtggaatagtgtaccggcactgatgcgtatgctggtcgaacactctgaagggcgccctgattcgctggcacgtagcctgcgcctcagcctgctgagtggtgattggatccctgtggggctcccgggtgaacttcaggctatccgtccgggcgtcagtgttattagcctggggggtgccacagaggctagcatctggagcattggctatcctgttcgcaacgtggacccgtcctgggcatcaattccgtatggccgcccgcttcgcaatcagacgttccacgtgcttgacgaggcgctggagccacggccggtatgggtgccaggccaactgtatatcggtggcgttggcctggcactgggctattggcgtgacgaggaaaaaactcgtaactcttttctcgtccatccggaaacgggggaacgcctgtataaaaccggggatctcgggcgctaccttccggatggcaatattgaatttatgggccgcgaggataaccaaattaaactgcggggctatcgcgtggaattgggtgaaatcgaagaaaccctgaaaagccatcctaacgtgcgcgatgcggtcatcgtgccggttggcaatgatgccgcaaataaattactgcttgcgtatgtggtaccggagggcacccgccgccgtgcggcggaacaggacgcatcacttaagacggaacgtgttgatgcgcgtgcgcatgcagccaaagcggacggcctgagcgacggtgagcgcgtccagttcaaactggcacgtcatggcctgcgtcgcgatctggatggcaaaccggtggtagacctgacgggtctggtaccgcgcgaagcggggctggatgtatatgctcgtcgtcgttcggtccgcactttcttagaggcaccgatcccgttcgtagaatttggtcgctttctgtcttgtcttagctcagtggagcctgatggcgcagctctccctaaattccgttacccttcggcgggtagtacctacccggtccaaacatacgcctatgcgaaaagcggccgtatcgagggtgtagacgaaggcttctattactatcatccattcgagcatcgtctgctgaaagttagtgatcacggtattgaacgtggcgcgcacgtgccgcagaacttcgacgtgtttgacgaagctgcctttggtttactctttgttggccgtatcgatgcgatcgagagcctgtacgggtcattgagccgcgaattttgtctgttggaagctggttatatggcccaactgctcatggagcaagcgccgtcgtgcaacattggggtctgccctgtagggcagtttgattttgaacaggtacgcccagttcttgatttacgccattccgatgtttacgtacacggtatgctgggcggtcgcgtggatcctcgccagtttcaggtctgtaccctcggccaggattccagcccacgtcgtgctacgacgcgcggtgccccaccgggtcgcgaccaacattttgctgacatccttcgggactttcttcgcactaaactgccggaatatatggtaccgaccgttttcgtcgagttggacgcgttaccgctcacttctaacggcaaagtggatcgcaaagcgctgcgggaacgcaaagatacatcatccccgcggcactccggtcacaccgccccgcgtgatgctctggaagagattctggtcgccgttgttcgtgaagttctcggtctggaagtggtcgggctgcaacagtcttttgtagacctgggtgctacttccatccatatcgttcgtatgcgcagcctgttgcagaaacgcctggaccgcgaaattgccattacagaacttttccagtacccaaatctgggttcgttagccagcggtctttctagtgatagtaaagatttagaacaacgtccgaatatgcaggaccgcgtcgaggctcgccgcaaaggccggcgtcgttcagggaattc

SEQ ID NO: 8 - DNA, 5504 bp, Artificial Sequence
  Synthetic construct - EpoC
atggaagaacaagaatccagtgcaattgccgtgattggcatgtcaggtcggtttccaggggcccgcgatctggatgagttctggcgcaatctgcgcgacggcaccgaggccgtccagcgctttagtgagcaggaactggcggcgtccggcgttgatccggctcttgtgttagatccgaactatgtgcgggcaggtagcgttctggaagatgtcgatcgttttgatgccgctttctttggtatctccccgcgtgaagcggaactgatggacccgcagcaccggatctttatggaatgcgcgtgggaagcactcgaaaacgccggctatgacccgactgcatacgagggtagcatcggcgtgtatgcgggggccaacatgagcagttatttaacctcaaatttacatgaacatccggcgatgatgcgttggccgggttggttccagacgctgatcgggaacgataaagattacttggcaacgcacgtgtcttaccgtctgaacttgcgtggcccgagtatctccgtccaaactgcgtgctcaacctcgcttgtcgctgttcatttagcttgtatgagcctcctggaccgggaatgcgacatggcactggcagggggcatcaccgtccgcatcccgcaccgtgctggttatgtgtacgcggaaggcggtattttctcaccagatggtcattgtcgcgcattcgatgccaaggctaatggaaccattatgggcaatggctgcggcgttgtgctgctgaagccgttagatcgtgcgctgtccgacggcgaccctgttcgcgccgtaattctgggcagcgcgaccaataatgacggtgcgcgcaagattgggtttaccgcgccttcagaggtgggtcaggcgcaagcgatcatggaggcgctggcgctggcgggtgttgaggcgcgtagtatccagtacattgaaacacatggcaccggcacactgctcggggacgcaatcgaaacggcagccttacgccgcgttttcgatcgcgacgcgtcgactcgccgctcttgcgccatcggctctgtaaaaaccggcatcggtcatctggaatctgccgctggcattgctggtttgattaagaccgtactggcgcttgaacatcgtcagctgccgccttccctcaacttcgaaagcccaaatccgtcgatcgattttgcctcatctccattctacgtgaacacgtcactgaaagactggaacactggtagcacaccacgccgcgccggggtatcaagctttggtattggcggtaccaacgcccatgtggtgctggaagaagctccggcagccaaattgccagctgccgctccagcccgtagcgccgaactgttcgttgtgtcagctaaatcagcagcagcgttggatgcagcggcggctcgtctgcgcgatcacctgcaagctcaccagggtttgtccctgggcgatgtcgcctttagtctggctactacacgctcccctatggaacatcgtttggcaatggcggccccgagtcgggaagcactgcgcgagggtttggatgcggcagcccgtggacaaacgcctcctggcgcggtccgcggtcgttgttcccctggcaacgtcccgaaagtcgtcttcgtctttcctggccagggtagccagtgggtgggtatgggtcgtcagttgttggccgaagaaccagtttttcatgccgcgctttccgcctgcgatcgtgcaatccaagctgaagctggttggagtttattggccgaactggctgccgatgaaggttctagccagatcgaacgtattgacgtggtgcaaccagttctgttcgccttagcagtagcattcgctgccctgtggagatcttggggcgttggtcctgacgtcgtaatcggccatagcatgggtgaggttgcagctgctcacgttgcaggcgctctgtccctcgaagacgcggtggcaatcatttgtcgccgcagccgtctgctgcggcgtatttcgggtcagggcgagatggctgttactgaactgagcctcgcggaagcagaagccgcgctgcgtggctatgaagaccgtgtctcggtcgcggtgagcaatagcccgcgctctaccgtgctgtcgggtgaacctgccgcaatcggggaggttttgtccagcttaaacgcgaagggggtattttgtcgtcgcgtgaaagtagatgtggctagccactcaccacaggtagatccattacgtgaagacctgctggcagcgctgggtggcttacgcccgcgtgcggcggccgtgccgatgcggtcaactgtcactggtgcgatggtggcaggcccggaactgggcgctaactactggatgaataatctgcgccaaccagttcgcttcgcggaagttgttcaagcgcagctccagggcggtcacggtctgtttgtcgaaatgtctccgcatccgattctgaccacctcggtcgaggaaatgcgtcgggcggcgcaacgcgcaggcgcggcagttggtagcttacgtcgcggccaggatgaacggcccgccatgctggaggcgttaggggcgctgtgggcccaaggttatccagttccgtgggggcgcctttttccggcaggcgggcgccgcgttccgttgccgacttacccttggcagcgtgaacgctactggctgcaggcgccagccaaaagcgccgcaggcgatcgtcgcggtgttcgtgcaggcggccatccgctcttgggcgaaatgcaaaccttatcaacgcaaacgtctacccgcctgtgggaaaccaccttggatttgaagcgcctgccatggctgggtgatcatcgcgtccagggcgcagtggtgtttccgggtgcggcctatctggagatggctatttcctcgggtgctgaagccctgggcgatggtccgctacagattacggacgttgttctggcggaggcacttgcgttcgcgggcgacgctgcggtactggttcaggtggtgacgacagaacagccgagcgggcgtttacagtttcagattgcaagccgtgcgccgggtgcgggccacgcgagttttcgtgttcacgcacgcggcgctttattacgtgtagagcgcactgaggtgcctgcggggcttacgctttctgcggtccgggctcgcttacaggcgtctatgccagccgcagcgacgtatgcggaacttacggagatggggctccagtacggtccggcatttcagggcattgccgaactgtggcgcggcgagggggaggcattgggccgcgtacgtttgccggacgcagcggggagcgccgcggaatatcggctccatccagcgctgctggatgcttgctttcaagtggtgggttctttatttgctggcggtggggaggctaccccgtgggtgccggtggaagttggttctctgcgtctgctgcaacgtccttctggggaattatggtgtcacgcacgcgtagttaaccatggccgtcagactccggaccgtcagggtgccgatttctgggtagtcgacagcagtggcgcggtggtagcggaagtgagtggcctggtggcacagcgtttgcctggcggtgtccgccgtcgcgaagaagatgactggtttcttgagcttgagtgggagccagccgccgtcgggacggctaaggttaatgcgggtcggtggttgctcctgggtggcggtggcgggctgggtgctgcacttcgttcgatgctggaagctggcggtcacgcggttgtgcatgcggccgagagcaatacatctgcggcgggcgtccgggccctgctagcgaaggcgttcgatgggcaagctcctacagccgtggttcacctgggctcgctggatggcggtggcgaacttgacccgggcctgggggcacagggggcgctggatgctcctcgtagtgcagatgtgtcgccagatgcactggatccggccctggtgcgcggctgcgatagtgtactgtggacggtccaagcgctggcaggtatgggctttcgcgacgccccgcgtctgtggttgctgactcggggtgcccaggcggtaggcgccggtgacgtgagtgtgacccaggcaccgctgctcggtttgggtcgtgttattgccatggaacacgctgacctccgttgtgctcgcgtggatctggatcctacccgtccggatggtgaactgggtgcgctgcttgcggaactccttgctgatgatgccgaagccgaagttgccttacgtggcggcgagcgctgtgtggctcgcattgttcgccgtcagccggaaacccgccctcgcggtcgcatcgaaagctgcgtcccaactgatgtgacaatccgtgcagatagcacctatctggtcaccggtggtcttggcggcttaggcttgtcggttgcgggttggctcgcggagcgcggtgcaggtcatctggtcctggtaggccgtagcggtgccgcctctgtggagcagagggctgcggtggcagctttggaagcacgcggggcgcgtgtgaccgtggctaaagctgacgtagctgatcgcgcccagttagaacgcattttacgggaagtgacgacctcgggcatgccgttacgcggcgtcgttcatgccgccgggattctggatgacgggttactgatgcagcaaacgcccgcacgctttcgtaaagtgatggcgccaaaagttcaaggcgcactccatcttcatgcactcacgcgcgaggcaccgctgagtttttttgtcctctacgcctccggcgtcggcctgttgggttctccgggtcaggggaattatgcggcggccaataccttcttggatgcgctggcgcaccaccgtcgtgctcaggggttaccagccttaagtgtggattggggcctgttcgcggaggttggtatggctgccgcacaagaagaccggggtgcacgtctggtatcgcgcggcatgcgctcgctgaccccggacgaaggtctgagcgctctggctcgtcttcttgaatcgggccgtgttcaagtgggggtcatgccagtgaaccctcgcctgtgggtggagttgtatccggcggctgcgagttcacgcatgctgtctcgtctcgtaacagcacatcgtgcatccgctggcggccctgcgggcgacggcgatcttctgcgtcgtctggctgcggcggagccttccgcacgttcgggtttactggaaccgctccttcgcgcccagatttcacaggtgctgcggctcccagagggcaaaattgaggtagatgcgccactgacatccctgggcatgaacagtctcatgggtctggagctgcggaaccgtattgaagccatgttgggcattacggttccggcgactcttctttggacgtatccgaccgtagcagcactttcggggcacttagcgcgtgaagcatctagtgctgcgccggtggagagtccgcatacaaccgcagatagcgcagttgaaatcgaagaaatgtcccaggatgacctgactcaactgattgccgcgaaatttaaagccctgacggggaattc

SEQ ID NO: 9 - DNA, 21779 bp, Artificial Sequence
  Synthetic construct - EpoD
atgaccacacgtggcccgaccgctcaacaaaatccactgaaacaagcagcaattatcattcagcgccttgaagaacgccttgcaggtctggcacaagcggaactggagcgtactgagccaattgcgatcgtaggcatcgggtgtcgttttccgggtggcgcagacgcgccggaagcattctgggaactgctcgatgctgagcgcgatgccgttcagcctttggaccgtcgctgggcactggtcggggtagcgccagtggaagcggtccctcattgggcgggtttattgaccgaaccgattgactgtttcgatgcggccttttttggtatttcgccgcgtgaagcacgtagcttggatccgcagcaccgtctgctccttgaagtagcatgggaggggctggaagacgccggcatcccaccgcgtagcattgacggctctcgcactggtgtctttgtgggtgcgttcaccgccgattatgcccgtactgttgctcgcctgcctcgtgaagaacgcgacgcgtacagcgcgacaggtaacatgttatccatcgcggctgggcgtttgtcgtatacgttgggcctccagggcccgtgtttgaccgttgataccgcatgctcgtcctctcttgttgctattcatctggcgtgccgctccttgcgggctggcgaaagtgacctggcccttgcaggcggcgtctcgacgttgttatcacctgatatgatggaagcggcggcacgcacccaggccctgtccccggatggccgctgtcgtactttcgatgcgtcggcgaatggctttgtacgtggtgagggttgtggtctggtcgttctcaaacgtttatccgacgcacagcgtgacggcgaccgtatttgggcgttaatccgcggctcagcgattaatcatgacggtcgctccacgggcctgacagcgccgaacgtccttgcgcaggaaacggtgctgcgcgaagcactgcgtagtgcgcacgttgaagcaggggccgtggattacgtggagactcatggcaccggcaccagcctgggcgatccgatcgaagtggaggccctgagagccaccgtcggcccagcccggagcgacggtactcgctgtgtgttaggcgcggtaaaaacgaacattggacacctggaggcagccgctggtgtagctgggctgattaaagctgcgctgtccttaacgcacgaacgcatcccgcgtaacctgaactttcgtaccttgaacccgcgtatccgtcttgaaggctctgcattggcgctcgcaaccgagccagttccttggccgcgcacagatcgcccacgctttgccggtgtgagttcatttggcatgtcgggtaccaatgctcacgtggtactggaggaggctccggccgtggaactgtggcctgcggcgccggaacgttccgctgaactgctggtgctgagcggcaaatctgaaggtgccctggatgctcaagctgcccgtctgcgtgaacatttggacatgcacccggaactggggttaggcgatgtggctttctccctggcaacgacccgctctgcgatgacacatcggttggctgttgcggtaacctcccgcgaaggtctgttggccgccttgtcagcggttgcacagggccaaacgccagcaggcgctgcacggtgcattgcgagctctagtcgcggtaagctggctctgctgtttactggccagggcgcccaaactccgggtatgggtcgcggcttatgtgccgcctggcccgcttttcgtgaagcctttgatcgctgtgtaacgttatttgaccgtgagctggatcggccactgcgggaggttatgtgggcggaagctgggtccgccgaatcattactgttagaccagaccgcgttcacgcagcccgcgctgttcgctgtcgaatatgccctgacggcgctctggagatcttggggtgtcgaaccagaactgctggttggacactctattggcgaactggtcgcggcgtgcgtggctggcgttttctctcttgaagacggtgtgcgcctcgtggcggctcggggtcgcctcatgcaggggctgagcgctggcggcgccatggtgtcactgggtgctccagaggcagaagtagcagcagccgtcgcaccacatgcggcatgggtttcaatcgccgccgtaaatggcccagagcaggtagttattgcaggcgtcgaacaagcggtgcaggcaatcgccgcagggtttgcggcgcgcggcgtgcgcactaaacgcctccacgtctctcatgcctttcactccccgctgatggaaccaatgctggaagagttcggtcgcgtggcagcgtctgttacctaccgtcgtcctagcgtctcgctcgtttccaacctgagtggtaaagtggttactgacgagctgagcgccccaggctactgggttcgtcatgtgcgcgaagccgtccgttttgctgatggtgtgaaagccctgcacgaagcgggcgcgggcacctttctggaagtcggtccgaaaccaaccctgctgggcctgctcccggcgtgcctgccagaagcagaacctacgttattagcgagcttgcgggcgggccgtgaagaagcagcgggtgttctggaggcccttgggcgtttgtgggcggcaggcggttccgtttcttggcctggcgtttttccaaccgctggtcgccgtgtgccgcttccgacctatccgtggcaacgtcagcgctattggctgcaggcaccggcggaagggctgggtgcgactgcggcagatgcgttagcccagtggttttatcgcgtggattggccggaaatgccacggagtagcgttgattctcgccgtgcgcgttcgggcggctggcttgtcctggcggaccgtggcggggtgggcgaagcagccgcagcggcactgagtagtcaaggctgctcatgtgcggtgttacatgctccggcggaggcgtccgccgtcgccgaacaggtgacccaggccctgggcgggcgcaatgattggcagggcgttctgtacttgtggggtctggatgcagtcgtcgaggcgggcgcatccgcagaggaggtgggtaaagtgacacacctggcgaccgctccggtgttagcactgattcaggccgtcgggactggcccgcgcagccctcgcctgtggattgtaacgcgtggggcttgtacggtcggtggcgagccggatgctgccccgtgtcaggctgcactgtgggggatgggtcgtgtggcagccttggaacatccgggctcctggggtggtctggttgatctggatccggaagaatctccaacggaagtagaagcgctggtggctgaactgctgtctccggatgccgaagatcagctcgcatttcgtcaaggccgtcgtcgtgccgcccgcttggtcgccgcgccaccggagggcaacgcagcgccggtgtcgttaagcgcggaaggttcatatttggttaccggtggtctgggcgctctgggtctgctggtggctcgctggctggtggaacgtggtgcgggtcatctggttttaatctctcggcacgggcttcctgatcgcgaagaatggggccgtgatcaaccacctgaggtacgggcccgtatcgcagcgattgaggccctcgaagctcaaggcgcacgcgtaacggttgccgccgtggatgttgcagacgctgaggggatggccgctcttttagcagccgtggagccgccactgcgcggcgtggtccatgccgctggcctgctggacgacggtctgttagcgcaccaggatgcaggtcgcctggctcgggtgttacgtccgaaagttgaaggtgcttgggttctgcataccctgacccgcgagcagcctcttgatctgtttgttctgtttagctccgcaagtggtgttttcggttccatcggccagggctcttatgcggcagggaacgcatttttggatgctctggcggatctgcgtcgtacacaaggcttggcggccttaagcattgcatggggcctgtgggcggaagggggtatgggctcacaagcccagcgccgcgagcatgaggcatccggtatctgggcgatgccgacgtctcgcgccctggcggcaatggaatggctcctgggcacccgcgccacgcagcgtgtggtaattcagatggactgggctcacgcgggtgcagcaccacgggatgcttccagagggcgtttctgggatcgtctcgtaaccgtcaccaaagcagctagtagcagtgctgtgcccgcagttgaacgctggcgtaatgcaagcgtggtcgaaacccgttcggctctgtatgagctggtgcgcggcgtggtagcaggtgtgatgggttttactgatcaaggcacattagatgtccggcgcggctttgcagagcagggtttagatagcctcatggcggttgaaattcgtaaacgtctgcaaggcgagctgggtatgccgttgtctgccacattggcgttcgatcatccgaccgtagaacgtttggtggaatatttacttagccaagcgtctagtttacaggaccgtacggatgtccgctccgtgcgtctgccagcaacggaagatccaattgcgattgttggggcggcatgccgttttccgggtggcgtcgaggacctggaatcttactggcagttgctgacggaaggtgtggtcgtttctaccgaagtaccggcagaccgttggaacggggcggacggccgtggccctggcagcggtgaagcaccgcgccagacctatgtcccgcgcggtggctttctccgcgaagtcgaaacttttgacgcggccttctttcacatctctccgcgtgaagctatgtccctggacccgcagcaacgcctgttgttagaagtctcgtgggaagcaatcgaacgtgccggccaggatccgagtgccctgcgtgaatctcctactggagtgtttgtgggtgcgggcccgaatgagtatgcagaacgtgttcaggacttagctgatgaagcagcagggctctactccggaactggcaatatgctgagcgtcgcggcagggcgtctttccttttttttggggttacacggcccgaccctggcagtcgacactgcctgtagtagcagtctggtcgcgttgcaccttggctgtcaatcactgcgccgtggcgagtgtgaccaagctttggtggggggcgttaatatgttactgtccccaaaaacgtttgccctgctttcacgcatgcatgcgctgtcacctggtggacgttgtaagactttctcggctgacgctgacgggtatgcccgcgccgaaggctgtgccgttgtcgtcctgaagcggctgtctgatgcacaacgggatcgcgatccgatcctggcagtaatccgcggtacagcaattaaccatgatggtccgagcagtggcttgacagtgccctcgggtccggcacaggaagccttacttcgtcaagcgctggcacatgcgggcgtagtgcctgctgatgtggacttcgttgaatgccatggcacggggaccgctttaggtgatccgattgaggttcgcgcactgtccgacgtatacggtcaggcccgcccggcggatcgtccgctcattctgggcgcggccaaagcgaatctcgggcacatggaaccggcagcaggcttagctgggctgttgaaggccgtgctggcgctgggccaggaacaaattccggctcagcctgaactgggtgaactgaacccgctgctgccatgggaagccctgcccgtggcggtggcacgtgcggcggtcccgtggccgcgcacggatcgtccgcgttttgcaggtgtgagttcgttcggtatgagcggtaccaacgcgcatgttgtccttgaagaagcgcccgccgtagaattatggcctgcggcgccggaacgctcggcggaattgctggttctttctggcaagagcgagggcgcactggacgcgcaggccgcacgcctgcgtgaacacttagacatgcatccggaactgggcctgggcgatgtagccttctccctggcaacaacgcgcagcgcgatgaaccatcgtctggccgtggctgtgacgagtcgcgaaggcttattagcagctctgagcgccgttgcgcagggtcaaaccccgccgggtgcggctcgttgcattgcgagctcaagccgtggtaagctggcctttctgttcactggccagggggcgcagaccccgggtatgggccgtgggctgtgcgcagcatggcctgctttccgcgaagcatttgatcgctgcgtcgccttgtttgatcgcgaactggaccgcccgctgtgtgaggttatgtgggccgagccgggttcggcggaatctctgttactcgatcaaacagcatttactcagccagccctgtttacggtagaatatgccctgaccgcgctgtggagatcttggggcgtcgaacctgaactggtggcggggcactcagcgggcgaactggtggcagcctgtgtagctggtgtgttctctctggaagatggtgtccgccttgtcgcggcgcgtggccgcctgatgcagggtctgtccgctggtggcgcgatggttagtctgggtgctccggaggcggaagttgctgccgccgtagctccacatgcggcttgggtatcaatcgcagcggtaaatggtccggaacaagttgtcattgcaggcgtggaacaggcagttcaggcaatcgcggcgggtttcgcagcacgcggggtccgtacgaaacggctgcacgttagtcatgctagccactctcctctgatggaacccatgctggaggagttcggccgcgttgctgcttctgttacctaccgccgcccatctgtgtcgctggttagcaacctgagtggtaaggttgtcaccgatgaactttctgccccgggttactgggtccgtcacgtgcgtgaagcggtccgctttgcggatggtgtgaaagcgttacatgaggctggggctggtacgtttctggaggtagggcctaaaccgaccctcctgggccttctgccagcatgcctgccggaagcggagccgacgctgttggcgagccttcgcgcaggacgtgaggaagcagcaggcgtcttagaggccctgggtcgtctttgggccgccggaggaagcgtctcgtggcccggtgtgtttccgaccgctggccgccgtgtcccccttccaacctatccttggcaacgccagcgctactggctgcagatcgaacctgatagtcgtcgccacgcggcggcggatccgacacaaggttggttttaccgcgtggattggccggaaattcctcggagtctccagaagtcagaggaggcttcacgtgggagctggctggttctggccgataaaggcggtgtaggcgaagcggttgcggcggctctgtctacacgcgggttaccgtgcgttgtcctgcatgccccagccgaaacgtcagcgactgcggagctggtgacggaggctgcgggcggtcgcagcgattggcaggttgtgctgtatttatgggggcttgatgcggtcgtcggtgctgaagcaagtatcgatgaaattggggatgctactcgtcgcgcgaccgccccggttctgggtctcgcgcgcttcctgtcgaccgttagttgtagccctcggctgtgggttgttacacgcggcgcgtgcatcgttggtgatgagcccgccatcgcgccgtgccaggcagcactgtgggggatgggtcgcgttgccgcacttgaacaccctggcgcatgggggggcctcgtggatttggatccgcgagcgtctccgcctcaggcttcaccaatcgacggtgaaatgttagttactgaactgcttagtcaagaaaccgaagatcagcttgcgttccgccacggccgccgccatgccgctcgcctcgtagccgcgccaccgcgtggggaggcagcgcctgcgtccttgagcgccgaagcaagttacctggtgaccggtggcctgggtggccttggcttgattgtcgcgcagtggctggtggaattaggcgcccgtcatctcgtgctgacttcacgtcgcgggttgccggatcgtcaggcttggcgcgaacagcaaccaccagaaatccgcgctcgtatcgccgctgtggaagcactggaagctcgtggtgcccgcgttactgtagcagccgtggatgtcgcagatgtcgaacctatgaccgccctcgtgtcttcagtggaaccgccgctgcgcggtgttgtccacgctgcgggcgtctcggttatgcgtccgctggctgaaacagatgagacgctgttagagtctgtgctgcgtcctaaggtggcggggagctggttattgcatcgcctgctgcacggccgtccgttggacctgtttgtgctgttctcaagcggtgccgccgtttggggcagtcacagccagggtgcgtatgctgctgcaaacgcgtttttggatggtctggcacatctgcgtcgctctcagtcactgcccgccttaagcgtagcctggggtctctgggccgaaggtggcatggcggatgctgaggcgcatgcccgcttatcagatattggtgtgcttccaatgtcgacctctgctgccttatccgcattgcagcgtctggtggaaaccggcgcagcacaacgtactgtcacgcggatggactgggcccgctttgcgccagtgtacacggcacgtggccgtcgtaacctgctgagcgctttagtggctggtcgcgatattattgcgcctagccctccggcagctgctacacgtaattggcggggcctcagtgtcgcggaggcccgcatggcgctgcatgaagtggtccatggtgcagttgcgcgtgttttaggctttttggacccttctgcactggatccgggcatgggctttaacgaacaaggtttggactctctgatggccgtggagattcggaaccttttgcaggcagaactggacgtgcgtctctcaacgacattagcgttcgatcaccctactgtgcagcgcctggtggagcatctgctcgtggatgtgtctagtttagaagaccgctctgatacgcagcatgtgcgctcgctggcctccgacgagccaattgcaatcgtgggcgctgcctgccgttttccgggcggcgtggaagacctggaaagctactggcagttactggcagaaggggtagtggtttcggccgaagtccctgcggaccgctgggacgcggccgattggtacgatccggatccggaaatcccagggcggacctatgttaccaaaggcgcgtttttgcgcgatcttcaacgcctggatgccacgttcttccgcattagcccgcgtgaggctatgagcctcgacccgcaacagcgcctgcttttggaagtgtcctgggaagcgctggagagcgccggcatcgccccggacaccttgcgtgacagtccgactggtgtcttcgtaggtgcgggcccaaacgagtattacacgcagcggttacggggttttactgacggcgccgctggtctctatggtggcactggcaacatgctctctgtggcagcagggcgcctttcgttttttttaggcttgcacgggccgacattggcgatggacacggcgtgttcgagctcgttagtagcgcttcatctggcttgtcagtcgctgcgtctgggtgaatgcgatcaggcattggttggcggcgtgaatgtccttttagcgccggaaacctttgtcctgctgtcacgtatgcgtgccttgtcaccagatggtcgttgtaaaacattcagcgccgatgcagatggctacgcacgtggtgaaggctgtgcagtggtggttctgaaacgcctccgtgatgcgcagagggccggtgactcgattctggcgctgatccgcggtagtgctgtaaaccatgatggtccgtcctcgggtctgaccgtacctaatggtccggcgcaacaggcactcttgcgtcaggctctgagccaagcaggtgtgtcccctgtggatgttgatttcgtcgaatgccatggcactggtacggctctgggtgacccgattgaagttcaagctctgagtgaagtatacggtccgggtcgtagcgaggatcgccctctcgtattaggcgccgttaaagccaatgttgcccacttggaagcagcgagcggcctggcatcattactgaaagcggtgcttgcgttacgccacgaacagattccagcgcagccagagctcggggagctgaacccgcacttgccgtggaatactctcccagtggcggttccacgtaaagccgtgccatggggccgtggcgctcgtccgcgccgtgcgggcgtgagtgcctttggtttatcgggtaccaacgttcatgtggtgttagaagaagcgccggaggtagagttagtgccagctgcacctgcgcgtccggtcgaactggtggtgttgagtgcgaaaagcgctgcggctctggacgctgcggcagaacgcctgagcgcccatctgagcgcacatccggagctgtcgttgggcgatgtagcctttagtctggctactactcggagcccgatggaacaccgcctggcgattgcgaccaccagtcgcgaagccttacgtggtgccctggatgccgcagcccagcgccagaccccgcaaggcgcagtgcgcggcaaagccgtatccagccgaggcaaattagccttcctgtttactggccagggggcccagatgccgggtatggggcgcggcctgtacgaagcttggcctgccttccgcgaggcgtttgaccgctgcgtagcgctgtttgaccgtgaactggatcagccgttgcgtgaagttatgtgggcggcgccaggtttggcgcaagctgcgcgtttagatcaaactgcctacgcgcagccagccctgtttgcacttgaatacgcactggctgcgctgtggagatcttggggtgtcgaacctcacgttcttctgggtcattcgattggtgaactcgttgcggcgtgcgtggctggtgtatttagcttagaggacgctgtgcgccttgtggccgcacgcgggcgtctgatgcaggcgttgcccgctggtggcgccatggtggctatcgcagcgagtgaagcggaggtagcggcgagtgtcgctccacacgcagccaccgtgagtatcgcagccgttaatggtccggatgccgtggtgatcgcaggcgcggaagttcaggttctggcgttgggtgctaccttcgcggcgcgcgggatccgtacgaaacgtctggccgtatctcacgcctttcattcaccgttgatggatcctatgctggaggattttcaacgtgtcgcggcgaccattgcctatcgtgcaccggatcgtccggtagtgtcgaacgttactggtcacgtggcaggtccggagatcgcgacacctgaatattgggttcgtcatgtgcgtagcgcggttcgctttggcgatggtgctaaagcccttcacgctgcgggcgcagcgacgtttgtagaaattgggccgaaacctgtattgctgggtctgctgccagcttgcctgggcgaagcggacgcggtacttgtgccaagtttacgcgctgatcgctcagagtgcgaagtggtgctggcagcattaggcacatggtacgcctggggtggcgcactggactggaaaggcgtatttccggatggggcccgccgcgtcgcgctgccgatgtatccgtggcagcgcgaacgtcattggctgcagctgacacctcgttctgcggctccagcgggcattgcgggtcgttggccgctggcgggcgtgggtctttgcatgccaggcgcggtgctccatcacgtgctgtcaatagggccacgtcatcagccattcctgggtgaccatctggtgtttggtaaagtcgtggtgccgggtgcattccatgtggcggtgattctgagtatcgcagcggaacgctggcctgaacgtgcaatcgaactgacaggcgttgaatttctgaaagccatcgctatggagccggatcaggaagtggaactgcatgctgtcctgacgccggaggcggcaggggacgggtatctgttcgaactggcaaccttggcggcaccagaaactgagcgtcgttggacgacccatgctcgcggccgtgtgcaaccgacagatggggcaccgggggccttaccgcgtttagaggtgttagaagatcgcgccattcaacctttggactttgcgggcttcctggatcgcctctcagcagtccgcattggctggggcccgttgtggcggtggcttcaggatggtcgtgtgggtgacgaagctagcctggcgacgctggtgccgacctatccaaacgcccatgacgtggcgccgctgcacccgattttgttagataacggtttcgcggtgtcactgttggcgacccggtcggaaccagaagacgatggtactccaccgctgccgtttgctgttgaacgcgtgcgctggtggcgtgcacctgttggtcgtgtccgctgtgggggcgttccgcgctcacaggcattcggcgtctcttcgttcgtacttgtggacgaaactggtgaagttgtcgctgaggtggaaggctttgtgtgtcgccgcgctcctcgcgaagtctttctgcgtcaggaatcaggggcgtctaccgctgccctgtatcgcctggattggcctgaggcgccgctgccggatgcgccagctgagcggatggaagaatcatgggtggtcgttgcagctccggggtccgaaatggcagccgcactggctacgcgcctcaaccgctgcgtgctcgccgaacctaaaggtctggaggcggcactggcaggcgttagccctgccggtgtgatttgcctgtgggaacctggcgcgcatgaagaagcacctgcggcagcgcagcgtgtcgccacggaaggtctgtccgtcgtgcaggcacttcgtgatcgcgccgtacgcctgtggtgggtaaccacaggggctgtggcggtggaagctggtgagcgcgtgcaggttgcaactgccccggtctgggggctcggccgcaccgtgatgcaagagcgtccggaactgtcttgtacgttagtggatctggaaccggaagtcgatgcagcccgtagcgccgacgttctgctccgggaattaggccgtgcggatgatgaaacgcaggtcgtcttccgttccggcgaacgccgtgtcgctcgcctggtcaaagcgaccacaccggaaggtcttcttgtgccggacgccgaatcttatcgtctcgaagcaggtcagaaaggcaccctggatcagctgcggttggcaccagcccaacggcgggctccgggcccaggcgaagtggaaatcaaagtaaccgcgagcggcctgaatttccgtactgttctcgctgttctggggatgtatcctggtgacgcaggcccgatgggcggggattgtgccggcatcgtcaccgccgtgggccagggtgtccatcacctgagcgtaggtgacgcggtgatgacgttaggcacattacaccgttttgtgacggtggatgctcggctggtggttcgtcaaccggctggcttgactcctgcccaagctgcgaccgtcccggttgcatttctgactgcgtggctggcactgcatgatctgggtaacctccgtcgtggtgaacgcgtgctgattcatgccgccgcaggtggcgtcggcatggcggccgtccaaatcgcacggtggatcggcgccgaagtttttgccaccgcctctccgtccaaatgggccgctgttcaggcgatgggtgtgccgcgtacgcacattgccagttctaggactctggagttcgctgaaaccttccgccaagttacgggtggccgtggtgtcgatgttgtacttaatgctttggcgggcgagtttgtggatgcatctctgagcctcttgaccactggtggtcgttttctggagatgggcaaaacggacattcgcgatcgcgccgccgtcgctgccgcccacccaggggtgcgctaccgcgtatttgacatcttagagctggcgccagatcggacccgtgagatcctggaacgcgtcgttgaaggtttcgcagcgggccatctccgcgctttgccggtgcatgcgtttgccattaccaaagccgaagcggcgttccgtttcatggcgcaggctcggcaccaaggcaaagtcgtcctgctccctgcgccaagcgcggccccactggccccaacggggacggttctgctgaccggtggcttaggggcgctcgggttgcatgtggcacgctggttggctcagcagggcgctccacacatggtcctgacgggtcgccgtggtttggataccccaggggcggccaaagcggttgccgaaattgaggctcttggtgcgcgtgtcactattgccgcatctgatgtggctgatcgcaacgctctggaggccgttttacaagcaatcccagcggaatggccgctccaaggcgtgattcatgcggctggcgcacttgatgatggtgtcctggatgaacagaccacggaccgtttcagccgtgtattagccccgaaagtaactggcgcctggaacctgcacgagttaactgcggggaatgatctggctttttttgtgttgtttagctcaatgagtggtctgctcggttcagctggtcagtcgaactatgccgccgccaacacctttctggatgcgctggcggctcaccgccgcgcagaagggctggcagctcagtcgctagcttggggtccgtggagtgatggcggtatggcggcgggtctttcagccgcccttcaagcacgtcttgcacgccacggtatgggcgccctttccccggcgcagggcaccgccctgctcggtcaagcgctggcacgcccggaaactcagctgggtgctatgtcccttgatgtgagagcggcctcccaggcgtccggcgccgcagttcctccagtttggcgtgccctggtgcgtgcagaggctcgccatgccgccgcaggcgcccagggtgccttagcggcacgcctcggggctttgcctgaagcccgccgcgcggacgaagtgcggaaagttgttcaagccgaaattgcacgcgtgctcagctggggggccgccagcgccgtacccgttgatcgcccgctgtctgatctgggtttagattcacttacagctgtcgaattacgcaatgttctcggccagcgtgttggtgcaaccctgccagcgacccttgcgtttgatcacccaactgtagacgcactgacccgttggctcctggacaaagtttctagtgtggcagaaccttccgtctccccagccaaaagctctccgcaggttgcgctcgatgaaccaattgcggttattgggatcggttgccgctttccgggtggtgttaccgatccggaaagcttctggcgcctgctggaagaaggtagcgatgcggtcgttgaggtcccgcatgagcgctgggacatcgatgccttctatgacccagatccggatgtgcgtgggaaaatgactacgcggtttggcgggtttttgtcggatattgaccgcttcgaacctgcatttttcggcatttccccgcgcgaagctacgaccatggatccgcagcagcgcctgctgctggaaacgagctgggaagcgtttgagcgtgccggcattctcccagagcgtcttatgggttcggatacgggtgtctttgtgggtcttttctatcaggaatatgcggccctggctggtggtattgaagcatttgacggttatctggggaccggcaccacggcatccgtcgcgagcggccgtatctcgtatgttctgggcttaaaaggtccgtcgttgactgttgatacggcgtgtagttcgtcgctggtggccgtacatctggcatgccaagcgctccggcggggcgaatgcagtgtcgccttagcaggtggggtggctttgatgttgaccccagctacatttgttgagttcagtcgtctgcgcggcttggcgccggacggtcgttgcaaatcattcagcgctgccgcagatggtgttggttggtccgaaggctgtgcgatgctgctcctcaaaccgctgcgcgatgcccaacgcgacggcgatccgatcttagcggtgatccgcgggaccgccgtaaaccaagatggccgtagcaacggtttaacggcgcctaatggctccagccagcaggaagtcatccgtcgcgcattagagcaggcaggcttagcgccagccgacgtgagttatgtcgagtgtcatggtacgggaaccaccctcggtgatccgatcgaagtgcaggcgttgggtgccgtattagcacagggccgcccgagtgatcgtccgctggtaattggtagcgtcaaaagcaacattgggcatacccaggctgcggcaggcgtggcgggtgtgatcaaagtagctctggctctcgaacggggcctgattccgcgctccttgcattttgatgccccgaacccgcacattccgtggtccgaactggccgtgcaggtcgcggccaaacctgtggagtggacacgcaacggcgcaccgcgtcgcgcaggcgtatcgagttttggtgtcagcggtaccaatgcccacgtcgtgttagaagaagccccagcagcggccttcgcaccggccgccgcccggtcagccgagttgtttgtgctgtcggcgaaatctgcggcggccctggatgcccaggcggcacgtctttctgcgcatgtcgttgcacatcctgaattgggcttaggcgatctggcctttagtctggcgactacccgctcaccaatgacgtatcgcttagcagtagctgcgaccagccgcgaggcgttgtctgcggccctggataccgccgcacaagggcaagcacctccagctgctgcgcgtggtcacgcgagtactggctcggcgccgaaagttgtatttgtgttccctggccaagggagccaatggttaggtatggggcagaaactgctgtccgaagaacctgtattccgtgacgctctgtcagcttgcgatcgtgcgattcaagcggaggctgggtggtccttactggcagaactggcagcagatgaaaccacctcacagttgggtcgcattgatgtggtgcagcctgcgctttttgccatcgaagtggcactgagcgcgctgtggagatcttggggtgtggaaccggatgccgtggttggtcattctatgggcgaagtggcggcggcccacgtagcaggcgcccttagtctggaagacgcggtagcgatcatttgcaggcgcagccttttgctgcgccgtattagcgggcaaggcgaaatggcagtggtcgaactgtccctggctgaagcggaagccgcgctgctgggttatgaagaccgtcttagcgttgctgtttcgaactcgccacgctcaaccgtgcttgcgggcgagcccgctgcgctggccgaagttttagcgatcctggcagcaaaaggcgtcttctgtcgtcgcgtgaaagtagatgtagctagccacagccctcagattgatccattacgtgacgaactgttagcggcgctgggcgaactggaaccacgtcaggccacggtctctatgcggtccacagtaacaagcacgattgtggcgggcccggaactggtggcgagctattgggcagataatgtgcgccaacccgtccgcttcgcggaagcggtgcaatctctcatggaaggcgggcatgggctgtttgtcgaaatgtcgccgcaccctattttgaccaccagcgtcgaagaaatccgtcgggctactaaacgtgaaggcgttgcggtagggtcgctgcgtcgcggccaagatgaacggttgtctatgctggaagcgctgggcgcactgtgggtgcatgggcaggctgtaggttgggaacgcctgtttagtgcgggcggcgcagggctgcgccgtgttccattaccaacgtacccgtggcagcgcgaacgctattggctgcaggcaccaacaggtggtgcggcgagcggcagccgttttgcgcatgctgggtcgcatccgctgctgggtgaaatgcagacccttagtacccagcgtagcacccgcgtctgggagaccacactcgatctgaaacggctgccgtggctgggtgatcaccgtgtacagggggctgtagttttcccgggtgctgcctatctggaaatggcgctgagttccggtgcggaggctctgggggatggtcctctccaggttagtgatgtggtcctggcggaagccctcgctttcgcggacgacaccccggtggctgtgcaggtaatggctacggaagagcgtccgggccgtttacaatttcatgtggcgtcacgtgttccgggccacggccgcgctgcttttcgctctcacgcacgcggcgtccttcgtcagaccgagcgcgcagaggtgccagcacgcctggacctggccgcgctgcgcgcacgccttcaggccagtgccccagctgccgccacctacgcagccctggccgaaatgggtttagaatacggccctgcctttcaaggtttagttgaactgtggcggggtgagggcgaggcgctgggtcgcgtacgtcttccggaggccgctggcagcccggccgcttgtcgtctgcatccagcactgctggacgcctgctttcacgtttcttctgcgtttgctgatcgcggggaggccacaccttgggtgccggtagaaatcggttctctgcgctggtttcagcggccgtcaggcgagctttggtgtcatgcccgtagcgtatcccatggcaaacctacgcctgatcgccgctcaacagacttttgggtggttgactcgactggcgcgatcgtggccgagatttccgggttggttgcacagcgtttggcaggcggcgttcgtcgccgggaagaggacgattggttcatggaacctgcttgggagccgacagctgtgcctggctctgaagttactgcgggccgttggctgttgattgggtcgggtggtgggctgggtgcagccctgtatagtgctctgacggaagcaggccacagcgtggtccacgccaccggccacggcaccagcgcggcgggcttgcaggctctgctgacggcatcgtttgacggtcaggctccgactagcgtcgttcacctaggttcactggatgaacgcggtgttcttgatgccgacgcaccgtttgatgctgacgccctggaagagtcgctggtgcgcggctgcgattccgtactgtggaccgtccaggcggttgcaggtgcggggttccgtgatccgccacgtctttggttagtgacgcgtggggcgcaggccattggcgccggtgatgtctctgtggcgcaagccccactgctgggtctcggccgtgtgatcgcattggagcacgccgaactgcgttgcgcccgcatcgacctggatccggcgcgtcgcgacggcgaagtcgatgagcttcttgcagagctgttggctgacgatgccgaggaagaagttgcgtttcgcggcggcgaacgccgggtggcccgcctcgtgcgtcgtttaccggagacagattgtcgtgaaaaaatcgaaccagctgaaggccgcccttttcgtctggagattgacggttcaggtgtcctggacgatttggttctgcgtgccacggaacgtcgtcctccgggcccgggggaagttgaaatcgccgtggaagccgccggcctgaattttttggatgtgatgcgtgcaatgggcatttaccctggtccgggcgacggtccagtagcactgggcgccgaatgtagtggtcgtattgttgctatgggcgaaggcgtcgaaagccttcggatcggccaagatgtcgtcgcggtcgcacctttctcttttggtactcatgtgacaatcgatgcccgtatggtcgccccgcgtccagcggcgctgaccgcagcgcaggcggctgccctgcctgtggccttcatgacggcatggtatggtttagtgcatctgggtcgtctgcgtgcgggcgaacgtgttttgattcatagcgccactggcggcactggccttgcggcagtacaaatcgcgcgccatctcggggcggagatatttgcgacagcaggcaccccggaaaaacgcgcatggctccgcgaacaaggtattgcgcatgtaatggattctaggtcattagactttgctgaacaggtcctggccgcgaccaaaggtgaaggcgtggatgtggttttaaactccctgtccggtgcggcaatcgatgcttcattagccactttagttccagacggccgtttcatcgaactgggtaaaacggacatttacgccgatcgcagcctggggctggcccacttccgcaaaagcctttcctacagcgcagtcgatctggctggtttagcggttcggcgcccggagcgtgttgcggctctgcttgctgaggtggtagacctgctggcacgtggtgcgcttcagccgttgccggtagaaatctttcctttgagccgcgcggccgacgcgtttcgcaaaatggcacaagctcaacatctgggtaaattggtcctggcattagaggatccggatgtgcgcattcgcgtcccaggcgagagtggggtagcaattcgcgcagacggcacgtacctggtgaccggtgggttaggtgggctgggtcttagcgtagcgggttggttggccgaacagggcgcgggccatctggttctggttggtcgctcgggtgccgtcagtgcagaacaacagaccgccgtagcggccctggaagcacacggggctcgcgttacagttgctcgtgccgacgttgcggatcgtgcacagatcgaacgtatccttcgcgaagtgaccgcgtcgggcatgccgcttcgtggtgtggtgcatgcagctggcatcctggatgacggcctgctgatgcagcagaccccggcacgttttcgcgcagttatggctccgaaagtcagaggtgcccttcacttgcatgcgctgacccgtgaagcgccactgagttttttcgtgttatatgcgagtggtgcgggccttttgggtagtccagggcagggcaactatgccgccgcgaacactttcttagatgcattagcacaccaccggcgcgcgcagggcctcccagccttaagtattgactggggtctgttcgctgatgtggggttggccgctggacagcagaatcgcggcgcgcgcctggtaacacgtgggactcgcagtctgaccccggatgaaggtctgtgggcacttgaacgtctcctggatggcgatcggactcaggcaggggtgatgccgttcgacgtgcgccaatgggtggagttctatccggccgctgcttcttcacgtcgcctgagtcgcttggttaccgcccgccgtgtggcgagcggccgtctggcaggcgatcgcgatctcttagagcgcctcgctacggcagaagcgggtgcccgtgcaggtatgctccaggaagttgttcgcgcacaagtgtctcaagtgcttcgtctcccggaagggaaacttgacgttgacgctccgctgacctccctgggcatggatagcttgatgggtcttgaattgcgtaaccgcattgaagctgttttggggatcaccatgcctgcgaccctgctgtggacttatcctaccgtcgcggccctgagtgcgcacctggcgtcccatgtgtctagtactggtgatggcgagtctgcccgtccaccggacacaggtaatgttgcccctatgacccatgaagtggcgtcattagatgaagatgggttgtttgctctgatcgacgaatccctggcgcgcgcaggcaaacgcgggaattc

SEQ ID NO: 10 - DNA, 11402 bp, Artificial Sequence
  Synthetic construct - EpoE
atgaccgaccgtgaaggccagcttttggaacgcctgcgtgaagtgacgttggccctgcggaaaactctgaacgagcgcgataccttagagttagaaaaaacggaaccaattgccattgtcggcattggctgccgttttccaggcggtgcggggactccggaagctttttgggagctgctggatgatggtcgtgatgcgatccggccacttgaggagcggtgggcgctggtcggggtcgatcctggtgatgacgtcccacgctgggctggccttctgactgaagcgattgacggctttgacgcggccttctttggcattgcgccgcgcgaagcccgctctctcgatcctcagcaccggctgctgctggaagttgcatgggaagggtttgaagacgccggcatcccgccgcgtagcctggtcgggagtcgcacgggtgtcttcgtaggcgtatgtgcaacagaatatttacatgcggcggtggctcaccagccgcgcgaggaacgcgatgcttatagcacaacgggtaacatgttgtctattgccgctggccgcttgtcatacacgcttggccttcagggcccttgcttgacagttgacacagcctgctcttcgagtctggtggcgatccacctggcgtgtcgctcactccgtgcgcgtgaatccgacttagcgctggcgggtggcgtcaatatgctgttatctcctgacaccatgcgcgcccttgctcgtacccaggcattgtccccgaacggtcgttgtcaaaccttcgatgcaagcgcgaacggttttgtccggggcgagggttgtggcctgatcgtgcttaaacgtctctccgatgcgcgtcgggacggcgaccgtatttgggccctgatccgcggcagcgctattaaccaggatggtcgctccacaggtctgaccgcaccgaatgtactggctcagggcgcactgctgcgtgaagctttacgtaatgcaggggtggaagccgaagctattggctacatcgagactcatggcgccgcgacttctttaggggatccgattgagatcgaagccctgcgcactgtggtgggcccggcgcgcgctgatggcgcccgttgcgtgctcggcgcggtgaaaaccaacctgggccatttggaaggcgcggccggggttgctgggctgatcaaagcaaccctgtctttgcaccatgaacgtattccgcgcaacctgaatttccgtacacttaatccgcgtatccgcattgaagggacggcattagccctcgctaccgaaccagttccatggcctcgcaccggccgtacgcggttcgccggtgtttcaagctttggcatgtcgggtaccaatgcgcatgttgttctggaggaagcccctgctgttgagccggaggcagcagcgccggaacgggctgccgagctgtttgtgttaagtgcgaaatcagttgccgccctggatgcccaagcagcgcgcctgcgtgatcacctggaaaaacatgtggaactgggtcttggtgacgtggcatttagcctggcgactacccgtagcgcaatggaacatcgcctggccgtggcagcgagctctcgtgaggcgctgcgcggggccctgtcggctgccgcccaaggccacacgccgccgggcgcggtgcggggccgcgcatccggtgggtcagcgccaaaagtggtcttcgtgttccctggccagggttcccagtgggtagggatgggccgtaaactgatggcggaagaacctgtctttcgcgcagcgctggagggctgcgaccgtgccatcgaagcagaagccggttggtccctgttaggtgagctgtcggcagatgaagccgcaagccagcttggccgtatcgacgttgtccagccggtactgtttgctatggaagtggccttatcggccctgtggagatcttggggtgtggagccagaggccgtagtgggtcactcaatgggcgaggtagccgctgcgcatgtggcaggtgccctgtctctggaagacgcggtggctattatttgccgtcgctcacgcctgctccgtcggatctcggggcaaggtgaaatggcactcgtggagctgtccctggaggaagccgaagcagccctgcgcggccatgaaggtcgcctgtctgttgctgtgtccaatagcccacgcagcaccgtactggccggtgaaccggccgcactgtcggaagttctggcagcgttgaccgcgaaaggcgttttctggcgtcaagttaaagtcgatgtggctagccactcgccgcaggtggacccgttgcgtgaagaactcattgccgccctgggtgccatccgcccacgcgcagccgctgttccaatgcgttccaccgtgaccggcggtgttattgcaggcccggaactgggcgcgtcttattgggctgataacttgcgccaacccgtacggtttgcggctgccgcgcaagcactgctggaaggtggtccgacgctgttcatcgaaatgagtccgcatccgatccttgtcccgccgttggatgaaattcagacggcggtcgaacaaggtggtgcagcggttgggtcactgcgccgtggtcaggacgagcgtgcaactttactggaagcactggggaccctctgggcctcgggctacccggtatcgtgggctcgtctgtttccagcggggggtcgtcgcgtaccgcttccaacgtatccgtggcaacacgagcgttgttggctgcaggttgaaccagatgctcgtcgtttagctgctgccgacccaacgaaagattggttctatcgcactgactggccggaagttcctcgcgccgccccgaaaagtgaaacagcacacgggagctggcttctcctcgctgaccgtggcggcgttggtgaggcggtcgctgcggcacttagcacccgtggcctgagttgtaccgtgttacatgcgtccgctgatgcatcgacggttgcggagcaagtgagcgaagccgccagccgtcgcaacgattggcagggggtattgtatctctggggtctggatgctgtcgttgatgctggcgcgagtgcagatgaagtttcggaagcgacacgccgcgcaaccgcgccggtgttaggtttggtgcgcttcctgtcagctgcgccgcatcctccccggttttgggttgtgaccagaggtgcgtgcaccgttggcggggagcctgaagttagtctgtgccaggccgcgttgtggggtctggcacgtgtggtagcgcttgaacatccggcggcctggggtggcctggtcgatctggatccgcagaaatcaccgaccgaaattgaaccactggtggctgagctgctgagccctgatgccgaagaccagttggcttttcgtagtggccgtcgtcacgcagcgcggcttgtcgcagcgccgccggaaggtgatgtcgcgccgatcagtcttagtgcggaaggctcttacttagtcaccggtggcttgggtggtctgggtcttctggtggcgcgctggttggtagagcgtggggcccgccacttggttctgacttcccgccatggcctgcctgaacgtcaagcatcgggtggtgaacagccgccggaagcccgcgcacgcattgccgccgtggaaggtctggaagctcagggggcacgtgttaccgtagcggcggtggacgtagctgaggcggaccctatgacggccttgttagctgctattgagcctccattgcgcggtgtcgttcacgccgcaggtgtgtttccggtccgtccgctggctgaaactgatgaggccctcttagaaagcgtattacgccctaaagttgccggtagttggttactgcatcggcttctgcgtgaccgtcctctggatttgtttgtactcttcagcagcggggcggcagtctgggggggcaaaggccagggcgcgtatgcagcagcaaatgcgttcctggatggcttggcacatcatcgtcgcgcacattctctgccagccttaagtctcgcatggggcctgtgggcggagggcggcgtggttgatgccaaagcgcatgcgcgcttatctgacatcggcgttctcccaatggcgacgggcccggctctcagcgcgctcgaacgcttagtgaacacaagtgcggtgcagcgcagcgtcacacgcatggattgggcccgctttgccccagtctacgccgctcgtggtcggcgtaacctgctttccgcgctggttgcggaagatgagcgcacggcaagccctccggttccaaccgcgaatcgcatttggcgcggtctgagcgtagcggaatcacgctcggcgctgtatgaactggtgcgtggtattgttgcacgggtgctgggcttctccgatccgggggcgctggacgtgggtcgcggcttcgcggagcagggcctggattcacttatggcgttggaaatccgcaatcgcttacagcgtgaactgggtgagcgtttaagcgccaccttagcttttgatcatccgacggtggaacgccttgtcgcgcacctgttgactgatgtgtctagtcttgaagaccgttccgatacgcgccatatccgcagcgtggccgccgatgacgacatcgcaattgtgggcgccgcatgtcgttttccggggggcgatgaggggctggagacctactggcgtcacttagctgagggcatggtcgtttcaaccgaggtgccagcagaccgttggcgcgctgcggactggtatgatccggatccggaagtaccaggtcgtacctacgtcgcgaaaggtgccttcctccgtgacgtgcgttcgttagatgcggcatttttttccatcagtccgcgtgaagctatgagtttggatccgcagcagcgcctgctgctggaggtctcatgggaagctatcgagcgcgccggccaggacccgatggccttacgcgagagcgccactggcgtctttgtcggtatgatcggtagtgaacacgccgaacgggtccaaggtttagatgacgatgccgcactgctgtacggcaccaccgggaatttgctgtctgtggcagcaggccgcctgagttttttcctgggcctgcatggcccgacgatgaccgtggataccgcttgctctagctccctggtcgccctgcacctggcttgccagtcattacgcctgggcgaatgcgatcaggcgctggctggcggttcctctgttctgctttcgcctcgctcatttgtggcggcctcccgtatgcgtttgctgagccctgatggtcgctgtaaaacgttcagcgcagccgccgatgggtttgcgcgtgccgaaggttgcgccgtggtggtattaaaacgcctgcgtgatgcccaacgtgaccgcgacccgattttggcggtggtaagatctacagccattaaccacgatgggcctagcagtggtctcaccgtcccgtctgggccagcccaacaggcactgttgggtcaagctcttgctcaagcaggggtagcgcctgccgaagttgactttgttgagtgtcacggaaccgggaccgcgctgggtgatccaatagaggtccaggctttgggcgcagtgtatggccgtggtcgcccggcggagcgcccactgtggttaggggcagtgaaagcgaatcttgggcatctggaggcagccgctggcttggcaggcgttctgaaagtgctgctggcattagaacatgaacaaattcctgcgcaaccggaactggatgagctgaaccctcatattccatgggcggaactgccggttgcggttgtccgcgccgcagtgccgtggcctcgtggcgcacggccacgtcgcgccggtgtgtcggcattcggtctcagcggtaccaacgctcacgtcgtgcttgaggaggcacctgctgttgaaccggaggcagccgcaccagaacgtgcggccgaactgttcgttctgagcgctaaaagtgtggccgcgctggatgctcaggccgcccgcctgcgtgatcatctggaaaaacacgtggaacttgggctgggcgatgtcgctttctcattggctaccacacgttctgccatggagcatcgtctggcggttgcagccagctctcgtgaagccctgcgtggtgcgttgagtgccgccgcgcagggtcacactccgccgggtgccgttcgcggccgtgcttctggtggcagcgccccaaaagtagtgttcgttttccctggccagggttcgcagtgggtaggcatgggccgtaaactgatggcggaggagcctgtatttcgtgccgcccttgaaggctgcgatcgtgccatcgaagccgaagcaggctggtccctgcttggggaactcagtgcggatgaagccgcctctcaacttggccgcattgatgtggtccagccggttctgtttgcggttgaagtggccctgtctgctctgtggagatcttggggcgttgaaccggaagctgttgtaggtcatagcatgggcgaagtcgcagcagcccatgttgctggtgccttgtctctggaggatgcggtggcgattatctgtcgtcgctctcgcctgctgcgccggatttcaggccaaggtgaaatggccttagtggaactgtcgttagaggaagcggaagcagcattgcgcgggcatgaaggtcgtctgagcgtggcagtctcaaactcgcctcgttctaccgttttagcaggtgaacctgctgctttaagtgaagttctggccgcgttgaccgccaaaggtgtcttctggcgtcaagtgaaagtggatgttgctagccacagtccgcaagtggaccctttgcgcgaggagctggtagctgcattaggcgccatccgcccgcgcgctgcggcggtgccaatgcgcagcaccgtgaccgggggtgtcattgcgggtcctgaactcggtgcgtcttattgggctgataacttgcgccagccagtccggtttgccgcagctgcacaagctttgttagaaggcgggccgactctcttcattgaaatgtccccgcatccgatcctggttccgcctctcgatgaaatccagacagctgtggaacaagggggtgcagcggttggttcactgcggcgtggtcaagatgaacgcgccacgctgctcgaagccttgggcactctgtgggcgtcgggctatccggtgtcatgggcacgtctgtttcctgctgggggccgtcgtgtgcctctgccgacatacccgtggcagcatgagcggtactggctgcaggattctgtacatggcagcaaaccgtcccttcgcctgcgccaactccacaatggtgcaacggatcatccgttactgggtgcgccgttactggtcagcgcgcgccctggtgcacacctgtgggaacaggctttgagcgacgaacgtctgtcttacctgtcagagcaccgtgtgcacggcgaagcggtgcttccaagcgctgcgtatgttgagatggcccttgccgcaggcgtcgacttgtatggcgcggcgactttagtcttagagcagttggcattggaacgcgccctggcagtgcctagcgaggggggccgcattgtacaggttgctctgtctgaagaaggcccgggccgtgcgtcttttcaggtctcgtcccgtgaggaagccggtcgttcttgggtacgtcatgcgactgggcacgtatgcagcgatcagtccagtgcggttggtgcgcttaaggaggcgccgtgggagattcaacagcgttgtccttccgttctgagctcggaagctctgtacccgttactgaacgaacatgctcttgactatgggccgtgttttcagggcgtagaacaggtttggctgggcactggcgaggtactggggcgcgtccgtctcccggaagacatggcttcgtccagcggtgcgtaccggatccatccggccttgttagacgcgtgctttcaagtcctgaccgcactgcttacaacgccagaaagtatcgaaatccgccgtcgcctgaccgatctgcacgagccagacctgccgcgtagccgtgcgccagtaaatcaggcagtgagcgatacctggctgtgggatgcagcattggatggtggtcgcagacagtctgcctctgtacccgttgacttggtacttggttcttttcacgctaaatgggaagtaatggaccgtttggcgcaaacttatatcattcggacgcttcgcacatggaacgtcttttgcgccgccggcgaacgtcacactatcgacgagttattggtgcgtttacagattagtgcggtgtatcgcaaagttattaaacgctggatggaccatctggtcgccattggcgtgctggtgggcgatggcgaacatctcgtatcatcgcagccactgccggaacacgactgggcggccgttttggaggaggcggccaccgtgtttgcggacttaccagttttactggagtggtgtaaattcgcaggtgaacgcctggctgatgtgctgaccggcaaaaccctggcgttggaaattctgtttccgggcggtagcttcgacatggcagaacgtatttatcaggactcccctattgcgcgttatagtaacggtatcgtccgtggtgtggtcgaatccgcagcccgcgtcgtggcgccttcgggcaccttttctatcttagaaattggcgcaggtacaggggcaacgacagcggccgttctgcctgttctgctgccggaccgtacggagtatcacttcaccgatgtatcgccgctgttcttagctcgtgcggaacaacgctttcgtgatcatccgttcctgaaatacggtattctggatattgatcaagagccagcgggccaggggtacgcccatcagaaattcgatgtgattgtggcagcgaatgtgattcacgcgacccgtgacatccgtgccactgcgaaacgtttgctgagcttgctcgcgccaggcgggctgctggtgctcgtggaagggaccggccacccgatctggtttgacattacgacgggcctgatcgaaggctggcagaaatatgaggatgatctgcgcacggatcatccgctgttgccagcacgtacctggtgtgatgtgcttcgccgcgttggcttcgcagatgccgtgagccttccgggcgatgggtctccagccgggatcctggggcagcacgtaatcttatcgcgcgcgccaggcatcgcgggcgctgcttgtgactcaagtggcgagtcggctactgagtctcccgcggcccgggccgtccgtcaagagtgggcggatggttcggctgatggcgttcaccgcatggcgctggaacgcatgtactttcatcgccgtccaggccgccaggtttgggtgcacggtcgcctccgtacagggggcggcgccttcacgaaagcactgacgggcgacctgctgcttttcgaagaaacgggccaggtggtggctgaggtgcagggcctgcgcctgccgcagcttgaggcatctgcttttgctccgcgcgacccacgtgaagagtggttatacgcgctggagtggcagcgcaaagatccgatccctgaagcgcctgccgcagcctcatccagcacggcgggcgcgtggcttgttcttatggatcagggcggcacgggcgcggccttagtgagcctgttggaaggcagaggtgaagcctgcgttcgcgtggttgcaggcacagcgtatgcatgcttggcgcctggcctgtatcaggttgatccggctcagccagatggctttcatactctgctgcgcgacgcttttggggaagaccgtatgtgccgcgcggtggtccacatgtggtcactcgatgctaaagccgctggtgagcgtaccacagcggaatcgctgcaagctgaccagctgcttggtagcctgtcggcccttagcctggtgcaggccctggtacggcgccgttggcgcaatatgccgcgtctttggctgctgacgcgtgcagtgcacgccgtgggtgcggaagacgctgcggcctctgtcgctcaggcaccagtctggggtcttggtcgcacactcgcactggaacatccggaattacggtgcactctcgtagatgttaatccggcgccgagtccagaagatgcggcggcgctggcagttgagttgggcgcgagtgatcgtgaggatcagattgccctgcgctccaacggtcgctacgttgcccggctggttcgttcaagtttctccggcaagccggcgaccgactgcggcattcgggccgatgggtcatacgtcatcaccgatgggatgggccgcgttggcctcagcgttgcgcagtggatggttatgcagggcgcgcggcatgttgttctcgtggaccgtggcggcgccagtgatgcctctcgtgatgcacttcgctcgatggcagaagctggtgcggaagtacaaatcgtcgaagcggacgtggcccgccgtgtagatgtagcccgtttactgtctaaaattgaaccgagtatgccgccgttgcggggcattgtgtatgtggacggtacgtttcagggggattccagcatgttggaactcgatgcccatcgcttcaaagagtggatgtatccgaaagttttgggtgcttggaacttgcacgccctgacacgtgaccgtagcttagattttttcgtcctgtatagcagcggtacatctttactgggccttccgggtcaaggtagccgcgccgcaggggatgccttcttagatgcgattgcacatcatcgctgtcgcctaggtcttaccgcgatgtcaattaattggggcctgcttagtgaagccagcagtccggccacgccaaacgatggtggtgcgcgtctccagtaccgtgggatggaagggcttaccttggagcaaggtgcggaagctctgggtcgtttacttgcgcaaccacgcgcgcaggtgggggttatgcgcctgaatctccgccagtggctggagttctacccgaatgcggcacgcctggcattatgggcggaactgctgaaagaacgtgatcgcaccgatcgcagtgcaagtaacgctagtaacctgcgggaagcgcttcaatccgcccgcccggaggatcggcagctggttctcgaaaaacacctgtcagaactgctgggccgtggtctccgtctgccaccagaacggattgaacgtcatgtcccttttagcaacctgggtatggacagtctcattggtttagagctgcgtaaccggattgaagcggccctgggtattaccgttcctgccactctgctgtggacgtatccgaccgttgccgcactgtccggtaatctcctggacattctttctagtaatgctggcgcgacgcatgctccggcgaccgagcgcgaaaaaagctttgaaaacgacgccgcagatttagaagccttgcgtgggatgactgatgaacagaaagatgcgctgcttgcggagaaactcgcacaactggcccagatcgtgggcgaagggaattc

SEQ ID NO: 11 - DNA, 7325 bp, Artificial Sequence
  Synthetic construct - EpoF
atggcgacgacgaacgcgggtaaactggaacatgctcttctgttaatggataagctggcgaagaagaacgcaagtttagagcaggaacgcactgaaccaattgcgattattgggatcggctgccgttttccgggtggtgcggacaccccggaagcgttttgggaactgttggatagtggccgcgatgctgtgcagccgctggatcgccgttgggcgctggtgggcgtccatccttcagaagaagtcccgcgctgggcggggttgctgaccgaggccgtggatgggtttgacgcggcgttctttggtacaagtccgcgcgaagcgcgtagcctcgatccgcaacagcgtctgctcctggaggtaacctgggaaggtctggaagatgccggcatcgcaccgcaatcgctggatggtagccgtacaggcgtctttcttggggcttgtagctccgactatagccatactgttgcgcagcagcgccgcgaagaacaggacgcctatgacattacgggcaacactctttccgtcgctgccgggcgtctcagctataccctcggtctacagggcccgtgcctcaccgtagacactgcgtgtagctcatcgttggtggcaattcacctggcgtgtcgcagcctccgcgcacgcgagtctgatctggccctggctggcggtgttaatatgctgctgtcaagcaaaaccatgatcatgctcggtcgcattcaagcactgagcccggatggacattgccgtacctttgatgcgtccgctaatggcttcgtacgcggcgaaggctgcggtatggtggtattaaaacgtctgagcgatgcccagcggcacggcgatcgcatttgggcattgatccgcggttcagccatgaaccaggacggccgttccaccgggttgatggcgccaaacgtcctcgcccaggaagcgctgctgcgtcaggcgctacagagcgcacgtgtggatgctggcgcgatcgattacgtggagacacatggcacaggcacctcgctgggcgatccaatagaagttgacgctctgcgtgcagtcatgggtccggctcgtgcggatgggagccgttgtgtgttgggtgcagtgaaaacaaacttaggccacctggagggcgccgctggggtggcgggtctgatcaaagccgcactggcgcttcaccacgaaagcattcctcgtaatctgcatttccacacactcaatccgcgtattcgtattgagggaaccgcgctggccctggcaaccgaaccagttccgtggcctcgcgcgggtcgtccacgctttgcgggtgtgtctgctttcggcctgagtggtaccaacgtgcatgttgtgttggaagaagcacctgccaccgtgttagccccggcaacgccgggccgttctgctgaactgcttgttttaagcgctaaatccacagccgctctggacgcacaggcggcgcggttatcggcccacatcgcggcatatccggagcaaggtctgggtgatgtggccttttccttagttgcgacccgcagtccgatggaacatcgtctcgccgttgccgccacgtctcgcgaagcgctgcgttctgcgttagaggcggcggcacagggccaaaccccggcaggcgcggctcgtggtcgtgcggcctcgtcaccgggtaaattggcatttctgttcgctggccagggcgcccaagtaccaggtatgggccgtggtctgtgggaagcctggcctgcgtttcgtgaaaccttcgaccgctgcgttactttgttcgaccgtgagctgcaccaacctctgtgtgaagttatgtgggcggaaccgggtagtagccgttcgtcgcttttagaccaaacggcgttcacccaaccagcgctgttcgcgcttgaatacgcgctggctgcgctgtttagatcttggggcgtggaaccggaactgatcgcgggccattctttgggcgagctggtggccgcgtgcgttgcgggcgtgttttcgctggaagacgctgttcgcttggtggtggcacgcgggcgcctgatgcaggcgctgccagctggcggtgccatggttagcattgccgctccggaagccgatgtcgccgcagctgttgcaccgcacgcggctagtgtctcaatcgccgccgtcaatggccctgagcaggttgtcattgctggcgcggagaaatttgtgcaacaaattgccgctgcctttgctgcgcgcggtgctcgcaccaaacctttgcatgtttcccacgcgttccactccccgctgatggatccaatgctggaagcatttcgccgcgtcactgaatctgtgacctatcgccgcccgtcgatggcgttagtaagcaatctgtcgggtaaaccgtgtaccgatgaggtgtgtgcgcctggttattgggtacgccatgctcgggaagcggtgcgcttcgcagatggcgttaaagcgctgcacgcagcaggcgcgggtatttttgttgaagttggtccgaaacctgccctgctgggtctgctgcctgcatgtctgccggatgcccgtccagtgttactgccagcaagccgcgcaggtcgtgacgaggccgcgtcagcattagaagcactgggtgggttttgggtggttggtggcagcgtaacgtggagtggtgtgttcccgtcaggtggtcgccgtgttcctctcccaacgtatccgtggcaacgggaacggtattggctgcaggcacctgtagacggtgaagcggatggtatcggtcgcgcacaagctggcgatcatccattgctgggtgaagccttcagtgtgtcaacccacgcaggtctgcgcctgtgggagactaccctcgatcgtaaacgtctgccgtggctgggtgagcatcgggcgcagggtgaagtagtgtttccgggggcaggctacctggaaatggccctttcctcaggcgccgagatattaggggatggtccgatccaggtaacggatgtggtgctgattgagaccctgacttttgctggcgatacggcagttcctgtgcaggttgtgacaactgaagaacgtccgggtcgtctgcggttccaggtcgcctcccgcgaaccaggggcccgtcgtgcaagttttcgcattcatgcccgtggtgttctgcgtcgcgtcggtcgtgcggaaacgcccgctcgtcttaatctcgccgcactgagagcccgcctgcatgcagcagtcccagccgctgctatctatggcgcattggcagaaatggggttacagtacgggcctgcactgcgtggtctggcagaactgtggcgtggcgagggtgaagctctgggtcgcgttcgtctgccagaatccgcgggttcggcgacagcctatcagctgcacccggtgctccttgatgcatgcgtacagatgattgtgggcgcgttcgcggaccgtgatgaagctacgccatgggccccggtggaggtcgggagcgtgcgtctcttccaacgctctcctggcgaattgtggtgccatgcccgtgttgtgtcagacggccaacaggcaccgagtcgctggagcgccgactttgagctgatggacggcacaggggctgtagttgcagagattagccgtctggtggttgaacgcttagcgtccggcgtccgccgccgtgacgcggacgattggtttctggagctcgattgggaaccggcagcattagagggtccgaaaatcacggccggtcgctggctgctgctgggggagggtgggggcttgggccgttctttatgtagtgcgctgaaagcggctggtcatgttgtggtacacgccgcaggggatgatacgtctgcggcaggcatgcgtgcgttgctggcgaacgcgttcgatggtcaggcgccgacggctgtcgtccacctcagctctctggacggcggcggtcaactggatcctggcttgggcgctcaaggcgcattggacgctccgagatctccagacgtggacgcagacgcccttgagtccgcattaatgcgcggttgcgattccgtgctgagcctggtgcaggcgctcgtcggtatggatctgcggaacgcaccacgtctgtggctgcttacccgtggcgcacaggcagctgccgcaggcgatgtctcggtggtgcaggctccgctgctggggctgggccgcacgatcgcgctggaacatgcagaacttcgctgtatctcagtagatttggatccggcacagccggaaggcgaagcggacgcgctgctggccgaactgctggctgacgacgcggaggaagaagtggcattgcgtggtggtgaacgctttgtggcacgtctggttcaccgcttgccggaagcgcaacgtcgggaaaaaattgcgccagcgggcgaccgcccgtttcgcttggaaatcgatgaaccgggtgttttagatcagttagttcttcgtgcaacgggtcgccgtgcgccgggcccgggcgaagtcgagatcgccgtagaggctgcgggcctggattctattgatattcagcttgccgtcggggtagcaccgaacgacttgcctggcggggagatcgagccgtcggtcctgggtagtgaatgcgccggccgcatcgtagcagtaggtgaaggcgtgaatgggttggtagtgggtcagccggttattgccttagcggcgggtgtttttgcgacgcatgttacgacttctgcgaccctggtgctgccgcgtccgctcgggttgagcgcgaccgaagcggcggcgatgccattggcgtatcttaccgcttggtatgcgcttgataaagttgctcaccttcaggcaggcgaacgtgttctgattcgggcggaggccgggggcattggtctgtgcgccgtccggtgggcgcagcgcgttggtgctgaggtctatgcgaccgccgacacgccagaaaaacgtgcctaccttgagtcgctgggtgtgcgctacgtgagcgatcctaggtctggtcgcttcgcagcggatgtccatgcgtggaccgatggggagggcgttgatgtggttctggactctctgtccggcgaacatatcgataaaagtctgatggttttacgcgcatgtgggcgcctcgttaaactgggtcgccgtgacgattgcgctgacacccaaccagggctgccaccgttgttgcgcaacttttcattttctcaggtggatctgcgtggcatgatgctggaccagcccgcgcggattcgtgctcttctggatgaattgtttggcctggtggcggccggtgcgatttcccctttagggagcggtctgcgggttggtggcagcctgaccccgccacctgtcgaaaccttcccaattagtcgtgccgctgaagccttccgtcgcatggcgcagggtcagcatctcggtaaactggtcctgaccctggatgatccagaggttcgtattcgtgcgccagccgaaagcagcgtggcagttcgtgcagatggcacctatttagttaccggtggtttaggtggcttgggcttacgtgttgctggctggctggcagaacgcggtgctgggcagttagtgttagtgggccgtagcggcgctgcctccgcagaacagagagccgccgtggccgccctggaggcccatggcgcccgcgtcaccgtagctaaagctgatgtagcggatcgttcacaaattgaacgcgtactgcgcgaagtcacggcttccggcatgccgctgcggggcgttgtccacgccgctggtttagtagacgacggcctgttgatgcaacagaccccggcccgccttcgtacggtaatgggccctaaagtgcaaggtgcccttcatctgcacactctgactcgggaagcacctttatctttctttgttctgtatgcaagtgcagcaggtttattcggcagcccgggtcagggtaattacgctgctgcaaacgcttttctggatgcgctgagtcatcaccggcgtgcgcatgggttgccagccttaagcattgactggggcatgtttaccgaagtggggatggcggtcgcacaagagaaccgtggcgcacgccttattagtcggggcatgcgcggtattacgccggacgaagggctgtcagcgttggcccgccttctcgaaggtgatcgtgttcaaacgggtgtgatcccgattacaccgcgtcagtgggtggagttctatccggccacagcggccagtcgtcgtctcagccgcctggtcacaactcagcgtgcggtcgctgatcgcaccgccggggatcgcgatctcctcgaacagttggcctcggcggaaccatccgctcgggctggcctgttgcaagatgtcgtacgcgtgcaggtgtcgcatgtgctccgcctgccggaggataaaatcgaggtggacgcaccgttatccagtatgggtatggatagtttgatgtcgctggaattacgcaatcgtatcgaagccgcgctgggcgtagcggctccggcagctctgggttggacttacccgacggtggcagctattacccgttggttactggatgatgctctttctagtcgcttaggcggcgggagcgatacggatgaatccactgcatcggcgggtagctttgttcacgtcctgcgttttcgcccggtagtaaaaccgcgtgcacgcctgttttgttttcacggttcggggggttctccagaaggcttccgtagctggtctgaaaaatcagagtggagtgacctcgaaattgtcgcgatgtggcatgatcgttccttggcatctgaggatgccccgggcaaaaaatatgttcaggaagctgccagtctcatccaacattatgcggatgccccatttgctcttgtgggtttctctttgggtgttcgctttgtaatgggcacagcggtggagctggcttctcggagtggggcgccagcaccattggcggtgttcgcactgggtggctccctgatttccagcagcgaaatcactccggagatggagaccgatattatcgcgaaactgttttttcgtaacgcggccggtttcgtgcgctcaacacagcaagtccaggctgacgcccgcgcggataaagtgattactgataccatggtcgcccctgcgccgggtgatagcaaagaaccgccgtcaaaaatcgcggtgccgatcgttgcaattgccggttcggatgacgtgatcgtccctccatcggacgttcaggacttacagagccgtaccaccgaacggttttacatgcatctgctgccgggcgaccatgagttcctggttgaccgcgggcgtgaaattatgcatattgtagattcacaccttaatccgctgttagctgcccgcaccacgtccagtggcccggccttcgaagcaaaagggaattc

SEQ ID NO: 12 - protein, 6 aa, Artificial Sequence
  Synthetic construct - Mfe I site
PIAIVG

SEQ ID NO: 13 - protein, 6 aa, Artificial Sequence
  Synthetic construct - Kpn I site
GTNAHV

SEQ ID NO: 14 - protein, 6 aa, Artificial Sequence
  Synthetic construct - Msc I site
PGQGAQ

SEQ ID NO: 15 - protein, 6 aa, Artificial Sequence
  Synthetic construct - Pst I site
PRPHRP

SEQ ID NO: 16 - protein, 6 aa, Artificial Sequence
  Synthetic construct - BsrB I site
PLRAGE

SEQ ID NO: 17 - protein, 6 aa, Artificial Sequence
  Synthetic construct - Age I site
TGGTGT

SEQ ID NO: 18 - protein, 6 aa, Artificial Sequence
  Synthetic construct - Xba I site
FADSAP

SEQ ID NO: 19 - protein, 6 aa, Artificial Sequence
  Synthetic construct of module 4 of erythromycin
EPIAIV

SEQ ID NO: 20 - protein, 9 aa, Artificial Sequence
  Synthetic construct - variable sequence region
YXFXXXRXW

SEQ ID NO: 21 - DNA, 69 bp, Artificial Sequence
  Synthetic construct - pKOS293-88-1_Synthon_vector
agctagcggccgccctcagctatatcgctatcgatgagctcaatgcatcgatcactagctgagggaatt

SEQ ID NO: 22 - DNA, 36 bp, Artificial Sequence
  Synthetic construct - pKOS293-88-1_Synthon_vector
agcggccgccctcagctatatcgctatcgatgagct

SEQ ID NO: 23 - DNA, 25 bp, Artificial Sequence
  Synthetic construct - pKOS293-88-1_Synthon_vector
caatgcatcgatcactagctgaggg

SEQ ID NO: 24 - DNA, 36 bp, Artificial Sequence; identical to SEQ ID NO: 22
  Synthetic construct - pKOS293-88-1_Synthon_vector

SEQ ID NO: 25 - DNA, 29 bp, Artificial Sequence
  Synthetic construct - pKOS293-88-1_Synthon_vector
ccctcagctagtgatcgatgcattgagct

SEQ ID NO: 26 - DNA, 42 bp, Artificial Sequence
  Synthetic Construct - Synthon 1
nnnggtctcnnnnnnnnnnnnnnnnngatcgngtcttcnnnn

SEQ ID NO: 27 - DNA, 42 bp, Artificial Sequence
  Synthetic Construct - Synthon 2
nnnctcttcngatcgnnnnnnnnnnnnnnnnngtcttcnnnn

SEQ ID NO: 28 - DNA, 26 bp, Artificial Sequence
  Synthetic construct - Synthon 1
nnnggtctcnnnnnnnnnnnnnnnnn

SEQ ID NO: 29 - DNA, 33 bp, Artificial Sequence
  Synthetic construct - Synthon 2
gatcgnnnnnnnnnnnnnnnnngtcttcnnnnn

SEQ ID NO: 30 - DNA, 59 bp, Artificial Sequence
  Synthetic construct - Synthon 1+2
nnnggtctcnnnnnnnnnnnnnnnnngatcgnnnnnnnnnnnnnnnnngtcttcnnnnn

SEQ ID NO: 31 - DNA, 32 bp, Artificial Sequence
  Synthetic construct - pKOS293-88-1_Synthon_vector
catcgatagcgatatagctgagggcggccgct

SEQ ID NO: 32 - DNA, 29 bp, Artificial Sequence
  Synthetic construct - pKOS293-88-1_Synthon_vector
ccctcagctagtgatcgatgcattgagct

SEQ ID NO: 33 - DNA, 14 bp, Artificial Sequence
  Synthetic construct - pKOS293-88-1_Synthon_vector
tgagggcggccgct

SEQ ID NO: 34 - DNA, 30 bp, Artificial Sequence
  Synthetic construct - Synthon 1
gatcnnnnnnnnnnnnnnnnngagaccnnn

SEQ ID NO: 35 - DNA, 29 bp, Artificial Sequence
  Synthetic construct - Synthon 2
nnnnngaagacnnnnnnnnnnnnnnnnnc

SEQ ID NO: 36 - DNA, 60 bp, Artificial Sequence
  Synthetic construct - Synthon 1+2
nnnnnngaagacnnnnnnnnnnnnnnnnncgatcnnnnnnnnnnnnnnnnngagaccnnn